(19) United States
(12) Patent Application Publication
(10) Pub. No.: US 2024/0217956 A1
(43) Pub. Date: Jul. 4, 2024

(54) ORGANIC COMPOUNDS, ORGANIC ELECTROLUMINESCENT DEVICE AND ELECTRONIC APPARATUS

(71) Applicant: SHAANXI LIGHTE OPTOELECTRONICS MATERIAL CO., LTD., Xi'an (CN)

(72) Inventors: Fumin Yue, Xi'an (CN); Yun Liu, Xi'an (CN); Youngkook Kim, Xi'an (CN); Xianbin Xu, Xi'an (CN)

(21) Appl. No.: 18/552,892

(22) PCT Filed: Sep. 7, 2022
(86) PCT No.: PCT/CN2022/117426

§ 371 Date: Sep. 27, 2023

(30) Foreign Application Priority Data
Dec. 30, 2021 (CN) 202111669905.4
Jun. 7, 2022 (CN) 202210635848.6

(51) Int. Cl. C07D 405/04; C07D 405/14; C07D 409/14; H10K 50/11; H10K 50/16; H10K 85/60

(52) U.S. Cl. CPC C07D 405/04; C07D 405/14; C07D 409/14; H10K 50/11; H10K 50/16; H10K 85/6574

(57) ABSTRACT

The present application provides organic compounds, an organic electroluminescent device. and an electronic apparatus. The structure of the organic compound is shown as formula 1, the organic compounds being used in an organic electroluminescent device to improve the performance of the device.

### CROSS-REFERENCE TO RELATED APPLICATIONS

This application claims the benefit of Chinese patent application No. 202111669905.4 filed on Dec. 30, 2021 and Chinese patent application No. 202210635848.6 filed on Jun. 7, 2022, which are incorporated herein by reference in their entirety.

### FIELD OF THE INVENTION

The present disclosure relates to the technical field of organic luminescent materials, and in particular provides an organic compound, an organic electroluminescent device comprising the organic compound, and an electronic apparatus.

### BACKGROUND OF THE INVENTION

Organic electroluminescent device (Organic light-emitting diode, OLED), as a new generation of display technology, have advantages of being emissive, and exhibiting wide viewing angle, low power consumption, high response rate, and full color, and are therefore of high research and development values and wide application prospects. An organic electroluminescent device typically consists of a cathode, an anode, and an organic functional layer disposed between the cathode and the anode. The device comprises an anode, a hole transport layer, an emissive layer, an electron transport layer, a cathode, etc. The luminescence principle of organic electroluminescent devices is as follows. By applying a voltage, holes and electrons are injected from the anode and the cathode respectively under the influence of a direct-current electric field. These charge carriers are transported through the hole transport layer and the electron transport layer respectively, and finally meet and recombine in the emissive layer to form excitons. During the process that the excitons in excited states return to their ground states, light is emitted.

A series of breakthroughs and successes have been made so far in the development of OLED display technology, but there are still many obstacles in the development. For example, the development of OLED organic materials is facing great difficulties and challenges. Although many organic materials have been developed and are well known to us, there is a great imbalance in the development of the various organic materials. In order to remove current constraints on organic materials of organic electroluminescent devices, it is of critical importance to develop highly efficient organic electroluminescent materials so as to improve the performance of organic electroluminescent devices.

### SUMMARY OF THE INVENTION

Directed against the above problems with the existing technologies, the present disclosure aims at providing an organic compound, an organic electroluminescent device comprising the organic compound, and an electronic apparatus. The organic compound, when used in an organic electroluminescent device, can improve the performance of the device.

To achieve the above objective, the present disclosure, in a first aspect, provides an organic compound having a structure shown in Formula 1:

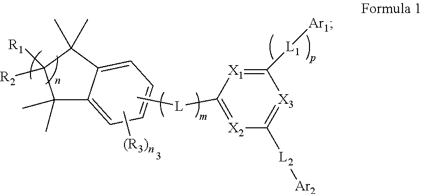

in Formula 1, R₁ and R₂ are identical or different, and are each independently selected from hydrogen or methyl;
X₁, X₂, and X₃ are each independently selected from C(H) or N atoms, and at least one of X₁, X₂, and X₃ is N;
p is selected from 1 or 2; each L₁ and each L₂ is identical or different, and is each independently selected from the group consisting of single bond, substituted or unsubstituted arylene having 6 to 20 carbon atoms, and substituted or unsubstituted heteroarylene having 5 to 20 carbon atoms;
m represents 1 or 2, and each L is independently selected from the group consisting of single bond, substituted or unsubstituted arylene having 6 to 20 carbon atoms, and substituted or unsubstituted heteroarylene having 8 to 20 carbon atoms;
Ar₁ and Ar₂ are identical or different, and are each independently selected from the group consisting of substituted or unsubstituted aryl having 6 to 40 carbon atoms, and substituted or unsubstituted heteroaryl having 5 to 40 carbon atoms;
substituents in L₁, L₂, L, Ar₁, and Ar₂, as well as R₃ are identical or different, and are each independently selected from the group consisting of deuterium, halogen, cyano, alkyl having 1 to 10 carbon atoms, haloalkyl having 1 to 10 carbon atoms, trialkylsiyl having 3 to 12 carbon atoms, deuterated alkyl having 1 to 10 carbon atoms, cycloalkyl having 3 to 10 carbon atoms, aryl having 6 to 18 carbon atoms, and heteroaryl having 5 to 15 carbon atoms; and optionally, in Ar₁ and Ar₂, any two adjacent substituents form a saturated or unsaturated 3 to 18-membered ring substituted or unsubstituted by alkyl having 1 to 4 carbon atoms;
n₃ represents the number of R₃, and is selected from 0, 1, 2, or 3, wherein when n₃ is greater than 1, each R₃ is identical or different.

The present disclosure, in a second aspect, provides an organic electroluminescent device comprising an anode and a cathode that are disposed opposite to each other, and a functional layer disposed between the anode and the cathode. The functional layer comprises the organic compound described in the first aspect of the present disclosure.

The present disclosure, in a third aspect, provides an electronic apparatus including the organic electroluminescent device described in the second aspect of the present disclosure.

In the structure of the organic compound of the present disclosure, a tetramethyl-substituted cycloalkylphenyl structure is introduced directly or introduced via an electron-rich aromatic group to one branch of a nitrogen-containing 6-membered heteroaryl group (such as triazinyl) having three branches, and then an aromatic group is introduced to each of the other two branches. The four methyl groups of the tetramethyl-substituted cycloalkylphenyl group show hyperconjugation effect, which can enhance the electron transport ability of the entire molecule. The organic compound can be used as an electron transport material or a host material of an organic emissive layer to improve the luminescence efficiency and prolong service life of an organic electroluminescent device.

### BRIEF DESCRIPTION OF THE DRAWINGS

The accompanying drawings are intended to provide a further understanding of the present disclosure and form a part of the specification. The accompanying drawings, together with the following specific embodiments, are used to illustrate the present disclosure, but do not constitute any limitation on the present disclosure.

FIG. 1 is a schematic structural diagram of an organic electroluminescent device according to an embodiment of the present disclosure.

FIG. 2 is a schematic structural diagram of an electronic apparatus according to an embodiment of the present disclosure.

### LIST OF REFERENCE SIGNS

100: anode; 200: cathode; 300: functional layer; 310: hole injection layer;
320: hole transport layer; 321: first hole transport layer;
322: second hole transport layer; 330: organic emissive layer;
340: electron transport layer; 350: electron injection layer; 400: electronic apparatus

### DETAILED DESCRIPTION OF THE EMBODIMENTS

Specific embodiments of the present disclosure are described in detail below with reference to the accompanying drawings. It should be appreciated that the specific embodiments described herein are intended only to illustrate and explain, rather than limiting, the present disclosure.

Exemplary embodiments will now be described more comprehensively with reference to the accompanying drawings. The exemplary embodiments, however, can be implemented in a variety of forms and should not be interpreted as being limited to the examples set forth herein. On the contrary, these embodiments are provided to make the present disclosure more comprehensive and complete, and to communicate the concepts of these exemplary embodiments fully to those skilled in the art. Features, structures, or characteristics described can be combined in one or more embodiments in any suitable manner. In the following description, many specific details are provided to give a full understanding of the embodiments of the present disclosure.

In the present disclosure, the expression “each . . . independently” may be used interchangeably with the expressions “ . . . each independently”, and “ . . . each independently selected from”, and all these expressions should be interpreted in a broad sense. They can not only mean that, for same symbols in a same group, the selection of a specific option for one of the symbols and the selection of a specific option for another one of the symbols do not affect each other, but also mean that for same symbols in different groups, the selection of a specific option for one of the symbols and the selection of a specific option for another one of the symbols do not affect each other. Taking

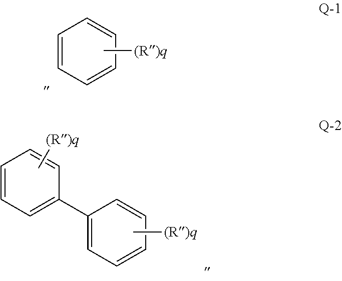

as an example, each q is independently 0, 1, 2, or 3, and each R″ is independently selected from the group consisting of hydrogen, deuterium, fluorine, and chlorine, which means: in Formula Q-1, there are q substituents R″ on the benzene ring, wherein each of the substituent R″ may be identical or different, with the selection of an option for one of the substituents R″ and the selection of an option for another one of the substituents R″ not affecting each other; and in Formula Q-2, there are q substituents R″ on each of the two benzene rings of biphenyl, wherein the number q of the substituent R″ on one benzene ring and the number q of the substituent R″ on the other benzene ring may be identical or different, and each substituent R″ may be identical or different, with the selection of an option for one of the substituents R″ and the selection of an option for another one of the substituents R″ not affecting each other.

In the present disclosure, the term “optional” or “optionally” means that the subsequently described event or circumstance may occur or may not occur, i.e., including instances where an event or circumstance occurs and instances where the event or circumstance does not occur. For example, “optionally, any two adjacent substituents form a ring” means that the two substituents may form a ring or may not form a ring, i.e., including instances where two adjacent substituents form a ring and instances where the two adjacent substituents do not form a ring.

In the present disclosure, the term “substituted or unsubstituted” means that the functional group defined by the term may have or may not have a substituent (hereinafter referred to as Rc for ease of description). For example, “substituted or unsubstituted aryl” refers to aryl having a substituent Rc or unsubstituted aryl. The above substituents, namely Rc, may be, for example, deuterium, halogen, cyano, heteroaryl, aryl, alkyl, haloalkyl, deuterated alkyl, cycloalkyl, trialkylsilyl, etc. In the present disclosure, a “substituted” functional group can be substituted by one or more than two of the above substituents Rc. When an atom has two substituents Rc, the two substituents Rc can exist independently or can be linked to each other to form a ring together with the atom; and when two adjacent substituents Rc exist on a functional group, the two substituents Rc can exist independently or can be fused into a ring together with the functional group to which they are attached.

In the present disclosure, in the expression “any two adjacent substituents form a saturated or unsaturated 3 to 18-membered ring substituted or unsubstituted by alkyl having 1 to 4 carbon atoms”, the saturated ring formed may be, for example, cyclopentane

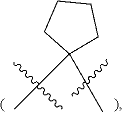 [SMILES: *C1(*)CCCC1]

cyclohexane

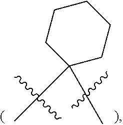 [SMILES: *C1(*)CCCCC1]

and the unsaturated ring formed may be, for example, a benzene ring, a naphthalene ring, a fluorene ring

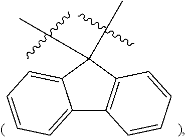 [SMILES: *C1(*)c2ccccc2-c2ccccc21]

an xanthene ring

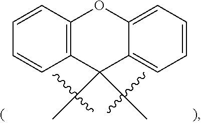 [SMILES: *C1(*)c2ccccc2Oc2ccccc21]

or a thioxanthene ring

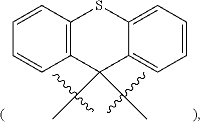 [SMILES: *C1(*)c2ccccc2Sc2ccccc21]

The saturated or unsaturated ring formed may be substituted by alkyl having 1 to 4 carbon atoms (e.g., methyl, ethyl, isopropyl, tert-butyl), or may not be substituted by the alkyl group. When the ring is substituted by alkyl having 1 to 4 carbon atoms, the number of the substituent (alkyl having 1 to 4 carbon atoms) may be one or more than two; and when the number of the substituent is greater than 1, the substituents may be identical or different. The ring substituted by alkyl having 1 to 4 carbon atoms may be, for example,

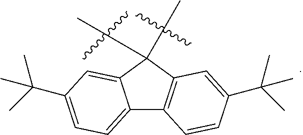 [SMILES: *C1(*)c2cc(C(C)(C)C)ccc2-c2ccc(C(C)(C)C)cc21]

In the present disclosure, the number of carbon atoms of a substituted or unsubstituted functional group refers to the number of all carbon atoms. As an example, if L₁ is substituted arylene having 12 carbon atoms, then the number of all carbon atoms of the arylene group and substituents thereon is 12. As another example, if Ar₁ is

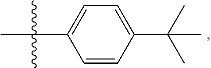 [SMILES: *c1ccc(C(C)(C)C)cc1]

then its carbon atom number is 10; and if L

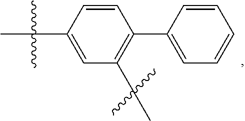 [SMILES: *c1ccc(-c2ccccc2)c(*)c1]

is then its carbon atom number is 12.

In the present disclosure, “aryl” refers to any functional group or substituent group derived from an aromatic carbon ring. An aryl group may be a monocyclic aryl group (e.g., phenyl) or a polycyclic aryl group. In other words, an aryl group may be a monocyclic aryl group, a fused aryl group, two or more monocyclic aryl groups linked by carbon-carbon bond conjugation, a monocyclic aryl group and a fused aryl group linked by carbon-carbon bond conjugation, or two or more fused aryl groups linked by carbon-carbon bond conjugation. That is, unless otherwise specified, two or more aromatic groups linked by carbon-carbon bond conjugation may also be regarded as an aryl group in the present disclosure. Among them, fused aryl groups may include, for example, bicyclic fused aryl groups (e.g., naphthyl), tricyclic fused aryl groups (e.g., phenanthryl, fluorenyl, anthryl), etc. An aryl group does not contain a heteroatom such as B, N, O, S, P, Se, Si, etc. It should be noted that biphenyl and fluorenyl are considered as aryl groups in the present disclosure. Examples of aryl may include, but are not limited to, phenyl, naphthyl, fluorenyl, anthryl, phenanthryl, biphenyl, terphenyl, benzo[9,10]phenanthryl, pyrenyl, benzofluoranthryl, chrysenyl, etc.

In the present disclosure, “substituted aryl” may mean that one or more than two hydrogen atoms of the aryl group are substituted by a group such as deuterium, halogen, cyano, aryl, heteroaryl, trialkylsilyl, haloalkyl, alkyl, cycloalkyl, etc. It should be appreciated that the number of carbon atoms of substituted aryl refers to the number of all carbon atoms of the aryl group and substituents on the aryl group. For example, substituted aryl having 18 carbon atoms means that the number of all carbon atoms of the aryl group and substituents thereon is 18. Further, in the present disclosure, fluorenyl may be substituted; and when it has two substituents, the two substituents can be bonded to each other to form a spiro structure. Specific examples of substituted fluorenyl include, but are not limited to:

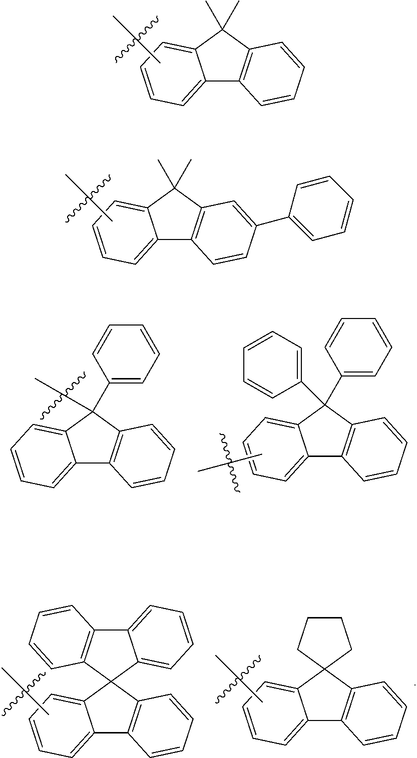

In the present disclosure, “arylene” refers to a divalent or higher-valent group formed by further removing one hydrogen atom from an aryl group.

In the present disclosure, the number of carbon atoms of substituted or unsubstituted aryl may be 6 to 40. Specifically, the number of carbon atoms of substituted or unsubstituted aryl may be 6, 7, 8, 9, 10, 11, 12, 13, 14, 15, 16, 17, 18, 19, 20, 21, 22, 23, 24, 25, 26, 27, 28, 29, 30, 33, 35, 36, 37, 38, 39, or 40.

In the present disclosure, “heteroaryl” refers to a monovalent aromatic ring containing 1, 2, 3, 4, 5, or more heteroatoms or a derivative thereof. The heteroatoms may be one or more selected from B, O, N, P, Si, Se, S, etc. A heteroaryl group may be a monocyclic heteroaryl group or polycyclic heteroaryl group. In other words, a heteroaryl group may be a single aromatic ring system, or a plurality of aromatic ring systems linked by carbon-carbon bond conjugation, with any of the aromatic ring systems being an aromatic monocyclic ring or a fused aromatic ring. For example, heteroaryl groups may include, but are not limited to, thienyl, furyl, pyrryl, imidazolyl, thiazolyl, oxazolyl, oxadiazolyl, triazolyl, pyridyl, dipyridyl, pyrimidyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothiophenyl, dibenzothienyl, thienothiophenyl, benzofuranyl, phenanthrolinyl, isoxazolinyl, thiadiazolyl, benzothiazolyl, phenothiazinyl, silafluorenyl, dibenzofuranyl, N-phenylcarbazolyl, N-pyridylcarbazolyl, N-methylcarbazolyl, etc. Among them, thienyl, furyl, phenanthrolinyl, and the like are each a heteroaryl group of a single aromatic ring system; and N-phenylcarbazolyl is a heteroaryl group of polycyclic systems linked by carbon-carbon bond conjugation. In the present disclosure, a heteroarylene group is a divalent or higher-divalent group formed by further removing one or more hydrogen atoms from a heteroaryl group.

In the present disclosure, “nitrogen-containing heteroaryl” refers to heteroaryl comprising N atoms in the ring.

In the present disclosure, “substituted heteroaryl” may mean that one or more hydrogen atoms in the heteroaryl group are substituted by a group such as deuterium, halogen, cyano, aryl, heteroaryl, trialkylsilyl, alkyl, cycloalkyl, etc. It should be appreciated that the number of carbon atoms of substituted heteroaryl refers to the number of all carbon atoms of the heteroaryl group and substituents on the heteroaryl group.

In the present disclosure, the number of carbon atoms of substituted or unsubstituted heteroaryl may be 5 to 40. For example, the number of carbon atoms of substituted or unsubstituted heteroaryl may be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, etc.

In the present disclosure, a non-positional bond is single bond “ [symbol] ” extending from a ring system, and it indicates that the linkage bond can be linked at one end thereof to any position in the ring system through which the bond passes, and linked at the other end thereof to the rest of the compound molecule. For example, as shown in Formula (f) below; the naphthalyl group represented by Formula (f) is linked to other positions of the molecule via two non-positional bonds passing through the two rings, which indicates any of possible linkages shown in Formulae (f-1) to (f-10):

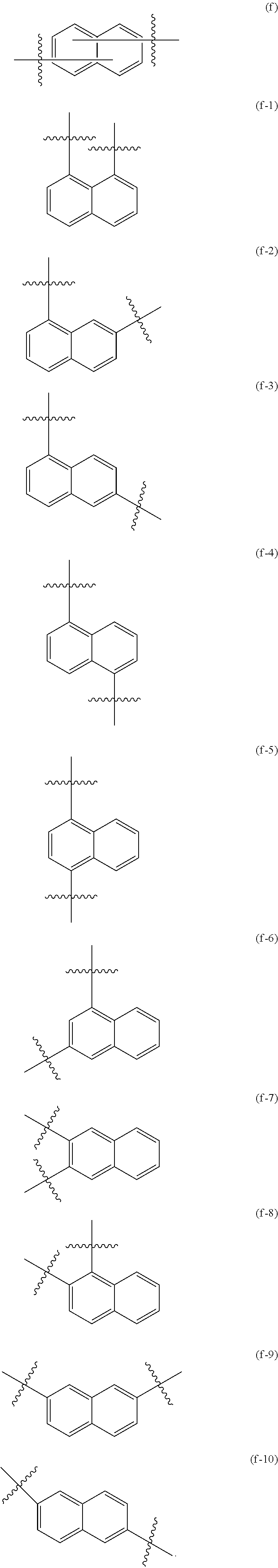

As another example, as shown in Formula (X′) below, the dibenzofuranyl group represented by Formula (X′) is linked to other positions of the molecule via a non-positional bond extending from the center of a side benzene ring, which indicates any of possible linkages shown in Formulae (X′-1) to (X′-4):

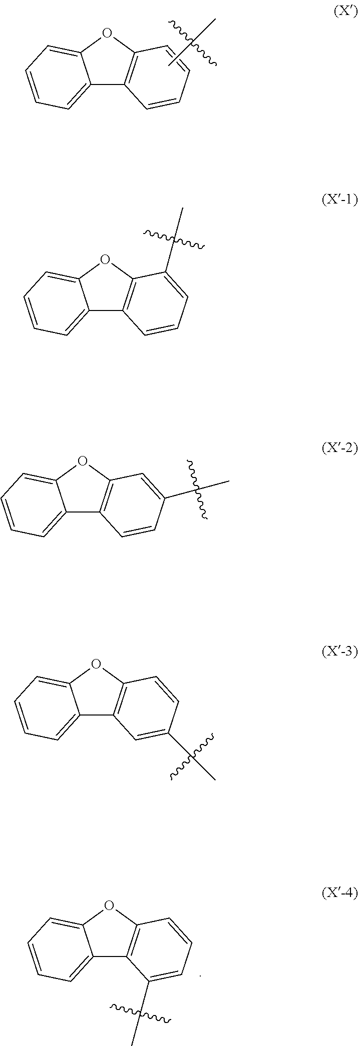

A non-positional substituent in the present disclosure refers to a substituent linked via single bond extending from the center of a ring system, and it means that the substituent may be linked to any possible position in the ring system. For example, as shown in Formula (Y) below, the substituent R′ represented by Formula (Y) is linked to a quinoline ring via a non-positional bond, which indicates any of possible linkages shown in Formulae (Y-1) to (Y-7):

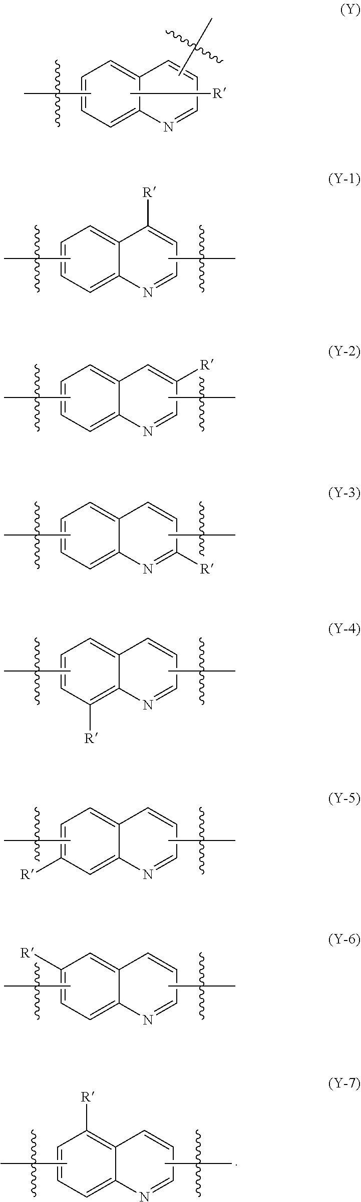

In the present disclosure, the number of carbon atoms of alkyl may be 1 to 10, and may specifically be 1, 2, 3, 4, 5, 6, 7, 8, 9, or 10. Alkyl may include straight-chain alkyl and branched-chain alkyl. Specific examples of alkyl include, but are not limited to, methyl, ethyl, n-propyl, isopropyl, n-butyl, isobutyl, tert-butyl, n-pentyl, isoamyl, n-hexyl, heptyl, n-octyl, 2-ethylhexyl, nonyl, decyl, 3,7-dimethyloctyl, etc.

In the present disclosure, halogen may include fluorine, iodine, bromine, chlorine.

In the present disclosure, the number of carbon atoms of aryl as a substituent may be 6 to 18, and may specifically be, for example, 6, 10, 12, 13, 14, 15, 16, 18, etc. Specific examples of aryl as substituents include, but are not limited to, phenyl, naphthyl, biphenyl, phenanthryl, anthryl, fluorenyl, etc.

In the present disclosure, the number of carbon atoms of heteroaryl as a substituent may be 5 to 15, and may specifically be, for example, 5, 8, 9, 10, 12, 13, 14, 15, etc. Specific examples of heteroaryl as substituents include, but are not limited to, pyridyl, quinolyl, dibenzofuranyl, dibenzothienyl, carbazolyl, etc.

In the present disclosure, the number of carbon atoms of trialkylsilyl as a substituent may be 3 to 12, for example, 3, 6, 7, 8, 9, etc. Specific examples of trialkylsilyl include, but are not limited to, trimethylsilyl, ethyldimethylsilyl, triethylsilyl, etc.

In the present disclosure, the number of carbon atoms of cycloalkyl as a substituent may be 3 to 10, for example, 5, 6, 8, or 10. Specific examples of cycloalkyl include, but are not limited to, cyclopentyl, cyclohexyl, adamantly, etc.

In the present disclosure, the number of carbon atoms of haloalkyl as a substituent may be 1 to 10. For example, haloalkyl may be fluoroalkyl having 1 to 4 carbon atoms. Specific examples of haloalkyl include, but are not limited to, trifluoromethyl.

In the present disclosure, the number of carbon atoms of deuterated alkyl as a substituent may be 1 to 10. For example, deuterated alkyl may be deuterated alkyl having 1 to 4 carbon atoms. Specific examples of deuterated alkyl include, but are not limited to, trideuteromethyl.

The present disclosure, in a first aspect, provides an organic compound having a structure shown in Formula 1:

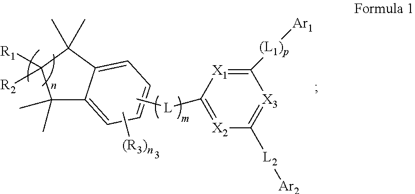

in Formula 1, R₁ and R₂ are identical or different, and are each independently selected from hydrogen or methyl;
X₁, X₂, and X₃ are each independently selected from C(H) or N atoms, and at least one of X₁, X₂, and X₃ is N;
p is selected from 1 or 2; each L₁ and each L₂ is identical or different, and is each independently selected from the group consisting of single bond, substituted or unsubstituted arylene having 6 to 20 carbon atoms, and substituted or unsubstituted heteroarylene having 5 to 20 carbon atoms;
m represents 1 or 2, and each L is independently selected from the group consisting of single bond, substituted or unsubstituted arylene having 6 to 20 carbon atoms, and substituted or unsubstituted heteroarylene having 8 to 20 carbon atoms;
Ar₁ and Ar₂ are identical or different, and are each independently selected from the group consisting of substituted or unsubstituted aryl having 6 to 40 carbon atoms, and substituted or unsubstituted heteroaryl having 5 to 40 carbon atoms;
substituents in L₁, L₂, L, Ar₁, and Ar₂, as well as R₃ are identical or different, and are each independently selected from the group consisting of deuterium, halogen, cyano, alkyl having 1 to carbon atoms, haloalkyl having 1 to 10 carbon atoms, trialkylsiyl having 3 to 12 carbon atoms, deuterated alkyl having 1 to 10 carbon atoms, cycloalkyl having 3 to 10 carbon atoms, aryl 10 having 6 to 18 carbon atoms, and heteroaryl having 5 to 15 carbon atoms; and optionally, in Ar₁ and Ar₂, any two adjacent substituents form a saturated or unsaturated 3 to 18-membered ring substituted or unsubstituted by alkyl having 1 to 4 carbon atoms;
n₃ represents the number of R₃, and is selected from 0, 1, 2, or 3, wherein when n₃ is greater than 1, each R₃ is identical or different.

Optionally, the structure of the organic compound is selected from the group consisting of the following structures:

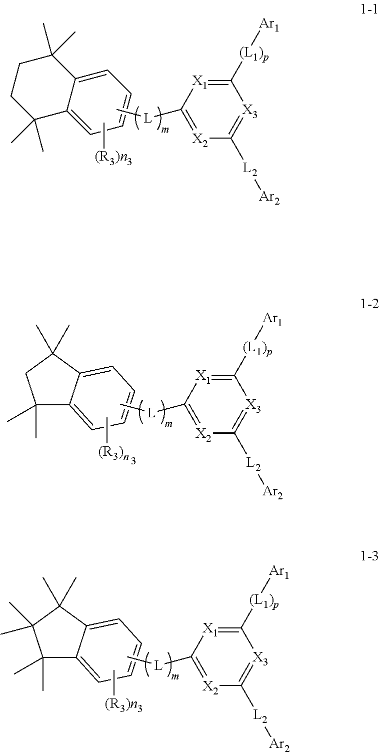

Optionally,

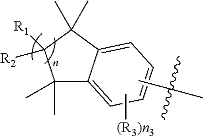

is selected from the group consisting of the following groups:

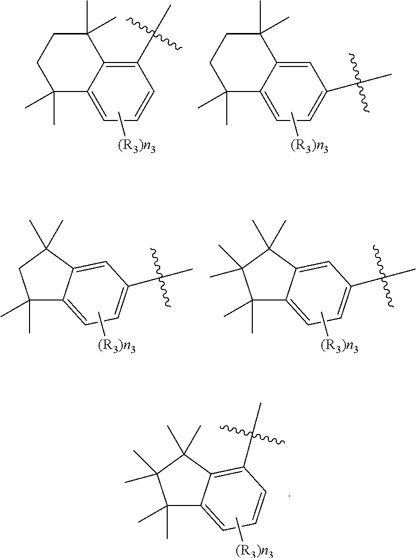

Optionally, each R₃ is independently selected from the group consisting of deuterium, fluorine, cyano, alkyl having 1 to 4 carbon atoms, fluoroalkyl having 1 to 4 carbon atoms, trialkylsilyl having 3 to 7 carbon atoms, aryl having 6 to 12 carbon atoms, and heteroaryl having 5 to 12 carbon atoms. Specific examples of R₃ include, but are not limited to, deuterium, fluorine, cyano, methyl, ethyl, isopropyl, tert-butyl, trifluoromethyl, trideuteromethyl, trimethylsilyl, phenyl, naphthyl, or pyridyl.

In the present disclosure, one of X₁, X₂, and X₃ is a N atom, and the remaining two are C(H); or, two of X₁, X₂, and X₃ are N atoms, and the remaining one is C(H); or, X₁, X₂, and X₃ are all N atoms.

In the present disclosure, in

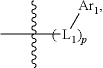

when p is 1,

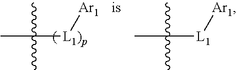

and when p is 2,

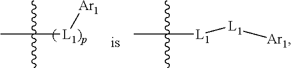

in which the two L₁ may be identical or different. Similarly, when m is 2, the two L may be identical or different.

Optionally, each L₁ and each L₂ is identical or different, and is each independently selected from the group consisting of single bond, substituted or unsubstituted arylene having 6 to 18 carbon atoms, and substituted or unsubstituted heteroarylene having 5 to 15 carbon atoms. For example, each L₁ and each L₂ may be independently selected from the group consisting of single bond, substituted or unsubstituted arylene having 6, 7, 8, 9, 10, 11, 12, 13, 14, 15, 16, 17, or 18 carbon atoms, and substituted or unsubstituted heteroarylene having 5, 7, 8, 9, 10, 11, 12, 13, 14, or 15 carbon atoms.

Optionally, each L₁ and each L₂ is identical or different, and is each independently selected from the group consisting of single bond, substituted or unsubstituted phenylene, substituted or unsubstituted naphthylene, substituted or unsubstituted anthrylene, substituted or unsubstituted phenanthrylene, substituted or unsubstituted pyridylidene, substituted or unsubstituted quinolylene, substituted or unsubstituted dibenzofuranylene, and substituted or unsubstituted carbazolylene.

Optionally, substituents in L₁ and L₂ are each independently selected from the group consisting of deuterium, fluorine, cyano, alkyl having 1 to 4 carbon atoms, fluoroalkyl having 1 to 4 carbon atoms, deuterated alkyl having 1 to 4 carbon atoms, trialkylsilyl having 3 to 7 carbon atoms, aryl having 6 to 12 carbon atoms, and heteroaryl having 5 to 12 carbon atoms.

Optionally, the substituents in L₁ and L₂ are each independently selected from the group consisting of deuterium, fluorine, cyano, methyl, ethyl, isopropyl, tert-butyl, trifluoromethyl, trideuteromethyl, trimethylsilyl, phenyl, naphthyl, pyridyl, dibenzofuranyl, dibenzothienyl, or carbazolyl.

Optionally, each L₁ and each L₂ is identical or different, and is each independently selected from the group consisting of single bond, and substituted or unsubstituted group Z; the unsubstituted group Z is selected from the group consisting of the following groups:

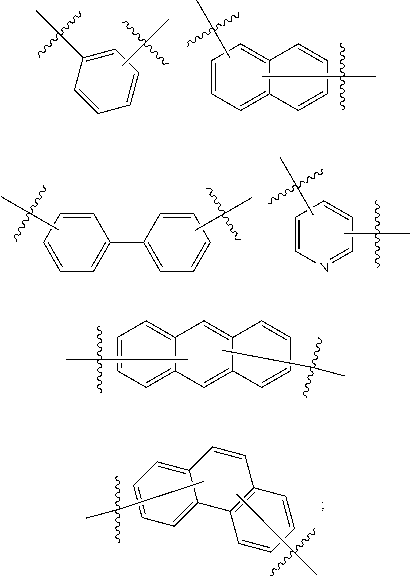

the substituted group Z each has one or more than two substituents, the substituents being each independently selected from the group consisting of deuterium, fluorine, cyano, methyl, ethyl, isopropyl, tert-butyl, trifluoromethyl, trideuteromethyl, trimethylsilyl, phenyl, naphthyl, or pyridyl; and when the number of the substituents is greater than 1, the substituents are identical or different.

In an embodiment, in

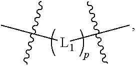

p is 2; one of the two L₁ is selected from single bond or phenylene, and the other L₁ is selected from naphthylene or anthrylene.

In some embodiments,

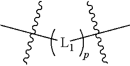

is selected from the group consisting of the following groups:

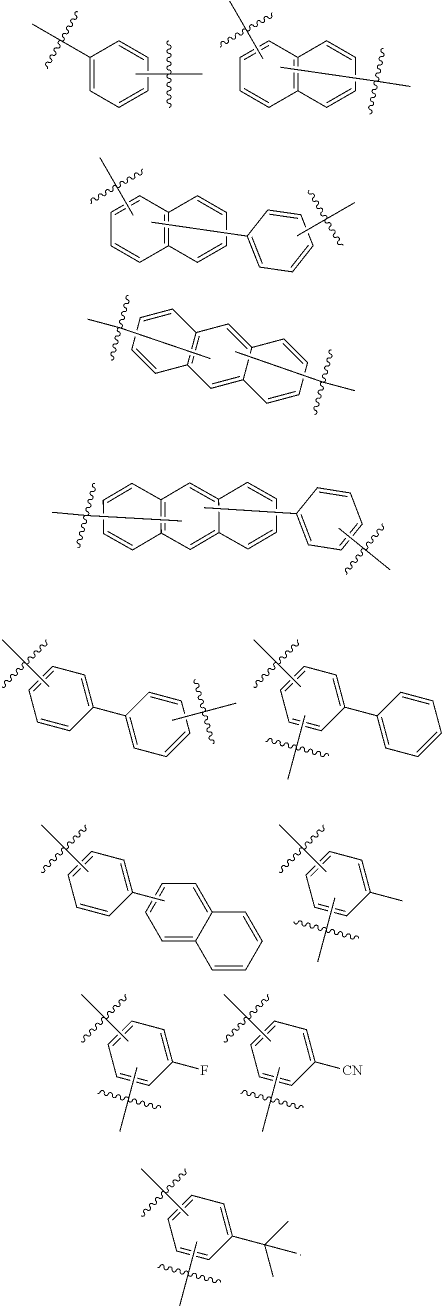

Optionally, each L₁ is identical or different, and is independently selected from the group consisting of single bond and the following groups:

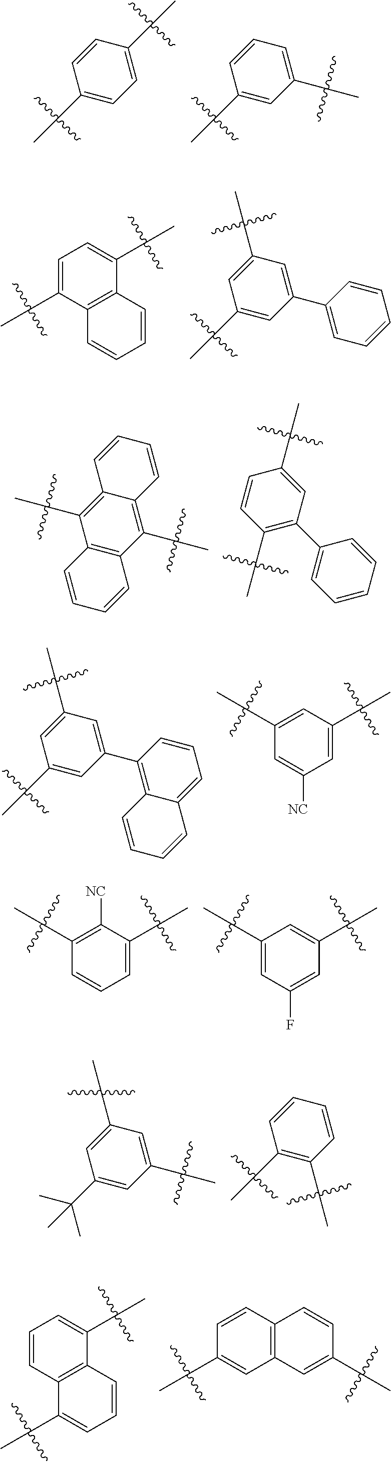

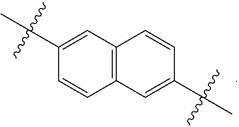 [SMILES: *c1ccc2cc(*)ccc2c1]

Optionally, each L is identical or different, and is independently selected from the group consisting of single bond, substituted or unsubstituted arylene having 6 to 18 carbon atoms, and substituted or unsubstituted heteroarylene having 8 to 15 carbon atoms. For example, each L may be independently selected from the group consisting of single bond, substituted or unsubstituted arylene having 6, 7, 8, 9, 10, 11, 12, 13, 14, 15, 16, 17, or 18 carbon atoms, and substituted or unsubstituted heteroarylene having 8, 9, 10, 11, 12, 13, 14, or 15 carbon atoms.

Optionally, each L is independently selected from the group consisting of single bond, substituted or unsubstituted phenylene, substituted or unsubstituted naphthylene, substituted or unsubstituted phenanthrylene, substituted or unsubstituted anthrylene, substituted or unsubstituted triphenylene

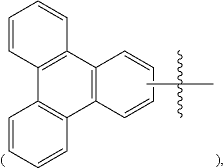

substituted or unsubstituted pyrenylene, substituted or unsubstituted quinolylene, substituted or unsubstituted isoquinolylene, and substituted or unsubstituted dibenzofuranylene.

Optionally, substituents in each L are each independently selected from the group consisting of deuterium, fluorine, cyano, alkyl having 1 to 4 carbon atoms, fluoroalkyl having 1 to 4 carbon atoms, deuterated alkyl having 1 to 4 carbon atoms, trialkylsilyl having 3 to 7 carbon atoms, aryl having 6 to 12 carbon atoms, or heteroaryl having 5 to 12 carbon atoms.

Optionally, the substituents in each L are independently selected from the group consisting of deuterium, fluorine, cyano, methyl, ethyl, isopropyl, tert-butyl, trifluoromethyl, trideuteromethyl, trimethylsilyl, phenyl, naphthyl, biphenyl, pyridyl, dibenzofuranyl, dibenzothienyl, or carbazolyl.

In a specific embodiment, each L is independently selected from the group consisting of single bond, substituted or unsubstituted phenylene, substituted or unsubstituted naphthylene, substituted or unsubstituted quinolylene, substituted or unsubstituted isoquinolylene, and substituted or unsubstituted dibenzofuranylene.

In an embodiment, each L is identical or different, and is independently selected from the group consisting of single bond, and substituted or unsubstituted group V; the unsubstituted group V is selected from the group consisting of the following groups:

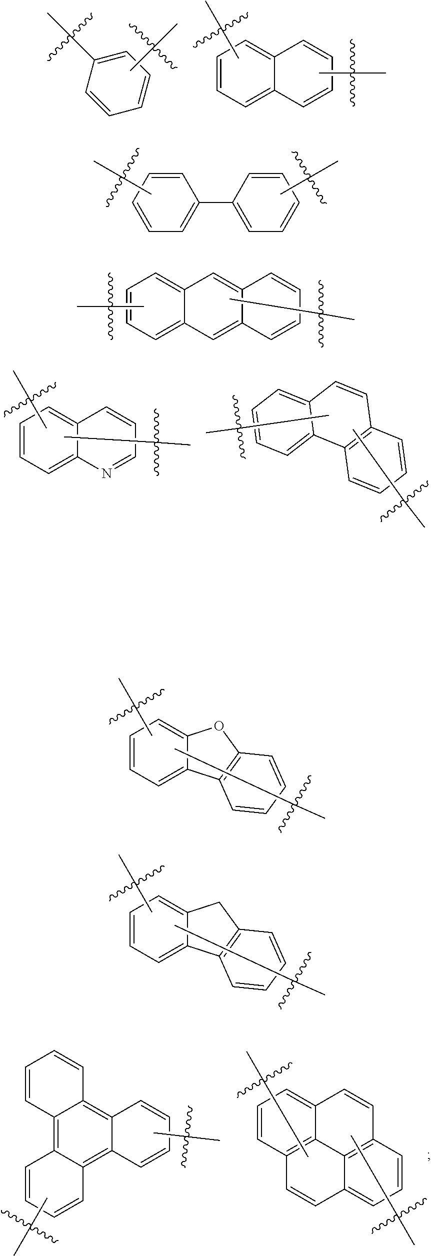

the substituted group V each has one or more substituents, the substituents each being independently selected from the group consisting of deuterium, fluorine, cyano, methyl, ethyl, isopropyl, tert-butyl, trifluoromethyl, trideuteromethyl, trimethylsilyl, phenyl, naphthyl, pyridyl, quinolyl, isoquinolyl, dibenzofuranyl, dibenzothienyl, or carbazolyl; and when the number of the substituents is greater than 1, the substituents are identical or different.

In a specific embodiment, in

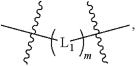

m is 1, and L is dibenzofuranylene.

In an embodiment, in

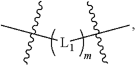

m is 2; one of the two L is selected from single bond or phenylene, and the other L is selected from naphthylene or anthrylene.

In another embodiment, in

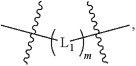

m is 2; one of the two L is selected from phenylene or naphthylene, and the other L is dibenzofuranylene.

In a more specific embodiment,

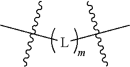

is selected from the group consisting of the following groups:

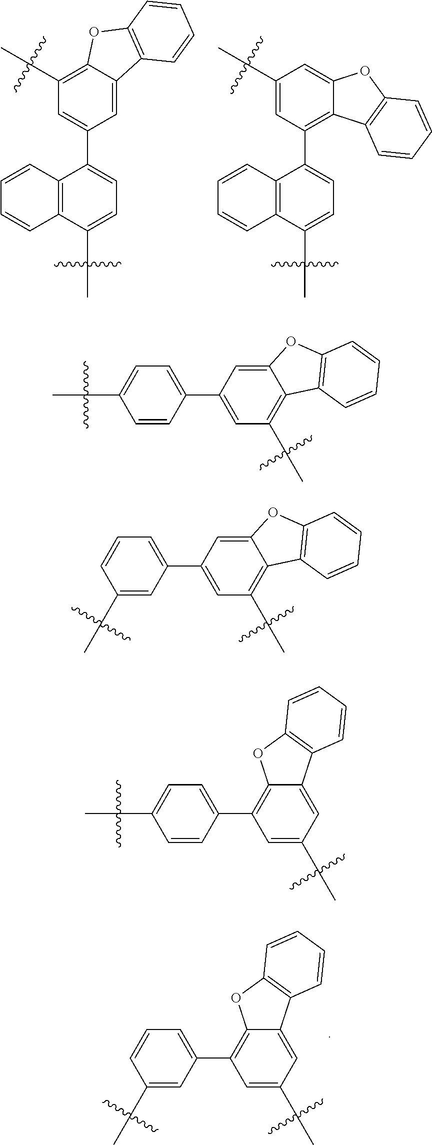 [SMILES: *c1ccc(-c2cc(*)c3oc4ccccc4c3c2)c2ccccc12 ; *c1cc(-c2ccc(*)c3ccccc23)c2c(c1)oc1ccccc12 ; *c1ccc(-c2cc(*)c3c(c2)oc2ccccc23)cc1 ; *c1cccc(-c2cc(*)c3c(c2)oc2ccccc23)c1 ; *c1ccc(-c2cc(*)cc3c2oc2ccccc23)cc1 ; *c1cccc(-c2cc(*)cc3c2oc2ccccc23)c1]

In some embodiments,

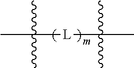

is selected from the group consisting of single bond and the following groups:

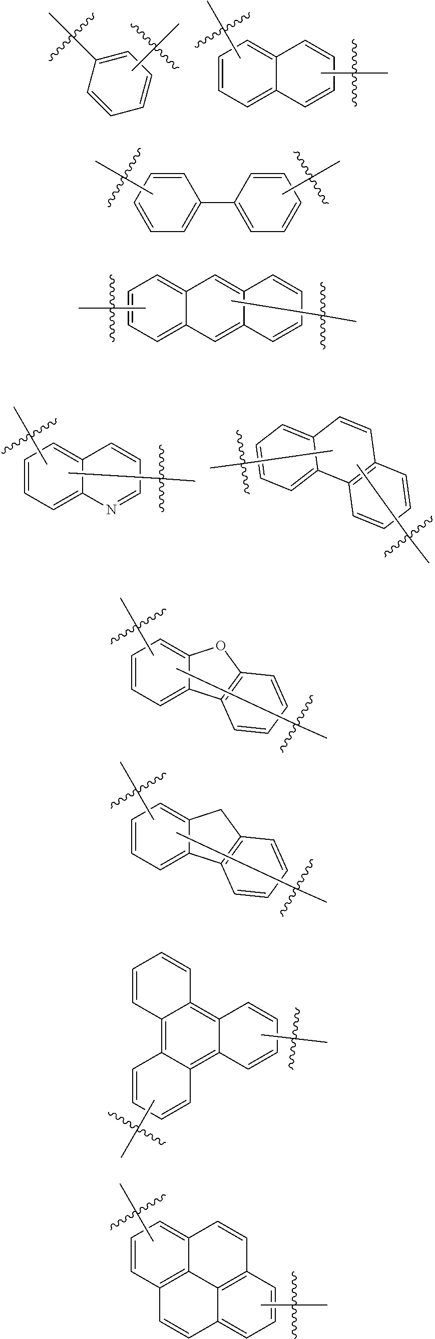

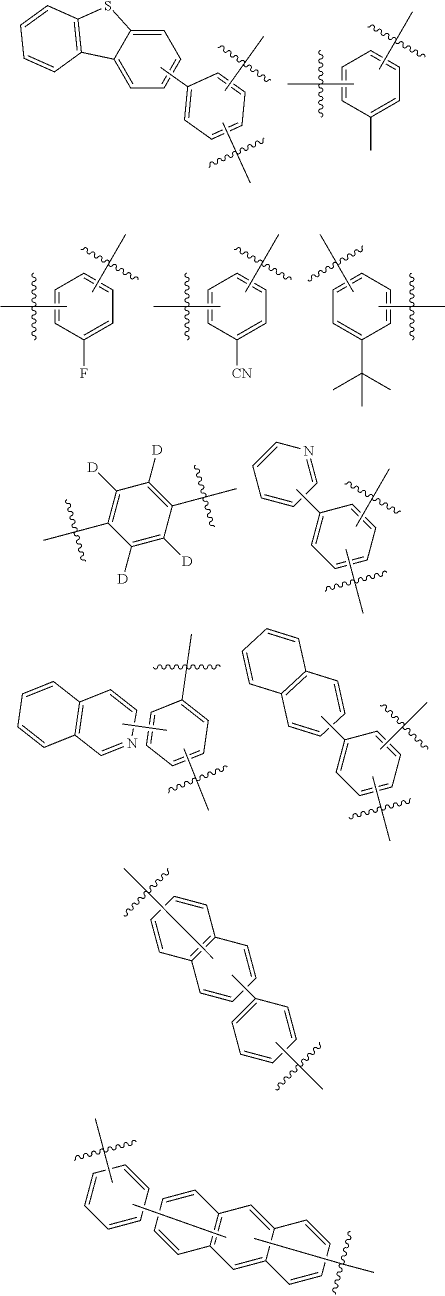

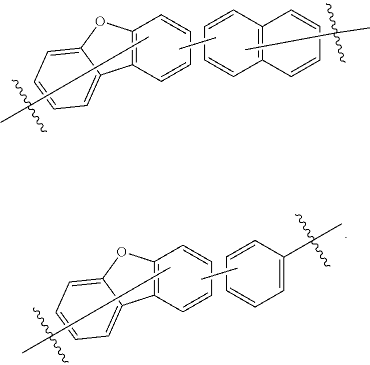

Optionally, each L is identical or different, and is independently selected from the group consisting of single bond and the following groups:

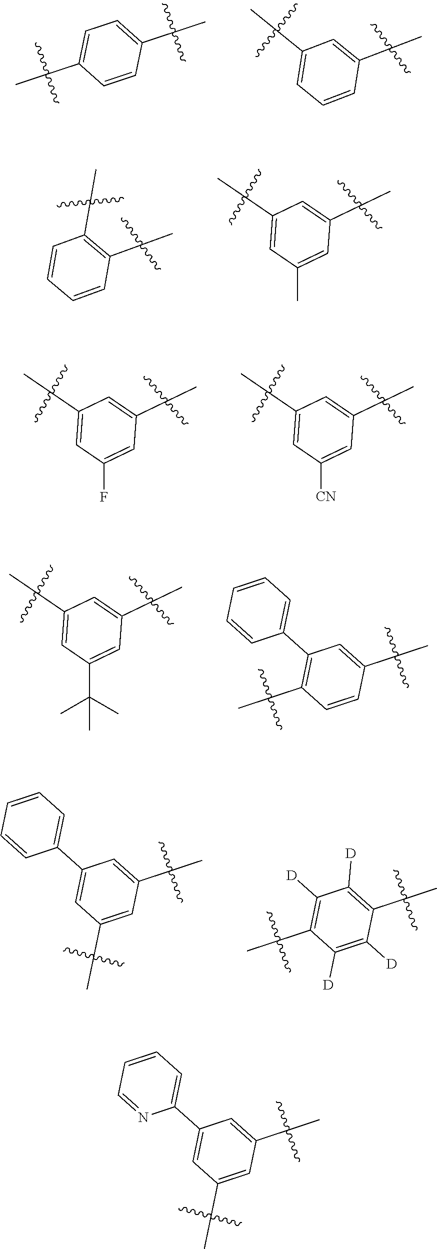

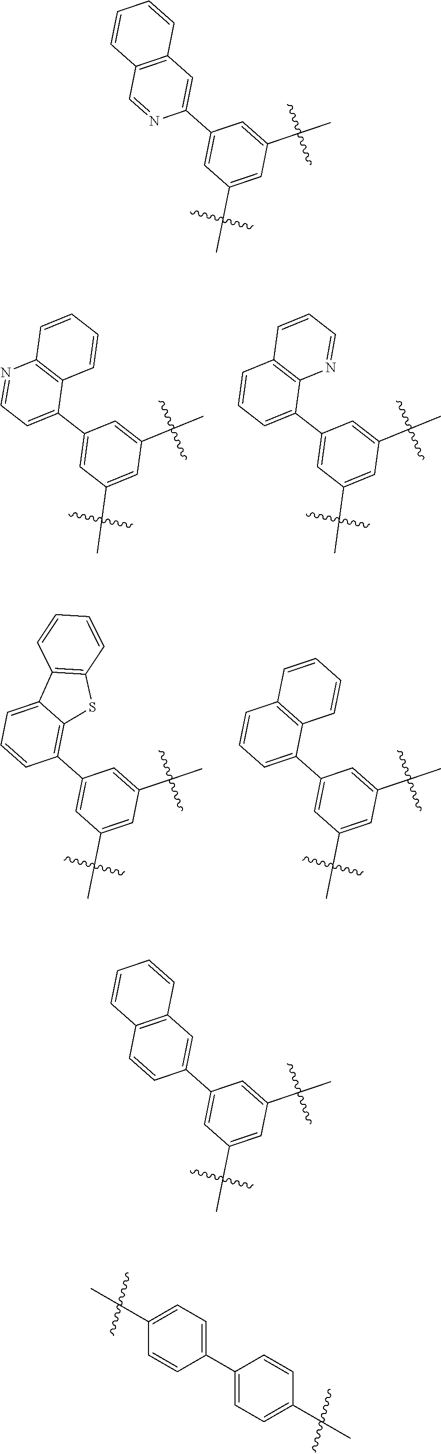

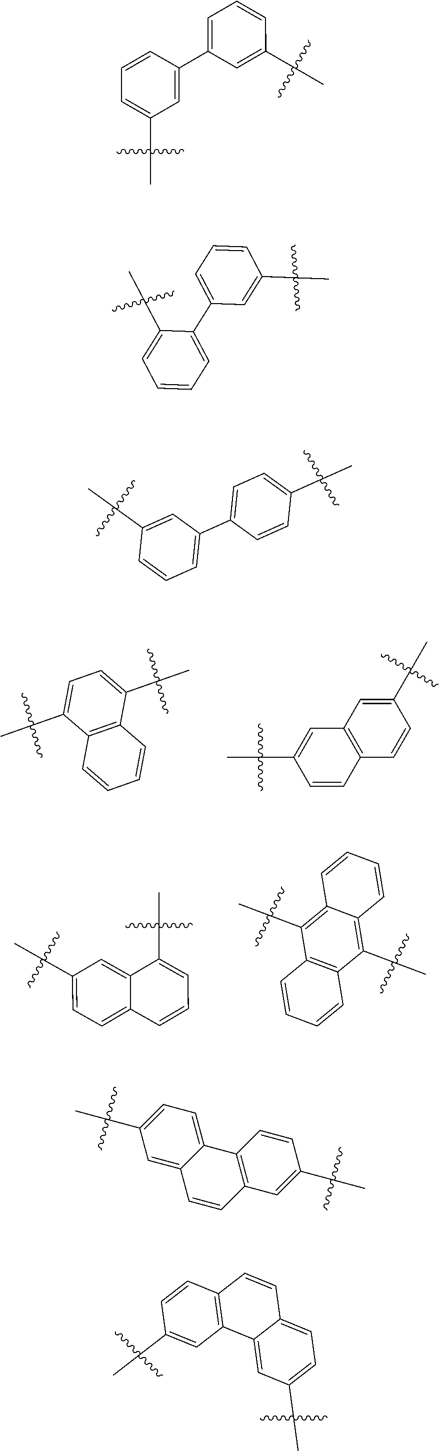

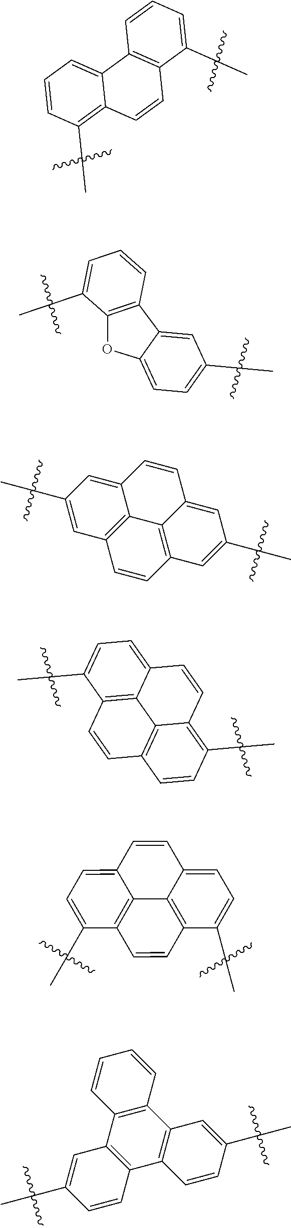 [SMILES: *c1cccc2c1ccc1c(*)cccc12 ; *c1ccc2oc3c(*)cccc3c2c1 ; *c1cc2ccc3cc(*)cc4ccc(c1)c2c34 ; *c1ccc2ccc3c(*)ccc4ccc1c2c43 ; *c1ccc2ccc3ccc(*)c4ccc1c2c34 ; *c1ccc2c3ccc(*)cc3c3ccccc3c2c1]

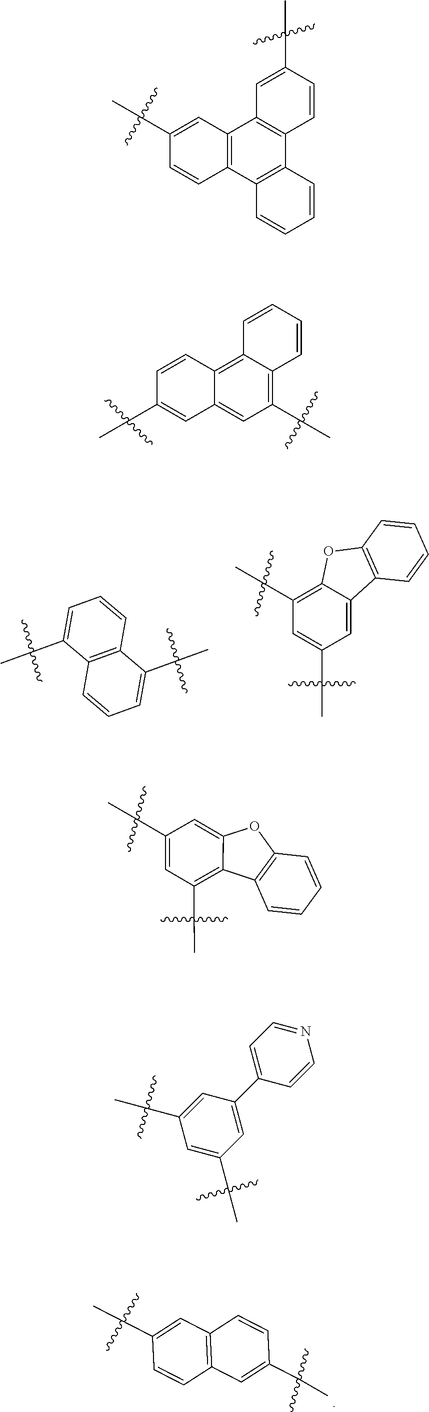

Further optionally, each L is identical or different, and is independently selected from the group consisting of the following groups:

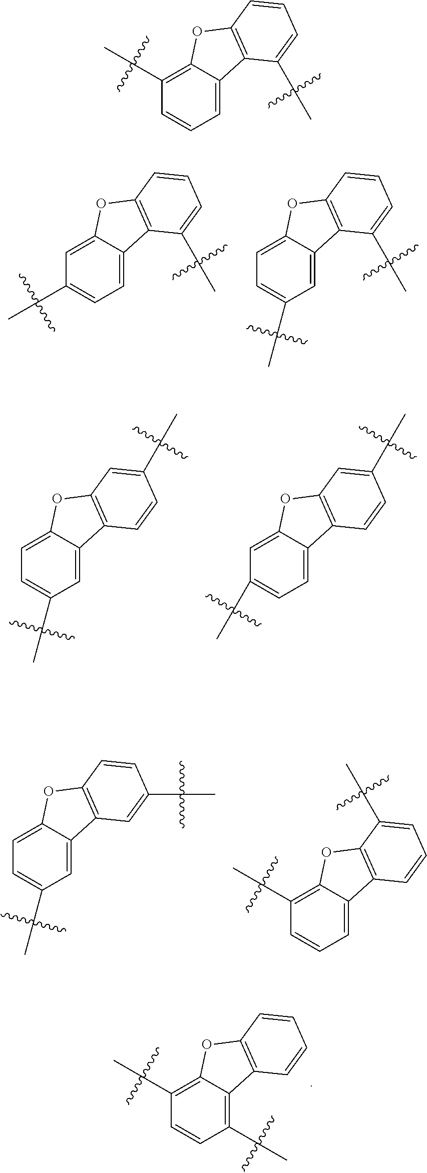

Optionally, Ar₁ and Ar₂ are identical or different, and are each independently selected from the group consisting of substituted or unsubstituted aryl having 6 to 33 carbon atoms, and substituted or unsubstituted heteroaryl having 5 to 25 carbon atoms. For example, Ar₁ and Ar₂ each may be independently selected from the group consisting of substituted or unsubstituted aryl having 6, 7, 8, 9, 10, 11, 12, 13, 14, 15, 16, 17, 18, 19, 20, 21, 22, 23, 24, 25, 26, 27, 28, 29, 30, 31, 32, or 33 carbon atoms, and substituted or unsubstituted heteroaryl having 5, 6, 7, 8, 9, 10, 11, 12, 13, 14, 15, 16, 17, 18, 19, 21, 22, 23, 24, or 25 carbon atoms.

Optionally, substituents in Ar₁ and Ar₂ are each independently selected from the group consisting of deuterium, fluorine, cyano, alkyl having 1 to 4 carbon atoms, fluoroalkyl having 1 to 4 carbon atoms, deuterated alkyl having 1 to 4 carbon atoms, trialkylsilyl having 3 to 7 carbon atoms, cycloalkyl having 5 to 10 carbon atoms, aryl having 6 to 12 carbon atoms, and heteroaryl having 5 to 12 carbon atoms; and optionally, any two adjacent substituents form a saturated or unsaturated 5 to 15-membered ring substituted or unsubstituted by alkyl having 1 to 4 carbon atoms.

Optionally, Ar₁ and Ar₂ are identical or different, and are each independently selected from the group consisting of substituted or unsubstituted phenyl, substituted or unsubstituted naphthyl, substituted or unsubstituted biphenyl, substituted or unsubstituted anthryl, substituted or unsubstituted phenanthryl, substituted or unsubstituted fluorenyl, substituted or unsubstituted triphenylene

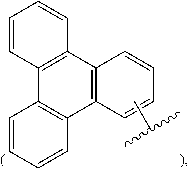

substituted or unsubstituted pyrenyl, substituted or unsubstituted pyridyl, substituted or unsubstituted quinolyl, substituted or unsubstituted isoquinolyl, substituted or unsubstituted benzoxazolyl, substituted or unsubstituted benzothiazolyl, substituted or unsubstituted quinoxalinyl, substituted or unsubstituted quinazolinyl, substituted or unsubstituted phenanthrolin, substituted or unsubstituted xanthenyl, substituted or unsubstituted thioxanthenyl, substituted or unsubstituted dibenzo-p-dioxin

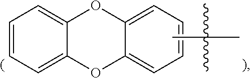

substituted or unsubstituted thianthrenyl, substituted or unsubstituted benzimidazolyl, substituted or unsubstituted dibenzofuranyl, substituted or unsubstituted dibenzothienyl, and substituted or unsubstituted carbazolyl.

Optionally, substituents in Ar₁ and Ar₂ are each independently selected from the group consisting of deuterium, fluorine, cyano, methyl, ethyl, isopropyl, tert-butyl, trifluoromethyl, trideuteromethyl, trimethylsilyl, cyclopentyl, cyclohexyl, phenyl, naphthyl, biphenyl, pyridyl, quinolyl, isoquinolyl, dibenzofuranyl, dibenzothienyl, or carbazolyl; and optionally, any two adjacent substituents form a benzene ring, a naphthalene ring, cyclopentane, cyclohexane, a fluorene ring, an xanthene ring, a thioxanthene ring, or a tert-butyl-substituted fluorene ring.

In an embodiment, Ar₁ and Ar₂ are identical or different, and are each independently selected from substituted or unsubstituted group W; the unsubstituted group W is selected from the group consisting of the following groups:

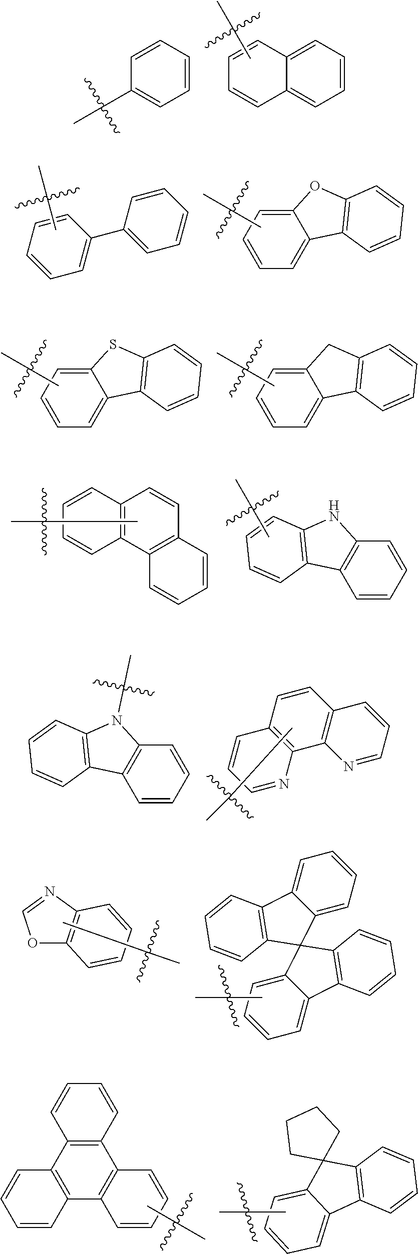

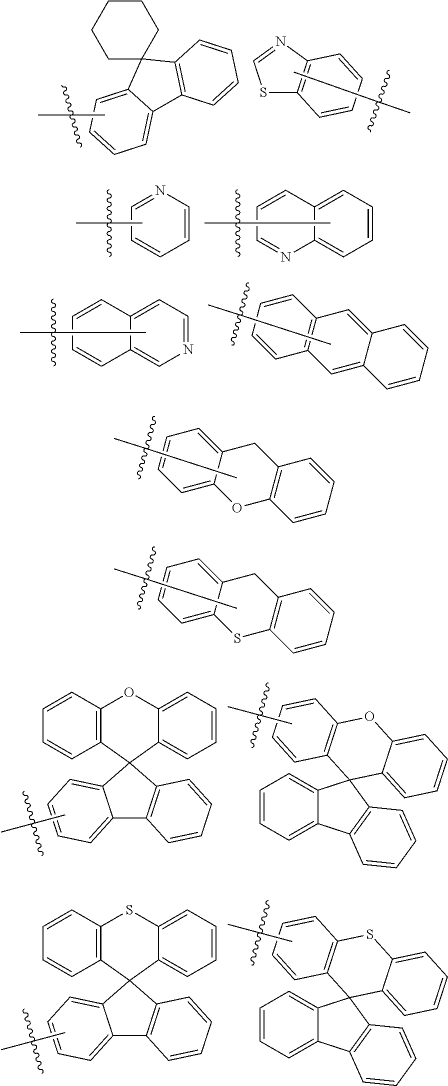

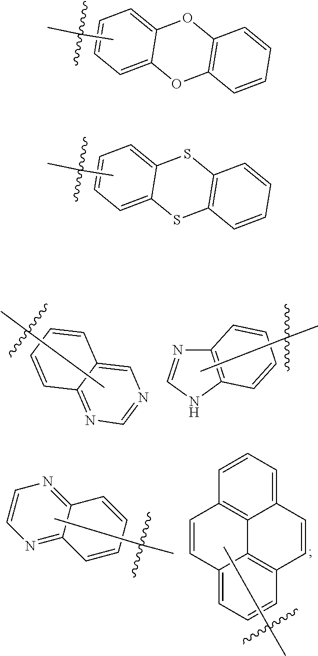

the substituted group W each has one or more substituents, the substituents being each independently selected from the group consisting of deuterium, fluorine, cyano, methyl, ethyl, isopropyl, tert-butyl, trifluoromethyl, trideuteromethyl, trimethylsilyl, cyclopentyl, cyclohexyl, phenyl, naphthyl, biphenyl, or pyridyl; and when the number of the substituents is greater than 1, the substituents are identical or different.

Optionally, Ar₁ and Ar₂ are each independently selected from the group consisting of the following groups:

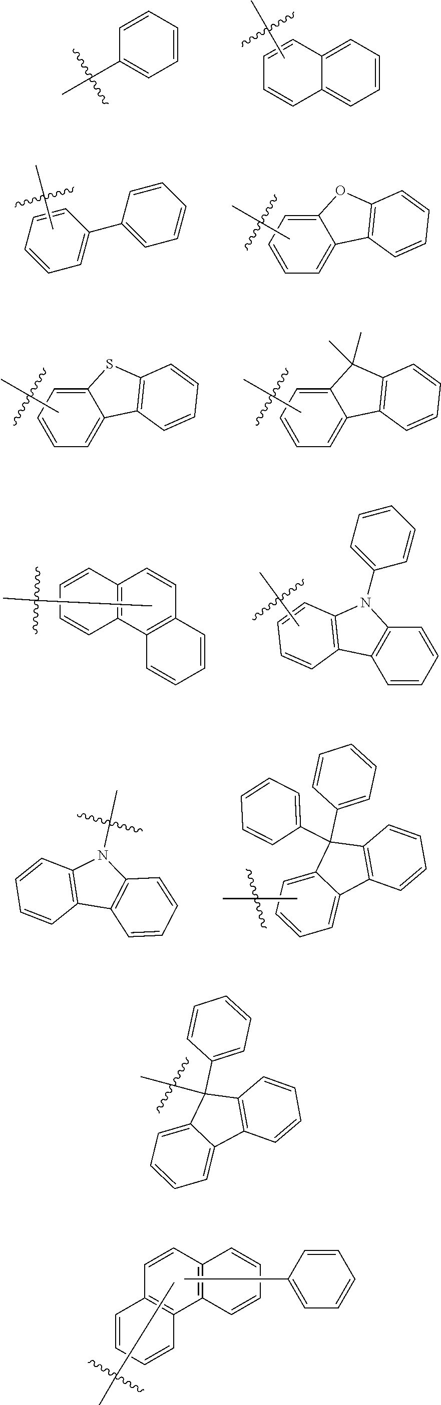

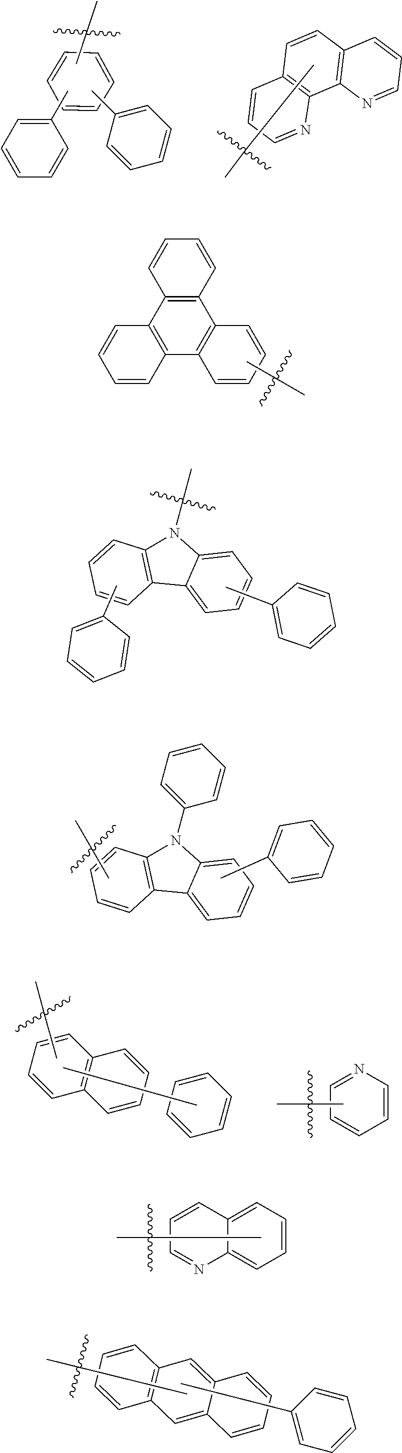

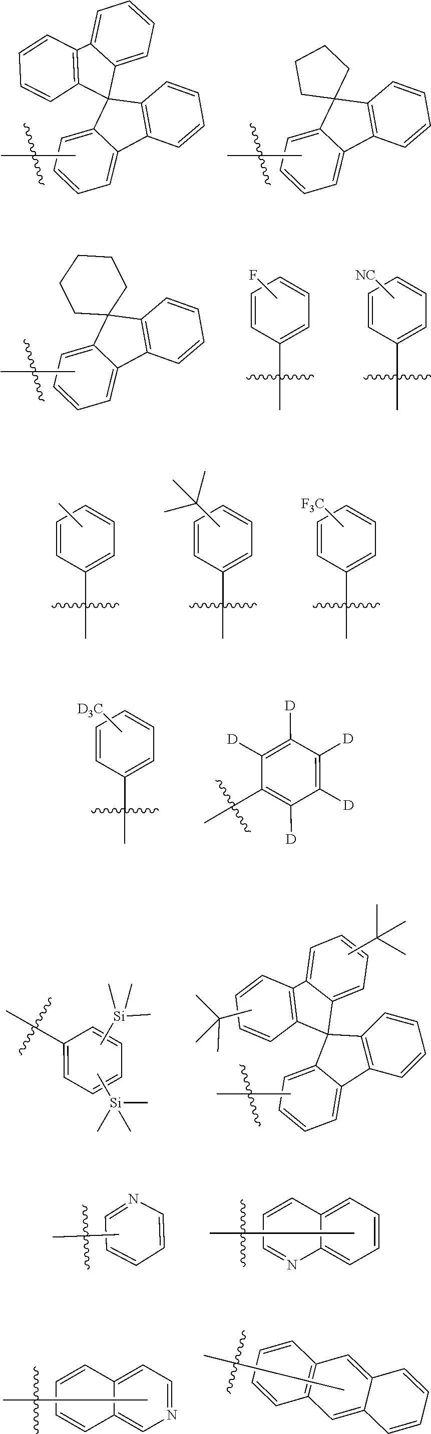

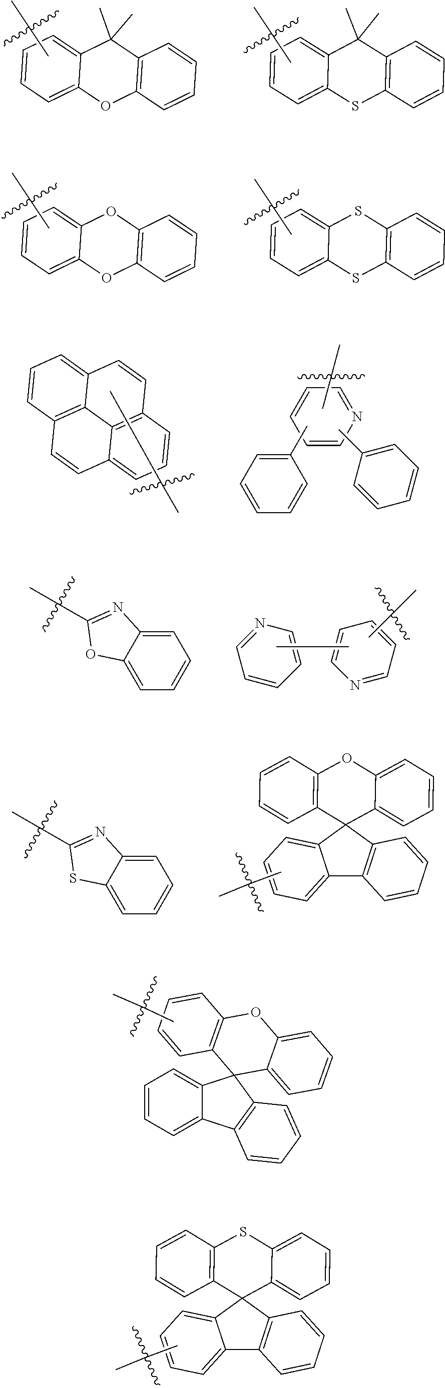

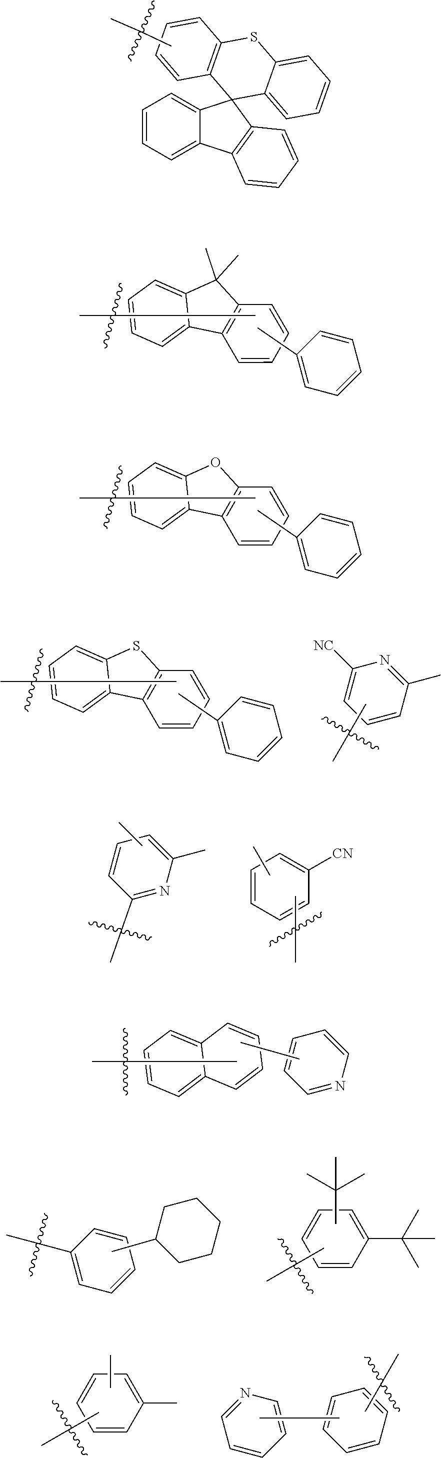

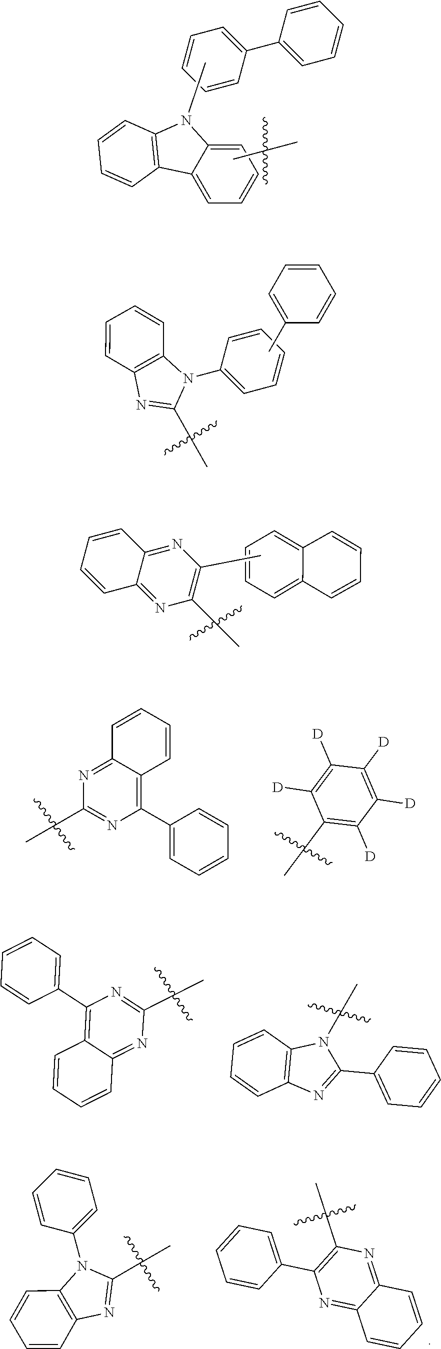

Optionally, Ar₁ and Ar₂ are each independently selected from the group consisting of the following groups:

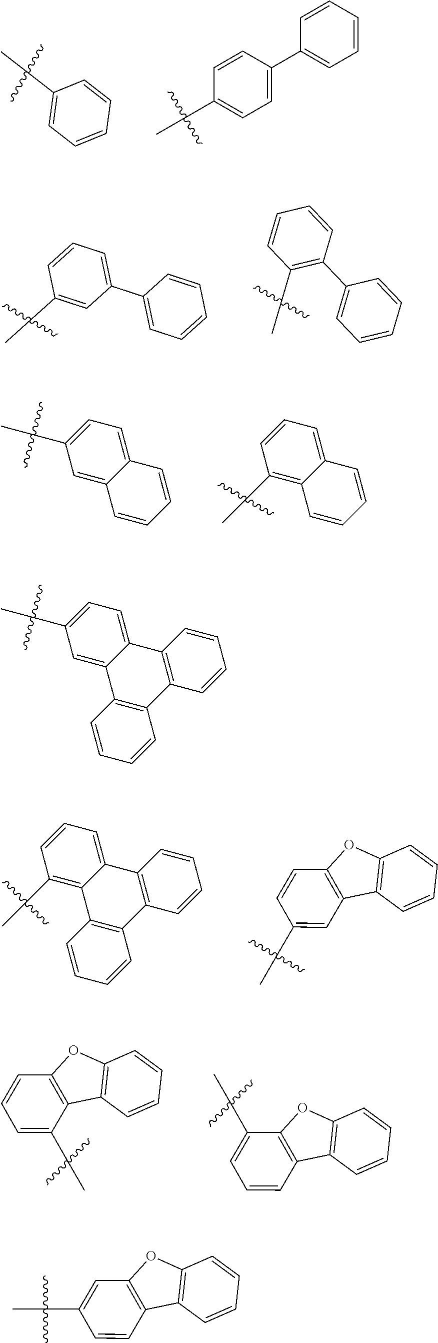

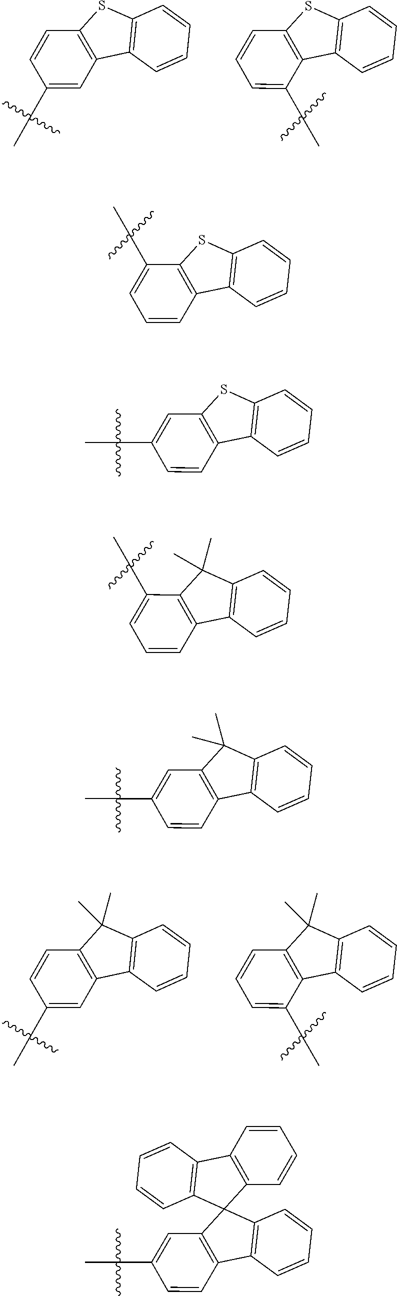

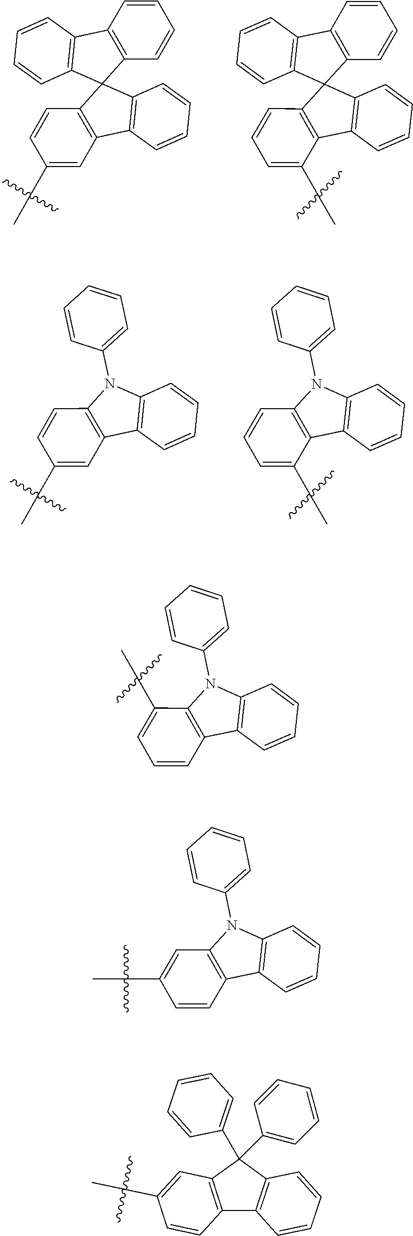

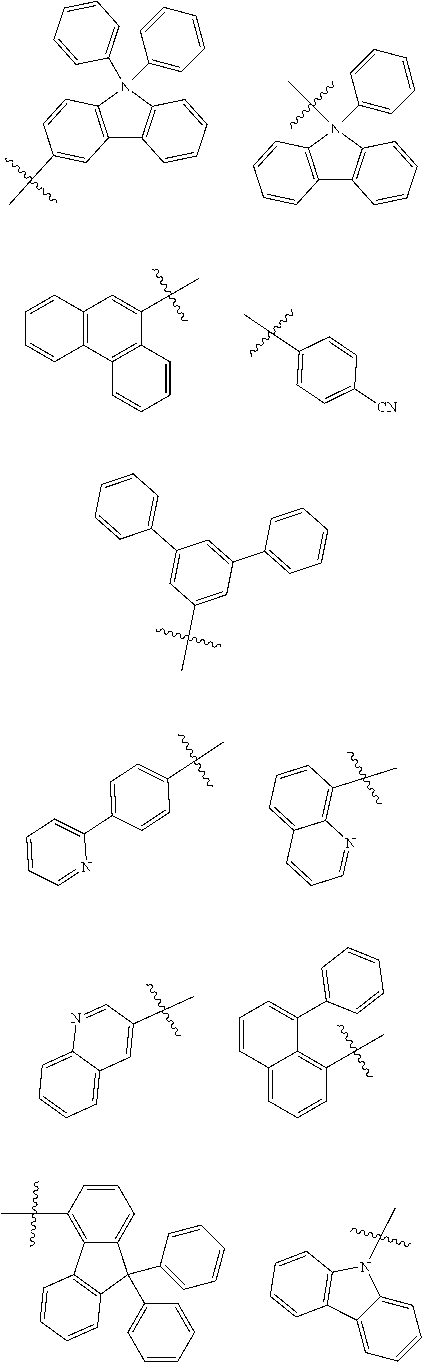

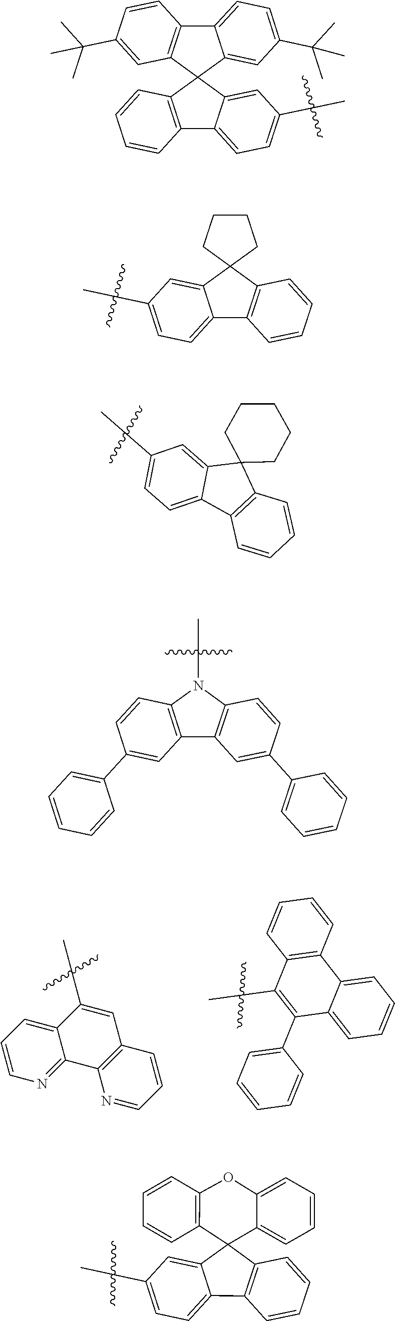

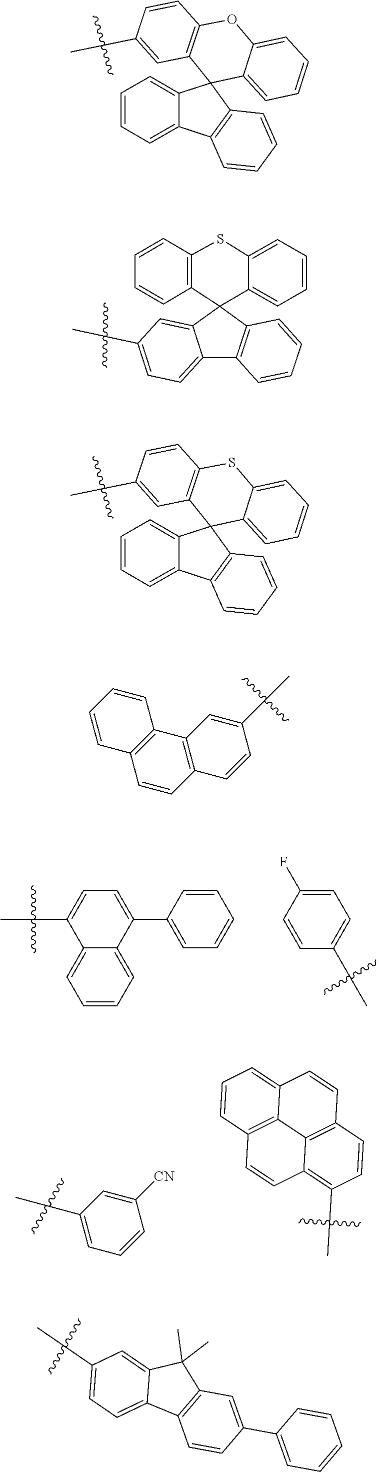

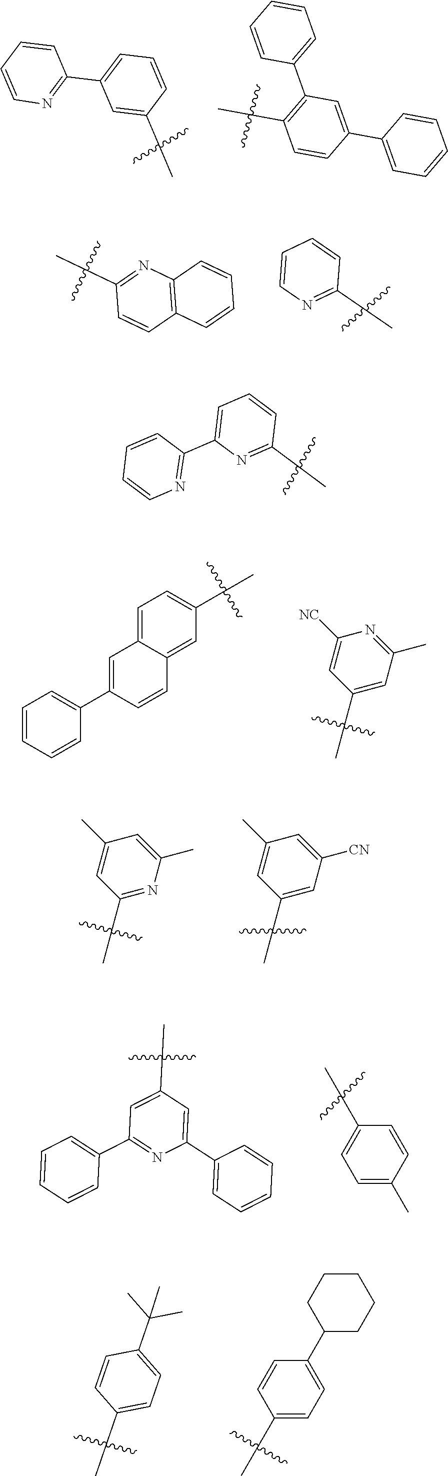

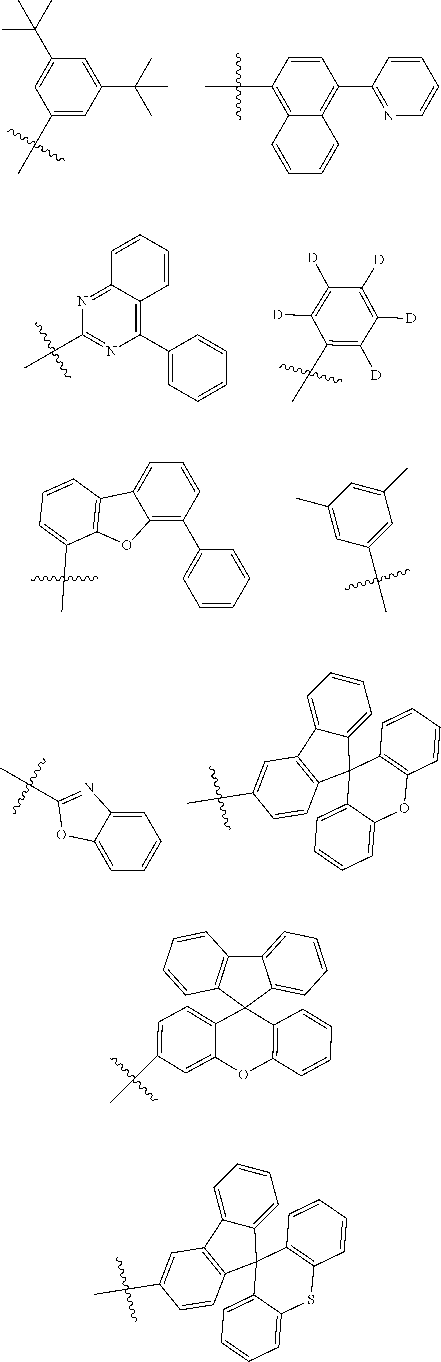

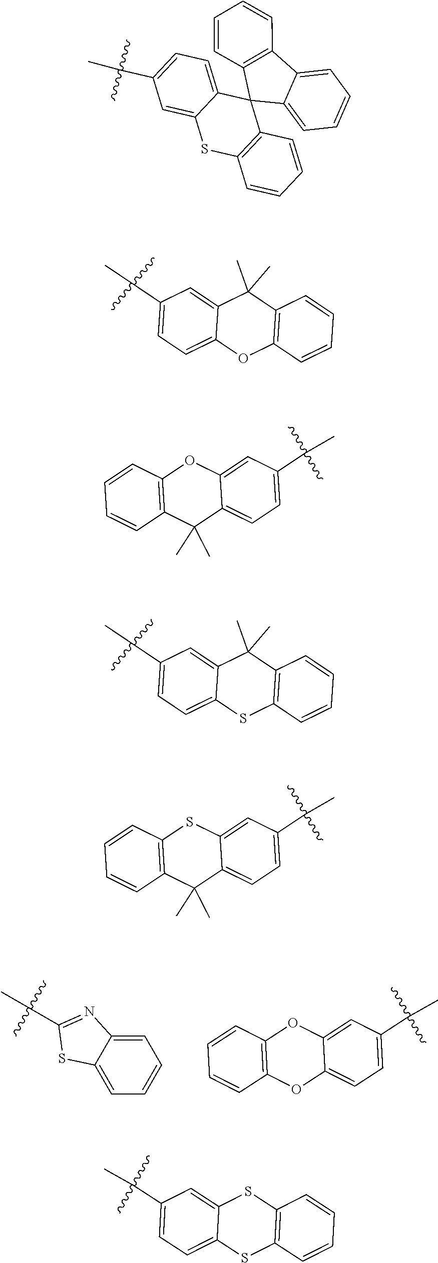

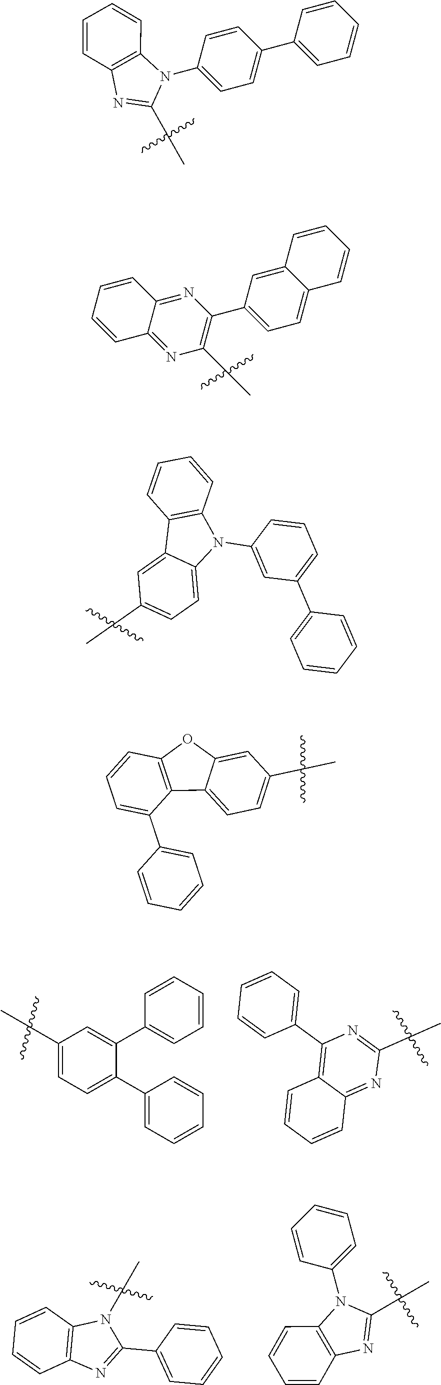

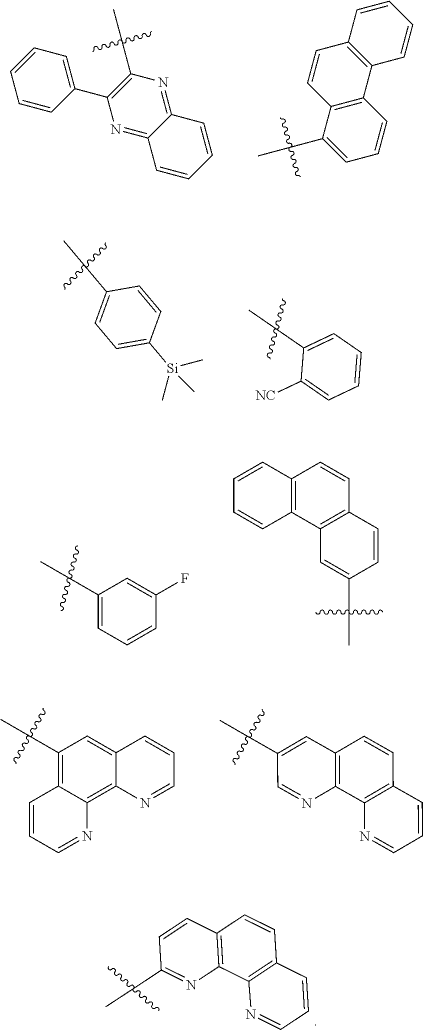

Optionally, the organic compound is selected from the group consisting of the following compounds:

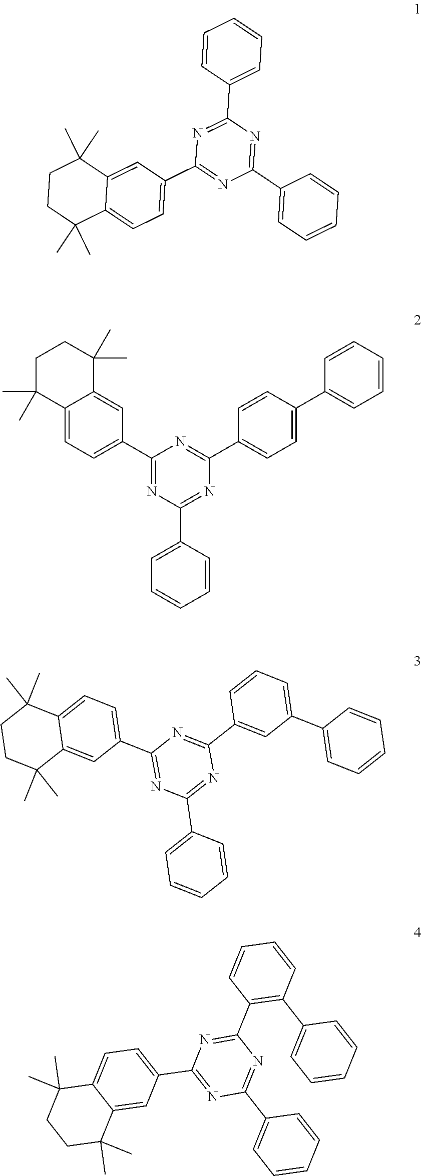 [SMILES: CC1(C)CCC(C)(C)c2cc(-c3nc(-c4ccccc4)nc(-c4ccccc4)n3)ccc21 ; CC1(C)CCC(C)(C)c2cc(-c3nc(-c4ccccc4)nc(-c4ccc(-c5ccccc5)cc4)n3)ccc21 ; CC1(C)CCC(C)(C)c2cc(-c3nc(-c4ccccc4)nc(-c4cccc(-c5ccccc5)c4)n3)ccc21 ; CC1(C)CCC(C)(C)c2cc(-c3nc(-c4ccccc4)nc(-c4ccccc4-c4ccccc4)n3)ccc21]

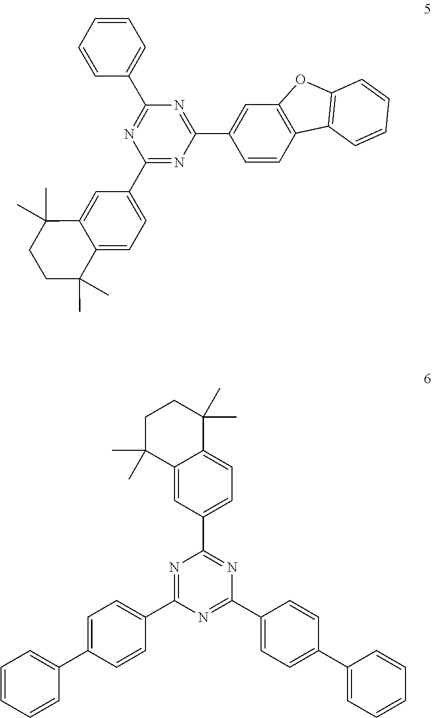 [SMILES: CC1(C)CCC(C)(C)c2cc(-c3nc(-c4ccccc4)nc(-c4ccc5c(c4)oc4ccccc45)n3)ccc21 ; CC1(C)CCC(C)(C)c2cc(-c3nc(-c4ccc(-c5ccccc5)cc4)nc(-c4ccc(-c5ccccc5)cc4)n3)ccc21]

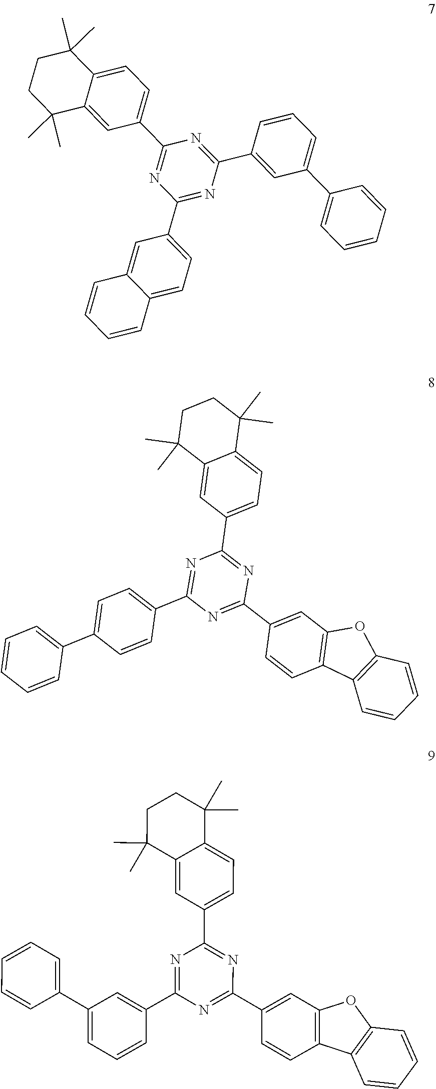 [SMILES: CC1(C)CCC(C)(C)c2cc(-c3nc(-c4cccc(-c5ccccc5)c4)nc(-c4ccc5ccccc5c4)n3)ccc21 ; CC1(C)CCC(C)(C)c2cc(-c3nc(-c4ccc(-c5ccccc5)cc4)nc(-c4ccc5c(c4)oc4ccccc45)n3)ccc21 ; CC1(C)CCC(C)(C)c2cc(-c3nc(-c4cccc(-c5ccccc5)c4)nc(-c4ccc5c(c4)oc4ccccc45)n3)ccc21]

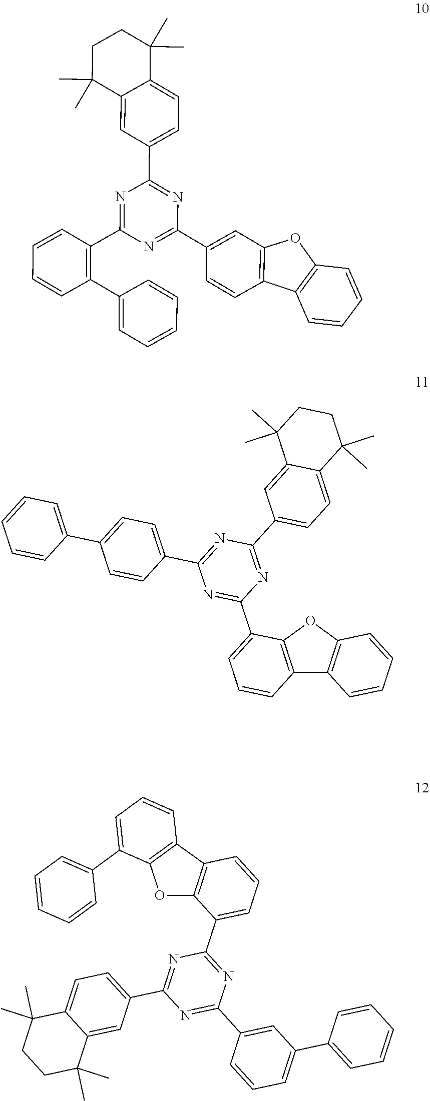 [SMILES: CC1(C)CCC(C)(C)c2cc(-c3nc(-c4ccc5c(c4)oc4ccccc45)nc(-c4ccccc4-c4ccccc4)n3)ccc21 ; CC1(C)CCC(C)(C)c2cc(-c3nc(-c4ccc(-c5ccccc5)cc4)nc(-c4cccc5c4oc4ccccc45)n3)ccc21 ; CC1(C)CCC(C)(C)c2cc(-c3nc(-c4cccc(-c5ccccc5)c4)nc(-c4cccc5c4oc4c(-c6ccccc6)cccc45)n3)ccc21]

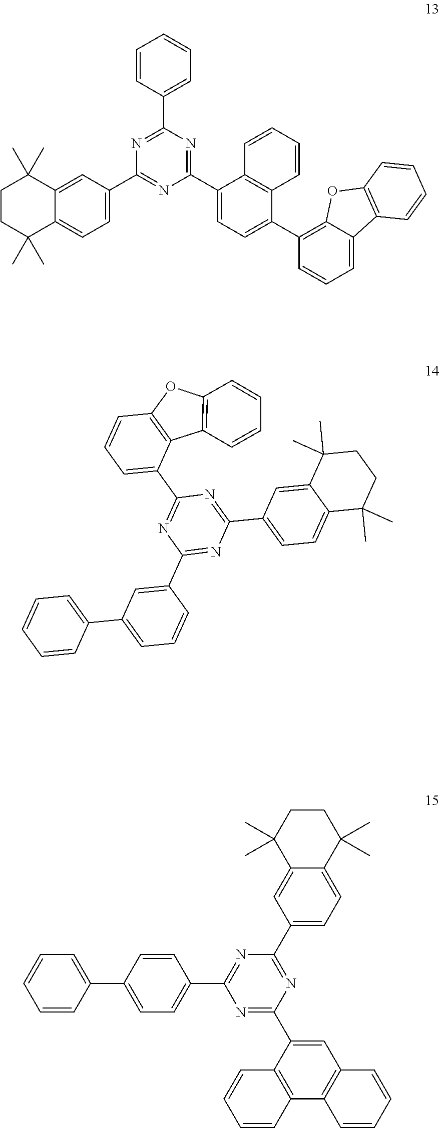 [SMILES: CC1(C)CCC(C)(C)c2cc(-c3nc(-c4ccccc4)nc(-c4ccc(-c5cccc6c5oc5ccccc56)c5ccccc45)n3)ccc21 ; CC1(C)CCC(C)(C)c2cc(-c3nc(-c4cccc(-c5ccccc5)c4)nc(-c4cccc5oc6ccccc6c45)n3)ccc21 ; CC1(C)CCC(C)(C)c2cc(-c3nc(-c4ccc(-c5ccccc5)cc4)nc(-c4cc5ccccc5c5ccccc45)n3)ccc21]

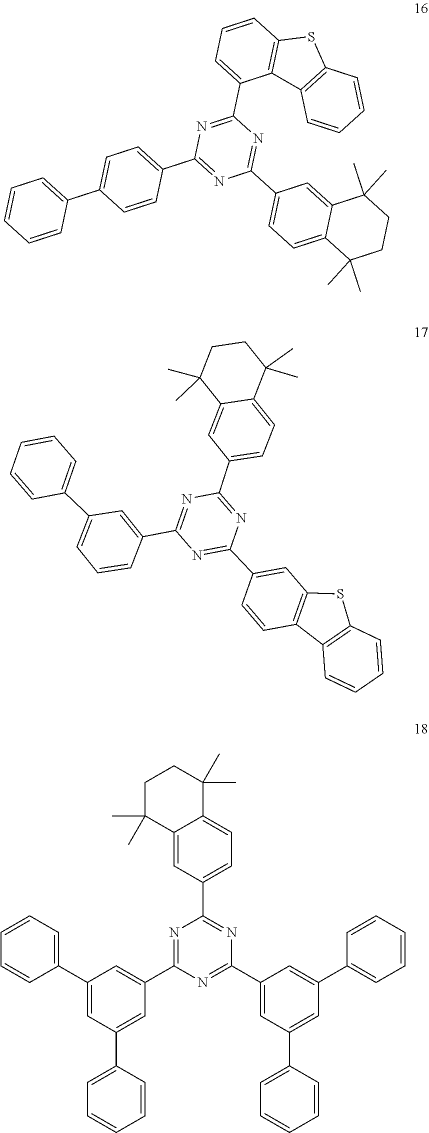 [SMILES: CC1(C)CCC(C)(C)c2cc(-c3nc(-c4ccc(-c5ccccc5)cc4)nc(-c4cccc5sc6ccccc6c45)n3)ccc21 ; CC1(C)CCC(C)(C)c2cc(-c3nc(-c4cccc(-c5ccccc5)c4)nc(-c4ccc5c(c4)sc4ccccc45)n3)ccc21 ; CC1(C)CCC(C)(C)c2cc(-c3nc(-c4cc(-c5ccccc5)cc(-c5ccccc5)c4)nc(-c4cc(-c5ccccc5)cc(-c5ccccc5)c4)n3)ccc21]

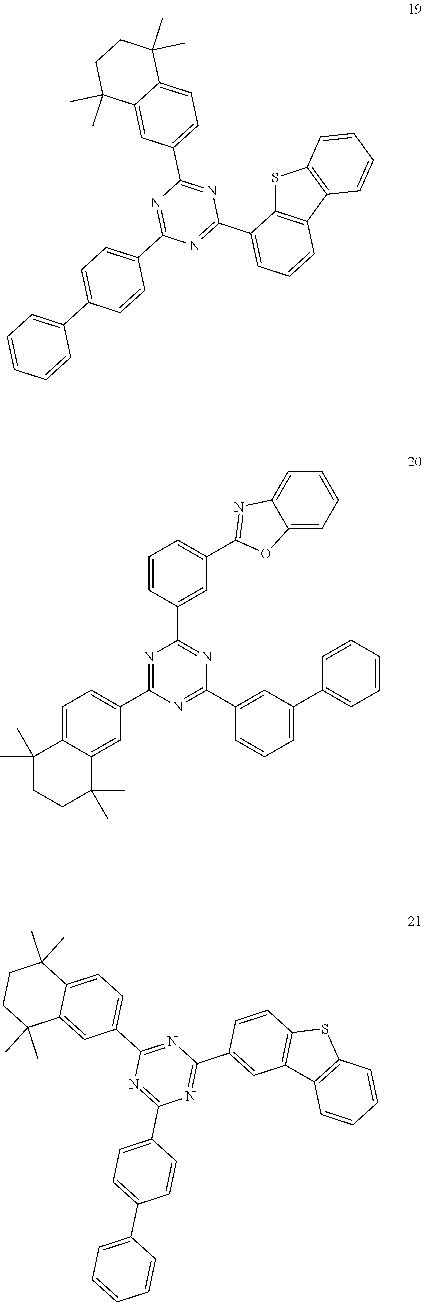 [SMILES: CC1(C)CCC(C)(C)c2cc(-c3nc(-c4ccc(-c5ccccc5)cc4)nc(-c4cccc5c4sc4ccccc45)n3)ccc21 ; CC1(C)CCC(C)(C)c2cc(-c3nc(-c4cccc(-c5ccccc5)c4)nc(-c4cccc(-c5nc6ccccc6o5)c4)n3)ccc21 ; CC1(C)CCC(C)(C)c2cc(-c3nc(-c4ccc(-c5ccccc5)cc4)nc(-c4ccc5sc6ccccc6c5c4)n3)ccc21]

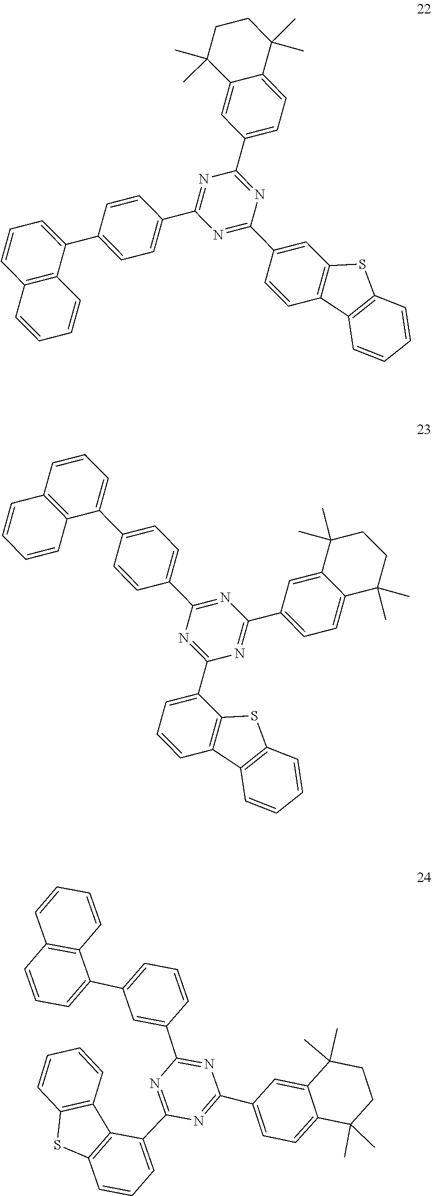 [SMILES: CC1(C)CCC(C)(C)c2cc(-c3nc(-c4ccc(-c5cccc6ccccc56)cc4)nc(-c4ccc5c(c4)sc4ccccc45)n3)ccc21 ; CC1(C)CCC(C)(C)c2cc(-c3nc(-c4ccc(-c5cccc6ccccc56)cc4)nc(-c4cccc5c4sc4ccccc45)n3)ccc21 ; CC1(C)CCC(C)(C)c2cc(-c3nc(-c4cccc(-c5cccc6ccccc56)c4)nc(-c4cccc5sc6ccccc6c45)n3)ccc21]

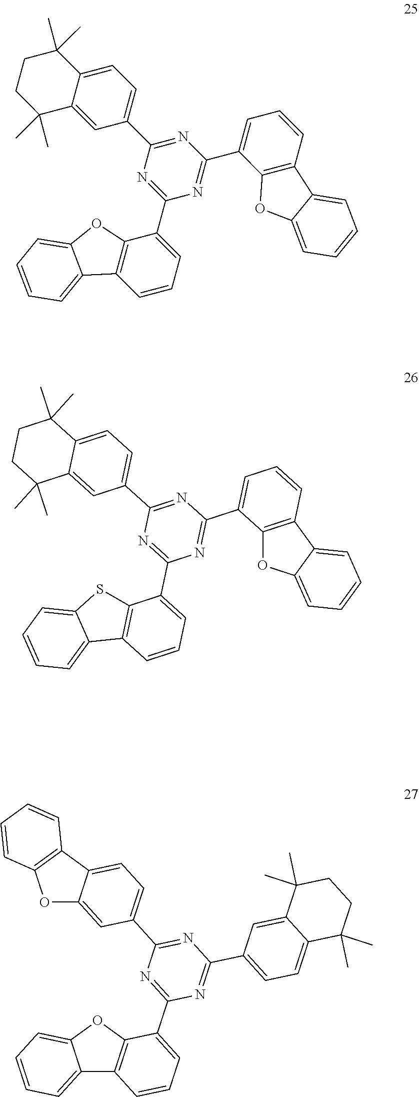 [SMILES: CC1(C)CCC(C)(C)c2cc(-c3nc(-c4cccc5c4oc4ccccc45)nc(-c4cccc5c4oc4ccccc45)n3)ccc21 ; CC1(C)CCC(C)(C)c2cc(-c3nc(-c4cccc5c4oc4ccccc45)nc(-c4cccc5c4sc4ccccc45)n3)ccc21 ; CC1(C)CCC(C)(C)c2cc(-c3nc(-c4ccc5c(c4)oc4ccccc45)nc(-c4cccc5c4oc4ccccc45)n3)ccc21]

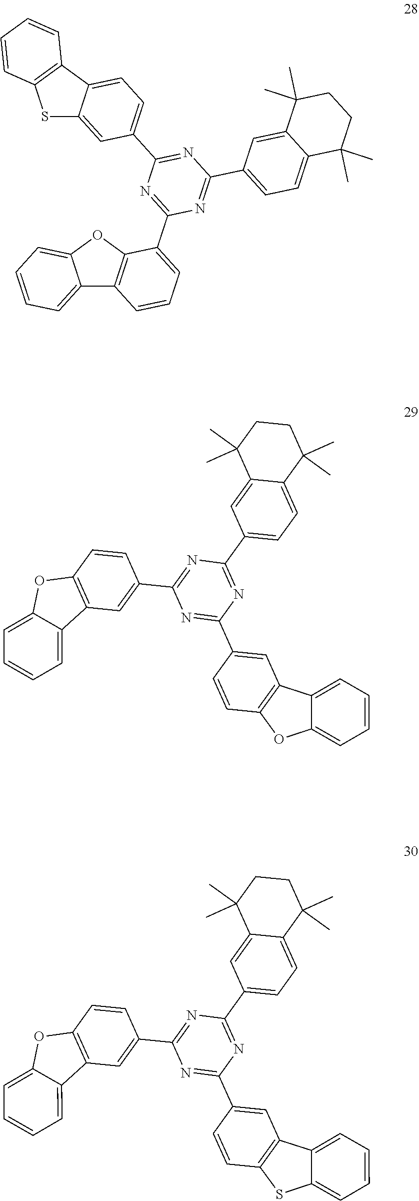 [SMILES: CC1(C)CCC(C)(C)c2cc(-c3nc(-c4ccc5c(c4)sc4ccccc45)nc(-c4cccc5c4oc4ccccc45)n3)ccc21 ; CC1(C)CCC(C)(C)c2cc(-c3nc(-c4ccc5oc6ccccc6c5c4)nc(-c4ccc5oc6ccccc6c5c4)n3)ccc21 ; CC1(C)CCC(C)(C)c2cc(-c3nc(-c4ccc5oc6ccccc6c5c4)nc(-c4ccc5sc6ccccc6c5c4)n3)ccc21]

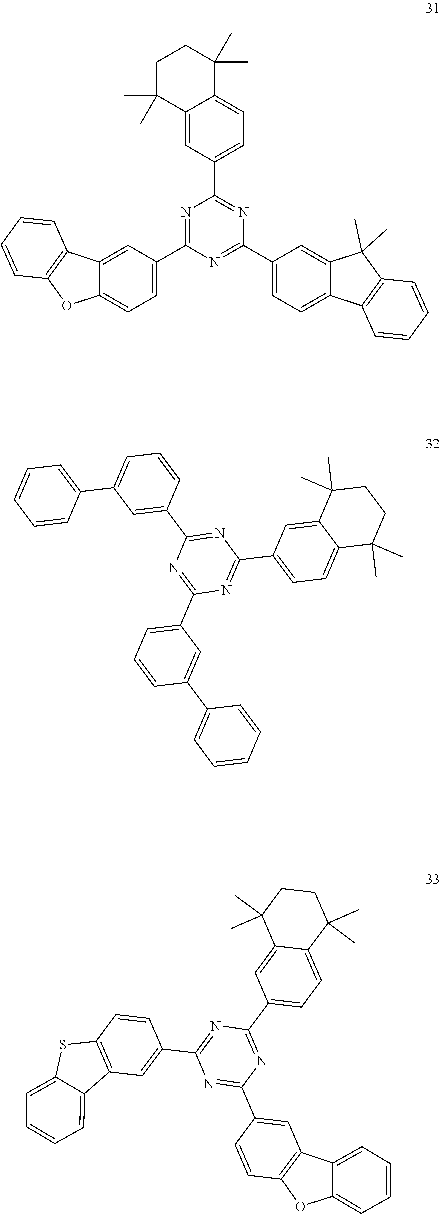 [SMILES: CC1(C)CCC(C)(C)c2cc(-c3nc(-c4ccc5c(c4)C(C)(C)c4ccccc4-5)nc(-c4ccc5oc6ccccc6c5c4)n3)ccc21 ; CC1(C)CCC(C)(C)c2cc(-c3nc(-c4cccc(-c5ccccc5)c4)nc(-c4cccc(-c5ccccc5)c4)n3)ccc21 ; CC1(C)CCC(C)(C)c2cc(-c3nc(-c4ccc5oc6ccccc6c5c4)nc(-c4ccc5sc6ccccc6c5c4)n3)ccc21]

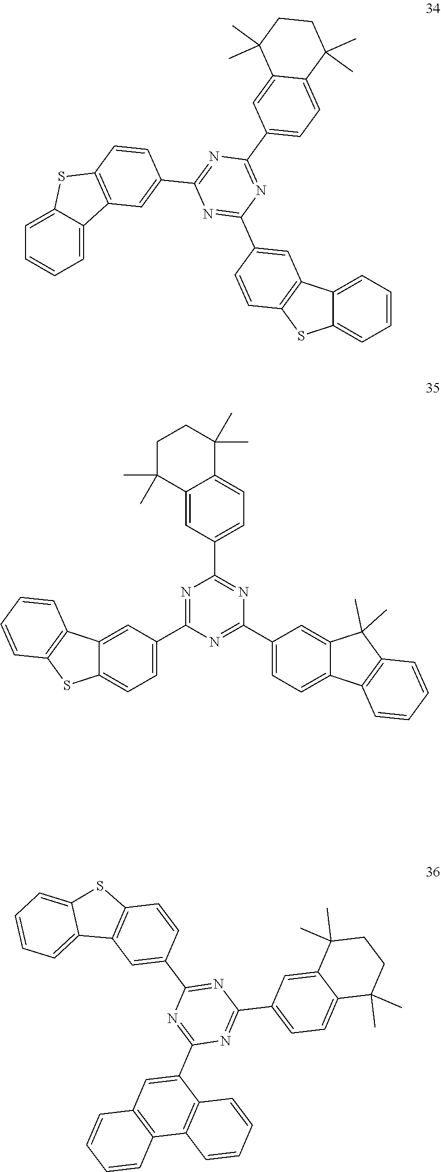 [SMILES: CC1(C)CCC(C)(C)c2cc(-c3nc(-c4ccc5sc6ccccc6c5c4)nc(-c4ccc5sc6ccccc6c5c4)n3)ccc21 ; CC1(C)CCC(C)(C)c2cc(-c3nc(-c4ccc5c(c4)C(C)(C)c4ccccc4-5)nc(-c4ccc5sc6ccccc6c5c4)n3)ccc21 ; CC1(C)CCC(C)(C)c2cc(-c3nc(-c4ccc5sc6ccccc6c5c4)nc(-c4cc5ccccc5c5ccccc45)n3)ccc21]

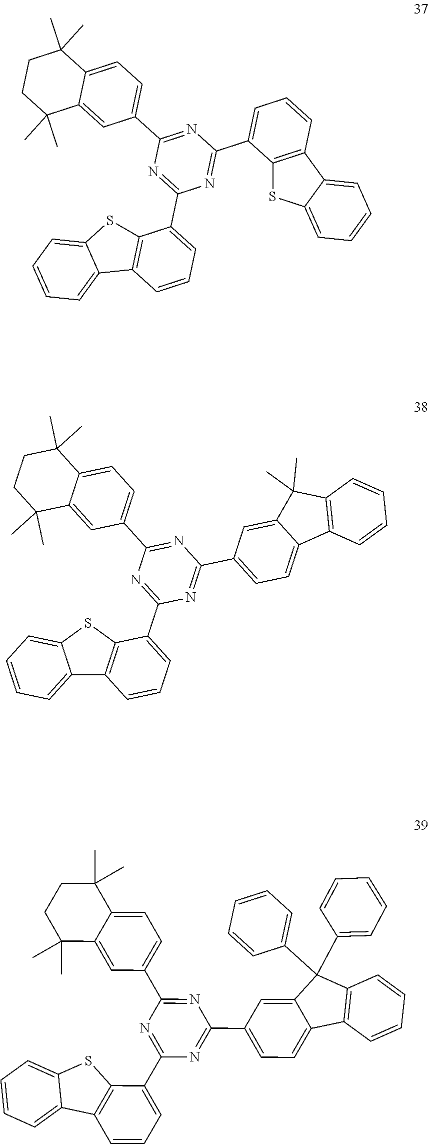 [SMILES: CC1(C)CCC(C)(C)c2cc(-c3nc(-c4cccc5c4sc4ccccc45)nc(-c4cccc5c4sc4ccccc45)n3)ccc21 ; CC1(C)CCC(C)(C)c2cc(-c3nc(-c4ccc5c(c4)C(C)(C)c4ccccc4-5)nc(-c4cccc5c4sc4ccccc45)n3)ccc21 ; CC1(C)CCC(C)(C)c2cc(-c3nc(-c4ccc5c(c4)C(c4ccccc4)(c4ccccc4)c4ccccc4-5)nc(-c4cccc5c4sc4ccccc45)n3)ccc21]

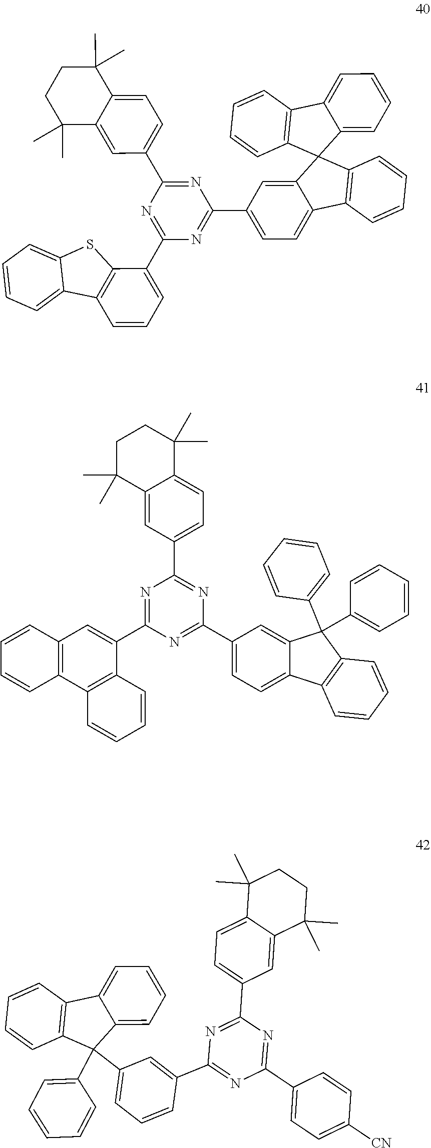 [SMILES: CC1(C)CCC(C)(C)c2cc(-c3nc(-c4ccc5c(c4)C4(c6ccccc6-c6ccccc64)c4ccccc4-5)nc(-c4cccc5c4sc4ccccc45)n3)ccc21 ; CC1(C)CCC(C)(C)c2cc(-c3nc(-c4ccc5c(c4)C(c4ccccc4)(c4ccccc4)c4ccccc4-5)nc(-c4cc5ccccc5c5ccccc45)n3)ccc21 ; CC1(C)CCC(C)(C)c2cc(-c3nc(-c4ccc(C#N)cc4)nc(-c4cccc(C5(c6ccccc6)c6ccccc6-c6ccccc65)c4)n3)ccc21]

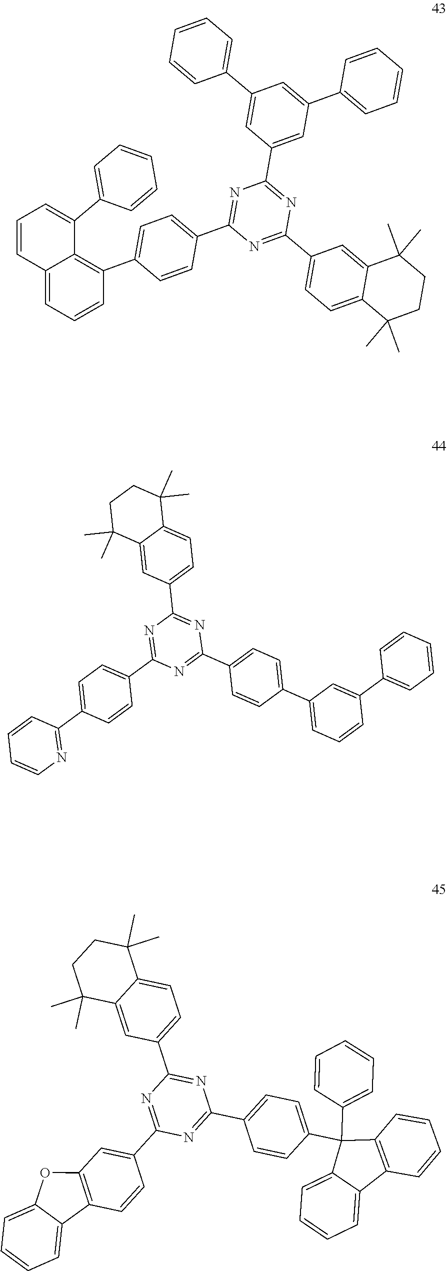 [SMILES: CC1(C)CCC(C)(C)c2cc(-c3nc(-c4ccc(-c5cccc6cccc(-c7ccccc7)c56)cc4)nc(-c4cc(-c5ccccc5)cc(-c5ccccc5)c4)n3)ccc21 ; CC1(C)CCC(C)(C)c2cc(-c3nc(-c4ccc(-c5cccc(-c6ccccc6)c5)cc4)nc(-c4ccc(-c5ccccn5)cc4)n3)ccc21 ; CC1(C)CCC(C)(C)c2cc(-c3nc(-c4ccc(C5(c6ccccc6)c6ccccc6-c6ccccc65)cc4)nc(-c4ccc5c(c4)oc4ccccc45)n3)ccc21]

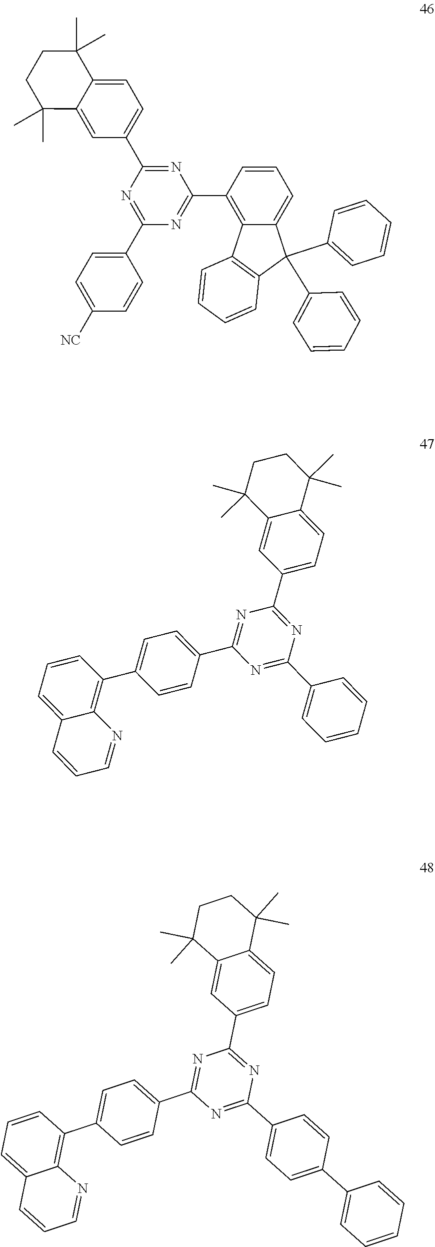 [SMILES: CC1(C)CCC(C)(C)c2cc(-c3nc(-c4ccc(C#N)cc4)nc(-c4cccc5c4-c4ccccc4C5(c4ccccc4)c4ccccc4)n3)ccc21 ; CC1(C)CCC(C)(C)c2cc(-c3nc(-c4ccccc4)nc(-c4ccc(-c5cccc6cccnc56)cc4)n3)ccc21 ; CC1(C)CCC(C)(C)c2cc(-c3nc(-c4ccc(-c5ccccc5)cc4)nc(-c4ccc(-c5cccc6cccnc56)cc4)n3)ccc21]

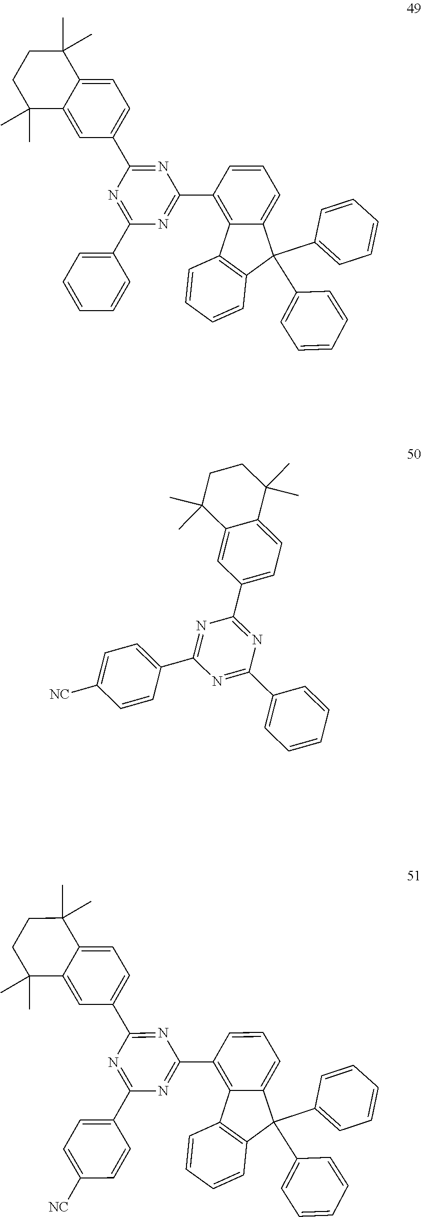 [SMILES: CC1(C)CCC(C)(C)c2cc(-c3nc(-c4ccccc4)nc(-c4cccc5c4-c4ccccc4C5(c4ccccc4)c4ccccc4)n3)ccc21 ; CC1(C)CCC(C)(C)c2cc(-c3nc(-c4ccccc4)nc(-c4ccc(C#N)cc4)n3)ccc21 ; CC1(C)CCC(C)(C)c2cc(-c3nc(-c4ccc(C#N)cc4)nc(-c4cccc5c4-c4ccccc4C5(c4ccccc4)c4ccccc4)n3)ccc21]

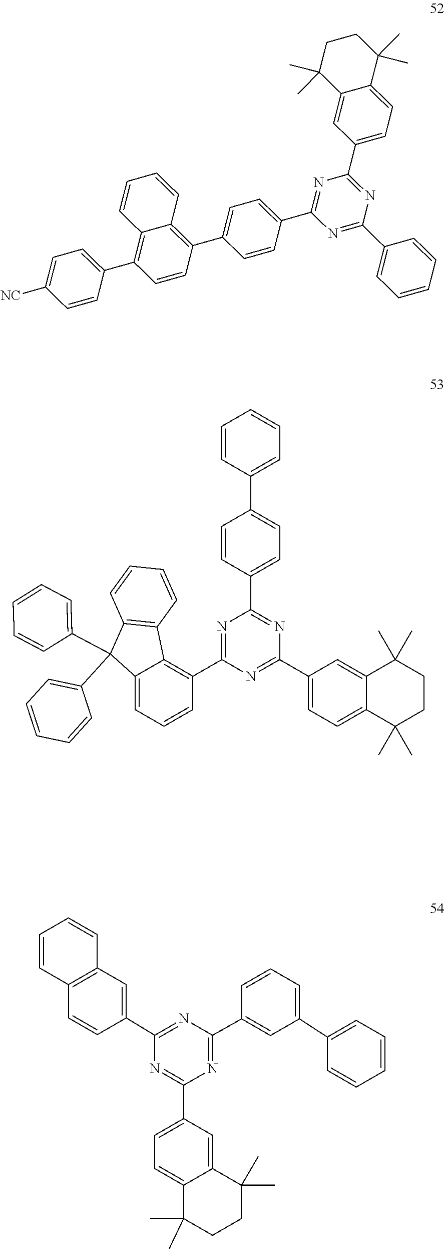 [SMILES: CC1(C)CCC(C)(C)c2cc(-c3nc(-c4ccccc4)nc(-c4ccc(-c5ccc(-c6ccc(C#N)cc6)c6ccccc56)cc4)n3)ccc21 ; CC1(C)CCC(C)(C)c2cc(-c3nc(-c4ccc(-c5ccccc5)cc4)nc(-c4cccc5c4-c4ccccc4C5(c4ccccc4)c4ccccc4)n3)ccc21 ; CC1(C)CCC(C)(C)c2cc(-c3nc(-c4cccc(-c5ccccc5)c4)nc(-c4ccc5ccccc5c4)n3)ccc21]

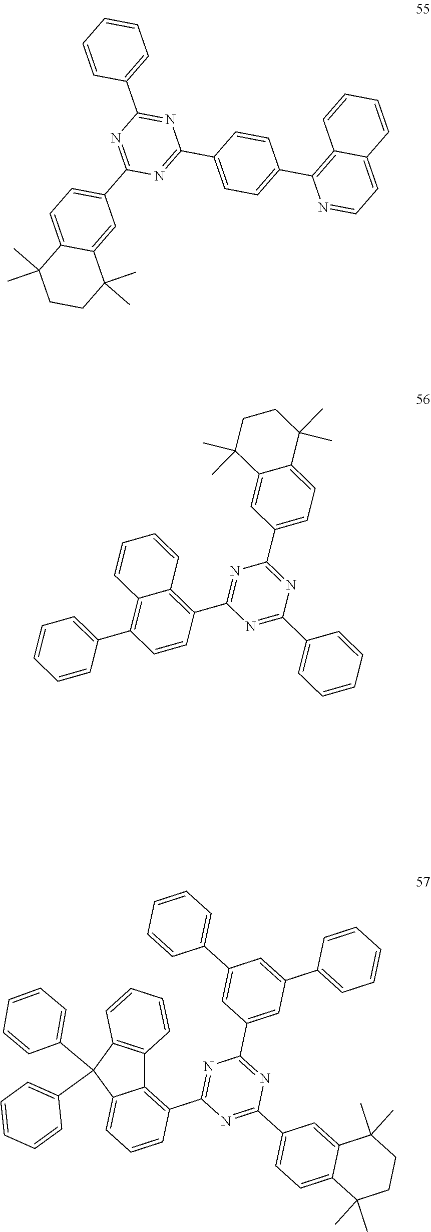 [SMILES: CC1(C)CCC(C)(C)c2cc(-c3nc(-c4ccccc4)nc(-c4ccc(-c5nccc6ccccc56)cc4)n3)ccc21 ; CC1(C)CCC(C)(C)c2cc(-c3nc(-c4ccccc4)nc(-c4ccc(-c5ccccc5)c5ccccc45)n3)ccc21 ; CC1(C)CCC(C)(C)c2cc(-c3nc(-c4cc(-c5ccccc5)cc(-c5ccccc5)c4)nc(-c4cccc5c4-c4ccccc4C5(c4ccccc4)c4ccccc4)n3)ccc21]

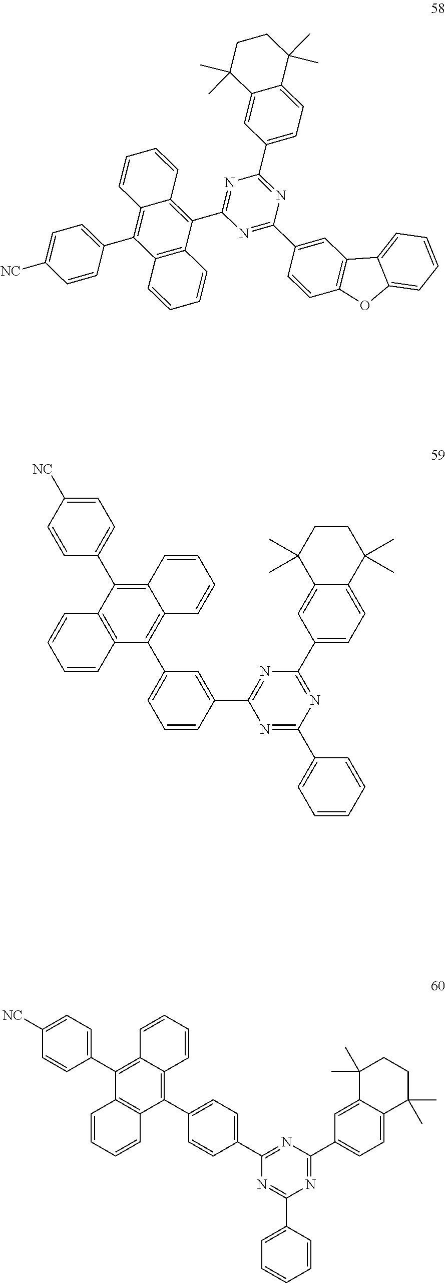 [SMILES: CC1(C)CCC(C)(C)c2cc(-c3nc(-c4ccc5oc6ccccc6c5c4)nc(-c4c5ccccc5c(-c5ccc(C#N)cc5)c5ccccc45)n3)ccc21 ; CC1(C)CCC(C)(C)c2cc(-c3nc(-c4ccccc4)nc(-c4cccc(-c5c6ccccc6c(-c6ccc(C#N)cc6)c6ccccc56)c4)n3)ccc21 ; CC1(C)CCC(C)(C)c2cc(-c3nc(-c4ccccc4)nc(-c4ccc(-c5c6ccccc6c(-c6ccc(C#N)cc6)c6ccccc56)cc4)n3)ccc21]

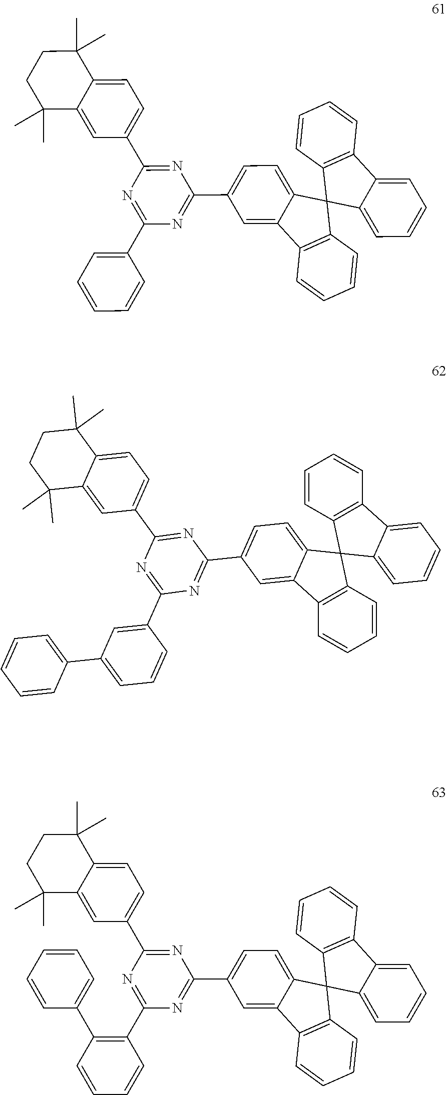 [SMILES: CC1(C)CCC(C)(C)c2cc(-c3nc(-c4ccccc4)nc(-c4ccc5c(c4)-c4ccccc4C54c5ccccc5-c5ccccc54)n3)ccc21 ; CC1(C)CCC(C)(C)c2cc(-c3nc(-c4cccc(-c5ccccc5)c4)nc(-c4ccc5c(c4)-c4ccccc4C54c5ccccc5-c5ccccc54)n3)ccc21 ; CC1(C)CCC(C)(C)c2cc(-c3nc(-c4ccc5c(c4)-c4ccccc4C54c5ccccc5-c5ccccc54)nc(-c4ccccc4-c4ccccc4)n3)ccc21]

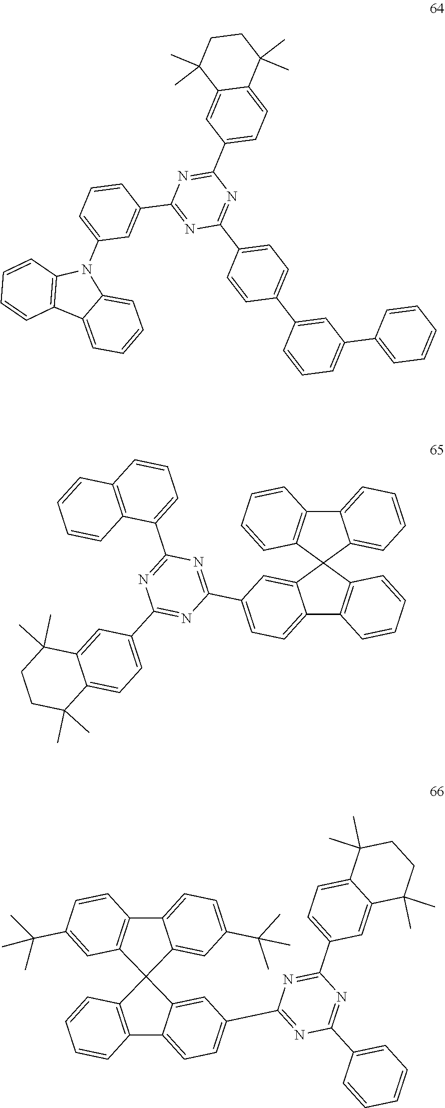 [SMILES: CC1(C)CCC(C)(C)c2cc(-c3nc(-c4ccc(-c5cccc(-c6ccccc6)c5)cc4)nc(-c4cccc(-n5c6ccccc6c6ccccc65)c4)n3)ccc21 ; CC1(C)CCC(C)(C)c2cc(-c3nc(-c4ccc5c(c4)C4(c6ccccc6-c6ccccc64)c4ccccc4-5)nc(-c4cccc5ccccc45)n3)ccc21 ; CC(C)(C)c1ccc2c(c1)C1(c3ccccc3-c3ccc(-c4nc(-c5ccccc5)nc(-c5ccc6c(c5)C(C)(C)CCC6(C)C)n4)cc31)c1cc(C(C)(C)C)ccc1-2]

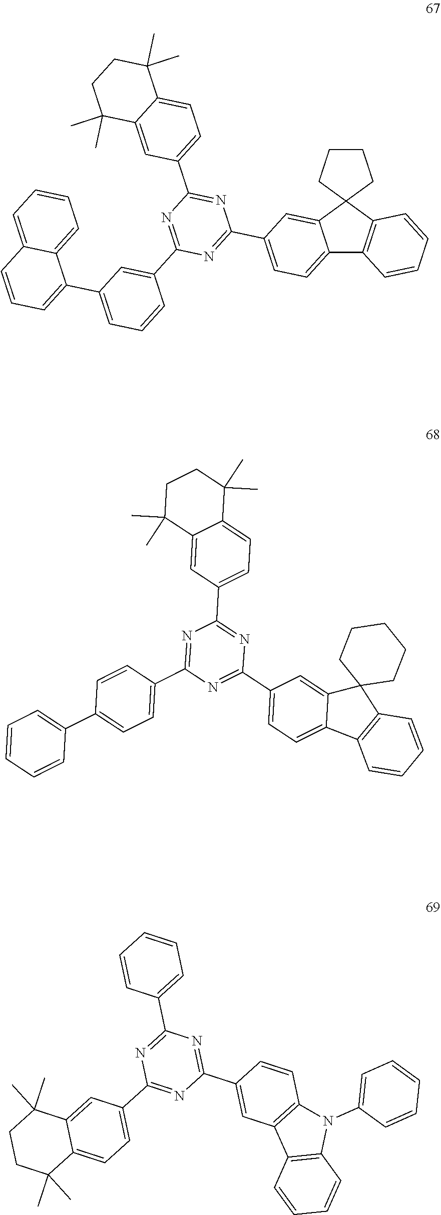 [SMILES: CC1(C)CCC(C)(C)c2cc(-c3nc(-c4cccc(-c5cccc6ccccc56)c4)nc(-c4ccc5c(c4)C4(CCCC4)c4ccccc4-5)n3)ccc21 ; CC1(C)CCC(C)(C)c2cc(-c3nc(-c4ccc(-c5ccccc5)cc4)nc(-c4ccc5c(c4)C4(CCCCC4)c4ccccc4-5)n3)ccc21 ; CC1(C)CCC(C)(C)c2cc(-c3nc(-c4ccccc4)nc(-c4ccc5c(c4)c4ccccc4n5-c4ccccc4)n3)ccc21]

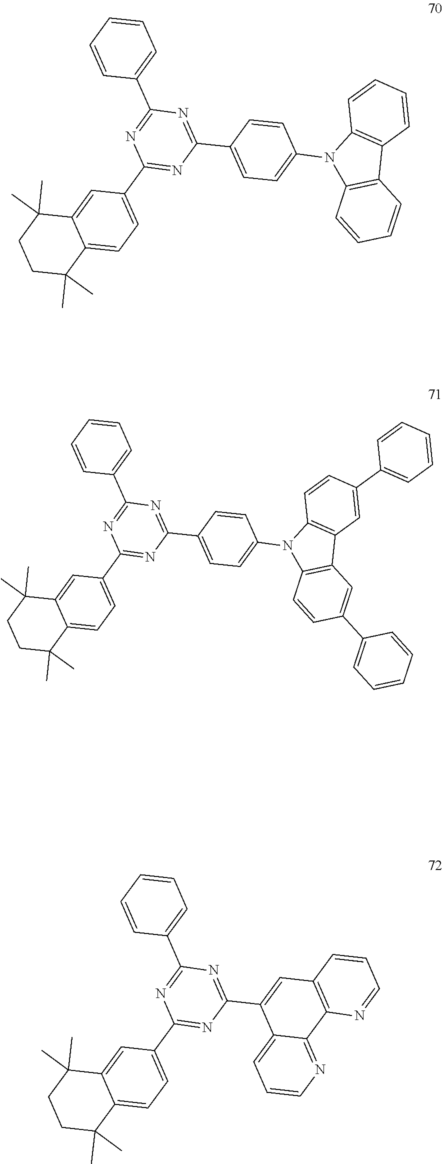 [SMILES: CC1(C)CCC(C)(C)c2cc(-c3nc(-c4ccccc4)nc(-c4ccc(-n5c6ccccc6c6ccccc65)cc4)n3)ccc21 ; CC1(C)CCC(C)(C)c2cc(-c3nc(-c4ccccc4)nc(-c4ccc(-n5c6ccc(-c7ccccc7)cc6c6cc(-c7ccccc7)ccc65)cc4)n3)ccc21 ; CC1(C)CCC(C)(C)c2cc(-c3nc(-c4ccccc4)nc(-c4cc5cccnc5c5ncccc45)n3)ccc21]

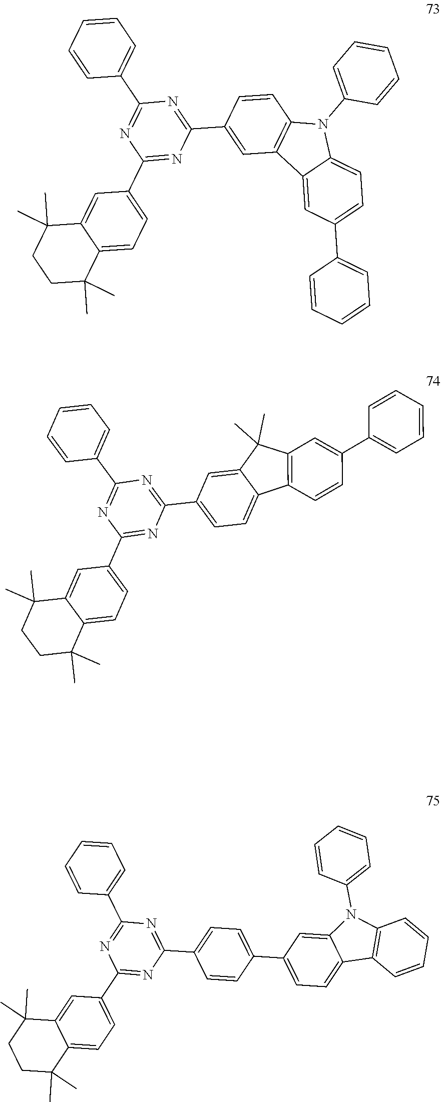 [SMILES: CC1(C)CCC(C)(C)c2cc(-c3nc(-c4ccccc4)nc(-c4ccc5c(c4)c4cc(-c6ccccc6)ccc4n5-c4ccccc4)n3)ccc21 ; CC1(C)CCC(C)(C)c2cc(-c3nc(-c4ccccc4)nc(-c4ccc5c(c4)C(C)(C)c4cc(-c6ccccc6)ccc4-5)n3)ccc21 ; CC1(C)CCC(C)(C)c2cc(-c3nc(-c4ccccc4)nc(-c4ccc(-c5ccc6c7ccccc7n(-c7ccccc7)c6c5)cc4)n3)ccc21]

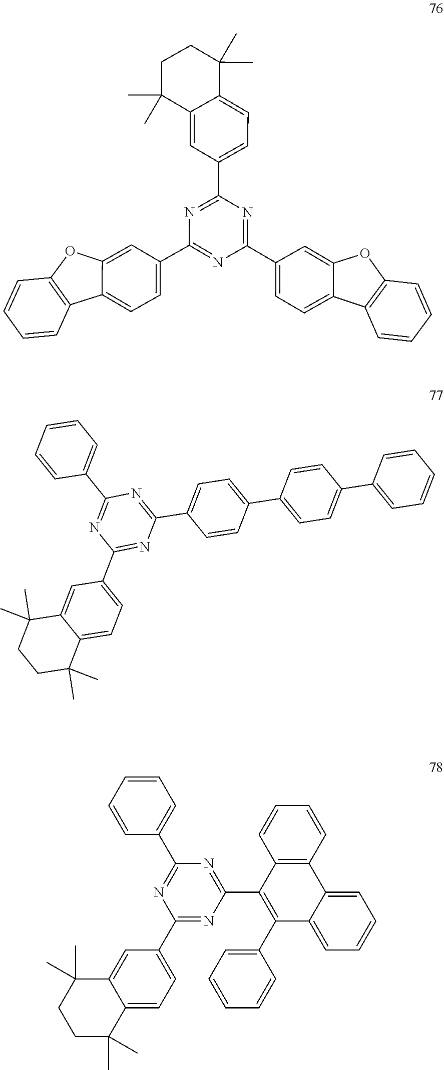 [SMILES: CC1(C)CCC(C)(C)c2cc(-c3nc(-c4ccc5c(c4)oc4ccccc45)nc(-c4ccc5c(c4)oc4ccccc45)n3)ccc21 ; CC1(C)CCC(C)(C)c2cc(-c3nc(-c4ccccc4)nc(-c4ccc(-c5ccc(-c6ccccc6)cc5)cc4)n3)ccc21 ; CC1(C)CCC(C)(C)c2cc(-c3nc(-c4ccccc4)nc(-c4c(-c5ccccc5)c5ccccc5c5ccccc45)n3)ccc21]

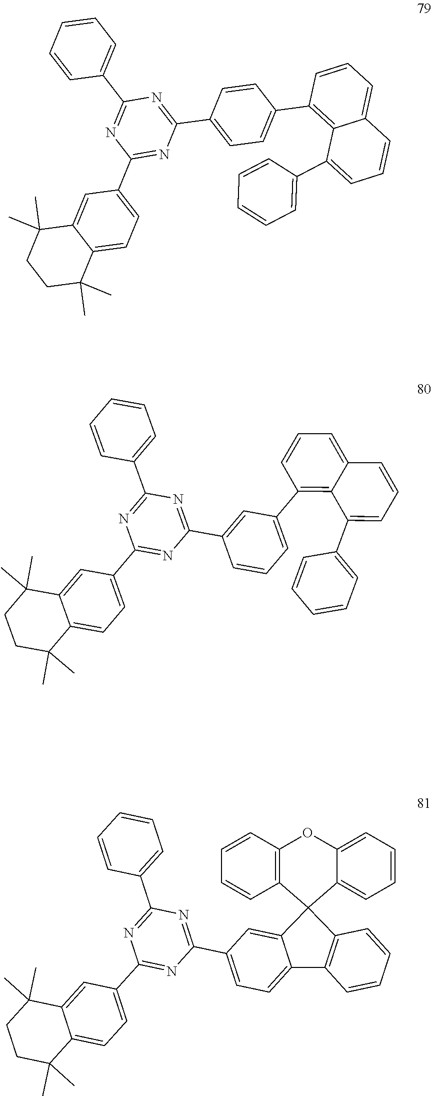 [SMILES: CC1(C)CCC(C)(C)c2cc(-c3nc(-c4ccccc4)nc(-c4ccc(-c5cccc6cccc(-c7ccccc7)c56)cc4)n3)ccc21 ; CC1(C)CCC(C)(C)c2cc(-c3nc(-c4ccccc4)nc(-c4cccc(-c5cccc6cccc(-c7ccccc7)c56)c4)n3)ccc21 ; CC1(C)CCC(C)(C)c2cc(-c3nc(-c4ccccc4)nc(-c4ccc5c(c4)C4(c6ccccc6Oc6ccccc64)c4ccccc4-5)n3)ccc21]

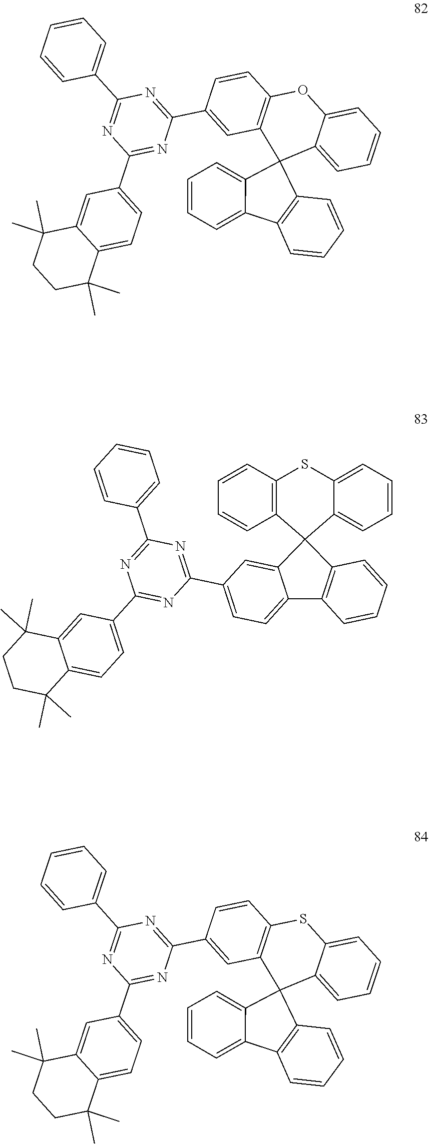 [SMILES: CC1(C)CCC(C)(C)c2cc(-c3nc(-c4ccccc4)nc(-c4ccc5c(c4)C4(c6ccccc6O5)c5ccccc5-c5ccccc54)n3)ccc21 ; CC1(C)CCC(C)(C)c2cc(-c3nc(-c4ccccc4)nc(-c4ccc5c(c4)C4(c6ccccc6Sc6ccccc64)c4ccccc4-5)n3)ccc21 ; CC1(C)CCC(C)(C)c2cc(-c3nc(-c4ccccc4)nc(-c4ccc5c(c4)C4(c6ccccc6S5)c5ccccc5-c5ccccc54)n3)ccc21]

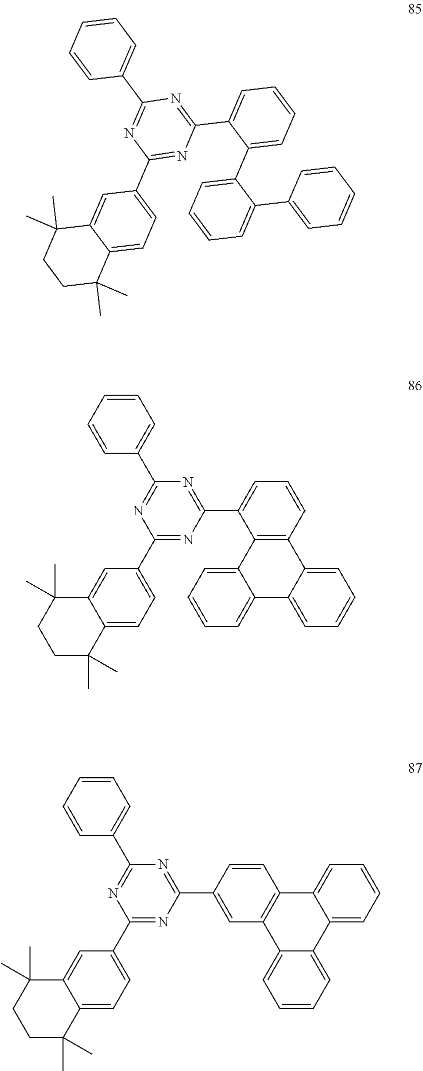 [SMILES: CC1(C)CCC(C)(C)c2cc(-c3nc(-c4ccccc4)nc(-c4ccccc4-c4ccccc4-c4ccccc4)n3)ccc21 ; CC1(C)CCC(C)(C)c2cc(-c3nc(-c4ccccc4)nc(-c4cccc5c6ccccc6c6ccccc6c45)n3)ccc21 ; CC1(C)CCC(C)(C)c2cc(-c3nc(-c4ccccc4)nc(-c4ccc5c6ccccc6c6ccccc6c5c4)n3)ccc21]

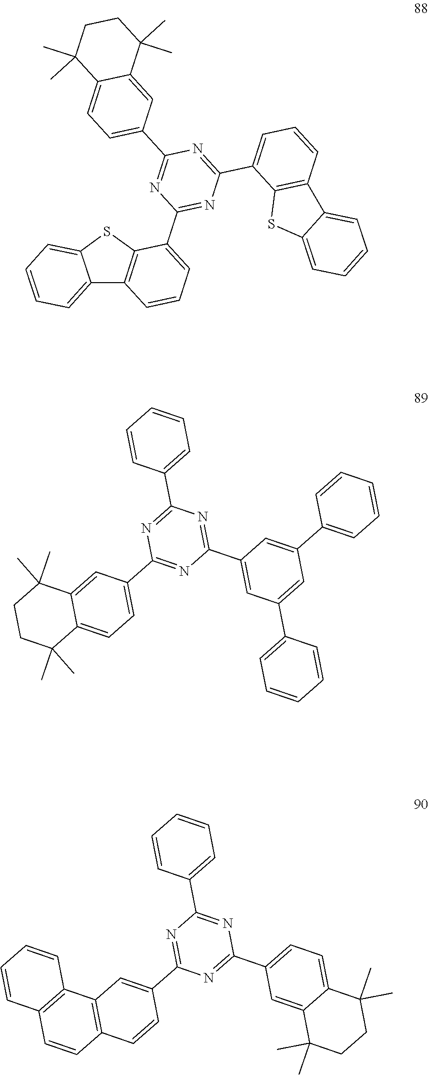 [SMILES: CC1(C)CCC(C)(C)c2cc(-c3nc(-c4cccc5c4sc4ccccc45)nc(-c4cccc5c4sc4ccccc45)n3)ccc21 ; CC1(C)CCC(C)(C)c2cc(-c3nc(-c4ccccc4)nc(-c4cc(-c5ccccc5)cc(-c5ccccc5)c4)n3)ccc21 ; CC1(C)CCC(C)(C)c2cc(-c3nc(-c4ccccc4)nc(-c4ccc5ccc6ccccc6c5c4)n3)ccc21]

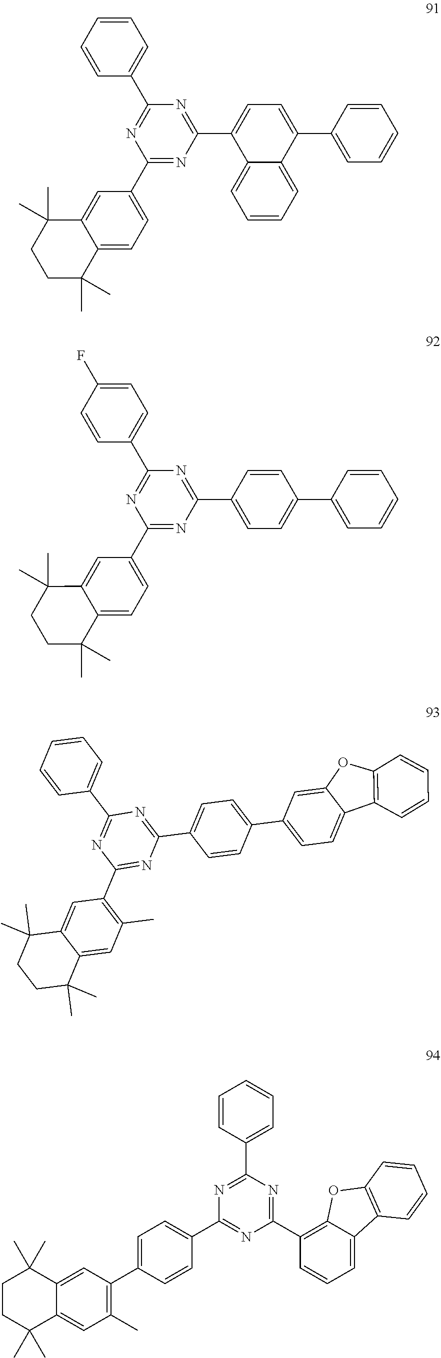 [SMILES: CC1(C)CCC(C)(C)c2cc(-c3nc(-c4ccccc4)nc(-c4ccc(-c5ccccc5)c5ccccc45)n3)ccc21 ; CC1(C)CCC(C)(C)c2cc(-c3nc(-c4ccc(F)cc4)nc(-c4ccc(-c5ccccc5)cc4)n3)ccc21 ; Cc1cc2c(cc1-c1nc(-c3ccccc3)nc(-c3ccc(-c4ccc5c(c4)oc4ccccc45)cc3)n1)C(C)(C)CCC2(C)C ; Cc1cc2c(cc1-c1ccc(-c3nc(-c4ccccc4)nc(-c4cccc5c4oc4ccccc45)n3)cc1)C(C)(C)CCC2(C)C]

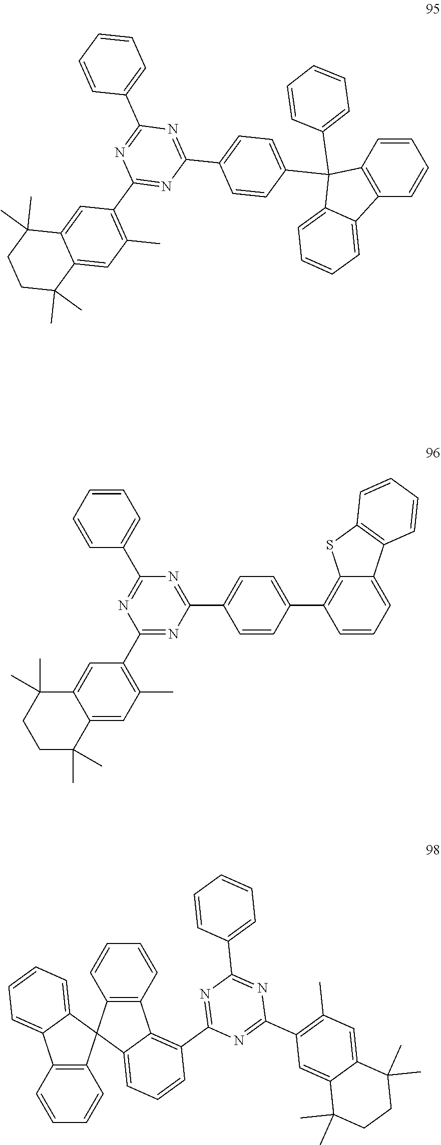 [SMILES: Cc1cc2c(cc1-c1nc(-c3ccccc3)nc(-c3ccc(C4(c5ccccc5)c5ccccc5-c5ccccc54)cc3)n1)C(C)(C)CCC2(C)C ; Cc1cc2c(cc1-c1nc(-c3ccccc3)nc(-c3ccc(-c4cccc5c4sc4ccccc45)cc3)n1)C(C)(C)CCC2(C)C ; Cc1cc2c(cc1-c1nc(-c3ccccc3)nc(-c3cccc4c3-c3ccccc3C43c4ccccc4-c4ccccc43)n1)C(C)(C)CCC2(C)C]

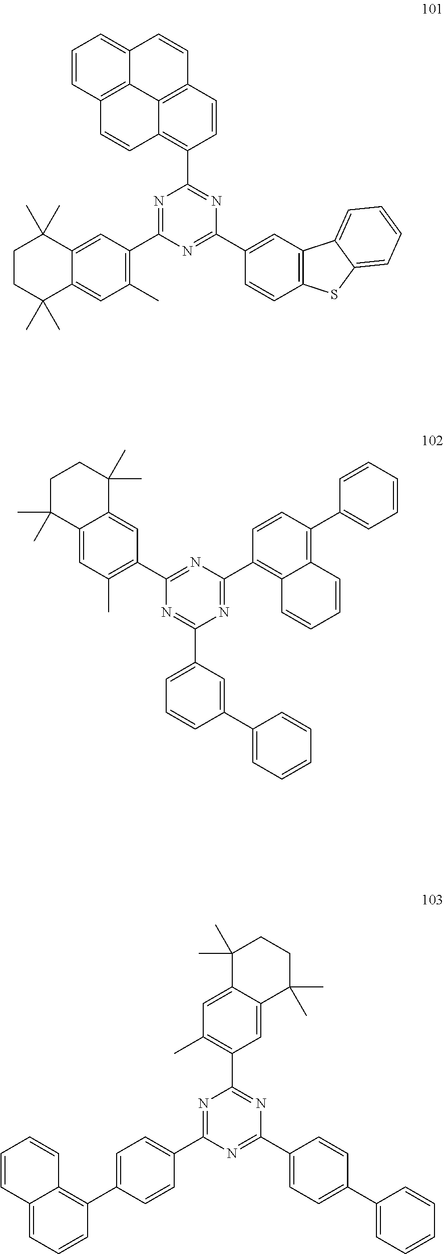 [SMILES: Cc1cc2c(cc1-c1nc(-c3ccc4sc5ccccc5c4c3)nc(-c3ccc4ccc5cccc6ccc3c4c56)n1)C(C)(C)CCC2(C)C ; Cc1cc2c(cc1-c1nc(-c3cccc(-c4ccccc4)c3)nc(-c3ccc(-c4ccccc4)c4ccccc34)n1)C(C)(C)CCC2(C)C ; Cc1cc2c(cc1-c1nc(-c3ccc(-c4ccccc4)cc3)nc(-c3ccc(-c4cccc5ccccc45)cc3)n1)C(C)(C)CCC2(C)C]

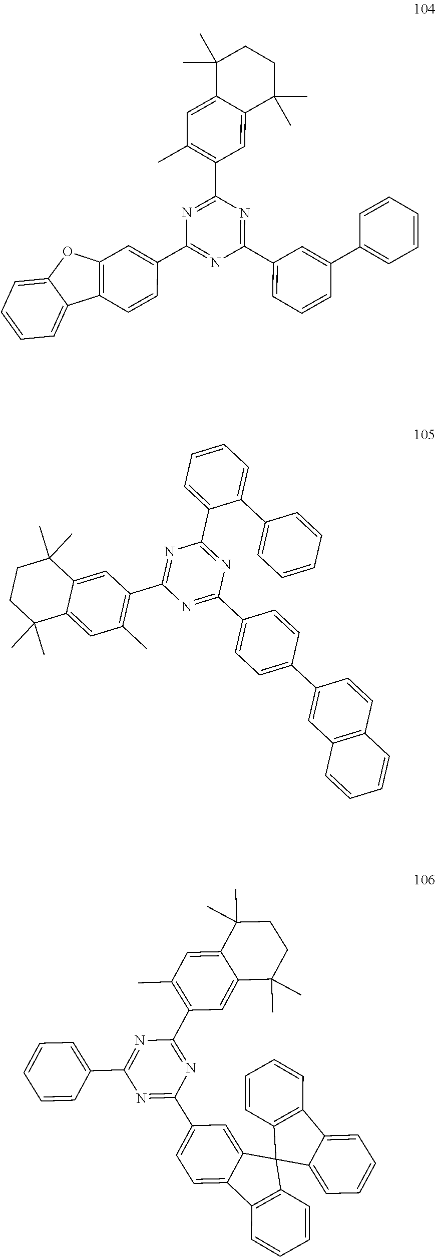 [SMILES: Cc1cc2c(cc1-c1nc(-c3cccc(-c4ccccc4)c3)nc(-c3ccc4c(c3)oc3ccccc34)n1)C(C)(C)CCC2(C)C ; Cc1cc2c(cc1-c1nc(-c3ccc(-c4ccc5ccccc5c4)cc3)nc(-c3ccccc3-c3ccccc3)n1)C(C)(C)CCC2(C)C ; Cc1cc2c(cc1-c1nc(-c3ccccc3)nc(-c3ccc4c(c3)C3(c5ccccc5-c5ccccc53)c3ccccc3-4)n1)C(C)(C)CCC2(C)C]

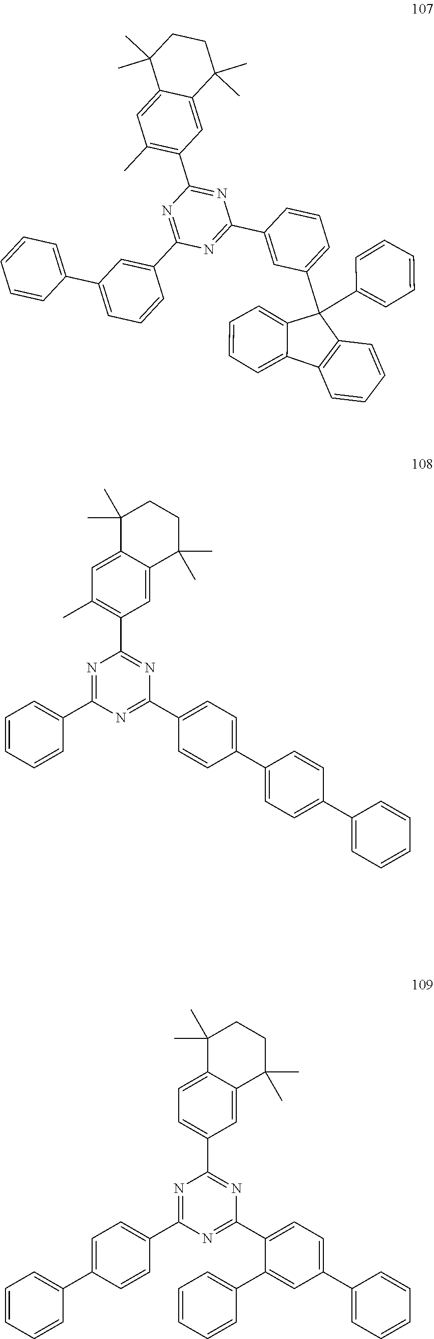 [SMILES: Cc1cc2c(cc1-c1nc(-c3cccc(-c4ccccc4)c3)nc(-c3cccc(C4(c5ccccc5)c5ccccc5-c5ccccc54)c3)n1)C(C)(C)CCC2(C)C ; Cc1cc2c(cc1-c1nc(-c3ccccc3)nc(-c3ccc(-c4ccc(-c5ccccc5)cc4)cc3)n1)C(C)(C)CCC2(C)C ; CC1(C)CCC(C)(C)c2cc(-c3nc(-c4ccc(-c5ccccc5)cc4)nc(-c4ccc(-c5ccccc5)cc4-c4ccccc4)n3)ccc21]

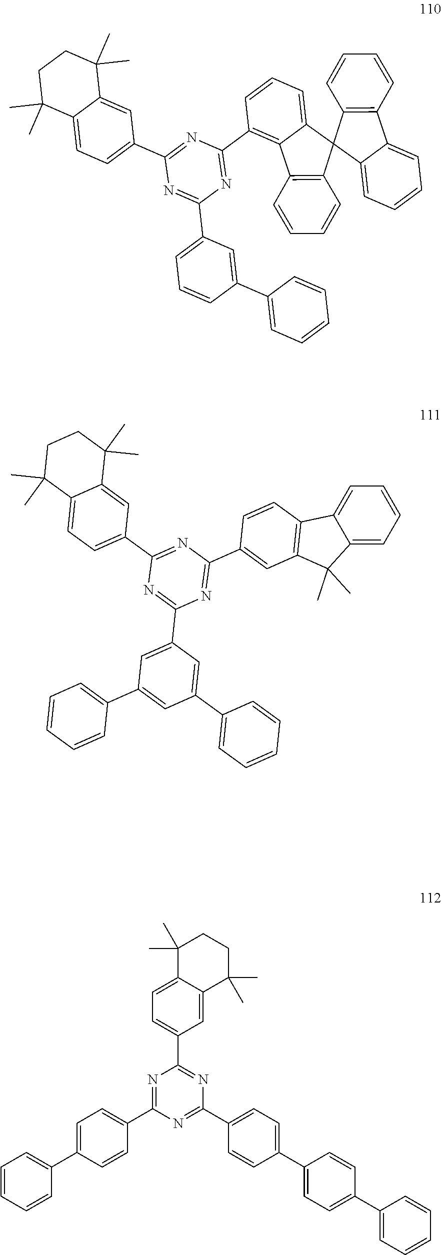 [SMILES: CC1(C)CCC(C)(C)c2cc(-c3nc(-c4cccc(-c5ccccc5)c4)nc(-c4cccc5c4-c4ccccc4C54c5ccccc5-c5ccccc54)n3)ccc21 ; CC1(C)CCC(C)(C)c2cc(-c3nc(-c4cc(-c5ccccc5)cc(-c5ccccc5)c4)nc(-c4ccc5c(c4)C(C)(C)c4ccccc4-5)n3)ccc21 ; CC1(C)CCC(C)(C)c2cc(-c3nc(-c4ccc(-c5ccccc5)cc4)nc(-c4ccc(-c5ccc(-c6ccccc6)cc5)cc4)n3)ccc21]

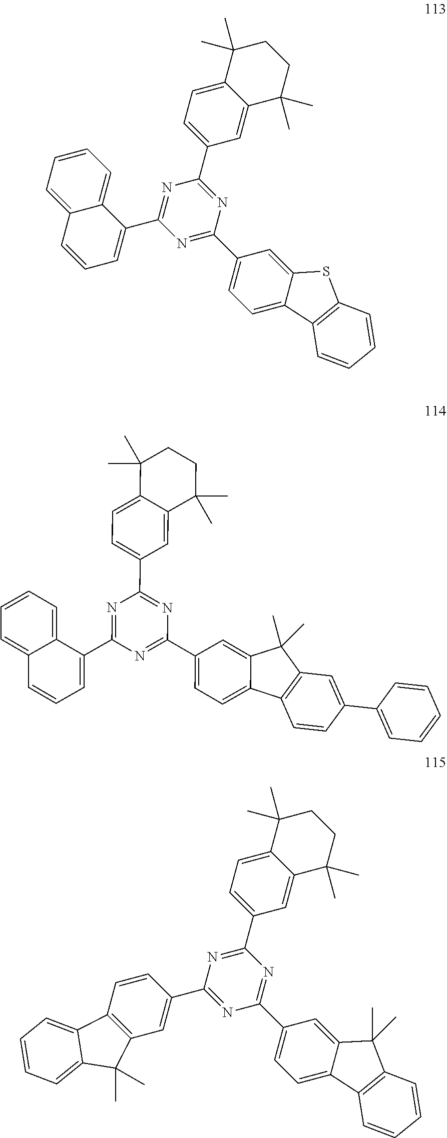 [SMILES: CC1(C)CCC(C)(C)c2cc(-c3nc(-c4ccc5c(c4)sc4ccccc45)nc(-c4cccc5ccccc45)n3)ccc21 ; CC1(C)CCC(C)(C)c2cc(-c3nc(-c4ccc5c(c4)C(C)(C)c4cc(-c6ccccc6)ccc4-5)nc(-c4cccc5ccccc45)n3)ccc21 ; CC1(C)CCC(C)(C)c2cc(-c3nc(-c4ccc5c(c4)C(C)(C)c4ccccc4-5)nc(-c4ccc5c(c4)C(C)(C)c4ccccc4-5)n3)ccc21]

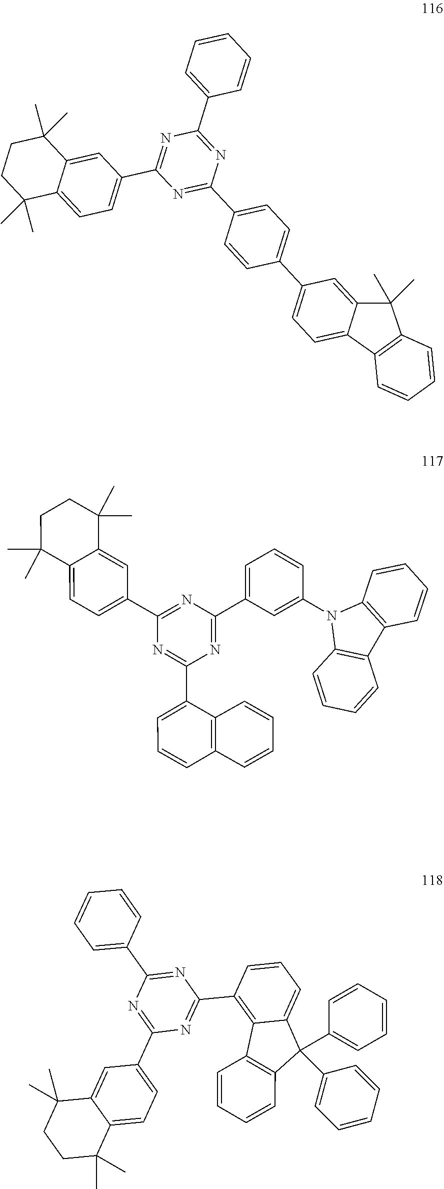 [SMILES: CC1(C)CCC(C)(C)c2cc(-c3nc(-c4ccccc4)nc(-c4ccc(-c5ccc6c(c5)C(C)(C)c5ccccc5-6)cc4)n3)ccc21 ; CC1(C)CCC(C)(C)c2cc(-c3nc(-c4cccc(-n5c6ccccc6c6ccccc65)c4)nc(-c4cccc5ccccc45)n3)ccc21 ; CC1(C)CCC(C)(C)c2cc(-c3nc(-c4ccccc4)nc(-c4cccc5c4-c4ccccc4C5(c4ccccc4)c4ccccc4)n3)ccc21]

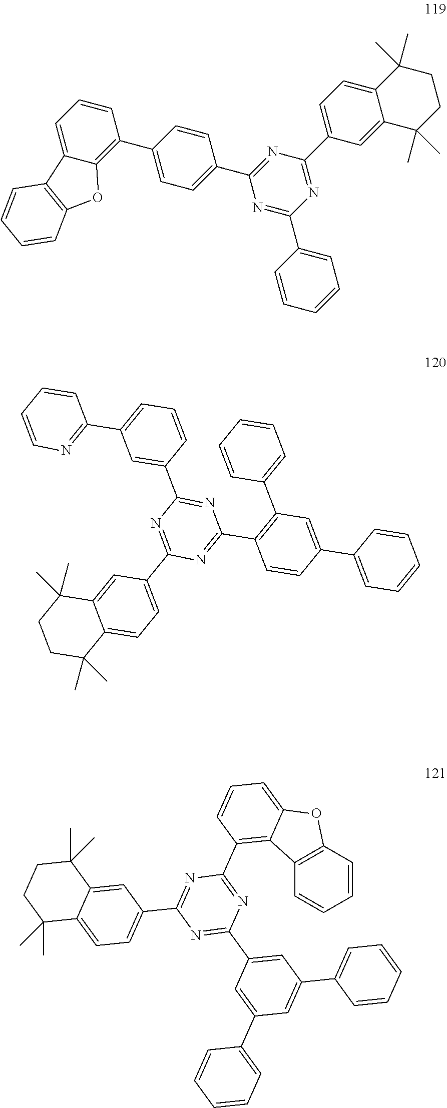

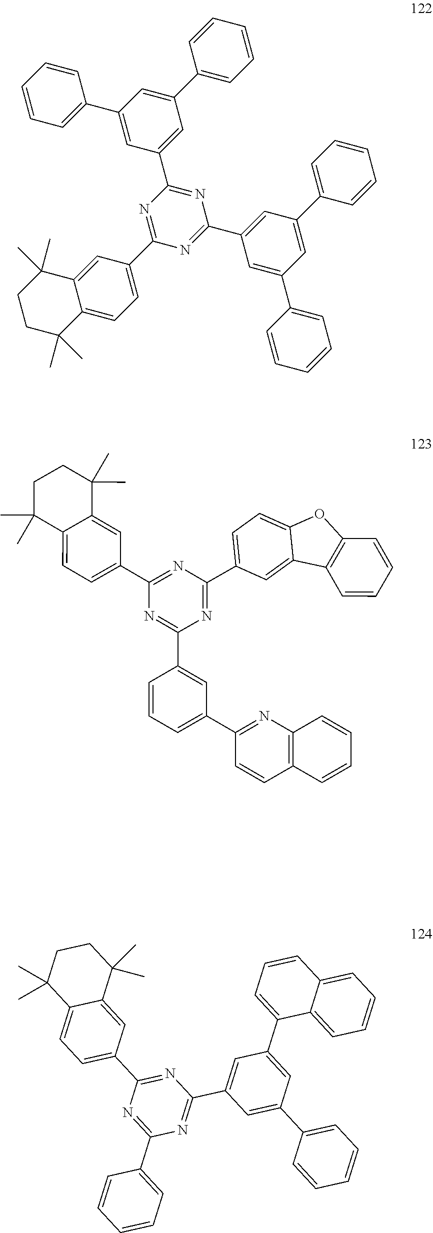 [SMILES: CC1(C)CCC(C)(C)c2cc(-c3nc(-c4cc(-c5ccccc5)cc(-c5ccccc5)c4)nc(-c4cc(-c5ccccc5)cc(-c5ccccc5)c4)n3)ccc21 ; CC1(C)CCC(C)(C)c2cc(-c3nc(-c4cccc(-c5ccc6ccccc6n5)c4)nc(-c4ccc5oc6ccccc6c5c4)n3)ccc21 ; CC1(C)CCC(C)(C)c2cc(-c3nc(-c4ccccc4)nc(-c4cc(-c5ccccc5)cc(-c5cccc6ccccc56)c4)n3)ccc21]

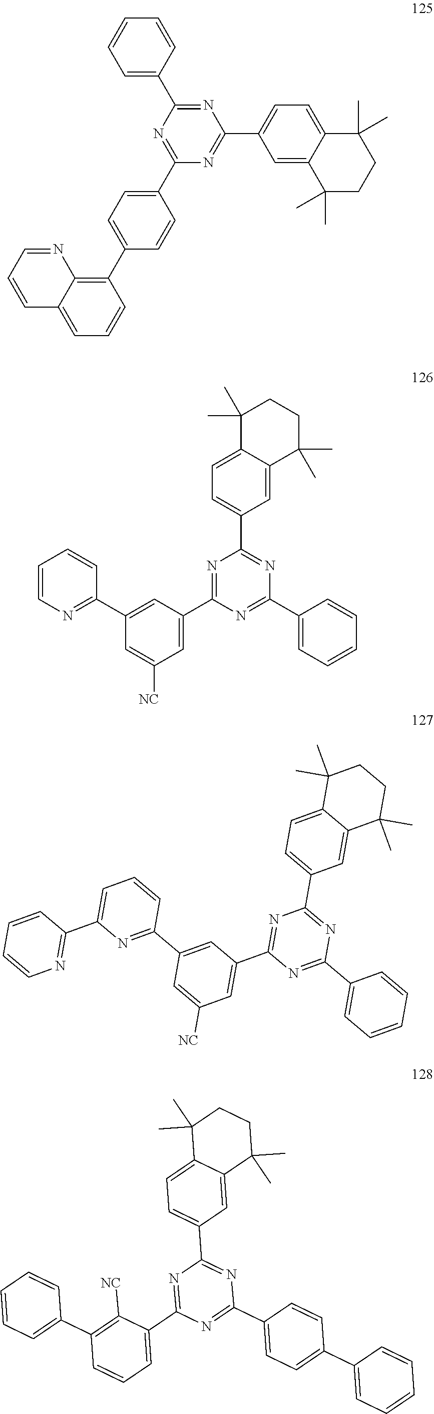 [SMILES: CC1(C)CCC(C)(C)c2cc(-c3nc(-c4ccccc4)nc(-c4ccc(-c5cccc6cccnc56)cc4)n3)ccc21 ; CC1(C)CCC(C)(C)c2cc(-c3nc(-c4ccccc4)nc(-c4cc(C#N)cc(-c5ccccn5)c4)n3)ccc21 ; CC1(C)CCC(C)(C)c2cc(-c3nc(-c4ccccc4)nc(-c4cc(C#N)cc(-c5cccc(-c6ccccn6)n5)c4)n3)ccc21 ; CC1(C)CCC(C)(C)c2cc(-c3nc(-c4ccc(-c5ccccc5)cc4)nc(-c4cccc(-c5ccccc5)c4C#N)n3)ccc21]

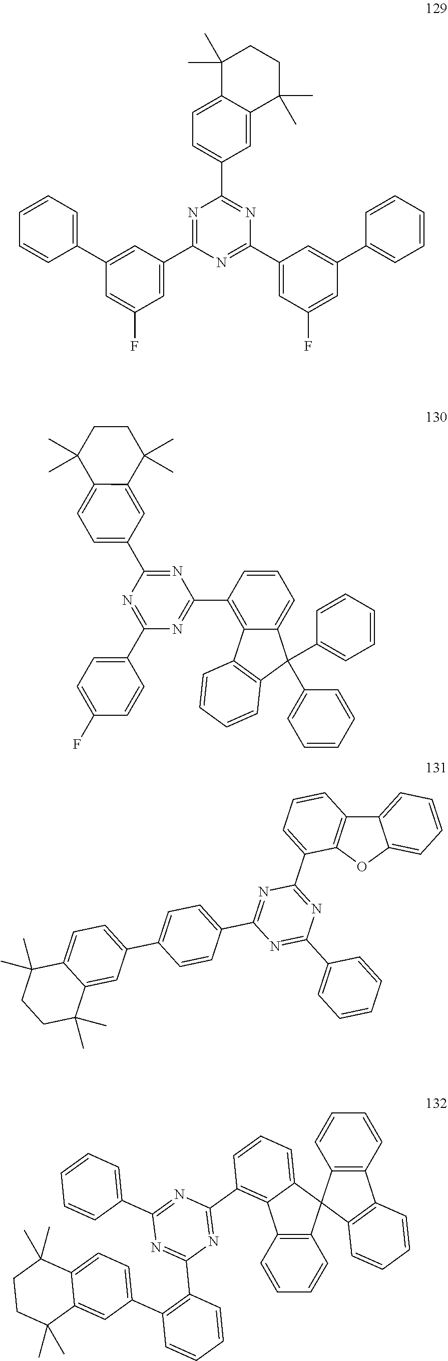 [SMILES: CC1(C)CCC(C)(C)c2cc(-c3nc(-c4cc(F)cc(-c5ccccc5)c4)nc(-c4cc(F)cc(-c5ccccc5)c4)n3)ccc21 ; CC1(C)CCC(C)(C)c2cc(-c3nc(-c4ccc(F)cc4)nc(-c4cccc5c4-c4ccccc4C5(c4ccccc4)c4ccccc4)n3)ccc21 ; CC1(C)CCC(C)(C)c2cc(-c3ccc(-c4nc(-c5ccccc5)nc(-c5cccc6c5oc5ccccc56)n4)cc3)ccc21 ; CC1(C)CCC(C)(C)c2cc(-c3ccccc3-c3nc(-c4ccccc4)nc(-c4cccc5c4-c4ccccc4C54c5ccccc5-c5ccccc54)n3)ccc21]

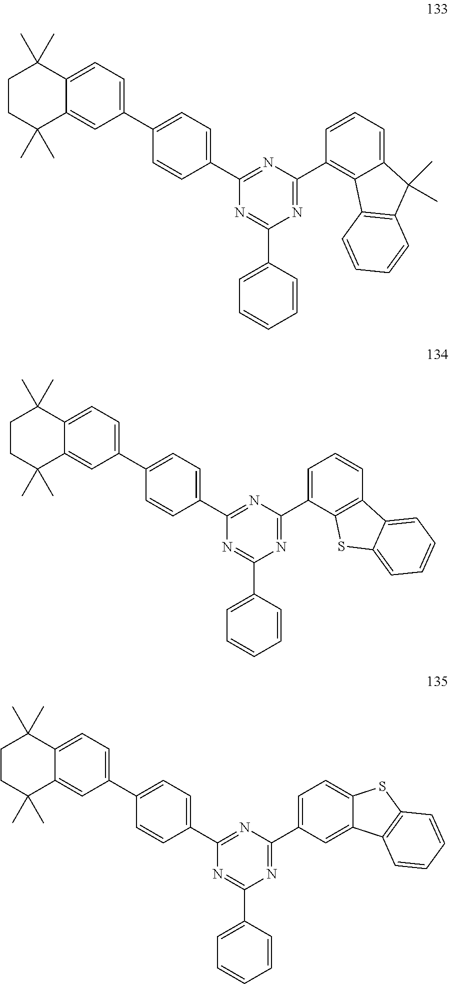 [SMILES: CC1(C)CCC(C)(C)c2cc(-c3ccc(-c4nc(-c5ccccc5)nc(-c5cccc6c5-c5ccccc5C6(C)C)n4)cc3)ccc21 ; CC1(C)CCC(C)(C)c2cc(-c3ccc(-c4nc(-c5ccccc5)nc(-c5cccc6c5sc5ccccc56)n4)cc3)ccc21 ; CC1(C)CCC(C)(C)c2cc(-c3ccc(-c4nc(-c5ccccc5)nc(-c5ccc6sc7ccccc7c6c5)n4)cc3)ccc21]

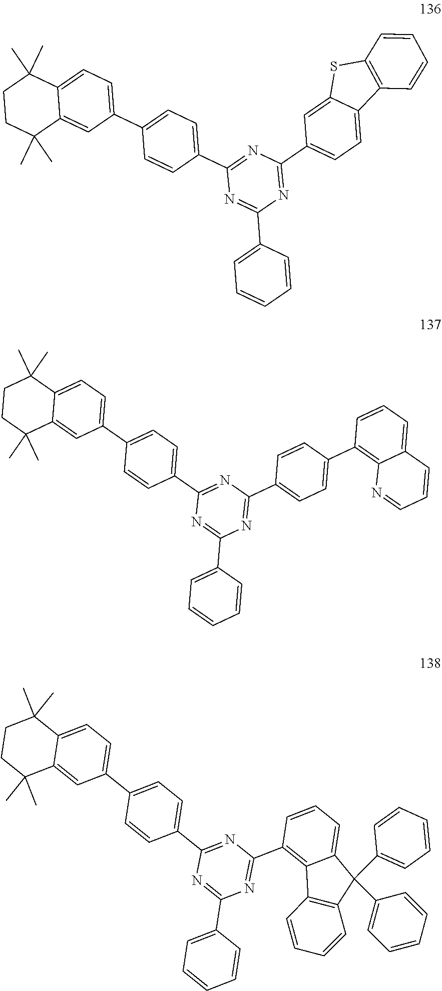 [SMILES: CC1(C)CCC(C)(C)c2cc(-c3ccc(-c4nc(-c5ccccc5)nc(-c5ccc6c(c5)sc5ccccc56)n4)cc3)ccc21 ; CC1(C)CCC(C)(C)c2cc(-c3ccc(-c4nc(-c5ccccc5)nc(-c5ccc(-c6cccc7cccnc67)cc5)n4)cc3)ccc21 ; CC1(C)CCC(C)(C)c2cc(-c3ccc(-c4nc(-c5ccccc5)nc(-c5cccc6c5-c5ccccc5C6(c5ccccc5)c5ccccc5)n4)cc3)ccc21]

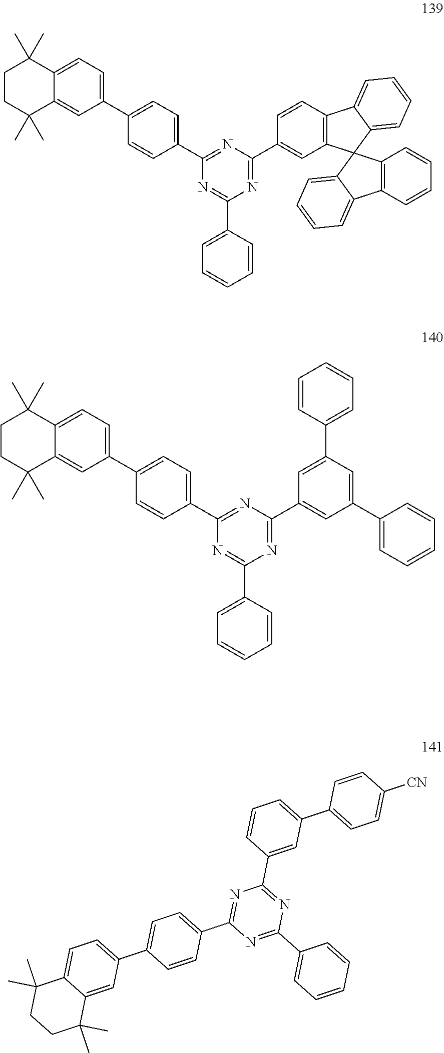 [SMILES: CC1(C)CCC(C)(C)c2cc(-c3ccc(-c4nc(-c5ccccc5)nc(-c5ccc6c(c5)C5(c7ccccc7-c7ccccc75)c5ccccc5-6)n4)cc3)ccc21 ; CC1(C)CCC(C)(C)c2cc(-c3ccc(-c4nc(-c5ccccc5)nc(-c5cc(-c6ccccc6)cc(-c6ccccc6)c5)n4)cc3)ccc21 ; CC1(C)CCC(C)(C)c2cc(-c3ccc(-c4nc(-c5ccccc5)nc(-c5cccc(-c6ccc(C#N)cc6)c5)n4)cc3)ccc21]

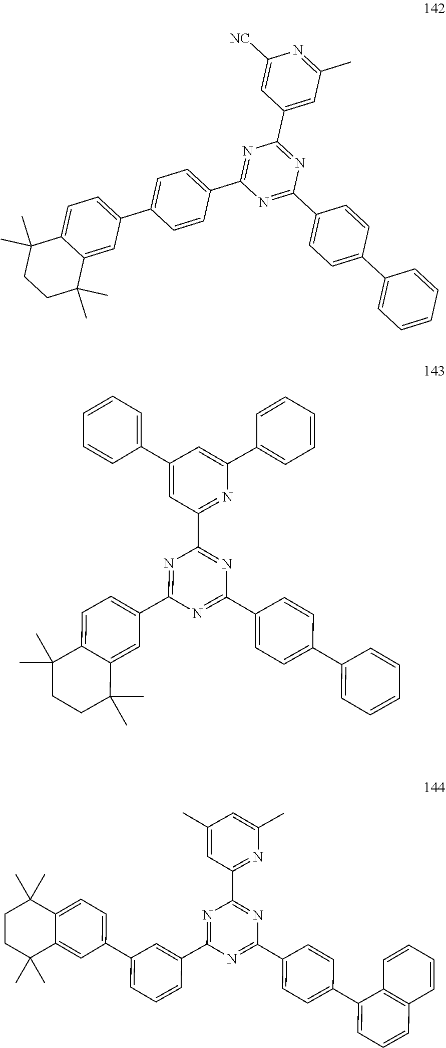 [SMILES: Cc1cc(-c2nc(-c3ccc(-c4ccccc4)cc3)nc(-c3ccc(-c4ccc5c(c4)C(C)(C)CCC5(C)C)cc3)n2)cc(C#N)n1 ; CC1(C)CCC(C)(C)c2cc(-c3nc(-c4ccc(-c5ccccc5)cc4)nc(-c4cc(-c5ccccc5)cc(-c5ccccc5)n4)n3)ccc21 ; Cc1cc(C)nc(-c2nc(-c3ccc(-c4cccc5ccccc45)cc3)nc(-c3cccc(-c4ccc5c(c4)C(C)(C)CCC5(C)C)c3)n2)c1]

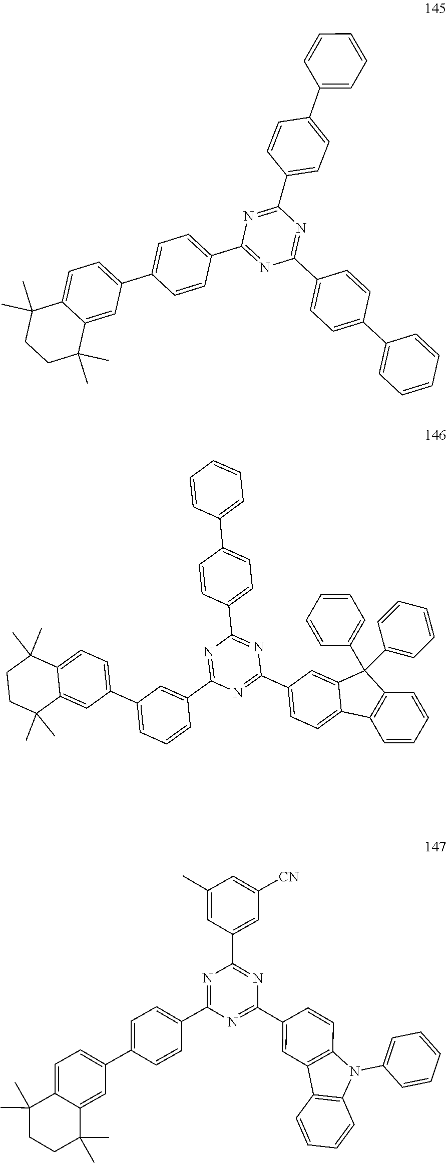 [SMILES: CC1(C)CCC(C)(C)c2cc(-c3ccc(-c4nc(-c5ccc(-c6ccccc6)cc5)nc(-c5ccc(-c6ccccc6)cc5)n4)cc3)ccc21 ; CC1(C)CCC(C)(C)c2cc(-c3cccc(-c4nc(-c5ccc(-c6ccccc6)cc5)nc(-c5ccc6c(c5)C(c5ccccc5)(c5ccccc5)c5ccccc5-6)n4)c3)ccc21 ; Cc1cc(C#N)cc(-c2nc(-c3ccc(-c4ccc5c(c4)C(C)(C)CCC5(C)C)cc3)nc(-c3ccc4c(c3)c3ccccc3n4-c3ccccc3)n2)c1]

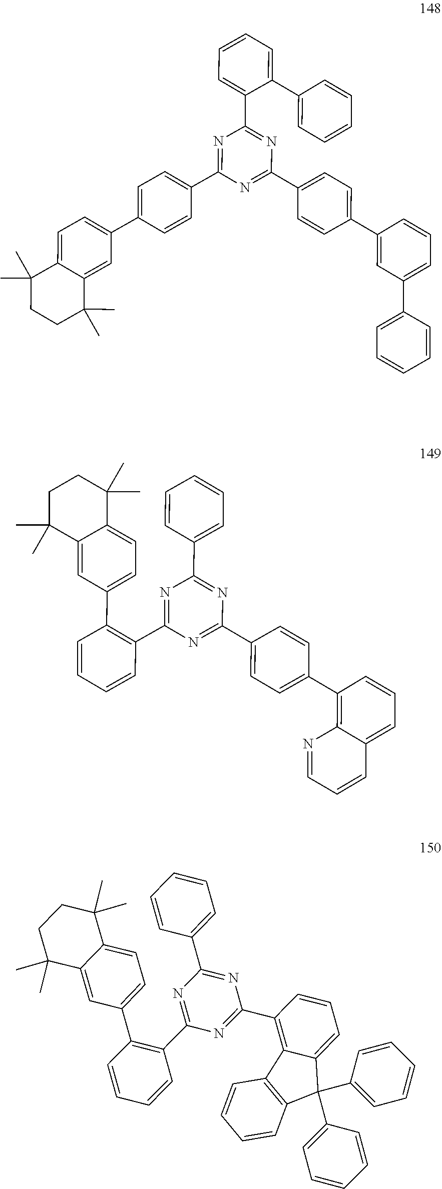 [SMILES: CC1(C)CCC(C)(C)c2cc(-c3ccc(-c4nc(-c5ccc(-c6cccc(-c7ccccc7)c6)cc5)nc(-c5ccccc5-c5ccccc5)n4)cc3)ccc21 ; CC1(C)CCC(C)(C)c2cc(-c3ccccc3-c3nc(-c4ccccc4)nc(-c4ccc(-c5cccc6cccnc56)cc4)n3)ccc21 ; CC1(C)CCC(C)(C)c2cc(-c3ccccc3-c3nc(-c4ccccc4)nc(-c4cccc5c4-c4ccccc4C5(c4ccccc4)c4ccccc4)n3)ccc21]

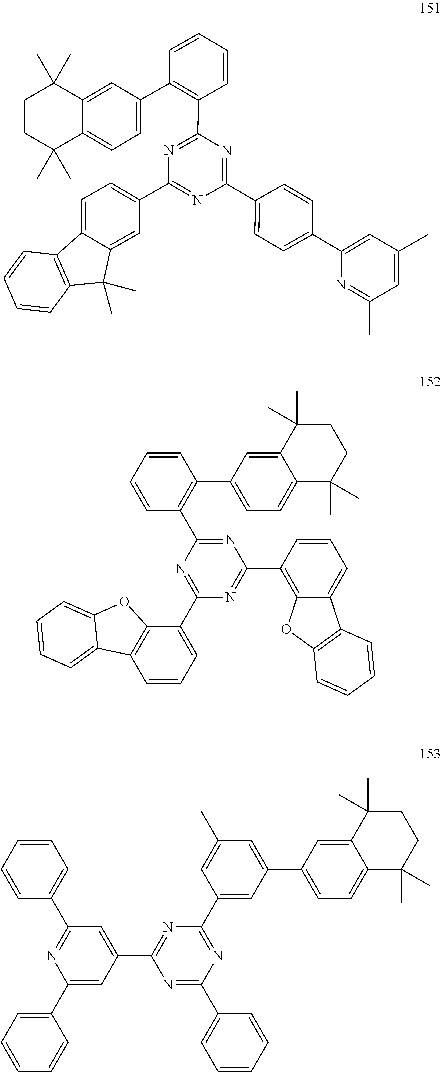 [SMILES: Cc1cc(C)nc(-c2ccc(-c3nc(-c4ccc5c(c4)C(C)(C)c4ccccc4-5)nc(-c4ccccc4-c4ccc5c(c4)C(C)(C)CCC5(C)C)n3)cc2)c1 ; CC1(C)CCC(C)(C)c2cc(-c3ccccc3-c3nc(-c4cccc5c4oc4ccccc45)nc(-c4cccc5c4oc4ccccc45)n3)ccc21 ; Cc1cc(-c2ccc3c(c2)C(C)(C)CCC3(C)C)cc(-c2nc(-c3ccccc3)nc(-c3cc(-c4ccccc4)nc(-c4ccccc4)c3)n2)c1]

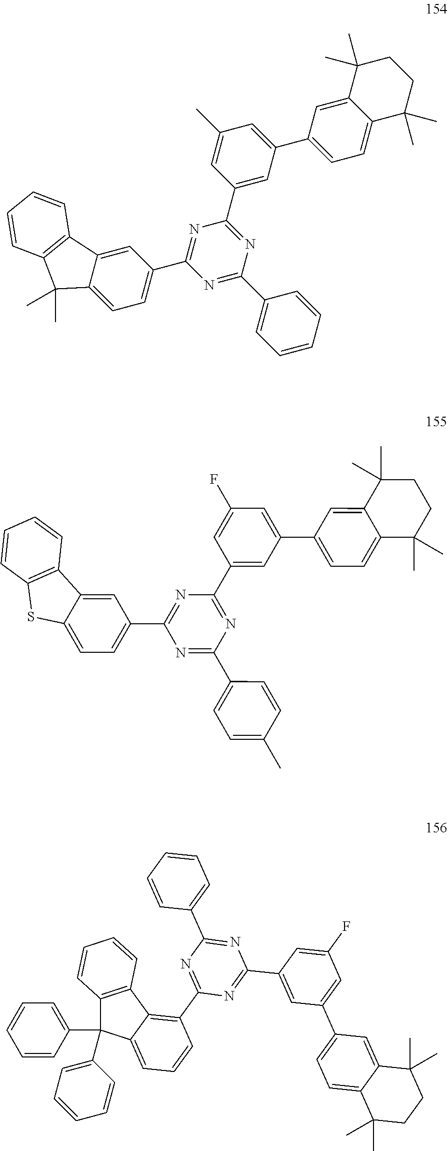 [SMILES: Cc1cc(-c2ccc3c(c2)C(C)(C)CCC3(C)C)cc(-c2nc(-c3ccccc3)nc(-c3ccc4c(c3)-c3ccccc3C4(C)C)n2)c1 ; Cc1ccc(-c2nc(-c3cc(F)cc(-c4ccc5c(c4)C(C)(C)CCC5(C)C)c3)nc(-c3ccc4sc5ccccc5c4c3)n2)cc1 ; CC1(C)CCC(C)(C)c2cc(-c3cc(F)cc(-c4nc(-c5ccccc5)nc(-c5cccc6c5-c5ccccc5C6(c5ccccc5)c5ccccc5)n4)c3)ccc21]

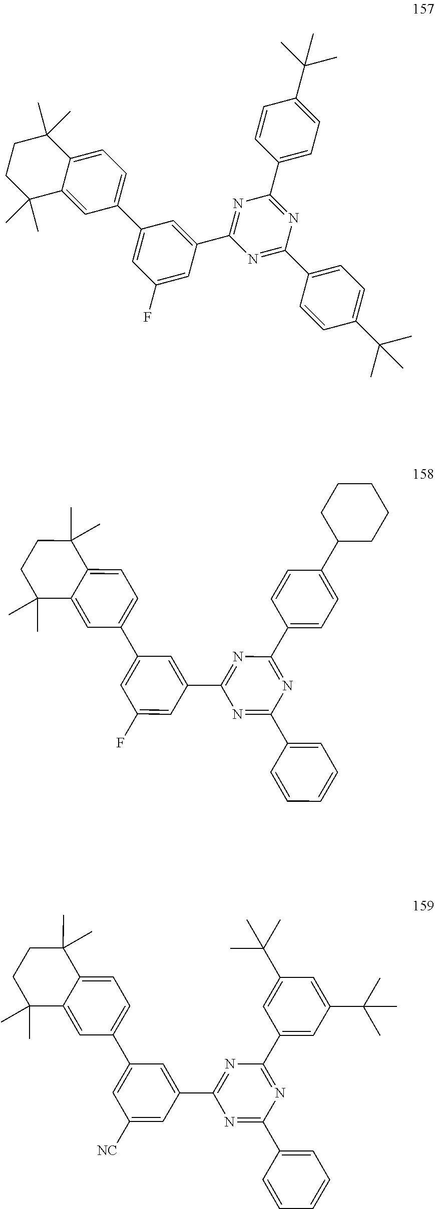

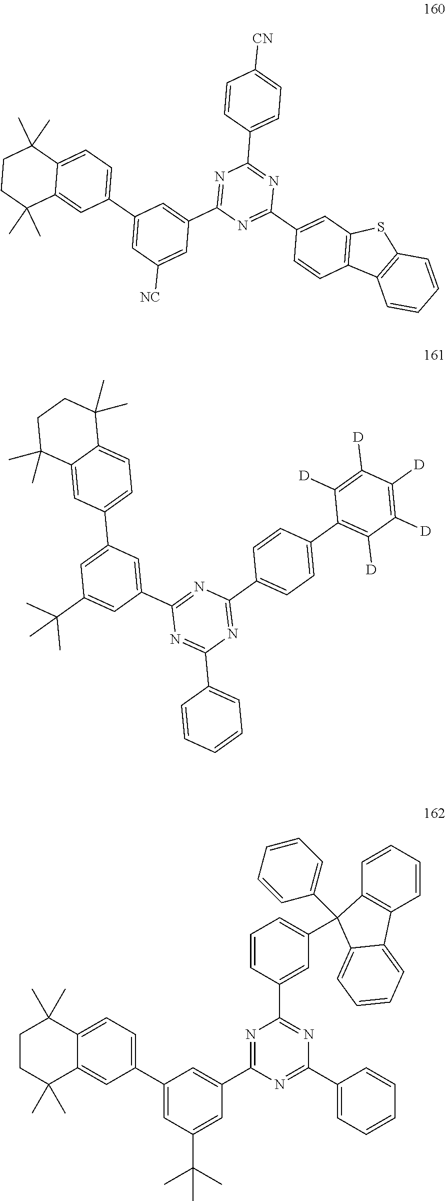 [SMILES: CC1(C)CCC(C)(C)c2cc(-c3cc(C#N)cc(-c4nc(-c5ccc(C#N)cc5)nc(-c5ccc6c(c5)sc5ccccc56)n4)c3)ccc21 ; [2H]c1c([2H])c([2H])c(-c2ccc(-c3nc(-c4ccccc4)nc(-c4cc(-c5ccc6c(c5)C(C)(C)CCC6(C)C)cc(C(C)(C)C)c4)n3)cc2)c([2H])c1[2H] ; CC(C)(C)c1cc(-c2ccc3c(c2)C(C)(C)CCC3(C)C)cc(-c2nc(-c3ccccc3)nc(-c3cccc(C4(c5ccccc5)c5ccccc5-c5ccccc54)c3)n2)c1]

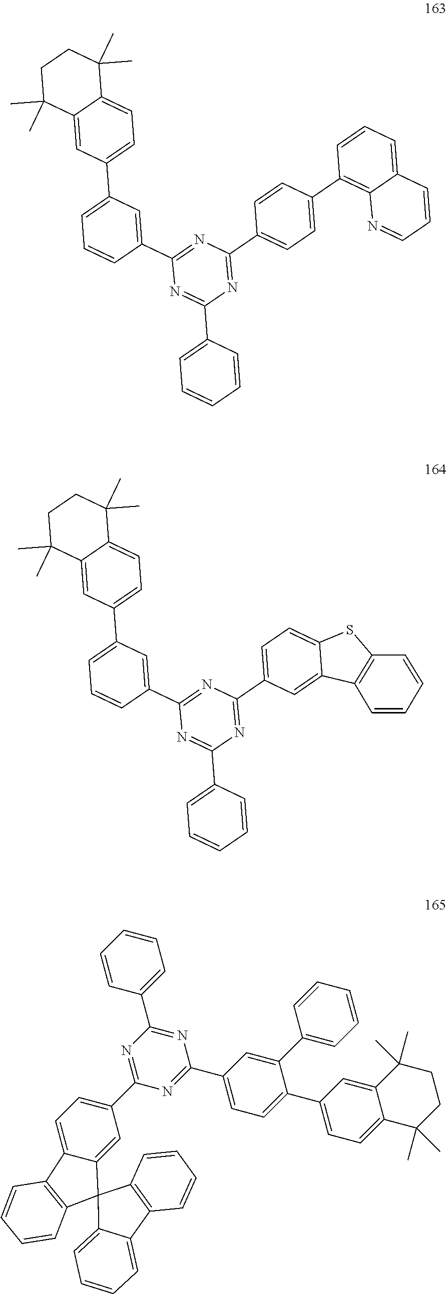 [SMILES: CC1(C)CCC(C)(C)c2cc(-c3cccc(-c4nc(-c5ccccc5)nc(-c5ccc(-c6cccc7cccnc67)cc5)n4)c3)ccc21 ; CC1(C)CCC(C)(C)c2cc(-c3cccc(-c4nc(-c5ccccc5)nc(-c5ccc6sc7ccccc7c6c5)n4)c3)ccc21 ; CC1(C)CCC(C)(C)c2cc(-c3ccc(-c4nc(-c5ccccc5)nc(-c5ccc6c(c5)C5(c7ccccc7-c7ccccc75)c5ccccc5-6)n4)cc3-c3ccccc3)ccc21]

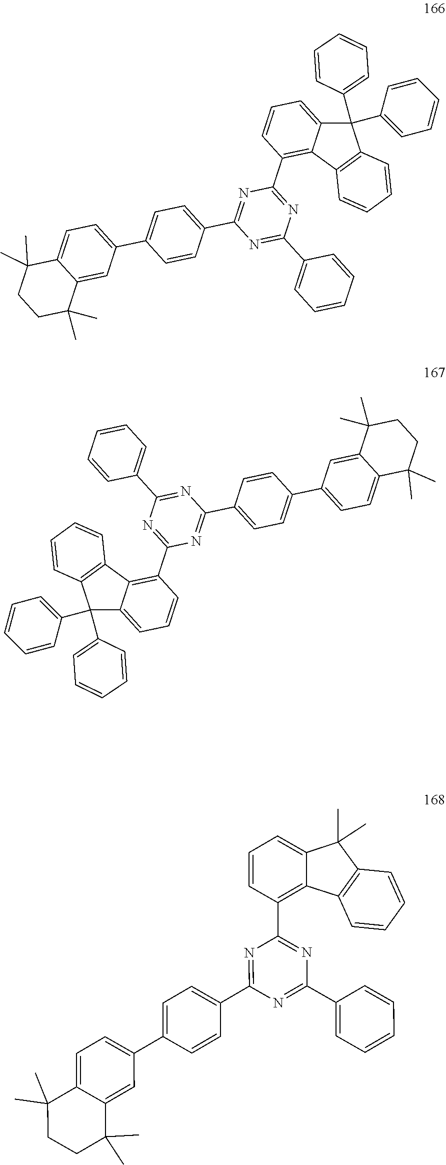

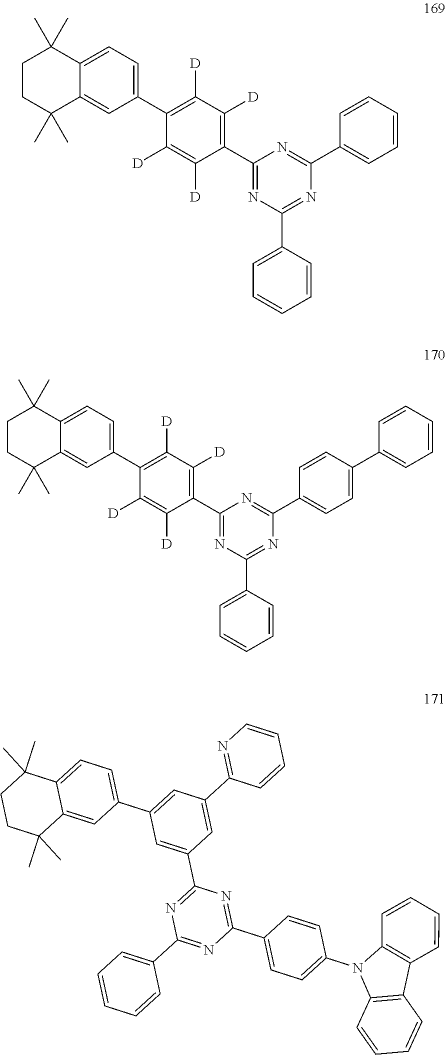 [SMILES: [2H]c1c([2H])c(-c2nc(-c3ccccc3)nc(-c3ccccc3)n2)c([2H])c([2H])c1-c1ccc2c(c1)C(C)(C)CCC2(C)C ; [2H]c1c([2H])c(-c2nc(-c3ccccc3)nc(-c3ccc(-c4ccccc4)cc3)n2)c([2H])c([2H])c1-c1ccc2c(c1)C(C)(C)CCC2(C)C ; CC1(C)CCC(C)(C)c2cc(-c3cc(-c4ccccn4)cc(-c4nc(-c5ccccc5)nc(-c5ccc(-n6c7ccccc7c7ccccc76)cc5)n4)c3)ccc21]

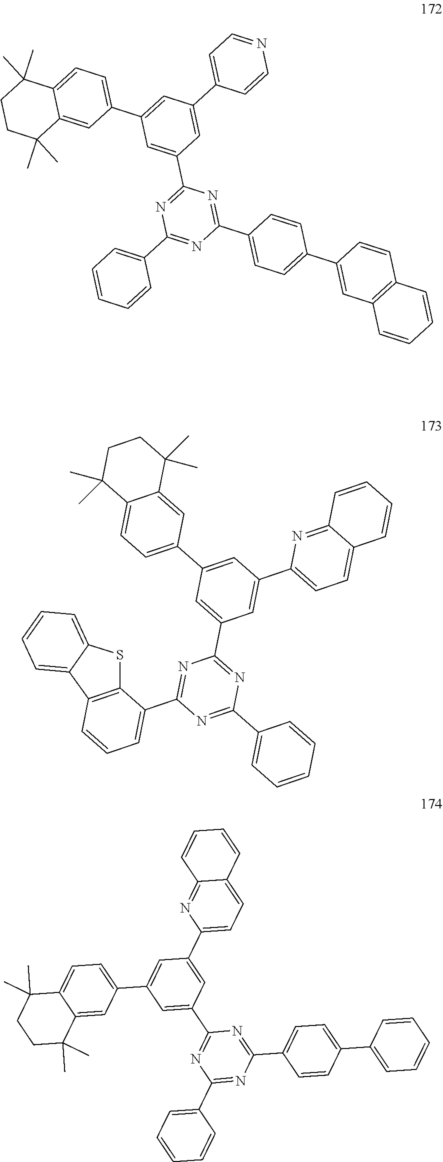 [SMILES: CC1(C)CCC(C)(C)c2cc(-c3cc(-c4ccncc4)cc(-c4nc(-c5ccccc5)nc(-c5ccc(-c6ccc7ccccc7c6)cc5)n4)c3)ccc21 ; CC1(C)CCC(C)(C)c2cc(-c3cc(-c4ccc5ccccc5n4)cc(-c4nc(-c5ccccc5)nc(-c5cccc6c5sc5ccccc56)n4)c3)ccc21 ; CC1(C)CCC(C)(C)c2cc(-c3cc(-c4ccc5ccccc5n4)cc(-c4nc(-c5ccccc5)nc(-c5ccc(-c6ccccc6)cc5)n4)c3)ccc21]

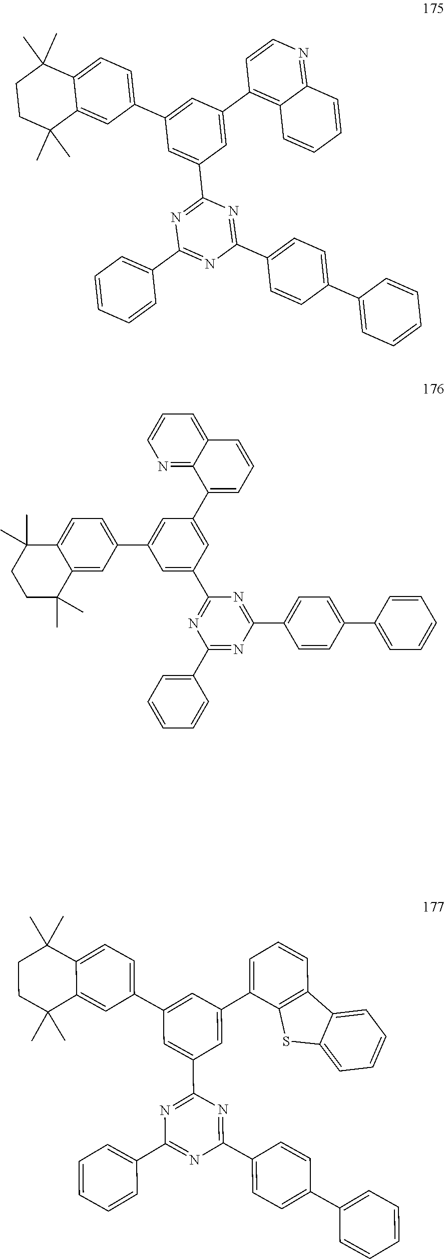 [SMILES: CC1(C)CCC(C)(C)c2cc(-c3cc(-c4nc(-c5ccccc5)nc(-c5ccc(-c6ccccc6)cc5)n4)cc(-c4ccnc5ccccc45)c3)ccc21 ; CC1(C)CCC(C)(C)c2cc(-c3cc(-c4nc(-c5ccccc5)nc(-c5ccc(-c6ccccc6)cc5)n4)cc(-c4cccc5cccnc45)c3)ccc21 ; CC1(C)CCC(C)(C)c2cc(-c3cc(-c4nc(-c5ccccc5)nc(-c5ccc(-c6ccccc6)cc5)n4)cc(-c4cccc5c4sc4ccccc45)c3)ccc21]

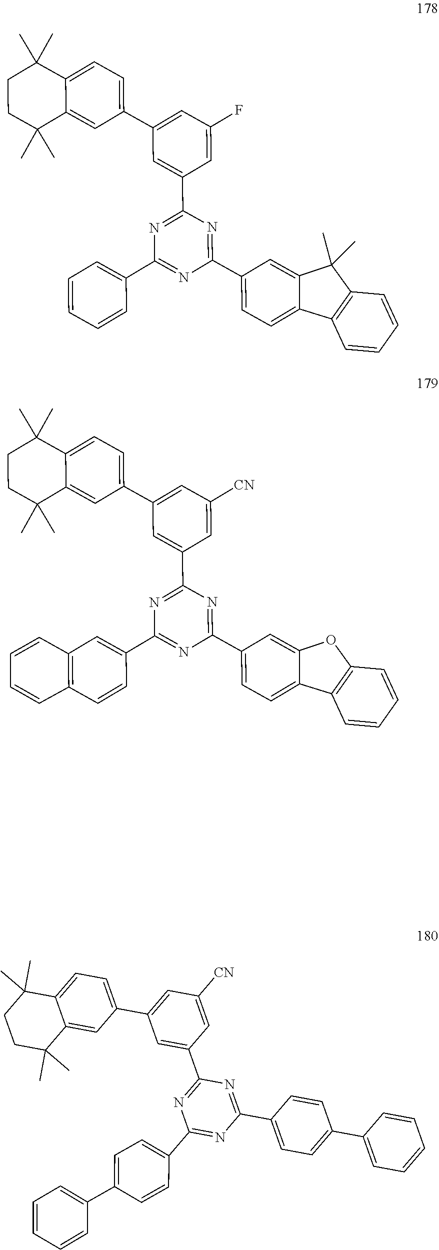 [SMILES: CC1(C)CCC(C)(C)c2cc(-c3cc(F)cc(-c4nc(-c5ccccc5)nc(-c5ccc6c(c5)C(C)(C)c5ccccc5-6)n4)c3)ccc21 ; CC1(C)CCC(C)(C)c2cc(-c3cc(C#N)cc(-c4nc(-c5ccc6ccccc6c5)nc(-c5ccc6c(c5)oc5ccccc56)n4)c3)ccc21 ; CC1(C)CCC(C)(C)c2cc(-c3cc(C#N)cc(-c4nc(-c5ccc(-c6ccccc6)cc5)nc(-c5ccc(-c6ccccc6)cc5)n4)c3)ccc21]

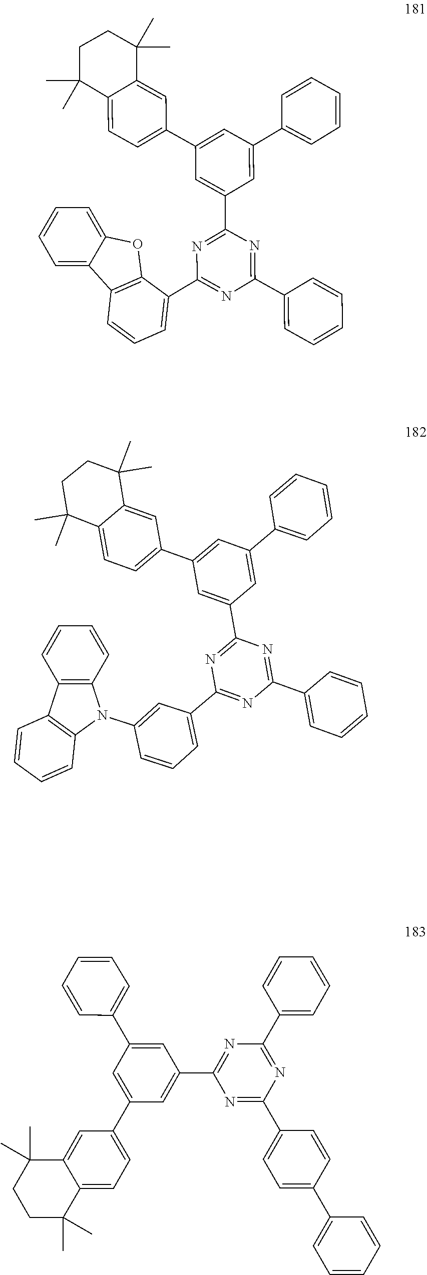 [SMILES: CC1(C)CCC(C)(C)c2cc(-c3cc(-c4ccccc4)cc(-c4nc(-c5ccccc5)nc(-c5cccc6c5oc5ccccc56)n4)c3)ccc21 ; CC1(C)CCC(C)(C)c2cc(-c3cc(-c4ccccc4)cc(-c4nc(-c5ccccc5)nc(-c5cccc(-n6c7ccccc7c7ccccc76)c5)n4)c3)ccc21 ; CC1(C)CCC(C)(C)c2cc(-c3cc(-c4ccccc4)cc(-c4nc(-c5ccccc5)nc(-c5ccc(-c6ccccc6)cc5)n4)c3)ccc21]

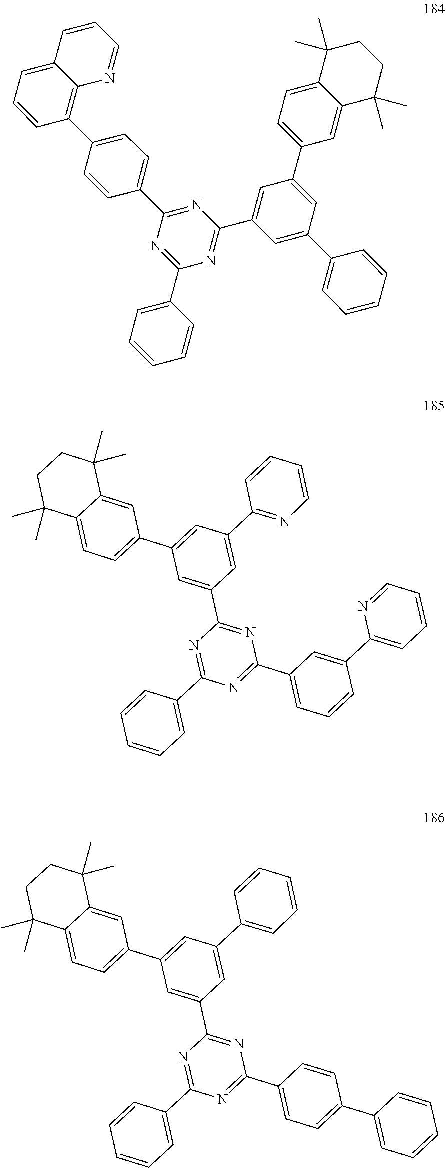

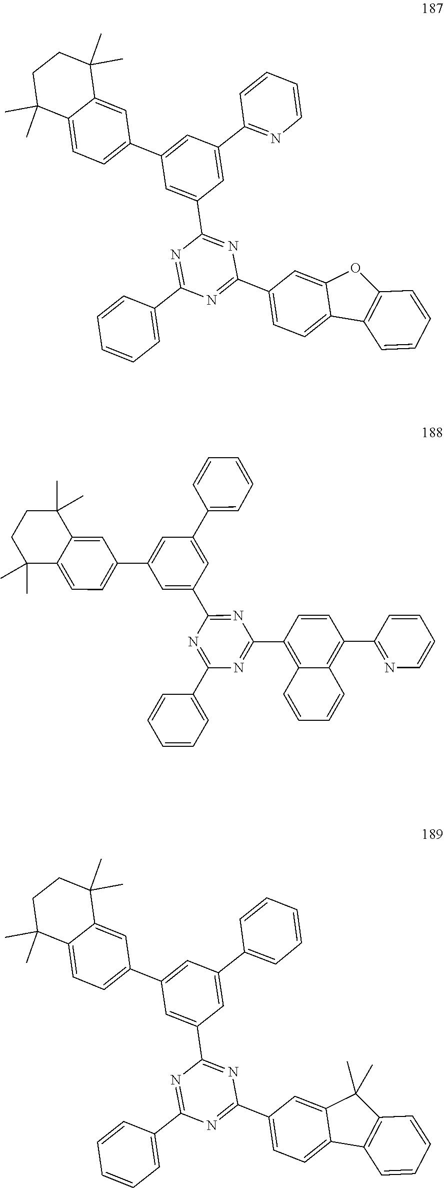 [SMILES: CC1(C)CCC(C)(C)c2cc(-c3cc(-c4ccccn4)cc(-c4nc(-c5ccccc5)nc(-c5ccc6c(c5)oc5ccccc56)n4)c3)ccc21 ; CC1(C)CCC(C)(C)c2cc(-c3cc(-c4ccccc4)cc(-c4nc(-c5ccccc5)nc(-c5ccc(-c6ccccn6)c6ccccc56)n4)c3)ccc21 ; CC1(C)CCC(C)(C)c2cc(-c3cc(-c4ccccc4)cc(-c4nc(-c5ccccc5)nc(-c5ccc6c(c5)C(C)(C)c5ccccc5-6)n4)c3)ccc21]

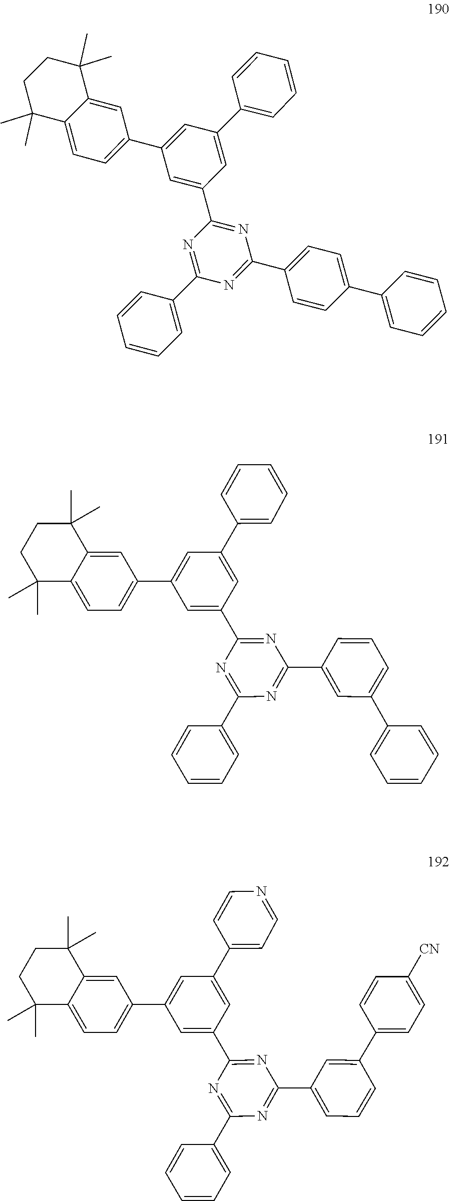 [SMILES: CC1(C)CCC(C)(C)c2cc(-c3cc(-c4ccccc4)cc(-c4nc(-c5ccccc5)nc(-c5ccc(-c6ccccc6)cc5)n4)c3)ccc21 ; CC1(C)CCC(C)(C)c2cc(-c3cc(-c4ccccc4)cc(-c4nc(-c5ccccc5)nc(-c5cccc(-c6ccccc6)c5)n4)c3)ccc21 ; CC1(C)CCC(C)(C)c2cc(-c3cc(-c4ccncc4)cc(-c4nc(-c5ccccc5)nc(-c5cccc(-c6ccc(C#N)cc6)c5)n4)c3)ccc21]

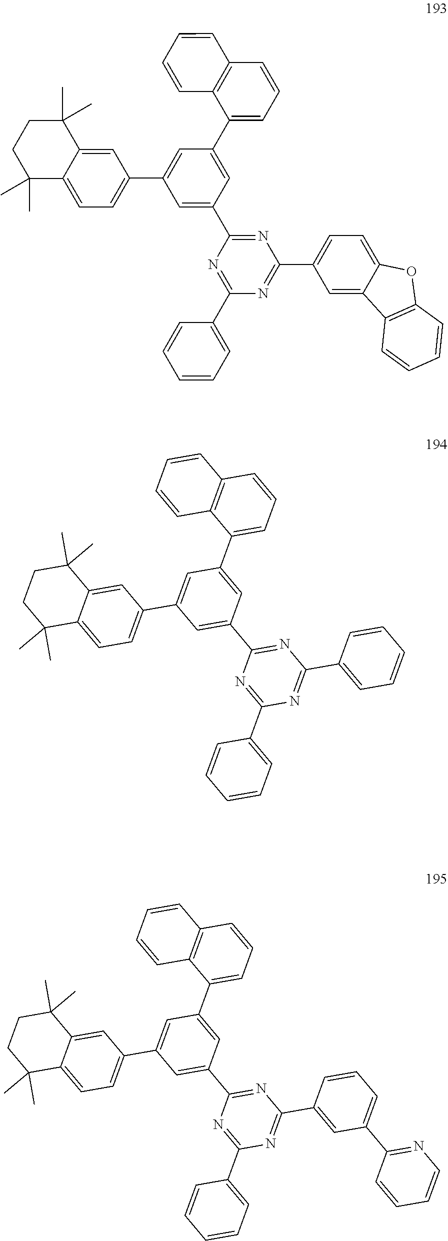 [SMILES: CC1(C)CCC(C)(C)c2cc(-c3cc(-c4nc(-c5ccccc5)nc(-c5ccc6oc7ccccc7c6c5)n4)cc(-c4cccc5ccccc45)c3)ccc21 ; CC1(C)CCC(C)(C)c2cc(-c3cc(-c4nc(-c5ccccc5)nc(-c5ccccc5)n4)cc(-c4cccc5ccccc45)c3)ccc21 ; CC1(C)CCC(C)(C)c2cc(-c3cc(-c4nc(-c5ccccc5)nc(-c5cccc(-c6ccccn6)c5)n4)cc(-c4cccc5ccccc45)c3)ccc21]

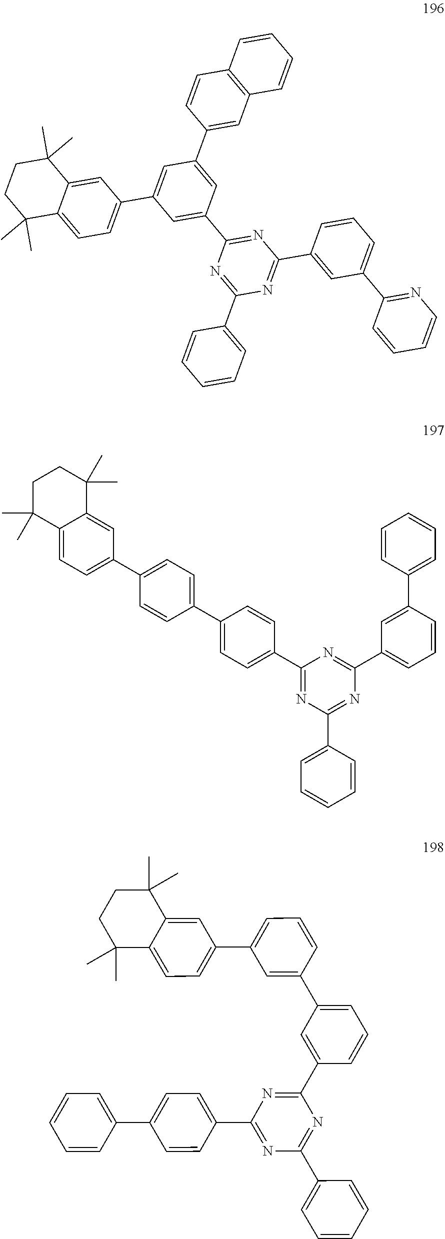 [SMILES: CC1(C)CCC(C)(C)c2cc(-c3cc(-c4ccc5ccccc5c4)cc(-c4nc(-c5ccccc5)nc(-c5cccc(-c6ccccn6)c5)n4)c3)ccc21 ; CC1(C)CCC(C)(C)c2cc(-c3ccc(-c4ccc(-c5nc(-c6ccccc6)nc(-c6cccc(-c7ccccc7)c6)n5)cc4)cc3)ccc21 ; CC1(C)CCC(C)(C)c2cc(-c3cccc(-c4cccc(-c5nc(-c6ccccc6)nc(-c6ccc(-c7ccccc7)cc6)n5)c4)c3)ccc21]

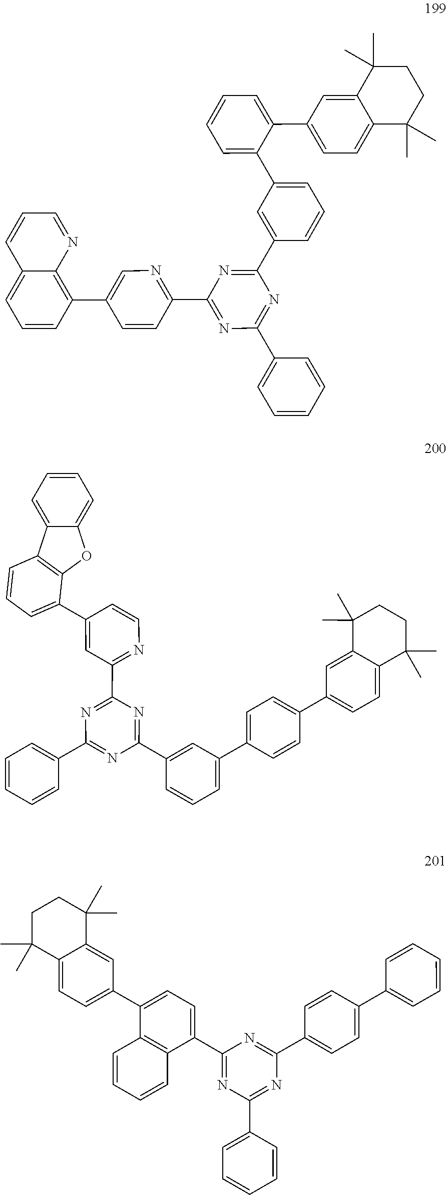

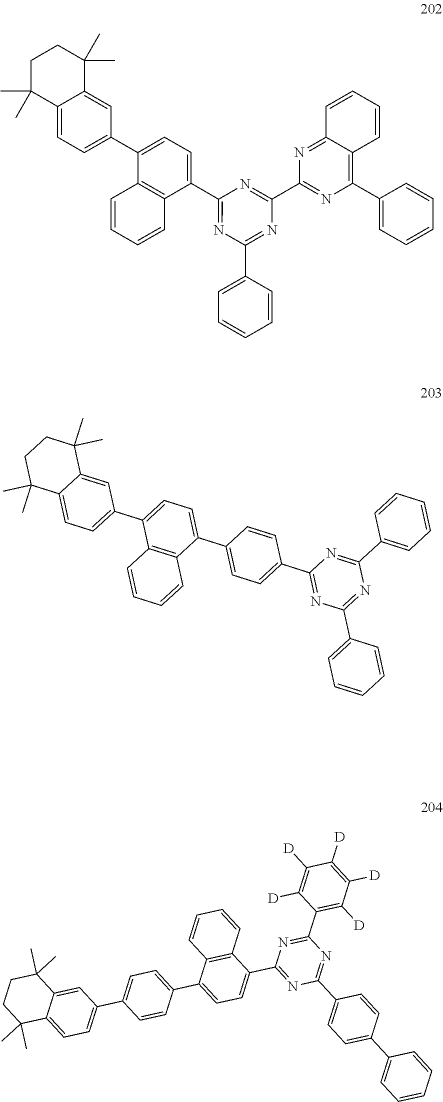 [SMILES: CC1(C)CCC(C)(C)c2cc(-c3ccc(-c4nc(-c5ccccc5)nc(-c5nc(-c6ccccc6)c6ccccc6n5)n4)c4ccccc34)ccc21 ; CC1(C)CCC(C)(C)c2cc(-c3ccc(-c4ccc(-c5nc(-c6ccccc6)nc(-c6ccccc6)n5)cc4)c4ccccc34)ccc21 ; [2H]c1c([2H])c([2H])c(-c2nc(-c3ccc(-c4ccccc4)cc3)nc(-c3ccc(-c4ccc(-c5ccc6c(c5)C(C)(C)CCC6(C)C)cc4)c4ccccc34)n2)c([2H])c1[2H]]

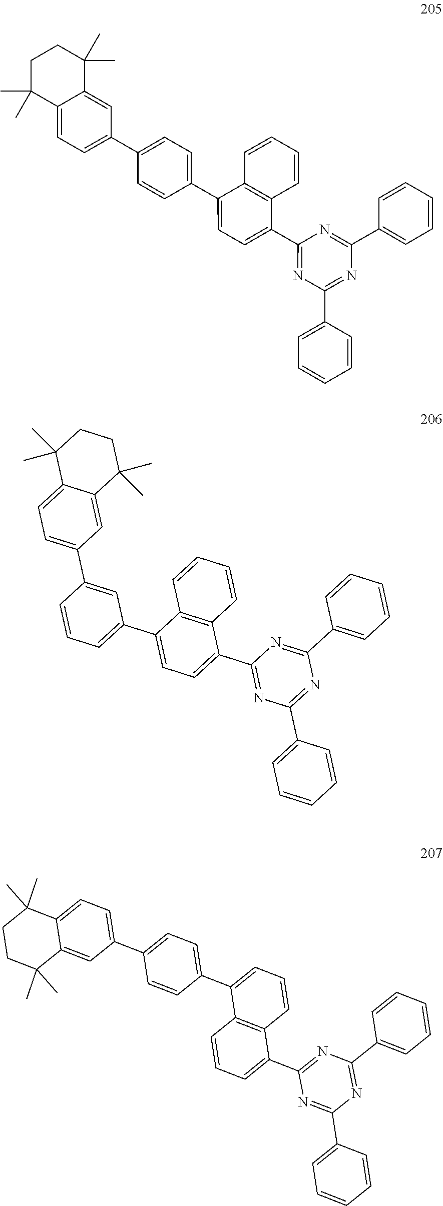 [SMILES: CC1(C)CCC(C)(C)c2cc(-c3ccc(-c4ccc(-c5nc(-c6ccccc6)nc(-c6ccccc6)n5)c5ccccc45)cc3)ccc21 ; CC1(C)CCC(C)(C)c2cc(-c3cccc(-c4ccc(-c5nc(-c6ccccc6)nc(-c6ccccc6)n5)c5ccccc45)c3)ccc21 ; CC1(C)CCC(C)(C)c2cc(-c3ccc(-c4cccc5c(-c6nc(-c7ccccc7)nc(-c7ccccc7)n6)cccc45)cc3)ccc21]

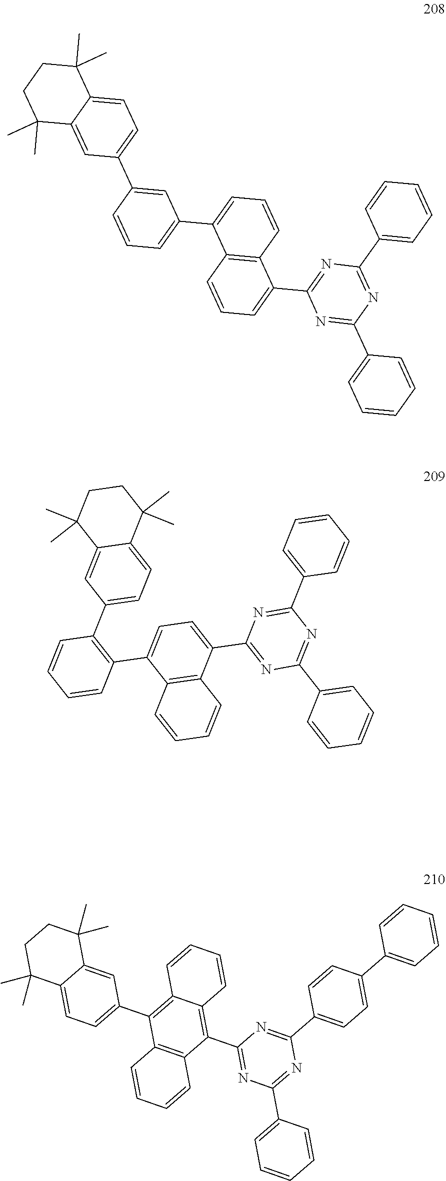 [SMILES: CC1(C)CCC(C)(C)c2cc(-c3cccc(-c4cccc5c(-c6nc(-c7ccccc7)nc(-c7ccccc7)n6)cccc45)c3)ccc21 ; CC1(C)CCC(C)(C)c2cc(-c3ccccc3-c3ccc(-c4nc(-c5ccccc5)nc(-c5ccccc5)n4)c4ccccc34)ccc21 ; CC1(C)CCC(C)(C)c2cc(-c3c4ccccc4c(-c4nc(-c5ccccc5)nc(-c5ccc(-c6ccccc6)cc5)n4)c4ccccc34)ccc21]

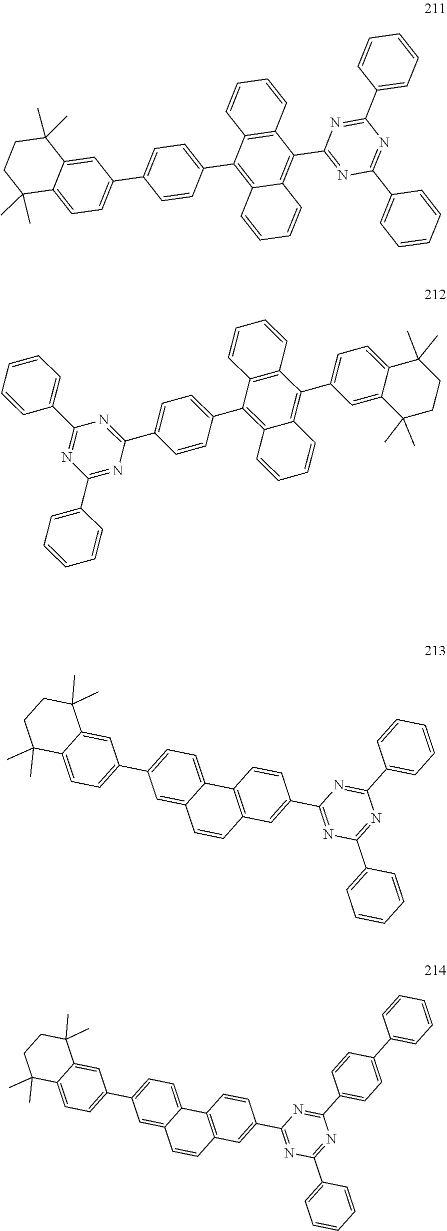 [SMILES: CC1(C)CCC(C)(C)c2cc(-c3ccc(-c4c5ccccc5c(-c5nc(-c6ccccc6)nc(-c6ccccc6)n5)c5ccccc45)cc3)ccc21 ; CC1(C)CCC(C)(C)c2cc(-c3c4ccccc4c(-c4ccc(-c5nc(-c6ccccc6)nc(-c6ccccc6)n5)cc4)c4ccccc34)ccc21 ; CC1(C)CCC(C)(C)c2cc(-c3ccc4c(ccc5cc(-c6nc(-c7ccccc7)nc(-c7ccccc7)n6)ccc54)c3)ccc21 ; CC1(C)CCC(C)(C)c2cc(-c3ccc4c(ccc5cc(-c6nc(-c7ccccc7)nc(-c7ccc(-c8ccccc8)cc7)n6)ccc54)c3)ccc21]

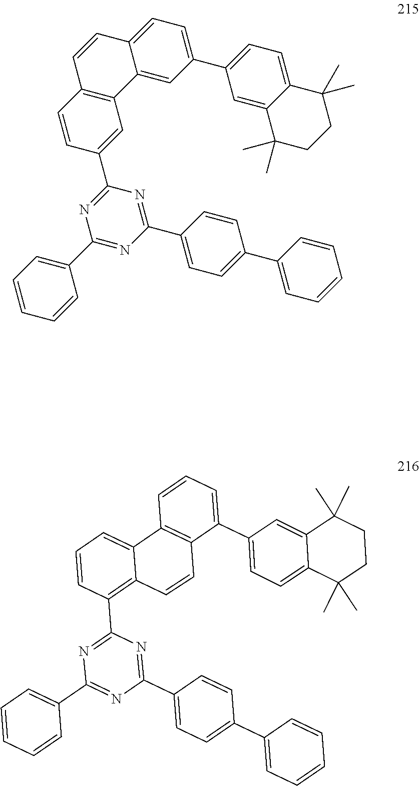 [SMILES: CC1(C)CCC(C)(C)c2cc(-c3ccc4ccc5ccc(-c6nc(-c7ccccc7)nc(-c7ccc(-c8ccccc8)cc7)n6)cc5c4c3)ccc21 ; CC1(C)CCC(C)(C)c2cc(-c3cccc4c3ccc3c(-c5nc(-c6ccccc6)nc(-c6ccc(-c7ccccc7)cc6)n5)cccc34)ccc21]

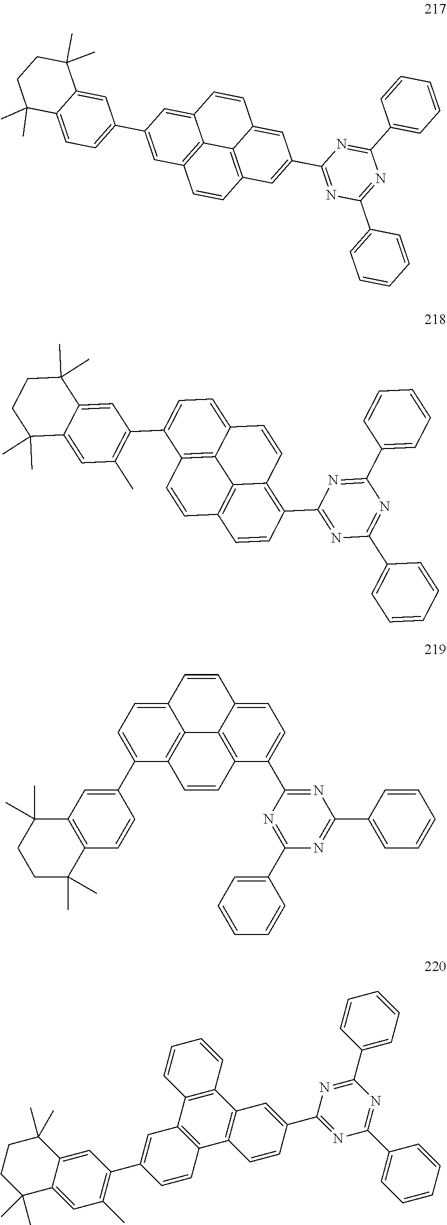 [SMILES: CC1(C)CCC(C)(C)c2cc(-c3cc4ccc5cc(-c6nc(-c7ccccc7)nc(-c7ccccc7)n6)cc6ccc(c3)c4c56)ccc21 ; Cc1cc2c(cc1-c1ccc3ccc4c(-c5nc(-c6ccccc6)nc(-c6ccccc6)n5)ccc5ccc1c3c54)C(C)(C)CCC2(C)C ; CC1(C)CCC(C)(C)c2cc(-c3ccc4ccc5ccc(-c6nc(-c7ccccc7)nc(-c7ccccc7)n6)c6ccc3c4c56)ccc21 ; Cc1cc2c(cc1-c1ccc3c4ccc(-c5nc(-c6ccccc6)nc(-c6ccccc6)n5)cc4c4ccccc4c3c1)C(C)(C)CCC2(C)C]

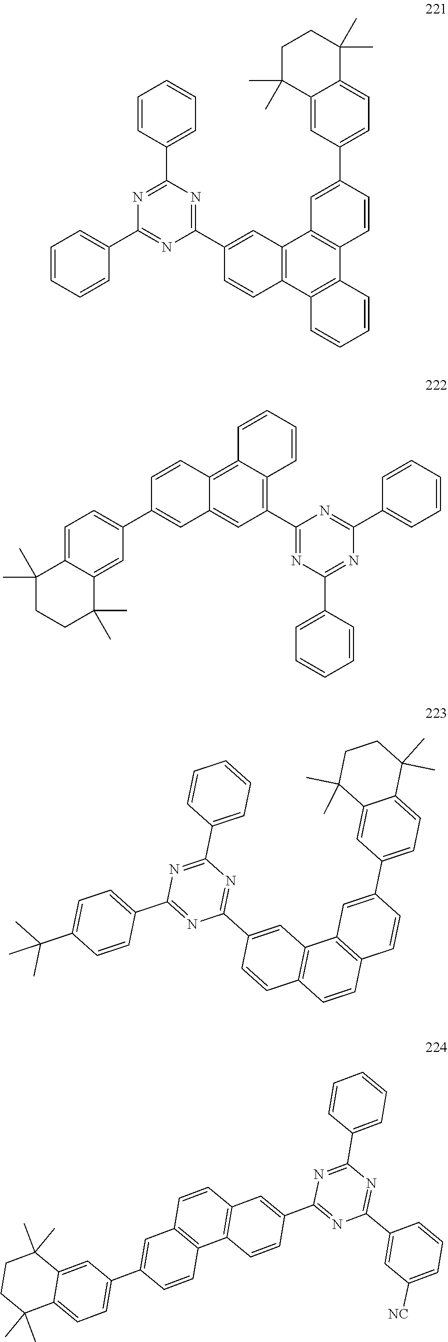

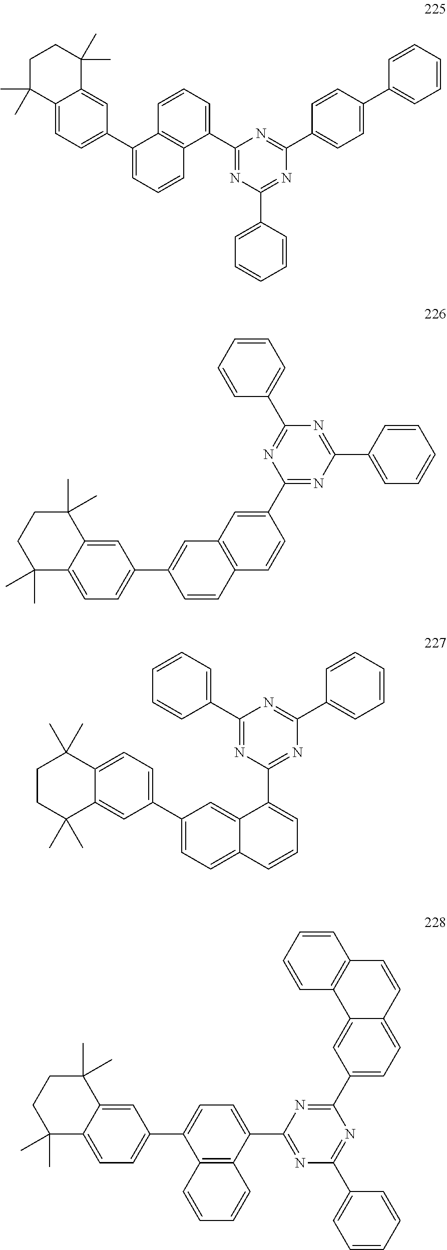 [SMILES: CC1(C)CCC(C)(C)c2cc(-c3cccc4c(-c5nc(-c6ccccc6)nc(-c6ccc(-c7ccccc7)cc6)n5)cccc34)ccc21 ; CC1(C)CCC(C)(C)c2cc(-c3ccc4ccc(-c5nc(-c6ccccc6)nc(-c6ccccc6)n5)cc4c3)ccc21 ; CC1(C)CCC(C)(C)c2cc(-c3ccc4cccc(-c5nc(-c6ccccc6)nc(-c6ccccc6)n5)c4c3)ccc21 ; CC1(C)CCC(C)(C)c2cc(-c3ccc(-c4nc(-c5ccccc5)nc(-c5ccc6ccc7ccccc7c6c5)n4)c4ccccc34)ccc21]

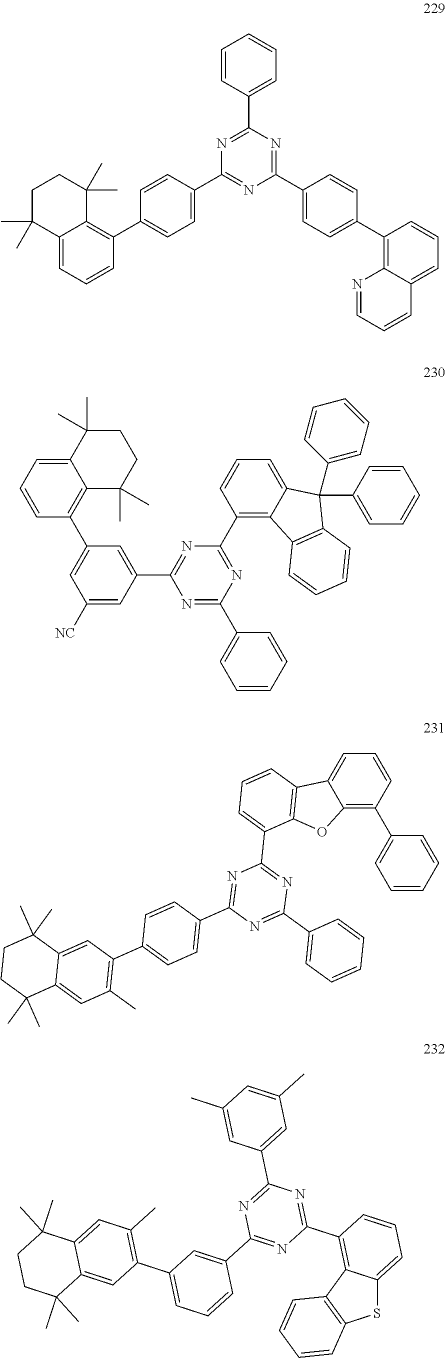 [SMILES: CC1(C)CCC(C)(C)c2c(-c3ccc(-c4nc(-c5ccccc5)nc(-c5ccc(-c6cccc7cccnc67)cc5)n4)cc3)cccc21 ; CC1(C)CCC(C)(C)c2c(-c3cc(C#N)cc(-c4nc(-c5ccccc5)nc(-c5cccc6c5-c5ccccc5C6(c5ccccc5)c5ccccc5)n4)c3)cccc21 ; Cc1cc2c(cc1-c1ccc(-c3nc(-c4ccccc4)nc(-c4cccc5c4oc4c(-c6ccccc6)cccc45)n3)cc1)C(C)(C)CCC2(C)C ; Cc1cc(C)cc(-c2nc(-c3cccc(-c4cc5c(cc4C)C(C)(C)CCC5(C)C)c3)nc(-c3cccc4sc5ccccc5c34)n2)c1]

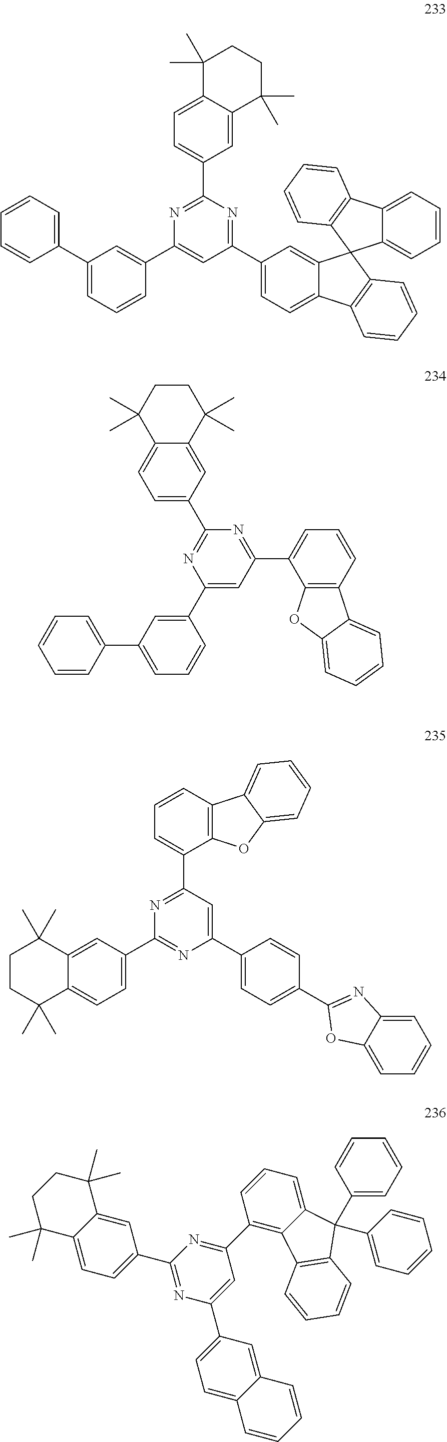 [SMILES: CC1(C)CCC(C)(C)c2cc(-c3nc(-c4cccc(-c5ccccc5)c4)cc(-c4ccc5c(c4)C4(c6ccccc6-c6ccccc64)c4ccccc4-5)n3)ccc21 ; CC1(C)CCC(C)(C)c2cc(-c3nc(-c4cccc(-c5ccccc5)c4)cc(-c4cccc5c4oc4ccccc45)n3)ccc21 ; CC1(C)CCC(C)(C)c2cc(-c3nc(-c4ccc(-c5nc6ccccc6o5)cc4)cc(-c4cccc5c4oc4ccccc45)n3)ccc21 ; CC1(C)CCC(C)(C)c2cc(-c3nc(-c4ccc5ccccc5c4)cc(-c4cccc5c4-c4ccccc4C5(c4ccccc4)c4ccccc4)n3)ccc21]

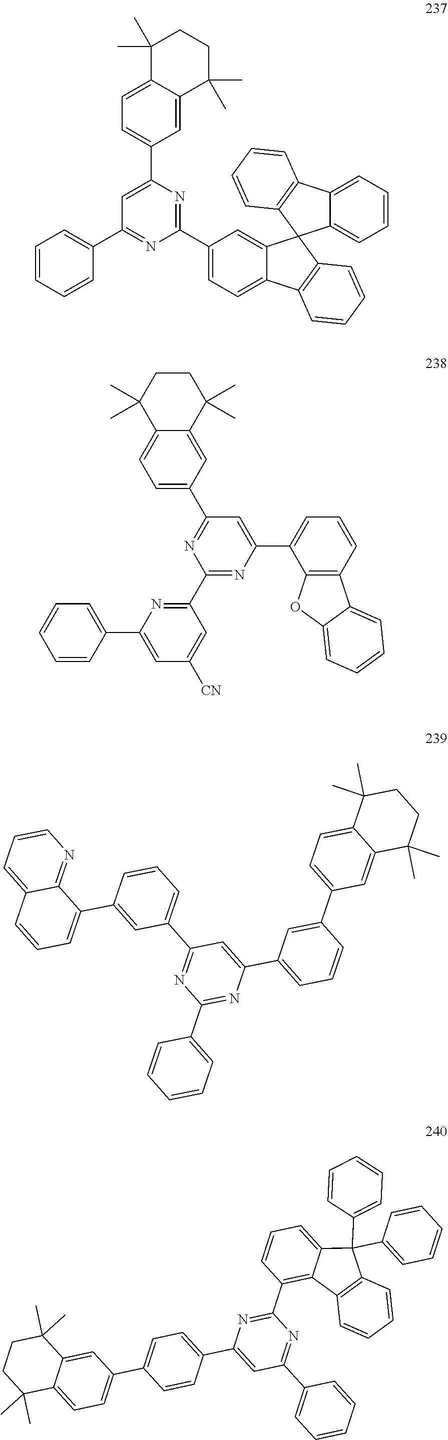

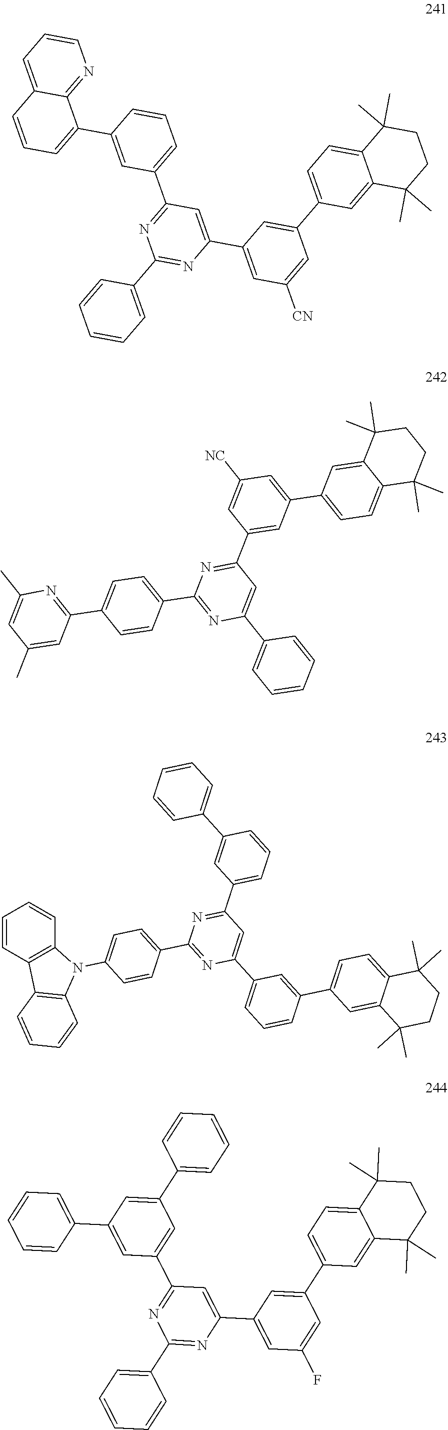

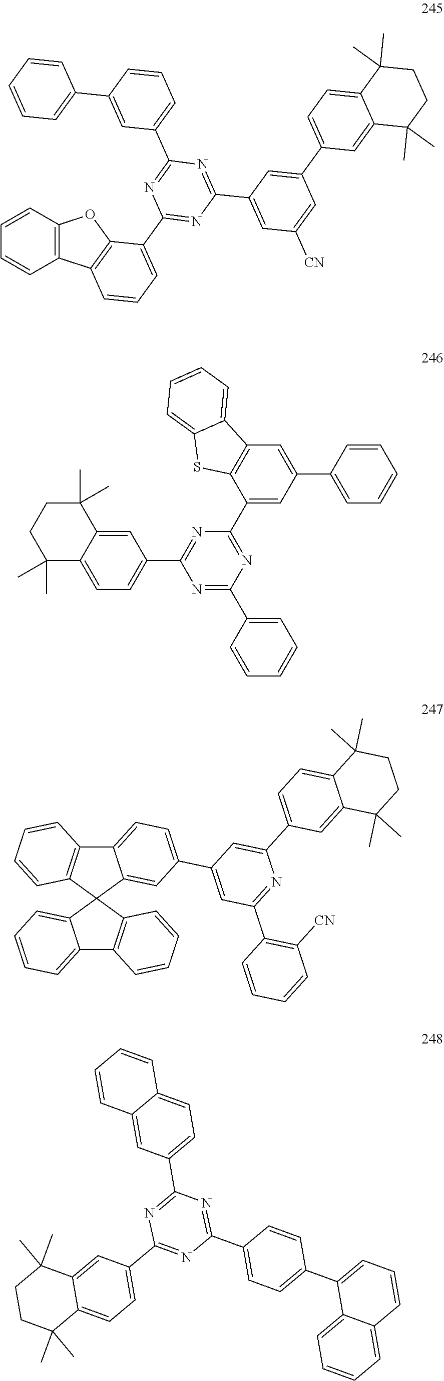

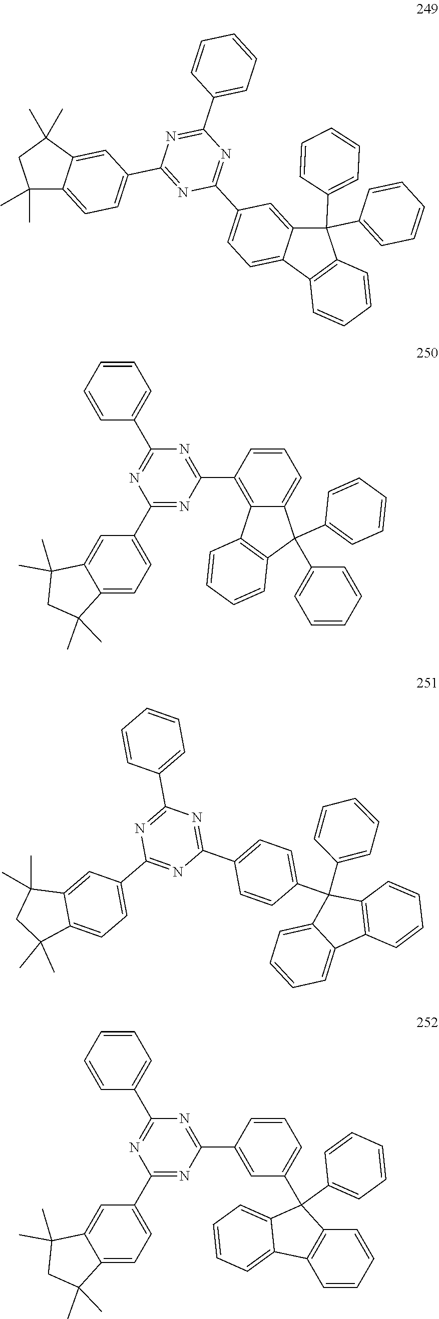 [SMILES: CC1(C)CC(C)(C)c2cc(-c3nc(-c4ccccc4)nc(-c4ccc5c(c4)C(c4ccccc4)(c4ccccc4)c4ccccc4-5)n3)ccc21 ; CC1(C)CC(C)(C)c2cc(-c3nc(-c4ccccc4)nc(-c4cccc5c4-c4ccccc4C5(c4ccccc4)c4ccccc4)n3)ccc21 ; CC1(C)CC(C)(C)c2cc(-c3nc(-c4ccccc4)nc(-c4ccc(C5(c6ccccc6)c6ccccc6-c6ccccc65)cc4)n3)ccc21 ; CC1(C)CC(C)(C)c2cc(-c3nc(-c4ccccc4)nc(-c4cccc(C5(c6ccccc6)c6ccccc6-c6ccccc65)c4)n3)ccc21]

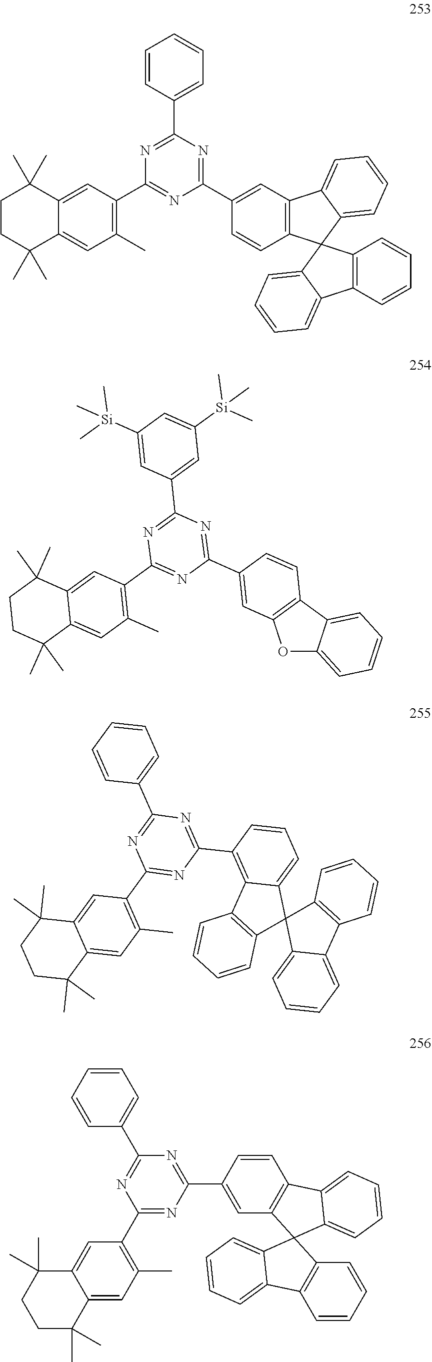 [SMILES: Cc1cc2c(cc1-c1nc(-c3ccccc3)nc(-c3ccc4c(c3)-c3ccccc3C43c4ccccc4-c4ccccc43)n1)C(C)(C)CCC2(C)C ; Cc1cc2c(cc1-c1nc(-c3cc([Si](C)(C)C)cc([Si](C)(C)C)c3)nc(-c3ccc4c(c3)oc3ccccc34)n1)C(C)(C)CCC2(C)C ; Cc1cc2c(cc1-c1nc(-c3ccccc3)nc(-c3cccc4c3-c3ccccc3C43c4ccccc4-c4ccccc43)n1)C(C)(C)CCC2(C)C ; Cc1cc2c(cc1-c1nc(-c3ccccc3)nc(-c3ccc4c(c3)C3(c5ccccc5-c5ccccc53)c3ccccc3-4)n1)C(C)(C)CCC2(C)C]

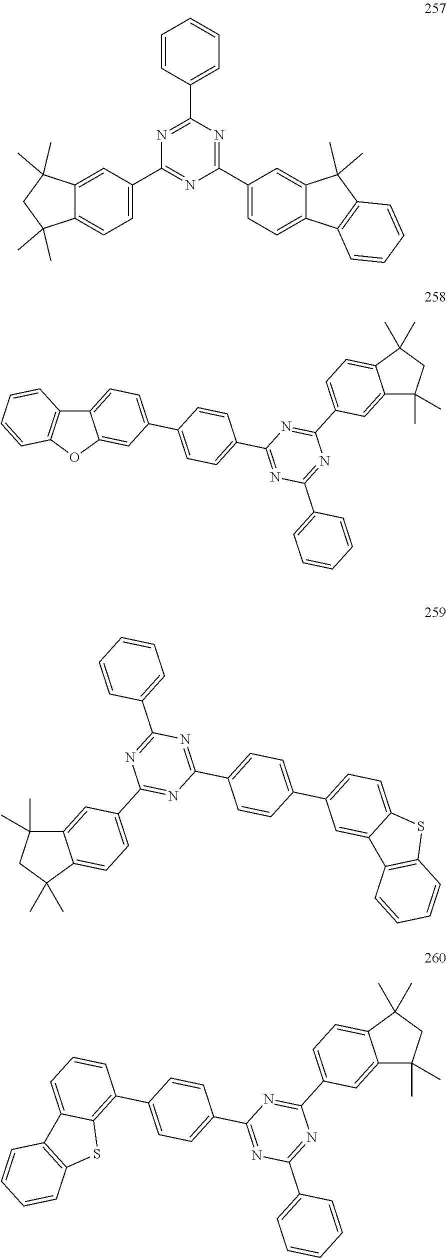

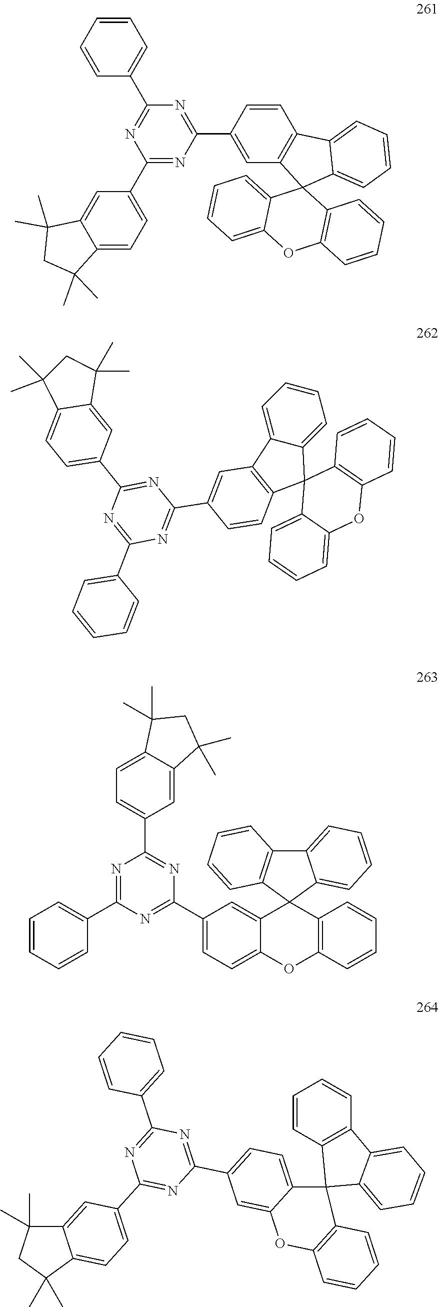

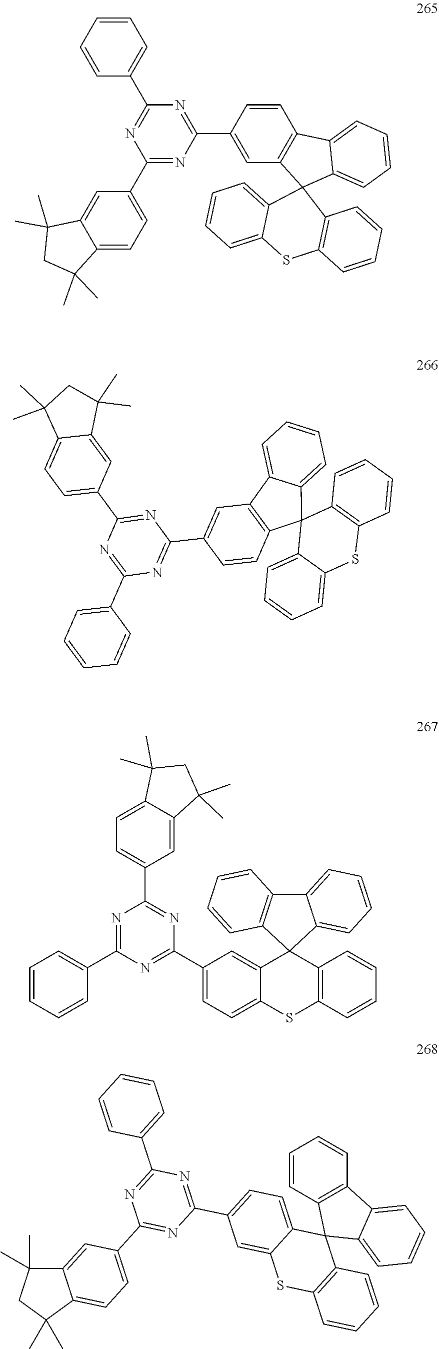 [SMILES: CC1(C)CC(C)(C)c2cc(-c3nc(-c4ccccc4)nc(-c4ccc5c(c4)C4(c6ccccc6Sc6ccccc64)c4ccccc4-5)n3)ccc21 ; CC1(C)CC(C)(C)c2cc(-c3nc(-c4ccccc4)nc(-c4ccc5c(c4)-c4ccccc4C54c5ccccc5Sc5ccccc54)n3)ccc21 ; CC1(C)CC(C)(C)c2cc(-c3nc(-c4ccccc4)nc(-c4ccc5c(c4)C4(c6ccccc6S5)c5ccccc5-c5ccccc54)n3)ccc21 ; CC1(C)CC(C)(C)c2cc(-c3nc(-c4ccccc4)nc(-c4ccc5c(c4)Sc4ccccc4C54c5ccccc5-c5ccccc54)n3)ccc21]

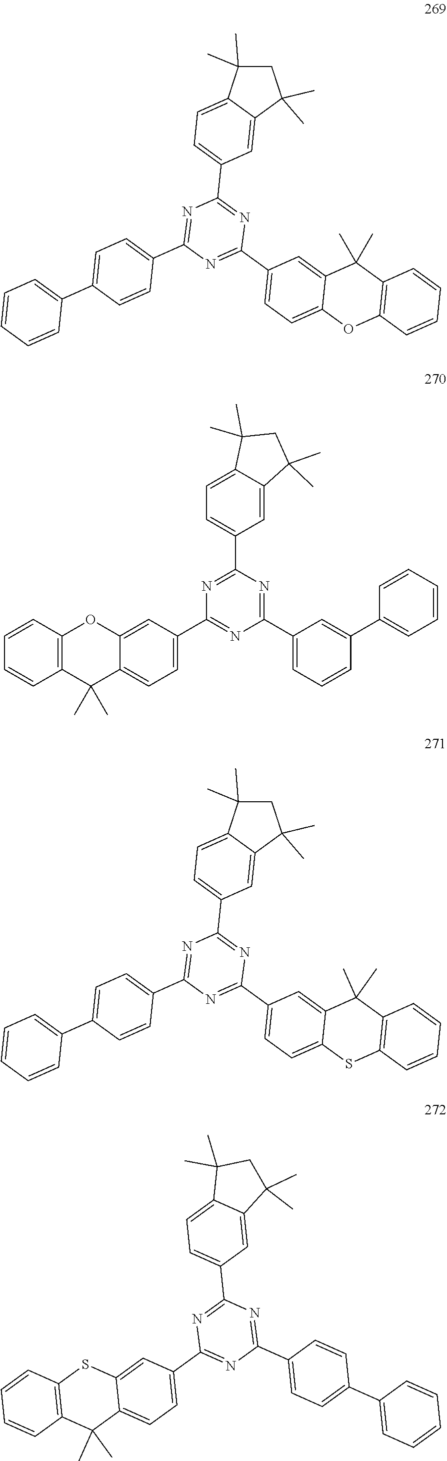 [SMILES: CC1(C)CC(C)(C)c2cc(-c3nc(-c4ccc(-c5ccccc5)cc4)nc(-c4ccc5c(c4)C(C)(C)c4ccccc4O5)n3)ccc21 ; CC1(C)CC(C)(C)c2cc(-c3nc(-c4cccc(-c5ccccc5)c4)nc(-c4ccc5c(c4)Oc4ccccc4C5(C)C)n3)ccc21 ; CC1(C)CC(C)(C)c2cc(-c3nc(-c4ccc(-c5ccccc5)cc4)nc(-c4ccc5c(c4)C(C)(C)c4ccccc4S5)n3)ccc21 ; CC1(C)CC(C)(C)c2cc(-c3nc(-c4ccc(-c5ccccc5)cc4)nc(-c4ccc5c(c4)Sc4ccccc4C5(C)C)n3)ccc21]

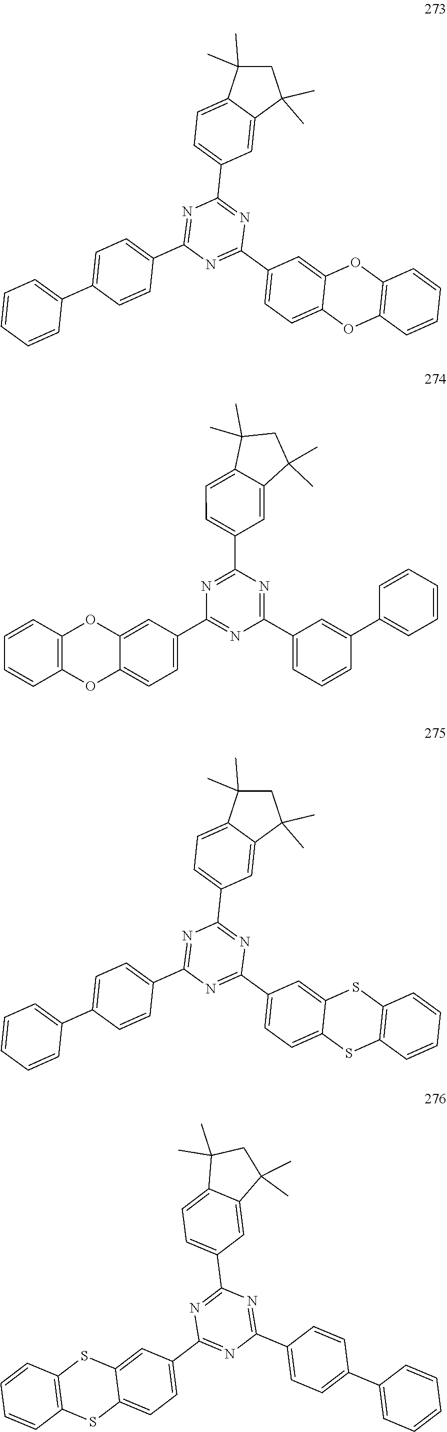 [SMILES: CC1(C)CC(C)(C)c2cc(-c3nc(-c4ccc(-c5ccccc5)cc4)nc(-c4ccc5c(c4)Oc4ccccc4O5)n3)ccc21 ; CC1(C)CC(C)(C)c2cc(-c3nc(-c4cccc(-c5ccccc5)c4)nc(-c4ccc5c(c4)Oc4ccccc4O5)n3)ccc21 ; CC1(C)CC(C)(C)c2cc(-c3nc(-c4ccc(-c5ccccc5)cc4)nc(-c4ccc5c(c4)Sc4ccccc4S5)n3)ccc21 ; CC1(C)CC(C)(C)c2cc(-c3nc(-c4ccc(-c5ccccc5)cc4)nc(-c4ccc5c(c4)Sc4ccccc4S5)n3)ccc21]

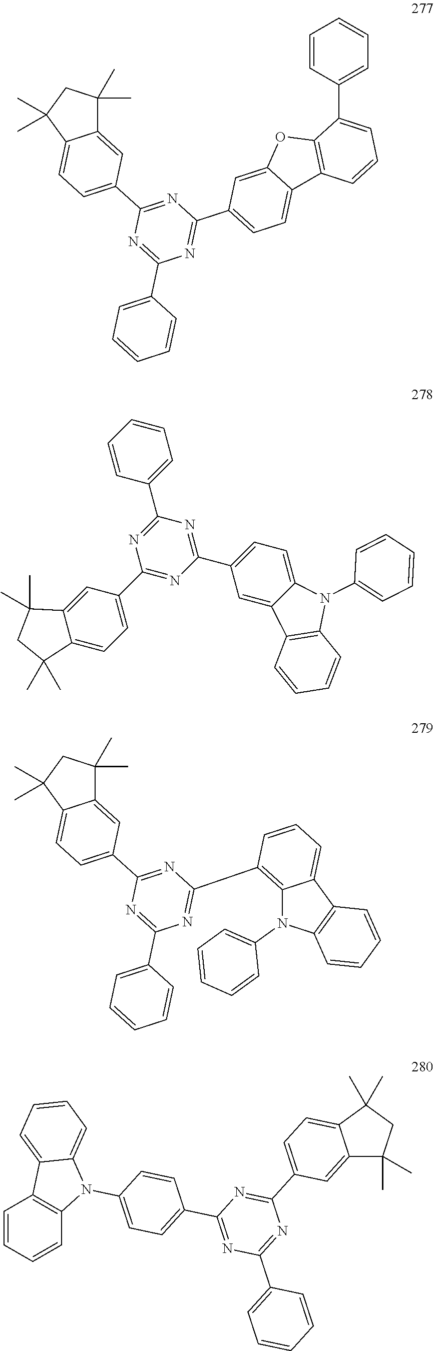 [SMILES: CC1(C)CC(C)(C)c2cc(-c3nc(-c4ccccc4)nc(-c4ccc5c(c4)oc4c(-c6ccccc6)cccc45)n3)ccc21 ; CC1(C)CC(C)(C)c2cc(-c3nc(-c4ccccc4)nc(-c4ccc5c(c4)c4ccccc4n5-c4ccccc4)n3)ccc21 ; CC1(C)CC(C)(C)c2cc(-c3nc(-c4ccccc4)nc(-c4cccc5c6ccccc6n(-c6ccccc6)c45)n3)ccc21 ; CC1(C)CC(C)(C)c2cc(-c3nc(-c4ccccc4)nc(-c4ccc(-n5c6ccccc6c6ccccc65)cc4)n3)ccc21]

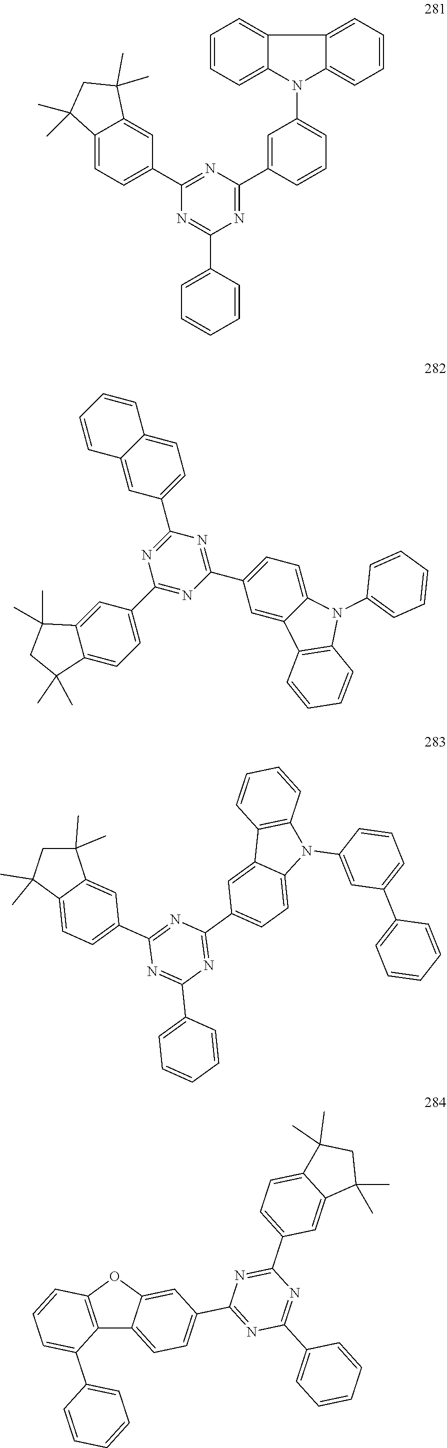 [SMILES: CC1(C)CC(C)(C)c2cc(-c3nc(-c4ccccc4)nc(-c4cccc(-n5c6ccccc6c6ccccc65)c4)n3)ccc21 ; CC1(C)CC(C)(C)c2cc(-c3nc(-c4ccc5ccccc5c4)nc(-c4ccc5c(c4)c4ccccc4n5-c4ccccc4)n3)ccc21 ; CC1(C)CC(C)(C)c2cc(-c3nc(-c4ccccc4)nc(-c4ccc5c(c4)c4ccccc4n5-c4cccc(-c5ccccc5)c4)n3)ccc21 ; CC1(C)CC(C)(C)c2cc(-c3nc(-c4ccccc4)nc(-c4ccc5c(c4)oc4cccc(-c6ccccc6)c45)n3)ccc21]

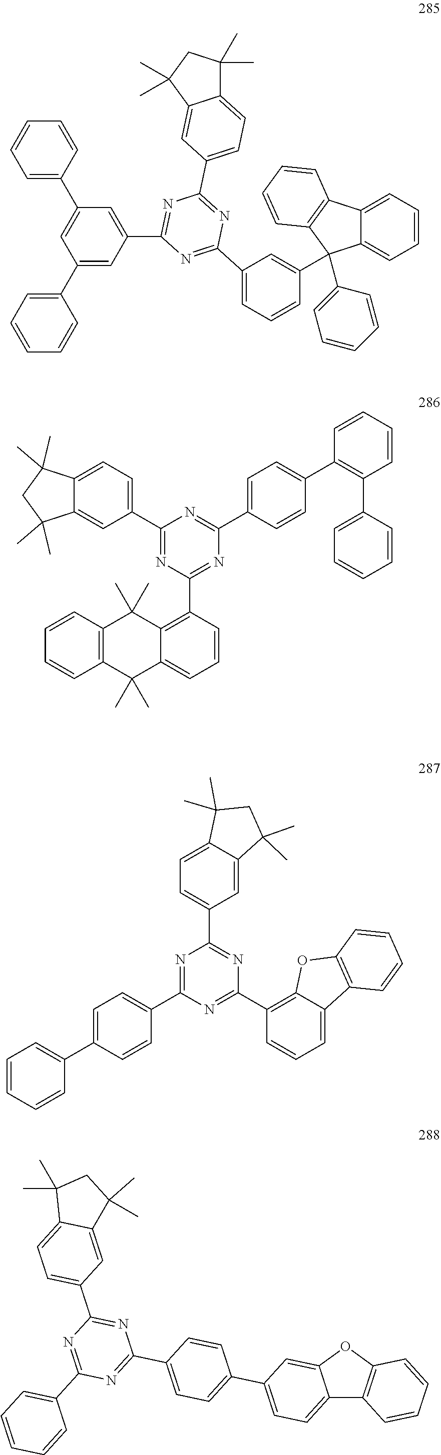 [SMILES: CC1(C)CC(C)(C)c2cc(-c3nc(-c4cc(-c5ccccc5)cc(-c5ccccc5)c4)nc(-c4cccc(C5(c6ccccc6)c6ccccc6-c6ccccc65)c4)n3)ccc21 ; CC1(C)CC(C)(C)c2cc(-c3nc(-c4ccc(-c5ccccc5-c5ccccc5)cc4)nc(-c4cccc5c4C(C)(C)c4ccccc4C5(C)C)n3)ccc21 ; CC1(C)CC(C)(C)c2cc(-c3nc(-c4ccc(-c5ccccc5)cc4)nc(-c4cccc5c4oc4ccccc45)n3)ccc21 ; CC1(C)CC(C)(C)c2cc(-c3nc(-c4ccccc4)nc(-c4ccc(-c5ccc6c(c5)oc5ccccc56)cc4)n3)ccc21]

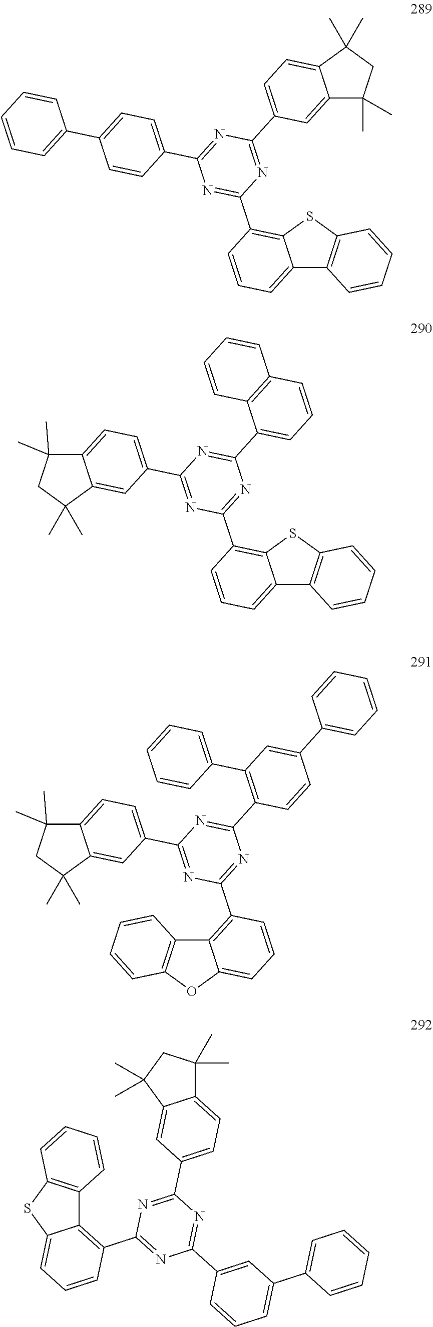 [SMILES: CC1(C)CC(C)(C)c2cc(-c3nc(-c4ccc(-c5ccccc5)cc4)nc(-c4cccc5c4sc4ccccc45)n3)ccc21 ; CC1(C)CC(C)(C)c2cc(-c3nc(-c4cccc5ccccc45)nc(-c4cccc5c4sc4ccccc45)n3)ccc21 ; CC1(C)CC(C)(C)c2cc(-c3nc(-c4ccc(-c5ccccc5)cc4-c4ccccc4)nc(-c4cccc5oc6ccccc6c45)n3)ccc21 ; CC1(C)CC(C)(C)c2cc(-c3nc(-c4cccc(-c5ccccc5)c4)nc(-c4cccc5sc6ccccc6c45)n3)ccc21]

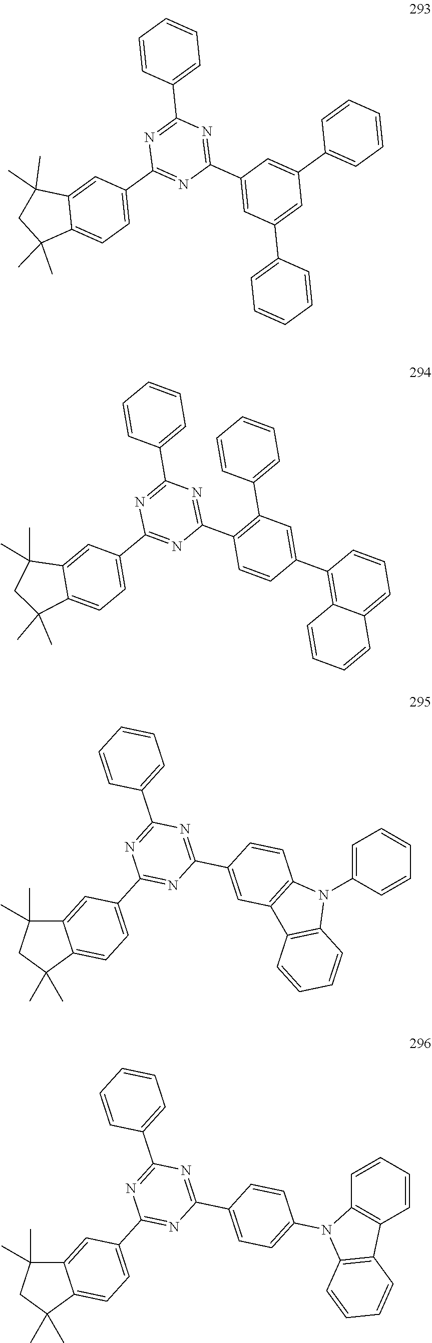 [SMILES: CC1(C)CC(C)(C)c2cc(-c3nc(-c4ccccc4)nc(-c4cc(-c5ccccc5)cc(-c5ccccc5)c4)n3)ccc21 ; CC1(C)CC(C)(C)c2cc(-c3nc(-c4ccccc4)nc(-c4ccc(-c5cccc6ccccc56)cc4-c4ccccc4)n3)ccc21 ; CC1(C)CC(C)(C)c2cc(-c3nc(-c4ccccc4)nc(-c4ccc5c(c4)c4ccccc4n5-c4ccccc4)n3)ccc21 ; CC1(C)CC(C)(C)c2cc(-c3nc(-c4ccccc4)nc(-c4ccc(-n5c6ccccc6c6ccccc65)cc4)n3)ccc21]

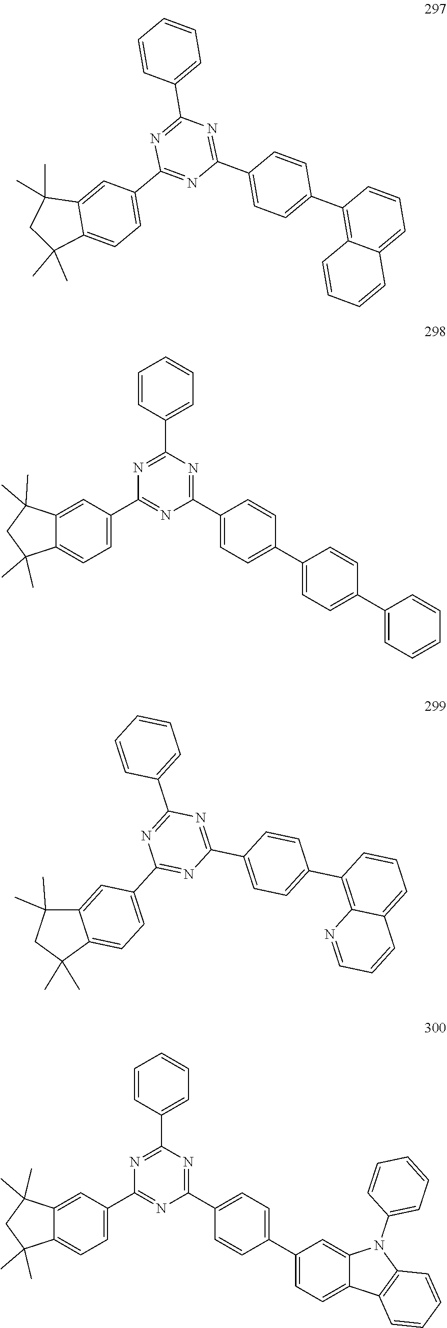 [SMILES: CC1(C)CC(C)(C)c2cc(-c3nc(-c4ccccc4)nc(-c4ccc(-c5cccc6ccccc56)cc4)n3)ccc21 ; CC1(C)CC(C)(C)c2cc(-c3nc(-c4ccccc4)nc(-c4ccc(-c5ccc(-c6ccccc6)cc5)cc4)n3)ccc21 ; CC1(C)CC(C)(C)c2cc(-c3nc(-c4ccccc4)nc(-c4ccc(-c5cccc6cccnc56)cc4)n3)ccc21 ; CC1(C)CC(C)(C)c2cc(-c3nc(-c4ccccc4)nc(-c4ccc(-c5ccc6c7ccccc7n(-c7ccccc7)c6c5)cc4)n3)ccc21]

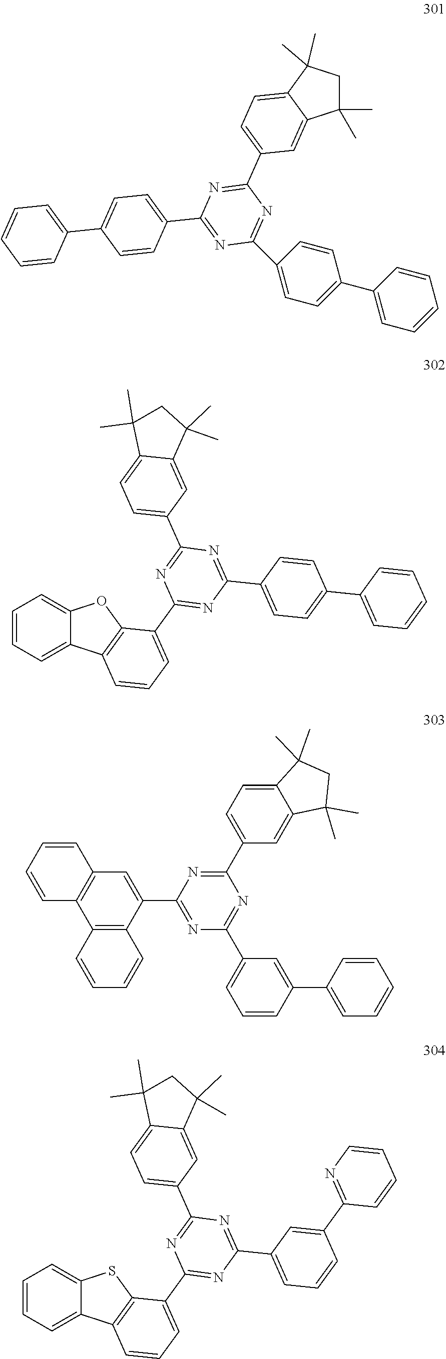 [SMILES: CC1(C)CC(C)(C)c2cc(-c3nc(-c4ccc(-c5ccccc5)cc4)nc(-c4ccc(-c5ccccc5)cc4)n3)ccc21 ; CC1(C)CC(C)(C)c2cc(-c3nc(-c4ccc(-c5ccccc5)cc4)nc(-c4cccc5c4oc4ccccc45)n3)ccc21 ; CC1(C)CC(C)(C)c2cc(-c3nc(-c4cccc(-c5ccccc5)c4)nc(-c4cc5ccccc5c5ccccc45)n3)ccc21 ; CC1(C)CC(C)(C)c2cc(-c3nc(-c4cccc(-c5ccccn5)c4)nc(-c4cccc5c4sc4ccccc45)n3)ccc21]

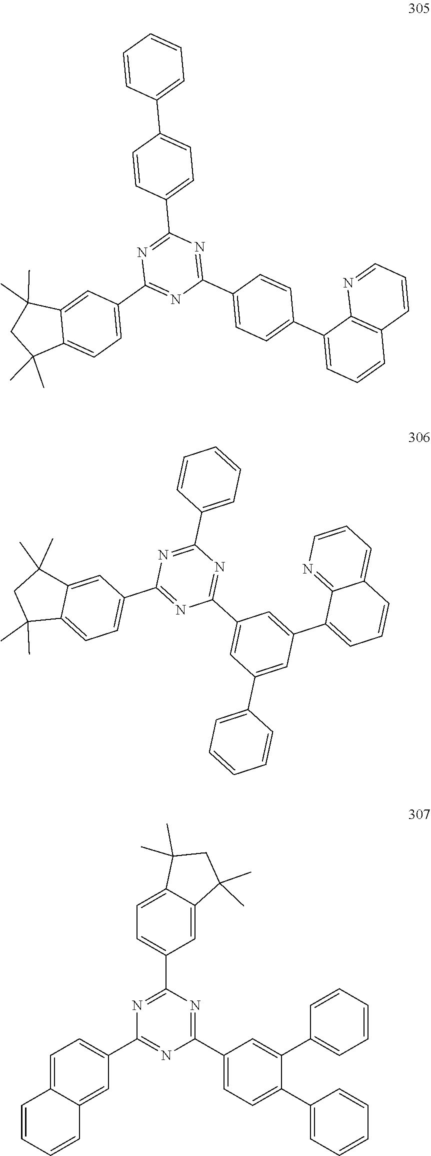 [SMILES: CC1(C)CC(C)(C)c2cc(-c3nc(-c4ccc(-c5ccccc5)cc4)nc(-c4ccc(-c5cccc6cccnc56)cc4)n3)ccc21 ; CC1(C)CC(C)(C)c2cc(-c3nc(-c4ccccc4)nc(-c4cc(-c5ccccc5)cc(-c5cccc6cccnc56)c4)n3)ccc21 ; CC1(C)CC(C)(C)c2cc(-c3nc(-c4ccc(-c5ccccc5)c(-c5ccccc5)c4)nc(-c4ccc5ccccc5c4)n3)ccc21]

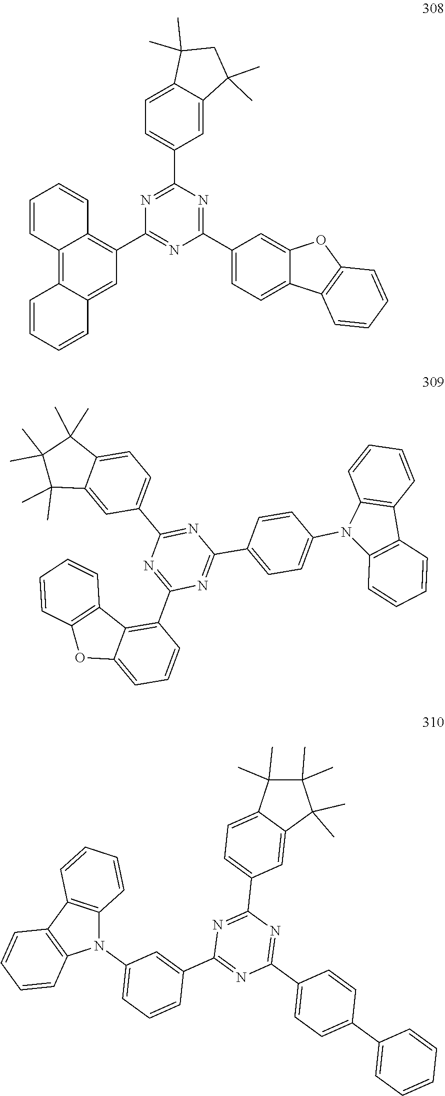 [SMILES: CC1(C)CC(C)(C)c2cc(-c3nc(-c4ccc5c(c4)oc4ccccc45)nc(-c4cc5ccccc5c5ccccc45)n3)ccc21 ; CC1(C)c2ccc(-c3nc(-c4ccc(-n5c6ccccc6c6ccccc65)cc4)nc(-c4cccc5oc6ccccc6c45)n3)cc2C(C)(C)C1(C)C ; CC1(C)c2ccc(-c3nc(-c4ccc(-c5ccccc5)cc4)nc(-c4cccc(-n5c6ccccc6c6ccccc65)c4)n3)cc2C(C)(C)C1(C)C]

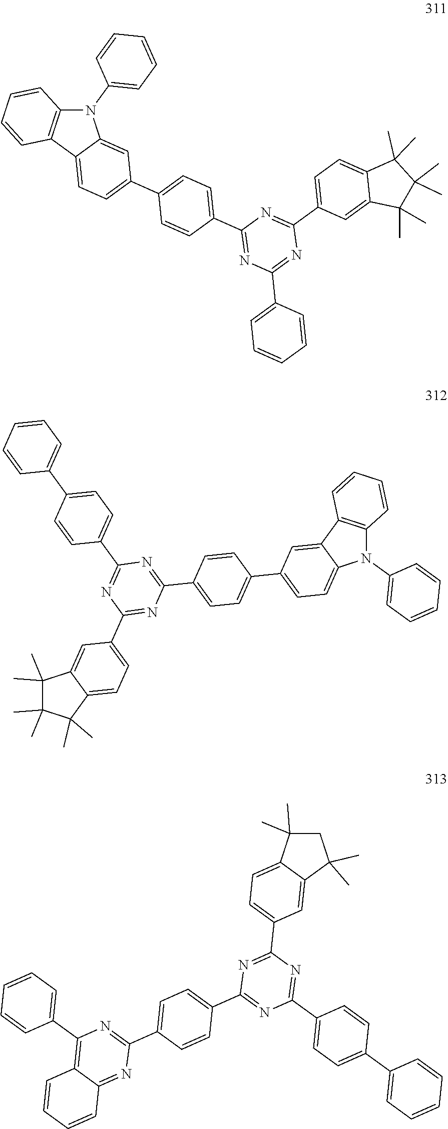 [SMILES: CC1(C)c2ccc(-c3nc(-c4ccccc4)nc(-c4ccc(-c5ccc6c7ccccc7n(-c7ccccc7)c6c5)cc4)n3)cc2C(C)(C)C1(C)C ; CC1(C)c2ccc(-c3nc(-c4ccc(-c5ccccc5)cc4)nc(-c4ccc(-c5ccc6c(c5)c5ccccc5n6-c5ccccc5)cc4)n3)cc2C(C)(C)C1(C)C ; CC1(C)CC(C)(C)c2cc(-c3nc(-c4ccc(-c5ccccc5)cc4)nc(-c4ccc(-c5nc(-c6ccccc6)c6ccccc6n5)cc4)n3)ccc21]

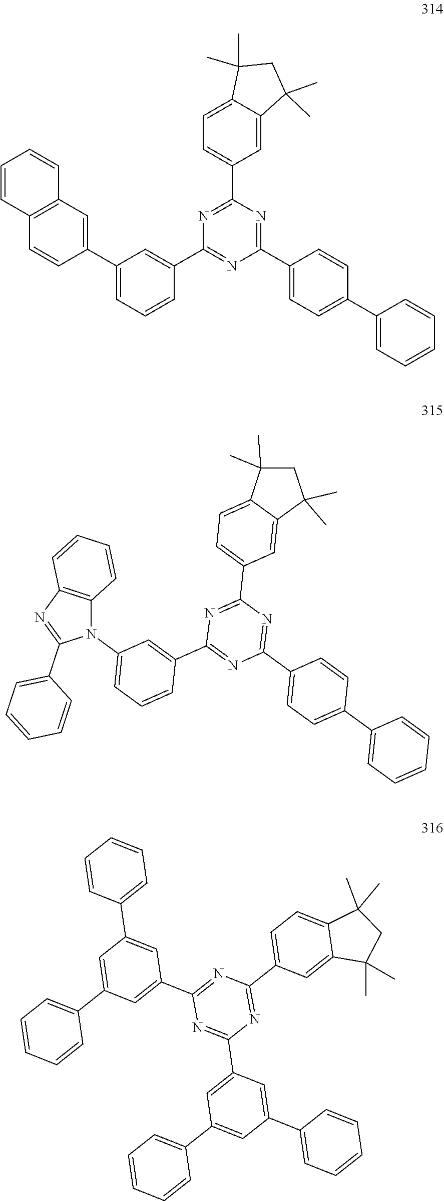 [SMILES: CC1(C)CC(C)(C)c2cc(-c3nc(-c4ccc(-c5ccccc5)cc4)nc(-c4cccc(-c5ccc6ccccc6c5)c4)n3)ccc21 ; CC1(C)CC(C)(C)c2cc(-c3nc(-c4ccc(-c5ccccc5)cc4)nc(-c4cccc(-n5c(-c6ccccc6)nc6ccccc65)c4)n3)ccc21 ; CC1(C)CC(C)(C)c2cc(-c3nc(-c4cc(-c5ccccc5)cc(-c5ccccc5)c4)nc(-c4cc(-c5ccccc5)cc(-c5ccccc5)c4)n3)ccc21]

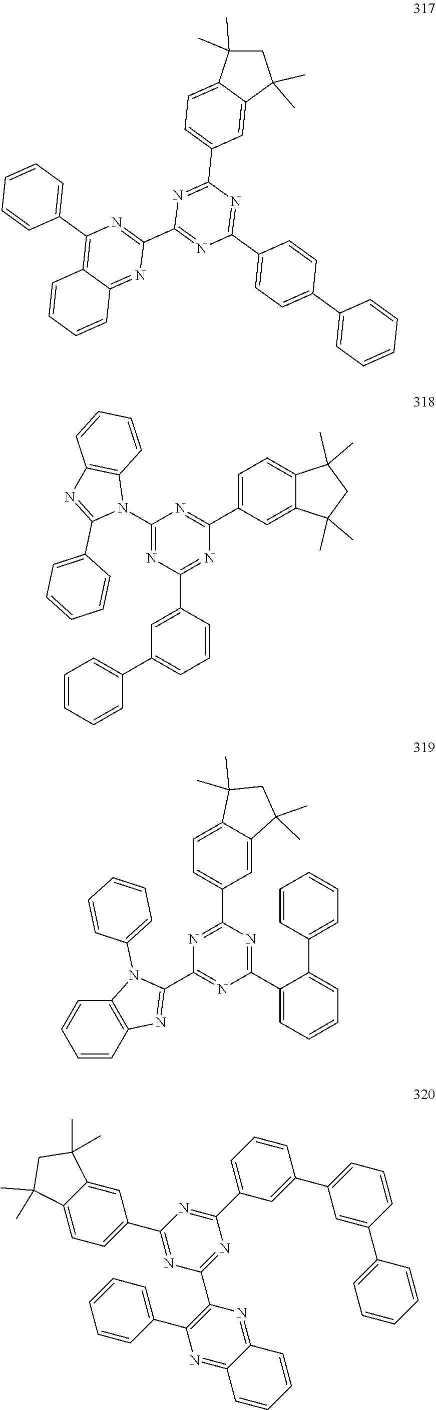

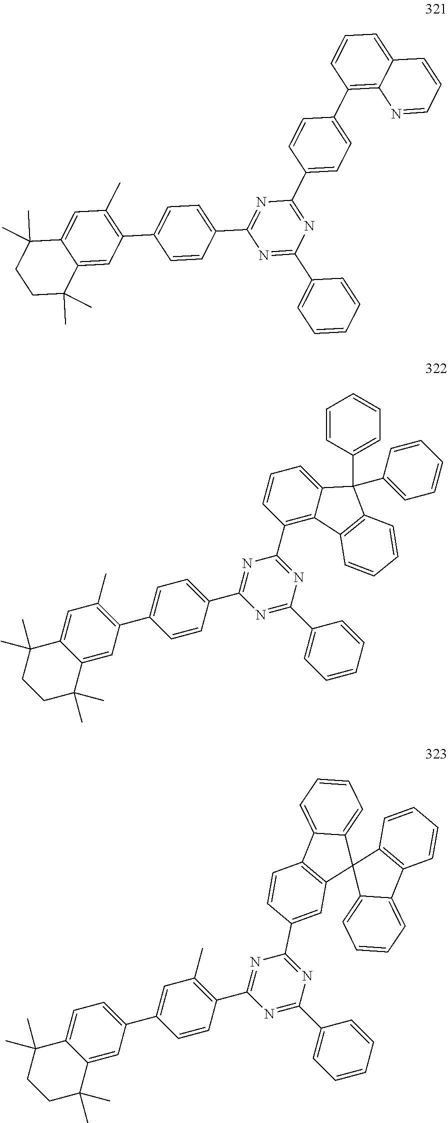 [SMILES: Cc1cc2c(cc1-c1ccc(-c3nc(-c4ccccc4)nc(-c4ccc(-c5cccc6cccnc56)cc4)n3)cc1)C(C)(C)CCC2(C)C ; Cc1cc2c(cc1-c1ccc(-c3nc(-c4ccccc4)nc(-c4cccc5c4-c4ccccc4C5(c4ccccc4)c4ccccc4)n3)cc1)C(C)(C)CCC2(C)C ; Cc1cc(-c2ccc3c(c2)C(C)(C)CCC3(C)C)ccc1-c1nc(-c2ccccc2)nc(-c2ccc3c(c2)C2(c4ccccc4-c4ccccc42)c2ccccc2-3)n1]

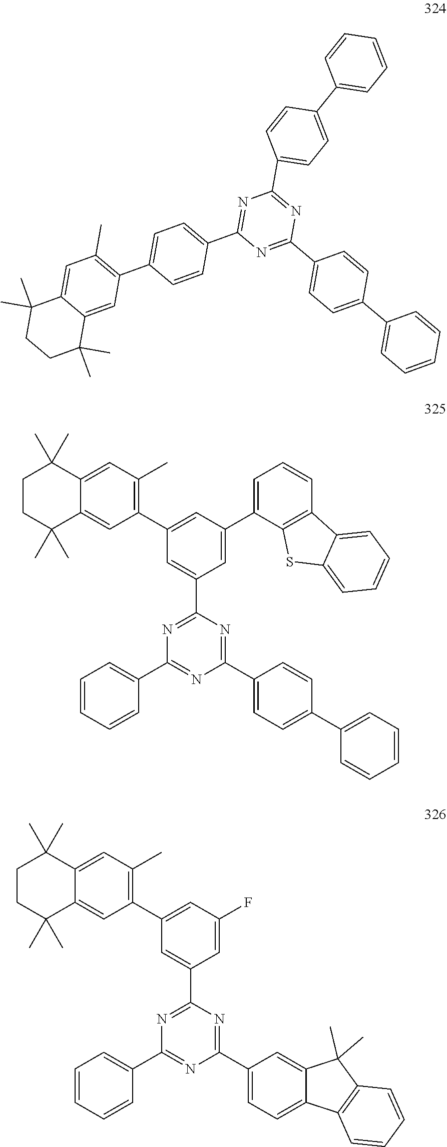 [SMILES: Cc1cc2c(cc1-c1ccc(-c3nc(-c4ccc(-c5ccccc5)cc4)nc(-c4ccc(-c5ccccc5)cc4)n3)cc1)C(C)(C)CCC2(C)C ; Cc1cc2c(cc1-c1cc(-c3nc(-c4ccccc4)nc(-c4ccc(-c5ccccc5)cc4)n3)cc(-c3cccc4c3sc3ccccc34)c1)C(C)(C)CCC2(C)C ; Cc1cc2c(cc1-c1cc(F)cc(-c3nc(-c4ccccc4)nc(-c4ccc5c(c4)C(C)(C)c4ccccc4-5)n3)c1)C(C)(C)CCC2(C)C]

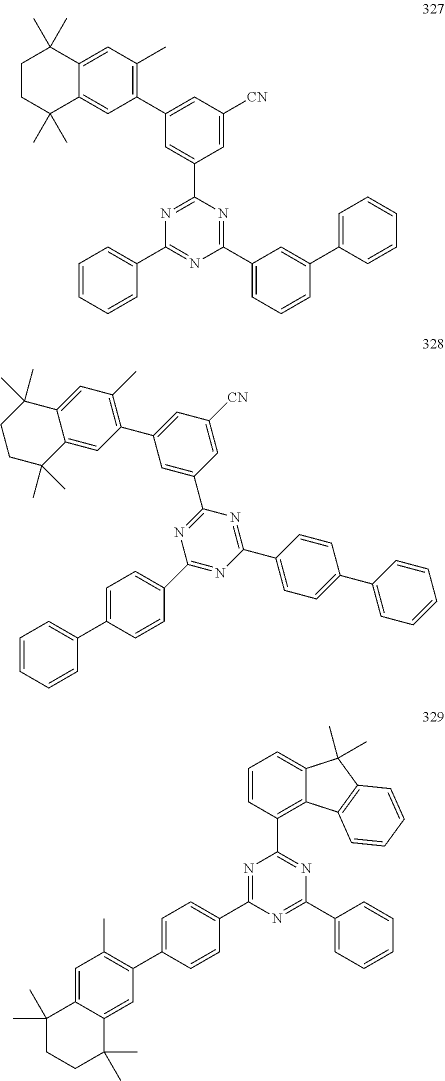 [SMILES: Cc1cc2c(cc1-c1cc(C#N)cc(-c3nc(-c4ccccc4)nc(-c4cccc(-c5ccccc5)c4)n3)c1)C(C)(C)CCC2(C)C ; Cc1cc2c(cc1-c1cc(C#N)cc(-c3nc(-c4ccc(-c5ccccc5)cc4)nc(-c4ccc(-c5ccccc5)cc4)n3)c1)C(C)(C)CCC2(C)C ; Cc1cc2c(cc1-c1ccc(-c3nc(-c4ccccc4)nc(-c4cccc5c4-c4ccccc4C5(C)C)n3)cc1)C(C)(C)CCC2(C)C]

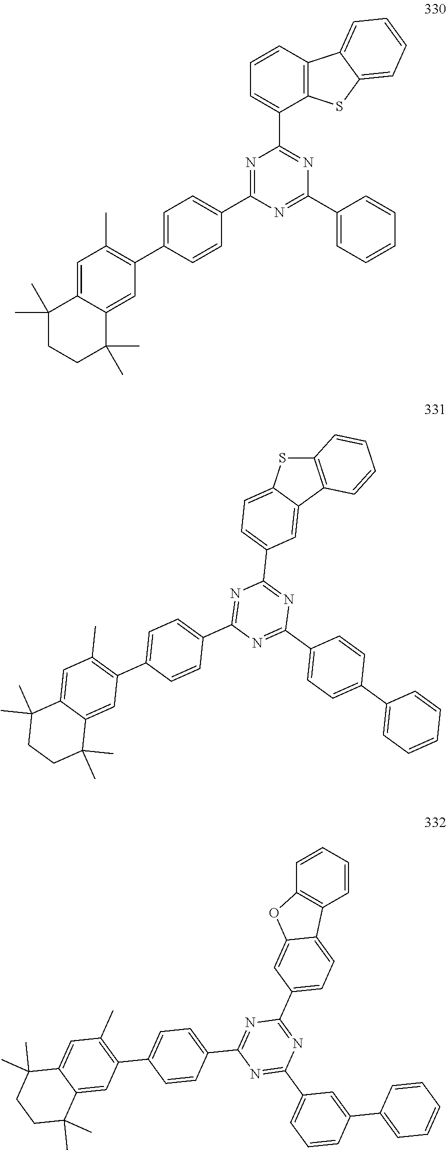 [SMILES: Cc1cc2c(cc1-c1ccc(-c3nc(-c4ccccc4)nc(-c4cccc5c4sc4ccccc45)n3)cc1)C(C)(C)CCC2(C)C ; Cc1cc2c(cc1-c1ccc(-c3nc(-c4ccc(-c5ccccc5)cc4)nc(-c4ccc5sc6ccccc6c5c4)n3)cc1)C(C)(C)CCC2(C)C ; Cc1cc2c(cc1-c1ccc(-c3nc(-c4cccc(-c5ccccc5)c4)nc(-c4ccc5c(c4)oc4ccccc45)n3)cc1)C(C)(C)CCC2(C)C]

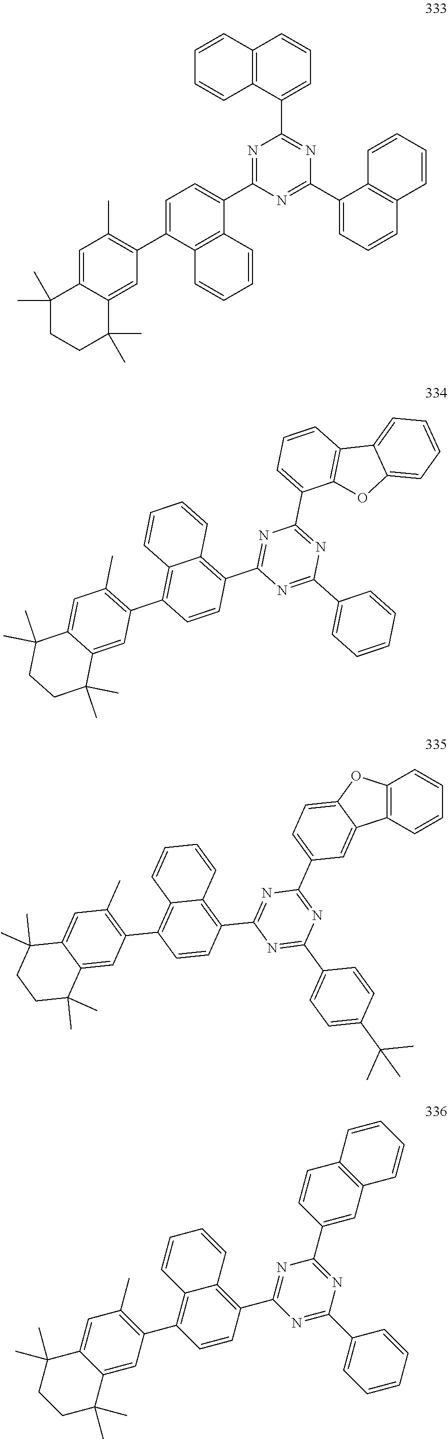

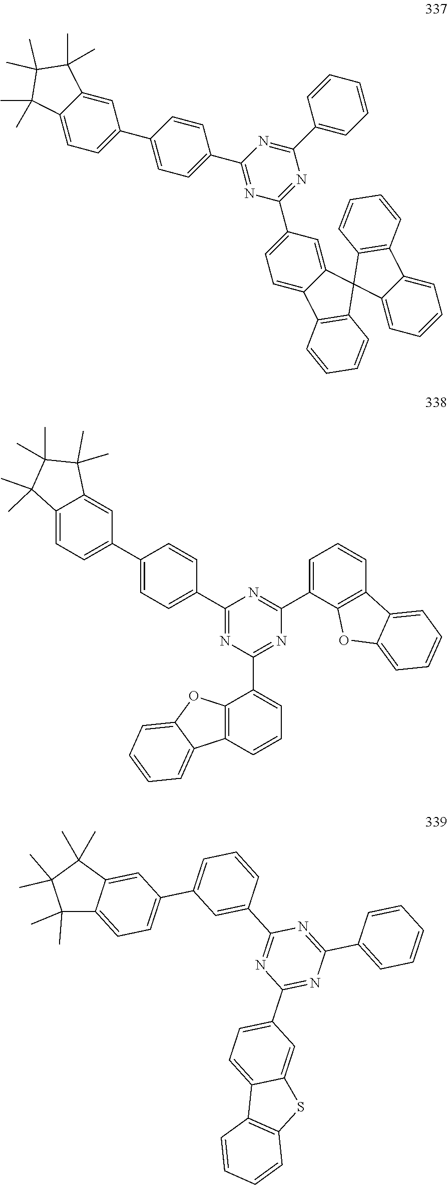 [SMILES: CC1(C)c2ccc(-c3ccc(-c4nc(-c5ccccc5)nc(-c5ccc6c(c5)C5(c7ccccc7-c7ccccc75)c5ccccc5-6)n4)cc3)cc2C(C)(C)C1(C)C ; CC1(C)c2ccc(-c3ccc(-c4nc(-c5cccc6c5oc5ccccc56)nc(-c5cccc6c5oc5ccccc56)n4)cc3)cc2C(C)(C)C1(C)C ; CC1(C)c2ccc(-c3cccc(-c4nc(-c5ccccc5)nc(-c5ccc6c(c5)sc5ccccc56)n4)c3)cc2C(C)(C)C1(C)C]

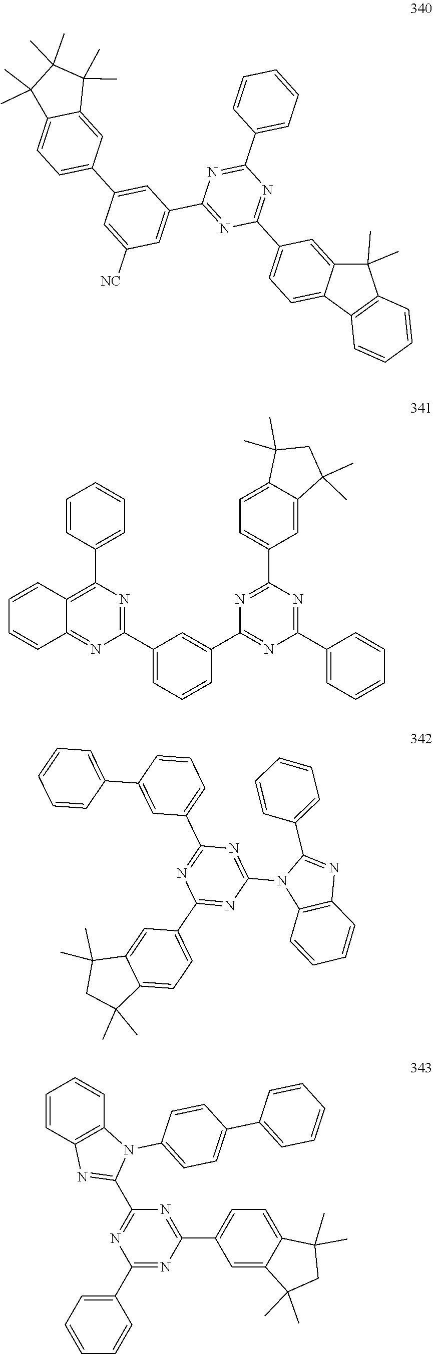 [SMILES: CC1(C)c2ccccc2-c2ccc(-c3nc(-c4ccccc4)nc(-c4cc(C#N)cc(-c5ccc6c(c5)C(C)(C)C(C)(C)C6(C)C)c4)n3)cc21 ; CC1(C)CC(C)(C)c2cc(-c3nc(-c4ccccc4)nc(-c4cccc(-c5nc(-c6ccccc6)c6ccccc6n5)c4)n3)ccc21 ; CC1(C)CC(C)(C)c2cc(-c3nc(-c4cccc(-c5ccccc5)c4)nc(-n4c(-c5ccccc5)nc5ccccc54)n3)ccc21 ; CC1(C)CC(C)(C)c2cc(-c3nc(-c4ccccc4)nc(-c4nc5ccccc5n4-c4ccc(-c5ccccc5)cc4)n3)ccc21]

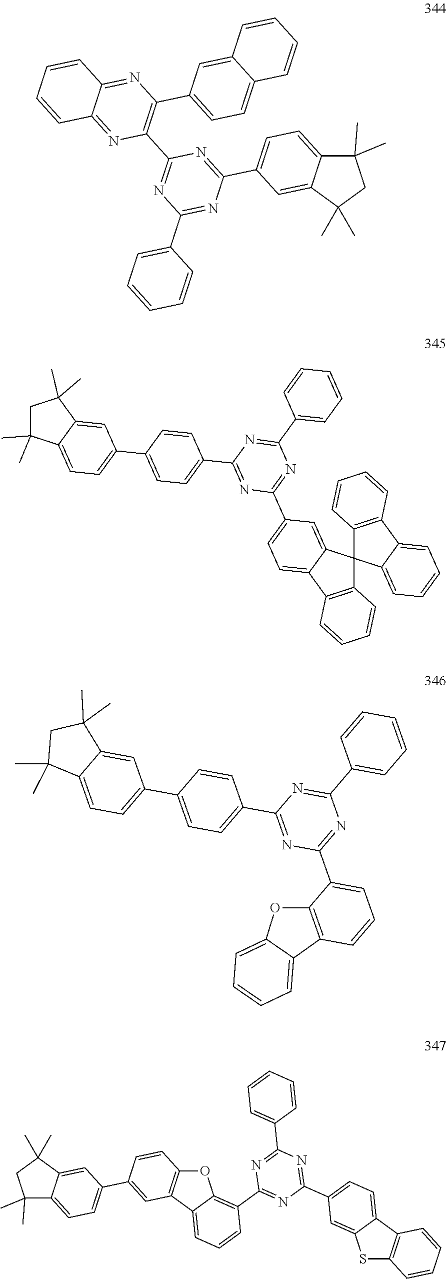

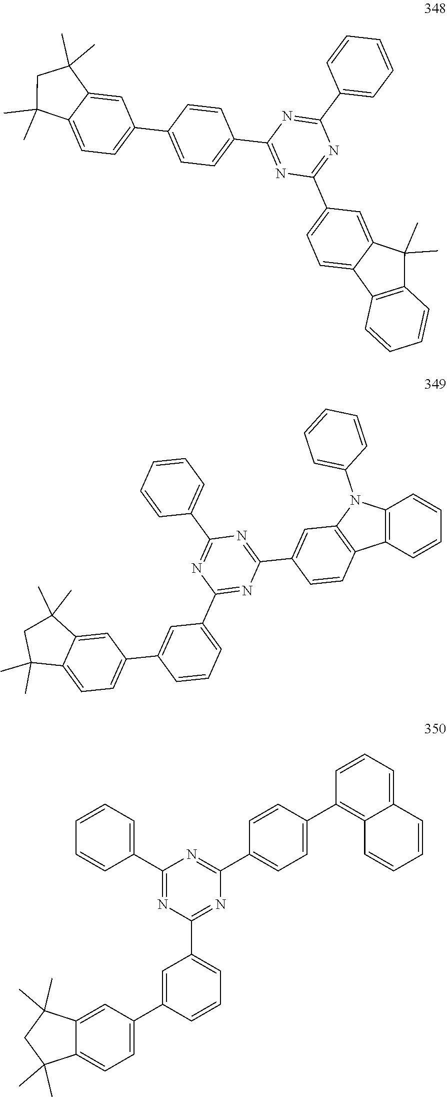

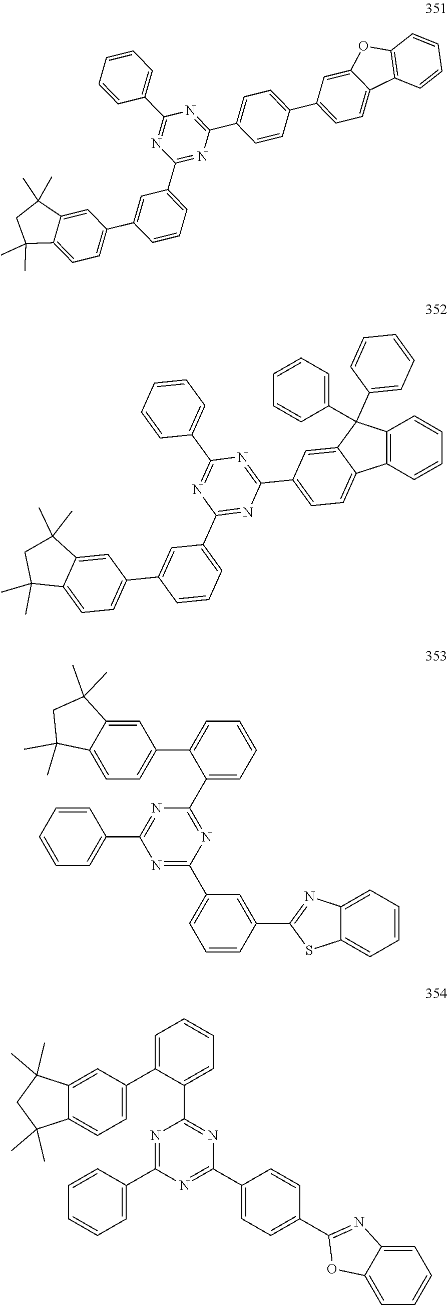 [SMILES: CC1(C)CC(C)(C)c2cc(-c3cccc(-c4nc(-c5ccccc5)nc(-c5ccc(-c6ccc7c(c6)oc6ccccc67)cc5)n4)c3)ccc21 ; CC1(C)CC(C)(C)c2cc(-c3cccc(-c4nc(-c5ccccc5)nc(-c5ccc6c(c5)C(c5ccccc5)(c5ccccc5)c5ccccc5-6)n4)c3)ccc21 ; CC1(C)CC(C)(C)c2cc(-c3ccccc3-c3nc(-c4ccccc4)nc(-c4cccc(-c5nc6ccccc6s5)c4)n3)ccc21 ; CC1(C)CC(C)(C)c2cc(-c3ccccc3-c3nc(-c4ccccc4)nc(-c4ccc(-c5nc6ccccc6o5)cc4)n3)ccc21]

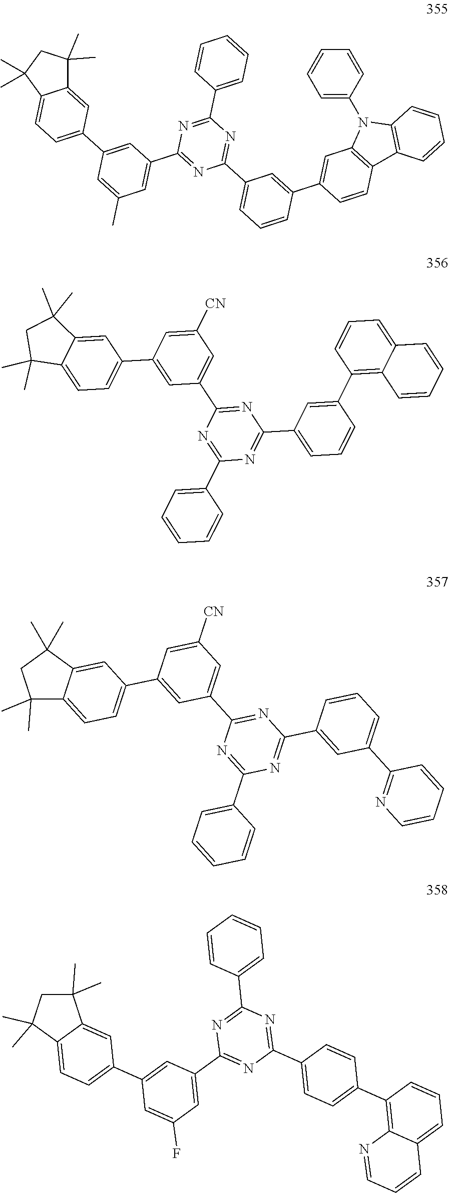 [SMILES: Cc1cc(-c2ccc3c(c2)C(C)(C)CC3(C)C)cc(-c2nc(-c3ccccc3)nc(-c3cccc(-c4ccc5c6ccccc6n(-c6ccccc6)c5c4)c3)n2)c1 ; CC1(C)CC(C)(C)c2cc(-c3cc(C#N)cc(-c4nc(-c5ccccc5)nc(-c5cccc(-c6cccc7ccccc67)c5)n4)c3)ccc21 ; CC1(C)CC(C)(C)c2cc(-c3cc(C#N)cc(-c4nc(-c5ccccc5)nc(-c5cccc(-c6ccccn6)c5)n4)c3)ccc21 ; CC1(C)CC(C)(C)c2cc(-c3cc(F)cc(-c4nc(-c5ccccc5)nc(-c5ccc(-c6cccc7cccnc67)cc5)n4)c3)ccc21]

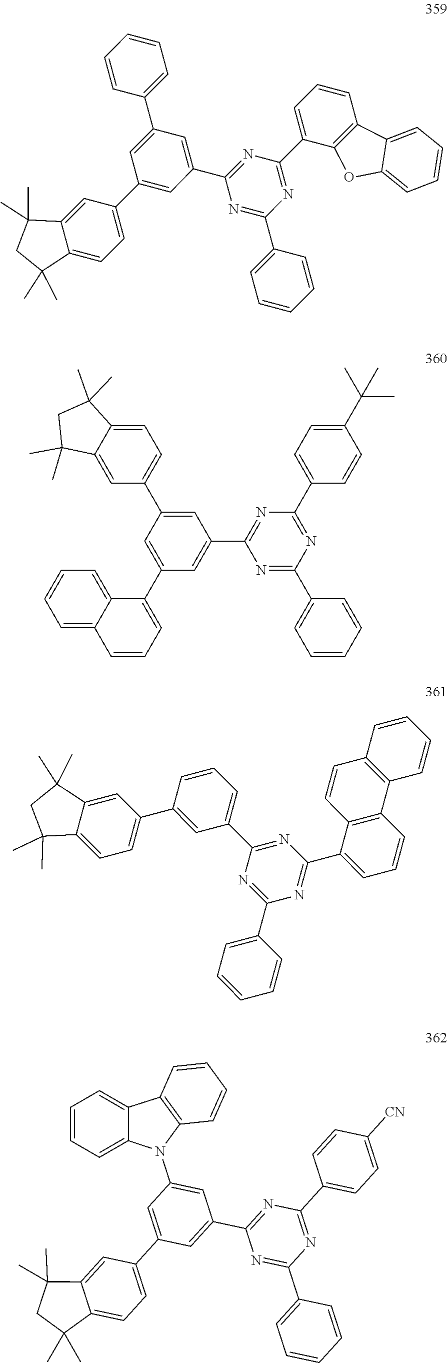

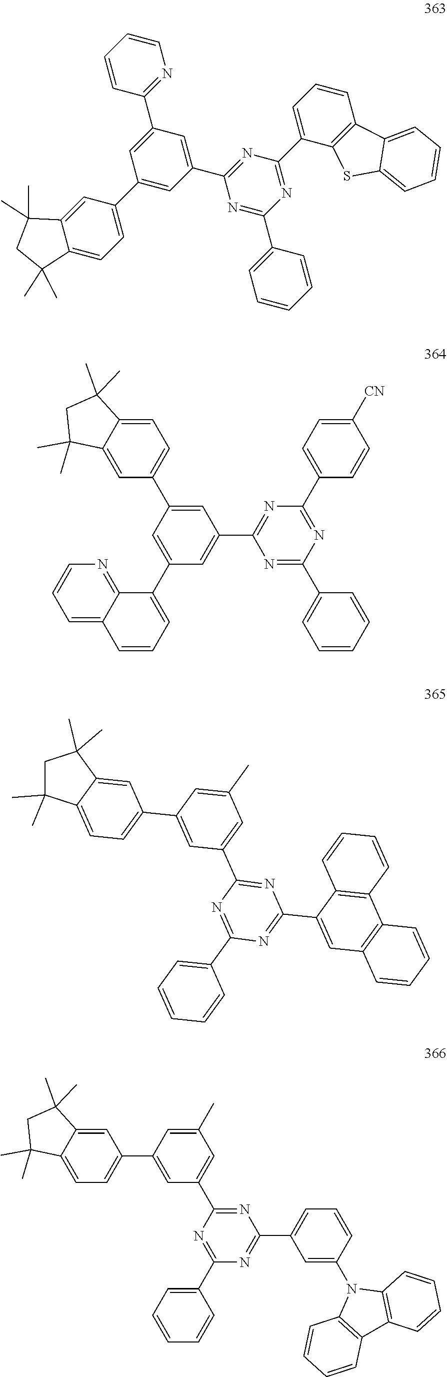 [SMILES: CC1(C)CC(C)(C)c2cc(-c3cc(-c4ccccn4)cc(-c4nc(-c5ccccc5)nc(-c5cccc6c5sc5ccccc56)n4)c3)ccc21 ; CC1(C)CC(C)(C)c2cc(-c3cc(-c4nc(-c5ccccc5)nc(-c5ccc(C#N)cc5)n4)cc(-c4cccc5cccnc45)c3)ccc21 ; Cc1cc(-c2ccc3c(c2)C(C)(C)CC3(C)C)cc(-c2nc(-c3ccccc3)nc(-c3cc4ccccc4c4ccccc34)n2)c1 ; Cc1cc(-c2ccc3c(c2)C(C)(C)CC3(C)C)cc(-c2nc(-c3ccccc3)nc(-c3cccc(-n4c5ccccc5c5ccccc54)c3)n2)c1]

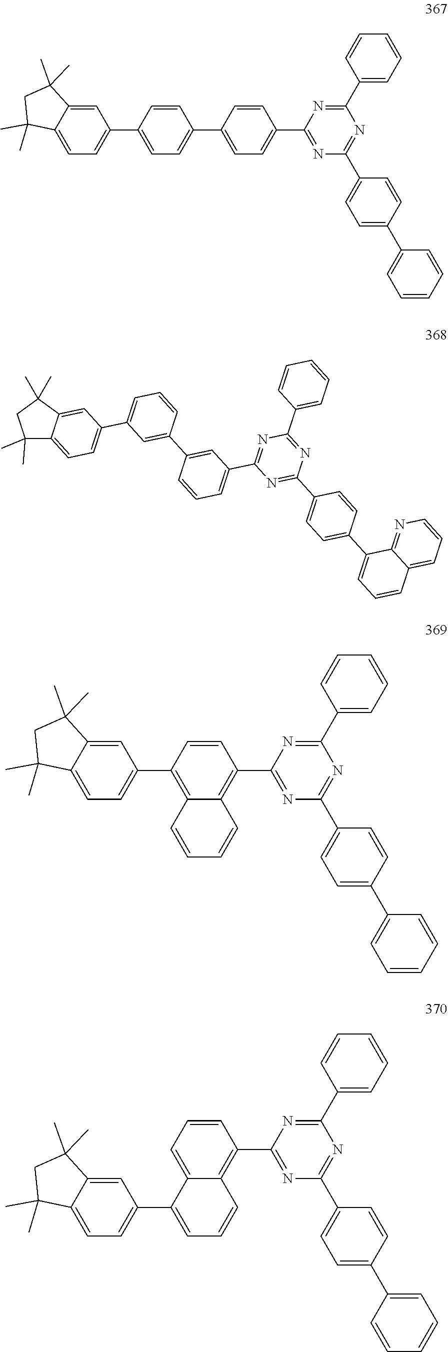 [SMILES: CC1(C)CC(C)(C)c2cc(-c3ccc(-c4ccc(-c5nc(-c6ccccc6)nc(-c6ccc(-c7ccccc7)cc6)n5)cc4)cc3)ccc21 ; CC1(C)CC(C)(C)c2cc(-c3cccc(-c4cccc(-c5nc(-c6ccccc6)nc(-c6ccc(-c7cccc8cccnc78)cc6)n5)c4)c3)ccc21 ; CC1(C)CC(C)(C)c2cc(-c3ccc(-c4nc(-c5ccccc5)nc(-c5ccc(-c6ccccc6)cc5)n4)c4ccccc34)ccc21 ; CC1(C)CC(C)(C)c2cc(-c3cccc4c(-c5nc(-c6ccccc6)nc(-c6ccc(-c7ccccc7)cc6)n5)cccc34)ccc21]

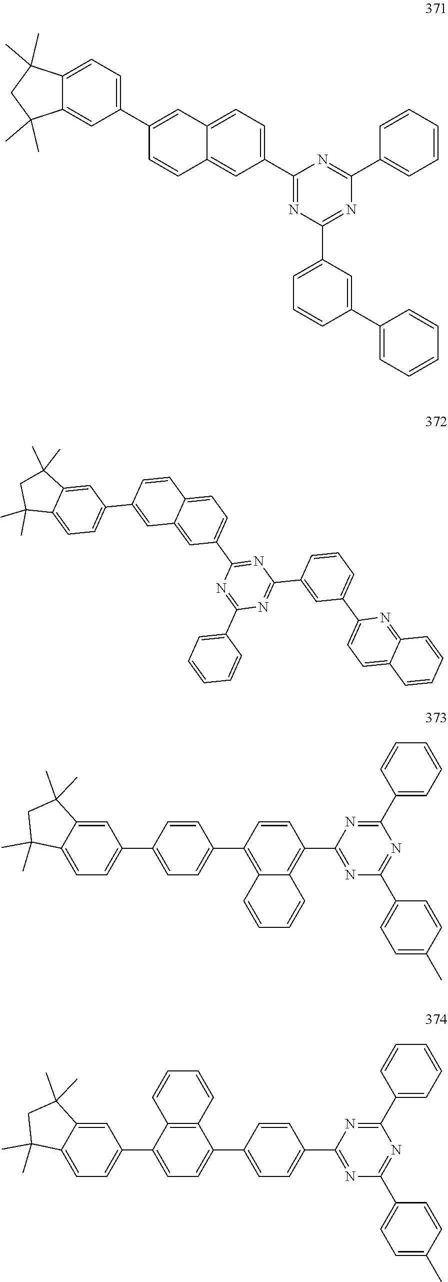

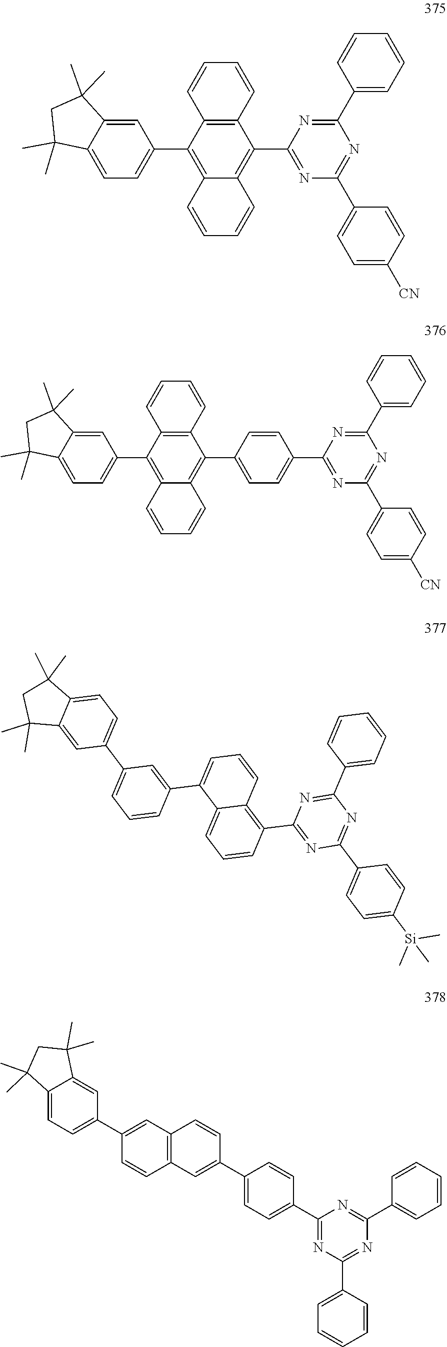

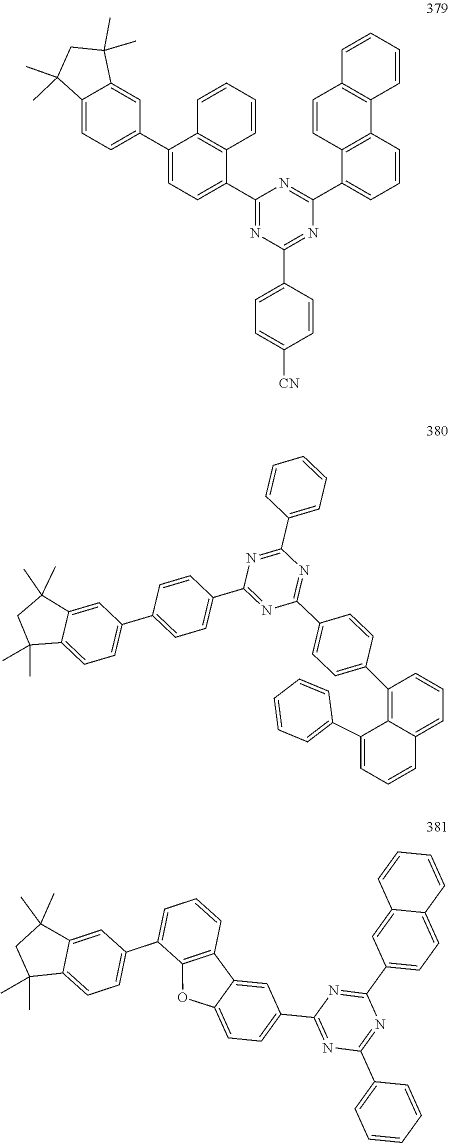 [SMILES: CC1(C)CC(C)(C)c2cc(-c3ccc(-c4nc(-c5ccc(C#N)cc5)nc(-c5cccc6c5ccc5ccccc56)n4)c4ccccc34)ccc21 ; CC1(C)CC(C)(C)c2cc(-c3ccc(-c4nc(-c5ccccc5)nc(-c5ccc(-c6cccc7cccc(-c8ccccc8)c67)cc5)n4)cc3)ccc21 ; CC1(C)CC(C)(C)c2cc(-c3cccc4c3oc3ccc(-c5nc(-c6ccccc6)nc(-c6ccc7ccccc7c6)n5)cc34)ccc21]

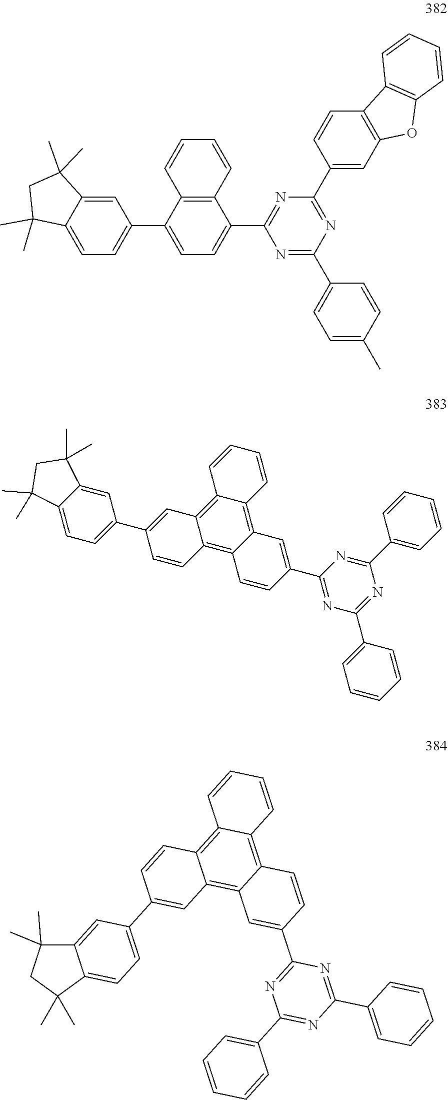 [SMILES: Cc1ccc(-c2nc(-c3ccc4c(c3)oc3ccccc34)nc(-c3ccc(-c4ccc5c(c4)C(C)(C)CC5(C)C)c4ccccc34)n2)cc1 ; CC1(C)CC(C)(C)c2cc(-c3ccc4c5ccc(-c6nc(-c7ccccc7)nc(-c7ccccc7)n6)cc5c5ccccc5c4c3)ccc21 ; CC1(C)CC(C)(C)c2cc(-c3ccc4c5ccccc5c5ccc(-c6nc(-c7ccccc7)nc(-c7ccccc7)n6)cc5c4c3)ccc21]

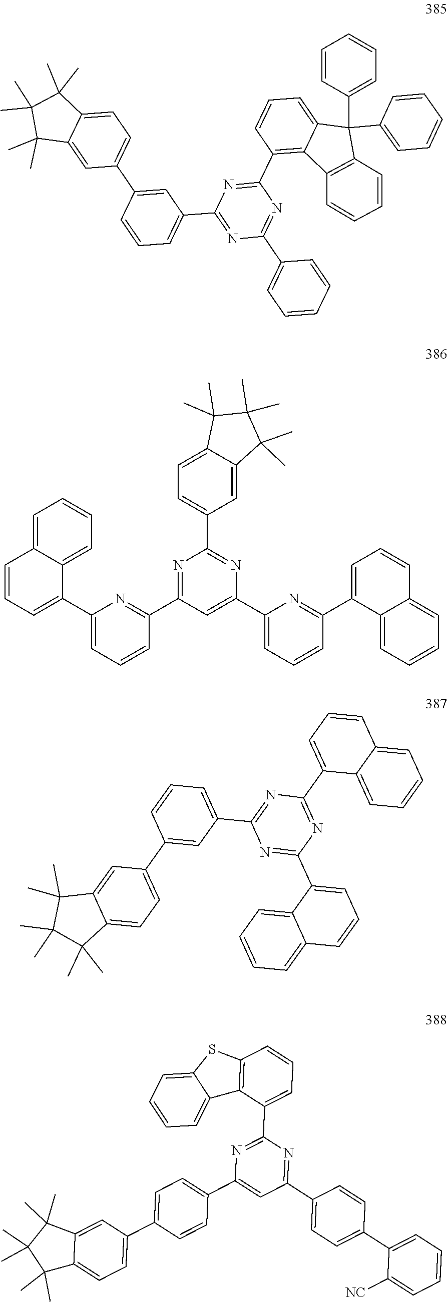 [SMILES: CC1(C)c2ccc(-c3cccc(-c4nc(-c5ccccc5)nc(-c5cccc6c5-c5ccccc5C6(c5ccccc5)c5ccccc5)n4)c3)cc2C(C)(C)C1(C)C ; CC1(C)c2ccc(-c3nc(-c4cccc(-c5cccc6ccccc56)n4)cc(-c4cccc(-c5cccc6ccccc56)n4)n3)cc2C(C)(C)C1(C)C ; CC1(C)c2ccc(-c3cccc(-c4nc(-c5cccc6ccccc56)nc(-c5cccc6ccccc56)n4)c3)cc2C(C)(C)C1(C)C ; CC1(C)c2ccc(-c3ccc(-c4cc(-c5ccc(-c6ccccc6C#N)cc5)nc(-c5cccc6sc7ccccc7c56)n4)cc3)cc2C(C)(C)C1(C)C]

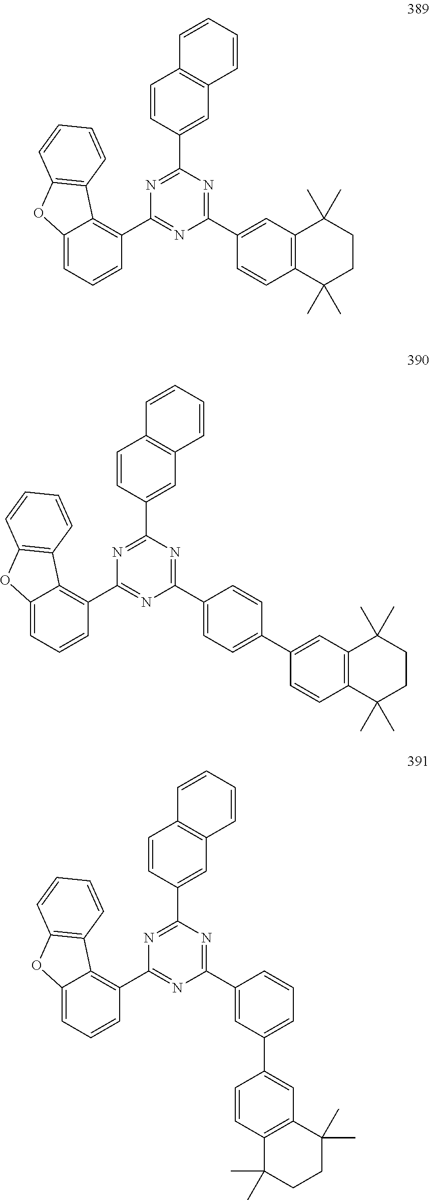 [SMILES: CC1(C)CCC(C)(C)c2cc(-c3nc(-c4ccc5ccccc5c4)nc(-c4cccc5oc6ccccc6c45)n3)ccc21 ; CC1(C)CCC(C)(C)c2cc(-c3ccc(-c4nc(-c5ccc6ccccc6c5)nc(-c5cccc6oc7ccccc7c56)n4)cc3)ccc21 ; CC1(C)CCC(C)(C)c2cc(-c3cccc(-c4nc(-c5ccc6ccccc6c5)nc(-c5cccc6oc7ccccc7c56)n4)c3)ccc21]

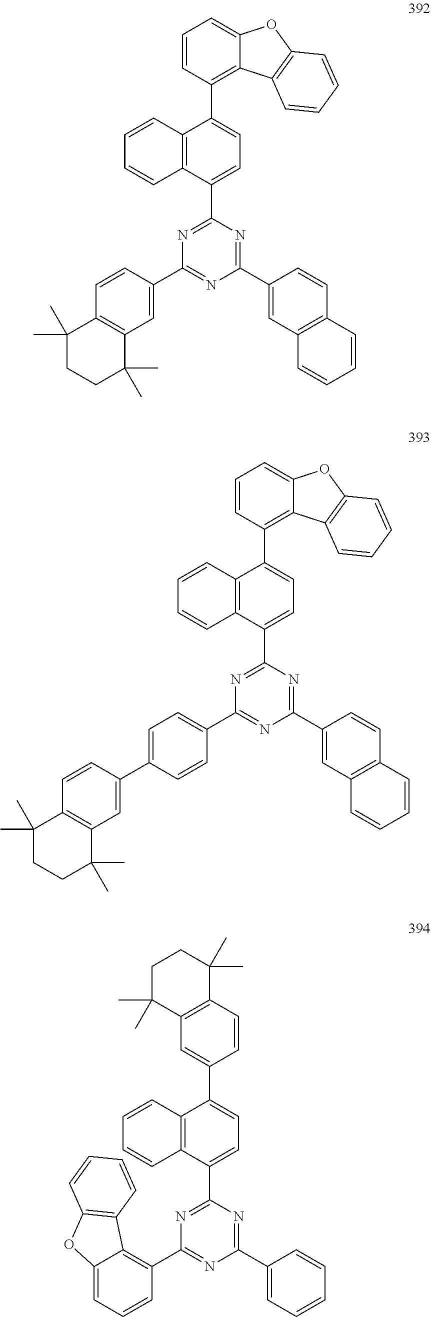 [SMILES: CC1(C)CCC(C)(C)c2cc(-c3nc(-c4ccc5ccccc5c4)nc(-c4ccc(-c5cccc6oc7ccccc7c56)c5ccccc45)n3)ccc21 ; CC1(C)CCC(C)(C)c2cc(-c3ccc(-c4nc(-c5ccc6ccccc6c5)nc(-c5ccc(-c6cccc7oc8ccccc8c67)c6ccccc56)n4)cc3)ccc21 ; CC1(C)CCC(C)(C)c2cc(-c3ccc(-c4nc(-c5ccccc5)nc(-c5cccc6oc7ccccc7c56)n4)c4ccccc34)ccc21]

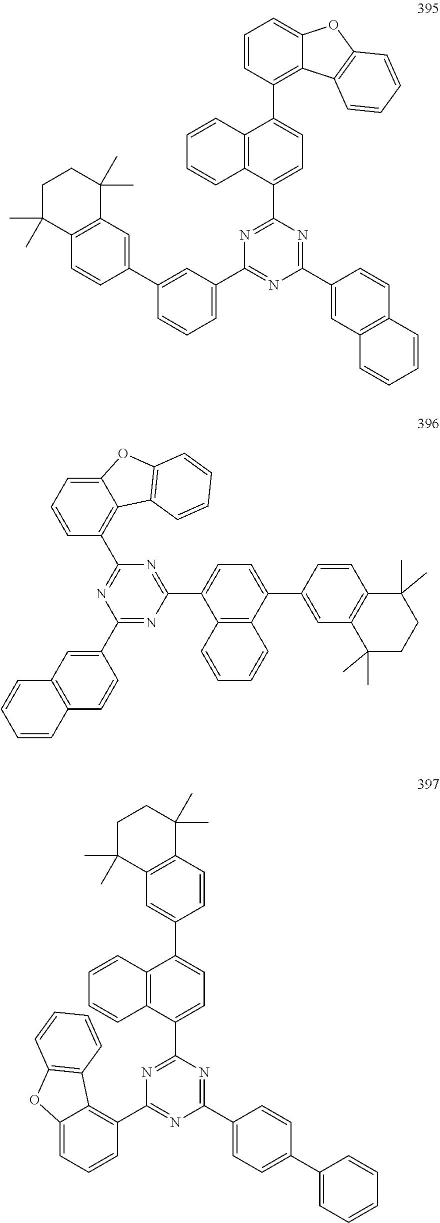 [SMILES: CC1(C)CCC(C)(C)c2cc(-c3cccc(-c4nc(-c5ccc6ccccc6c5)nc(-c5ccc(-c6cccc7oc8ccccc8c67)c6ccccc56)n4)c3)ccc21 ; CC1(C)CCC(C)(C)c2cc(-c3ccc(-c4nc(-c5ccc6ccccc6c5)nc(-c5cccc6oc7ccccc7c56)n4)c4ccccc34)ccc21 ; CC1(C)CCC(C)(C)c2cc(-c3ccc(-c4nc(-c5ccc(-c6ccccc6)cc5)nc(-c5cccc6oc7ccccc7c56)n4)c4ccccc34)ccc21]

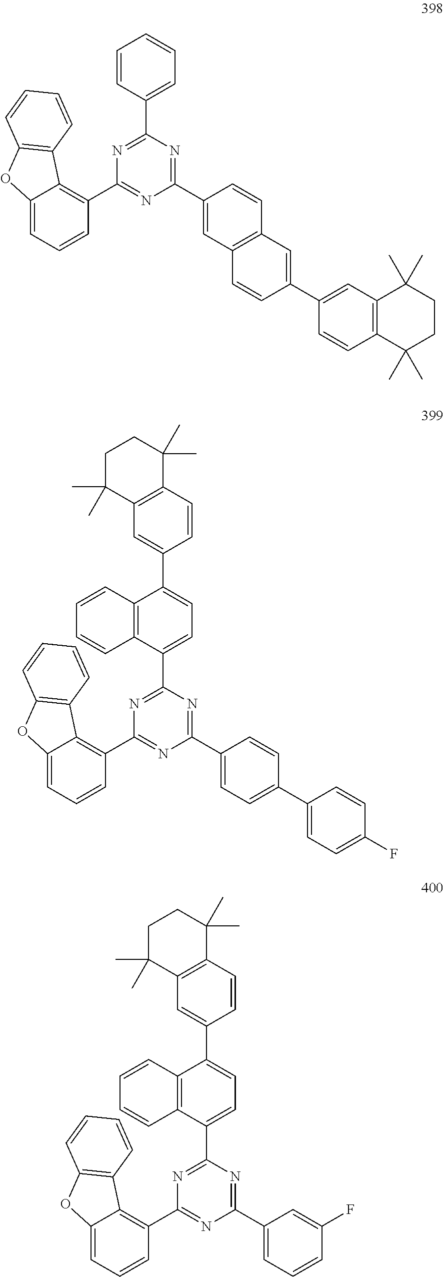 [SMILES: CC1(C)CCC(C)(C)c2cc(-c3ccc4cc(-c5nc(-c6ccccc6)nc(-c6cccc7oc8ccccc8c67)n5)ccc4c3)ccc21 ; CC1(C)CCC(C)(C)c2cc(-c3ccc(-c4nc(-c5ccc(-c6ccc(F)cc6)cc5)nc(-c5cccc6oc7ccccc7c56)n4)c4ccccc34)ccc21 ; CC1(C)CCC(C)(C)c2cc(-c3ccc(-c4nc(-c5cccc(F)c5)nc(-c5cccc6oc7ccccc7c56)n4)c4ccccc34)ccc21]

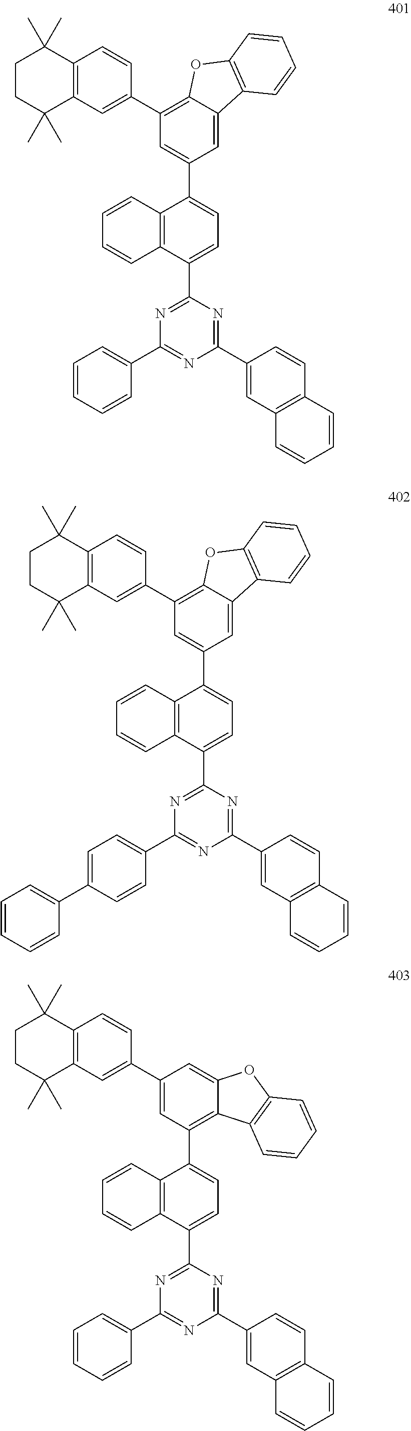

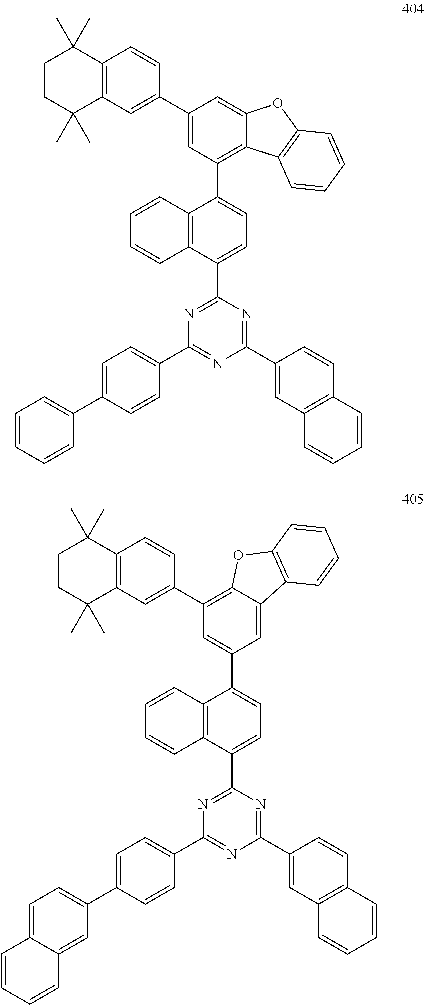 [SMILES: CC1(C)CCC(C)(C)c2cc(-c3cc(-c4ccc(-c5nc(-c6ccc(-c7ccccc7)cc6)nc(-c6ccc7ccccc7c6)n5)c5ccccc45)c4c(c3)oc3ccccc34)ccc21 ; CC1(C)CCC(C)(C)c2cc(-c3cc(-c4ccc(-c5nc(-c6ccc(-c7ccc8ccccc8c7)cc6)nc(-c6ccc7ccccc7c6)n5)c5ccccc45)cc4c3oc3ccccc34)ccc21]

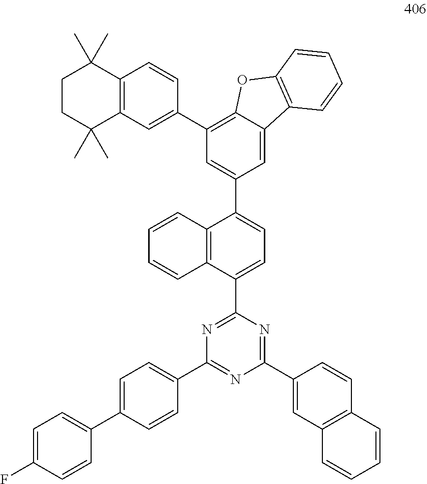 [SMILES: CC1(C)CCC(C)(C)c2cc(-c3cc(-c4ccc(-c5nc(-c6ccc(-c7ccc(F)cc7)cc6)nc(-c6ccc7ccccc7c6)n5)c5ccccc45)cc4c3oc3ccccc34)ccc21]

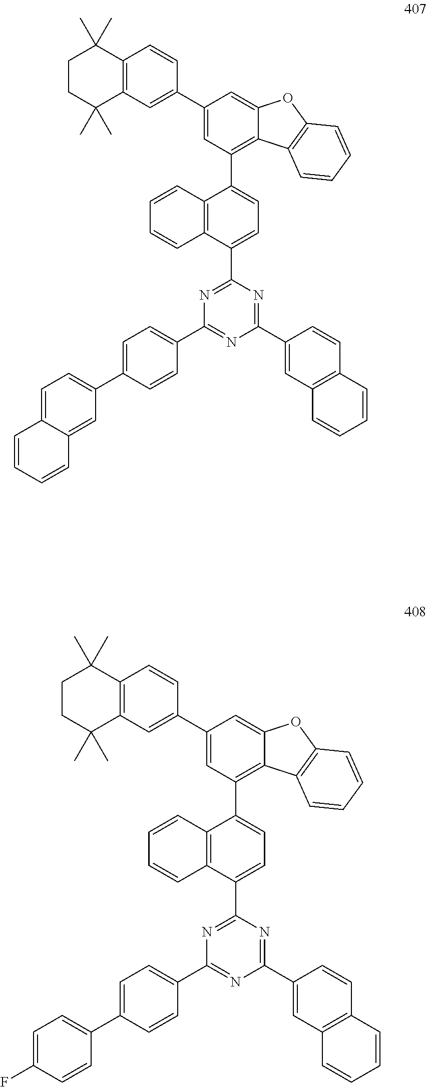 [SMILES: CC1(C)CCC(C)(C)c2cc(-c3cc(-c4ccc(-c5nc(-c6ccc(-c7ccc8ccccc8c7)cc6)nc(-c6ccc7ccccc7c6)n5)c5ccccc45)c4c(c3)oc3ccccc34)ccc21 ; CC1(C)CCC(C)(C)c2cc(-c3cc(-c4ccc(-c5nc(-c6ccc(-c7ccc(F)cc7)cc6)nc(-c6ccc7ccccc7c6)n5)c5ccccc45)c4c(c3)oc3ccccc34)ccc21]

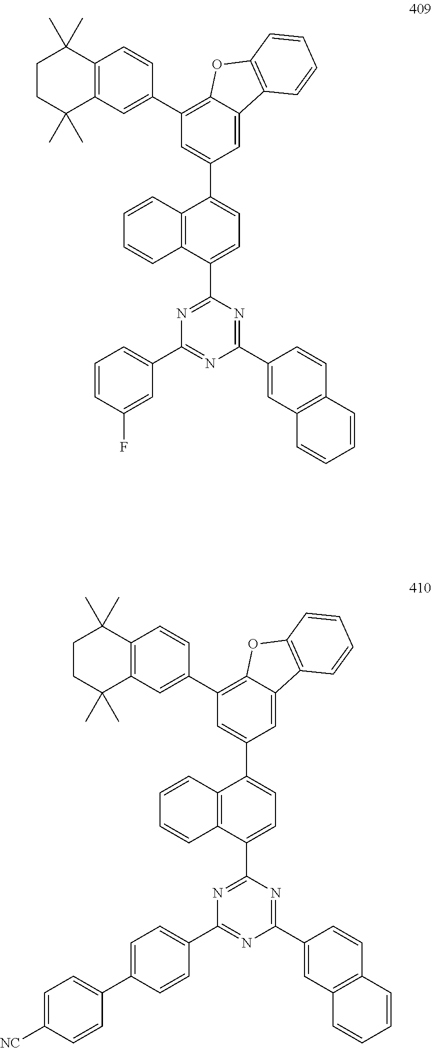 [SMILES: CC1(C)CCC(C)(C)c2cc(-c3cc(-c4ccc(-c5nc(-c6cccc(F)c6)nc(-c6ccc7ccccc7c6)n5)c5ccccc45)cc4c3oc3ccccc34)ccc21 ; CC1(C)CCC(C)(C)c2cc(-c3cc(-c4ccc(-c5nc(-c6ccc(-c7ccc(C#N)cc7)cc6)nc(-c6ccc7ccccc7c6)n5)c5ccccc45)cc4c3oc3ccccc34)ccc21]

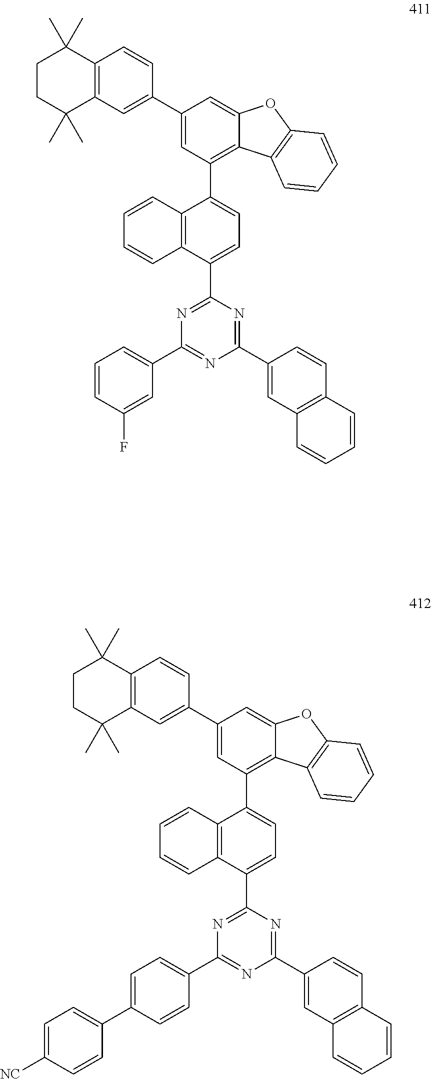 [SMILES: CC1(C)CCC(C)(C)c2cc(-c3cc(-c4ccc(-c5nc(-c6cccc(F)c6)nc(-c6ccc7ccccc7c6)n5)c5ccccc45)c4c(c3)oc3ccccc34)ccc21 ; CC1(C)CCC(C)(C)c2cc(-c3cc(-c4ccc(-c5nc(-c6ccc(-c7ccc(C#N)cc7)cc6)nc(-c6ccc7ccccc7c6)n5)c5ccccc45)c4c(c3)oc3ccccc34)ccc21]

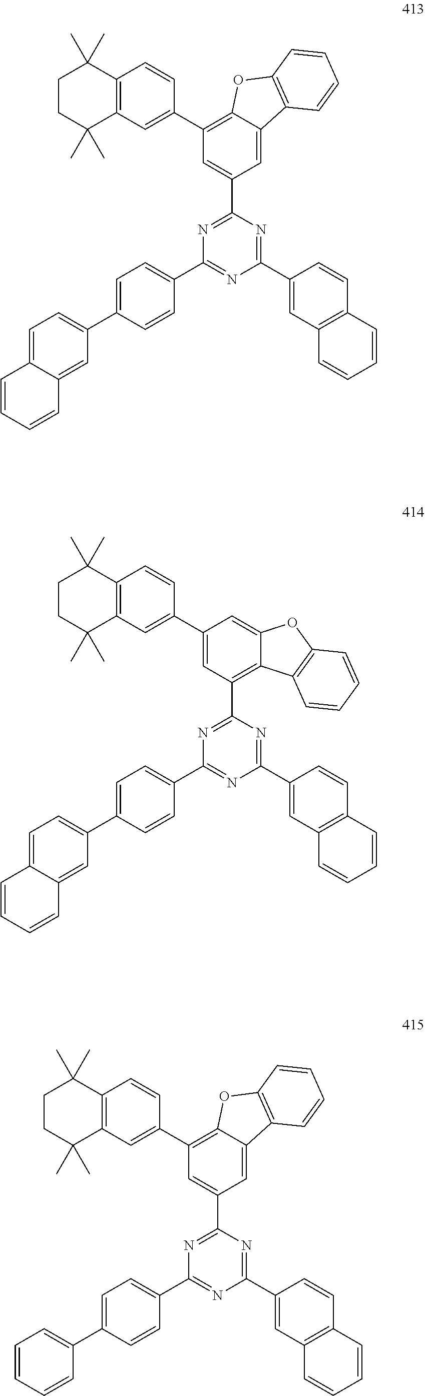 [SMILES: CC1(C)CCC(C)(C)c2cc(-c3cc(-c4nc(-c5ccc(-c6ccc7ccccc7c6)cc5)nc(-c5ccc6ccccc6c5)n4)cc4c3oc3ccccc34)ccc21 ; CC1(C)CCC(C)(C)c2cc(-c3cc(-c4nc(-c5ccc(-c6ccc7ccccc7c6)cc5)nc(-c5ccc6ccccc6c5)n4)c4c(c3)oc3ccccc34)ccc21 ; CC1(C)CCC(C)(C)c2cc(-c3cc(-c4nc(-c5ccc(-c6ccccc6)cc5)nc(-c5ccc6ccccc6c5)n4)cc4c3oc3ccccc34)ccc21]

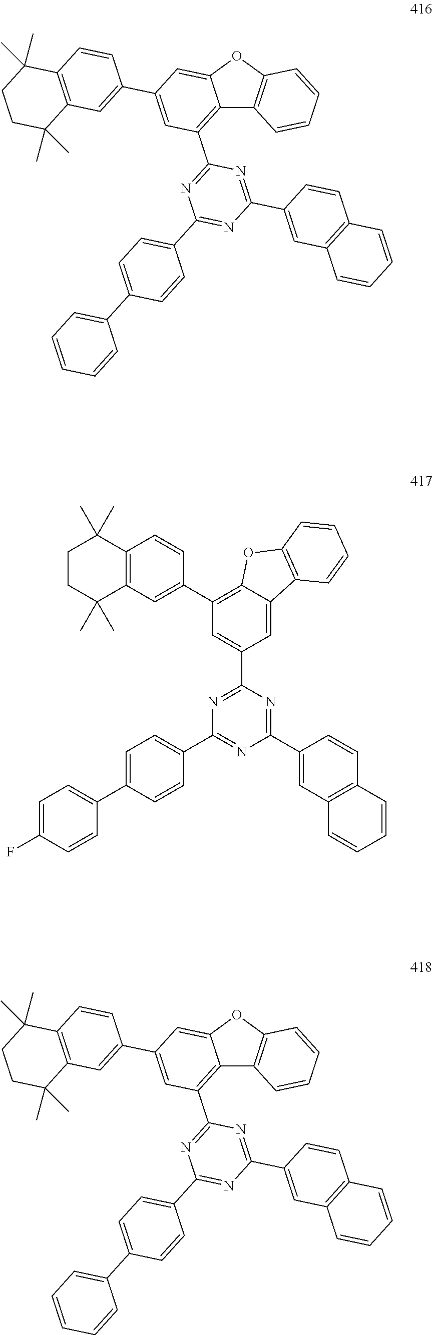 [SMILES: CC1(C)CCC(C)(C)c2cc(-c3cc(-c4nc(-c5ccc(-c6ccccc6)cc5)nc(-c5ccc6ccccc6c5)n4)c4c(c3)oc3ccccc34)ccc21 ; CC1(C)CCC(C)(C)c2cc(-c3cc(-c4nc(-c5ccc(-c6ccc(F)cc6)cc5)nc(-c5ccc6ccccc6c5)n4)cc4c3oc3ccccc34)ccc21 ; CC1(C)CCC(C)(C)c2cc(-c3cc(-c4nc(-c5ccc(-c6ccccc6)cc5)nc(-c5ccc6ccccc6c5)n4)c4c(c3)oc3ccccc34)ccc21]

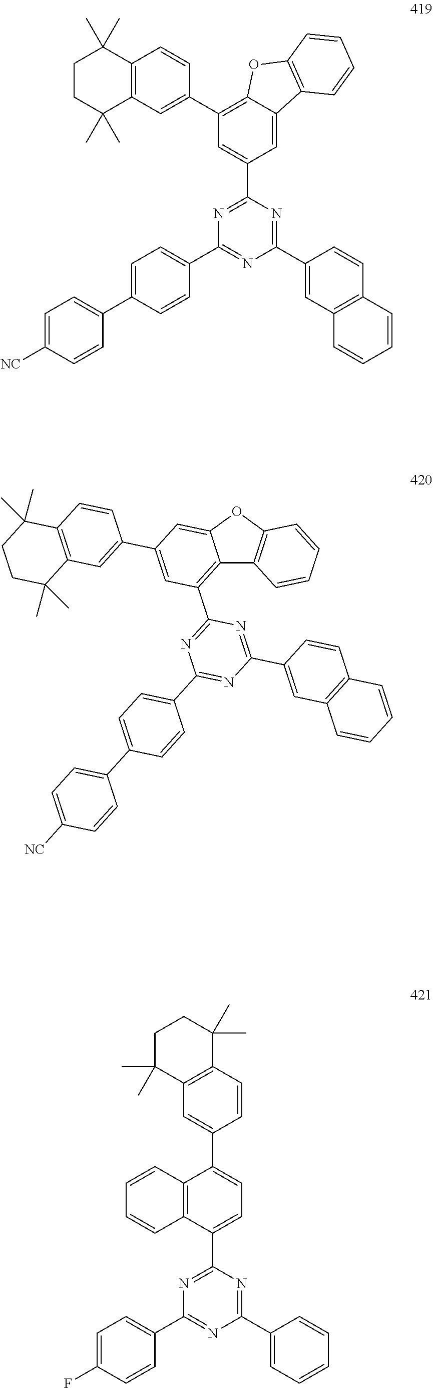 [SMILES: CC1(C)CCC(C)(C)c2cc(-c3cc(-c4nc(-c5ccc(-c6ccc(C#N)cc6)cc5)nc(-c5ccc6ccccc6c5)n4)cc4c3oc3ccccc34)ccc21 ; CC1(C)CCC(C)(C)c2cc(-c3cc(-c4nc(-c5ccc(-c6ccc(C#N)cc6)cc5)nc(-c5ccc6ccccc6c5)n4)c4c(c3)oc3ccccc34)ccc21 ; CC1(C)CCC(C)(C)c2cc(-c3ccc(-c4nc(-c5ccccc5)nc(-c5ccc(F)cc5)n4)c4ccccc34)ccc21]

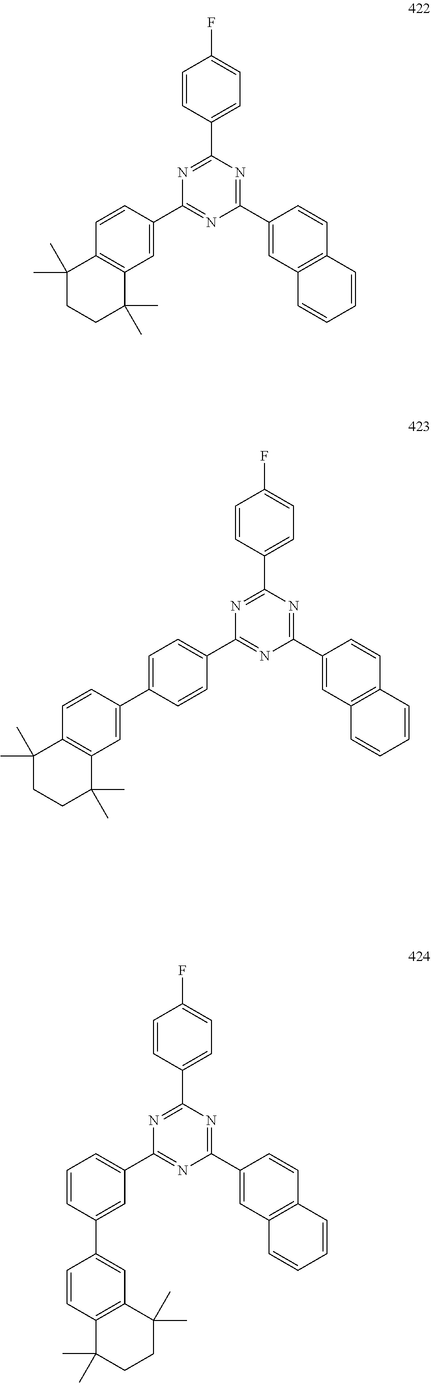 [SMILES: CC1(C)CCC(C)(C)c2cc(-c3nc(-c4ccc(F)cc4)nc(-c4ccc5ccccc5c4)n3)ccc21 ; CC1(C)CCC(C)(C)c2cc(-c3ccc(-c4nc(-c5ccc(F)cc5)nc(-c5ccc6ccccc6c5)n4)cc3)ccc21 ; CC1(C)CCC(C)(C)c2cc(-c3cccc(-c4nc(-c5ccc(F)cc5)nc(-c5ccc6ccccc6c5)n4)c3)ccc21]

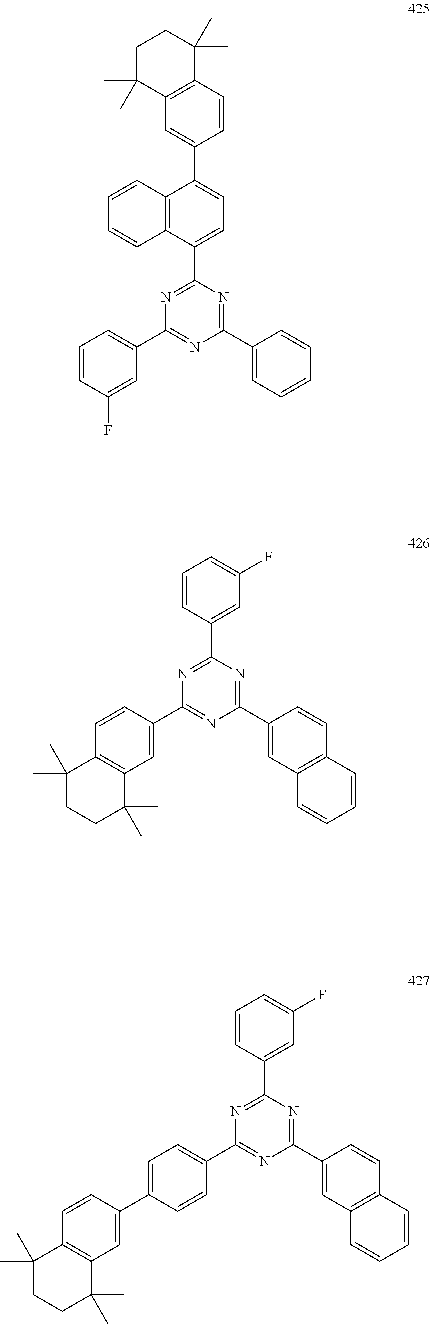 [SMILES: CC1(C)CCC(C)(C)c2cc(-c3ccc(-c4nc(-c5ccccc5)nc(-c5cccc(F)c5)n4)c4ccccc34)ccc21 ; CC1(C)CCC(C)(C)c2cc(-c3nc(-c4cccc(F)c4)nc(-c4ccc5ccccc5c4)n3)ccc21 ; CC1(C)CCC(C)(C)c2cc(-c3ccc(-c4nc(-c5cccc(F)c5)nc(-c5ccc6ccccc6c5)n4)cc3)ccc21]

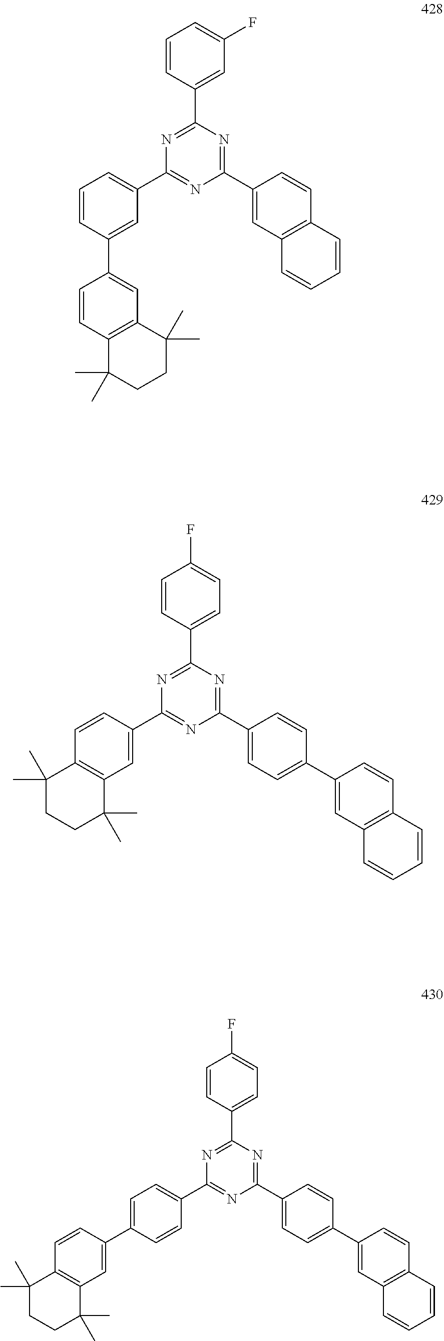 [SMILES: CC1(C)CCC(C)(C)c2cc(-c3cccc(-c4nc(-c5cccc(F)c5)nc(-c5ccc6ccccc6c5)n4)c3)ccc21 ; CC1(C)CCC(C)(C)c2cc(-c3nc(-c4ccc(F)cc4)nc(-c4ccc(-c5ccc6ccccc6c5)cc4)n3)ccc21 ; CC1(C)CCC(C)(C)c2cc(-c3ccc(-c4nc(-c5ccc(F)cc5)nc(-c5ccc(-c6ccc7ccccc7c6)cc5)n4)cc3)ccc21]

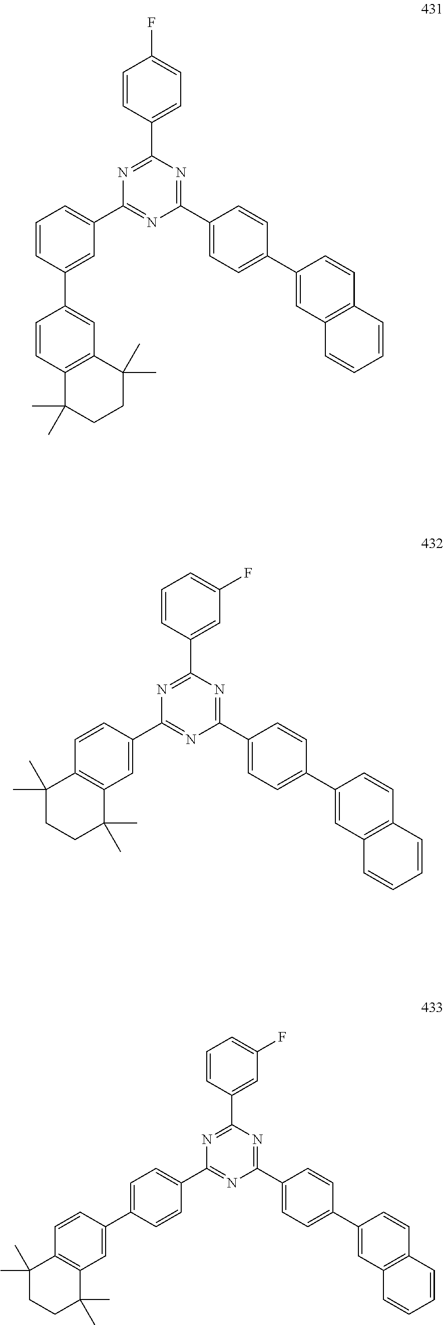 [SMILES: CC1(C)CCC(C)(C)c2cc(-c3cccc(-c4nc(-c5ccc(F)cc5)nc(-c5ccc(-c6ccc7ccccc7c6)cc5)n4)c3)ccc21 ; CC1(C)CCC(C)(C)c2cc(-c3nc(-c4ccc(-c5ccc6ccccc6c5)cc4)nc(-c4cccc(F)c4)n3)ccc21 ; CC1(C)CCC(C)(C)c2cc(-c3ccc(-c4nc(-c5ccc(-c6ccc7ccccc7c6)cc5)nc(-c5cccc(F)c5)n4)cc3)ccc21]

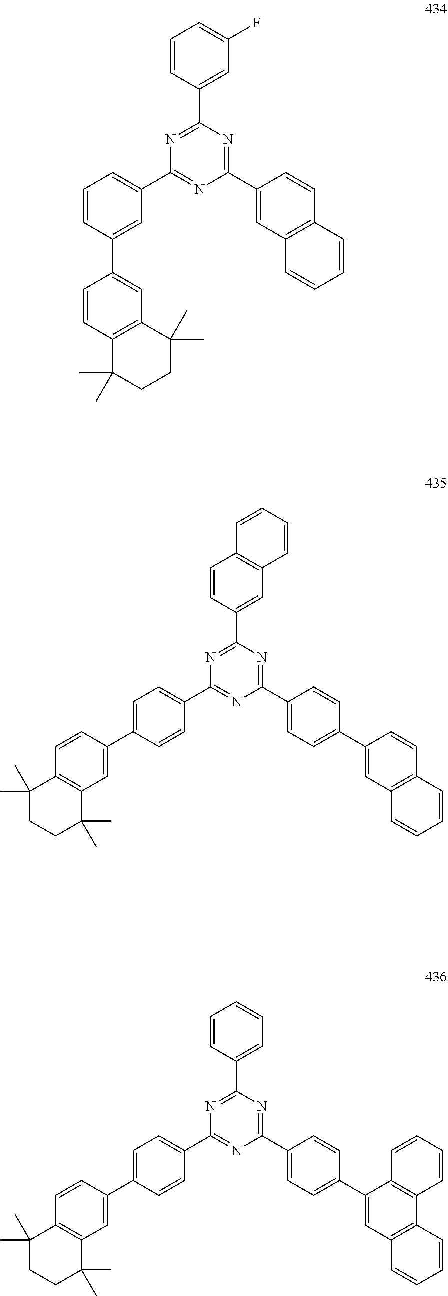 [SMILES: CC1(C)CCC(C)(C)c2cc(-c3cccc(-c4nc(-c5cccc(F)c5)nc(-c5ccc6ccccc6c5)n4)c3)ccc21 ; CC1(C)CCC(C)(C)c2cc(-c3ccc(-c4nc(-c5ccc(-c6ccc7ccccc7c6)cc5)nc(-c5ccc6ccccc6c5)n4)cc3)ccc21 ; CC1(C)CCC(C)(C)c2cc(-c3ccc(-c4nc(-c5ccccc5)nc(-c5ccc(-c6cc7ccccc7c7ccccc67)cc5)n4)cc3)ccc21]

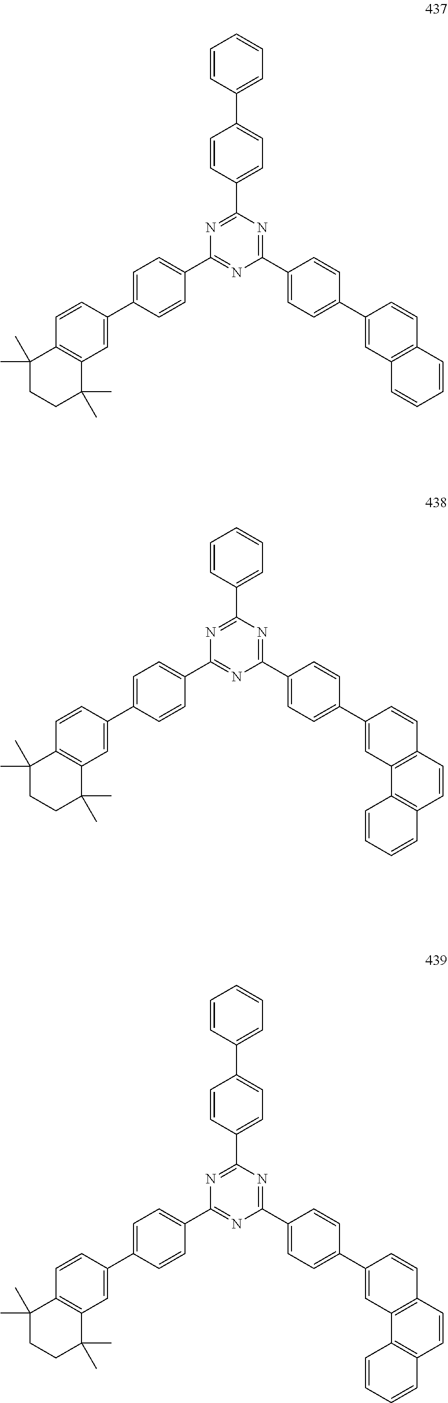 [SMILES: CC1(C)CCC(C)(C)c2cc(-c3ccc(-c4nc(-c5ccc(-c6ccccc6)cc5)nc(-c5ccc(-c6ccc7ccccc7c6)cc5)n4)cc3)ccc21 ; CC1(C)CCC(C)(C)c2cc(-c3ccc(-c4nc(-c5ccccc5)nc(-c5ccc(-c6ccc7ccc8ccccc8c7c6)cc5)n4)cc3)ccc21 ; CC1(C)CCC(C)(C)c2cc(-c3ccc(-c4nc(-c5ccc(-c6ccccc6)cc5)nc(-c5ccc(-c6ccc7ccc8ccccc8c7c6)cc5)n4)cc3)ccc21]

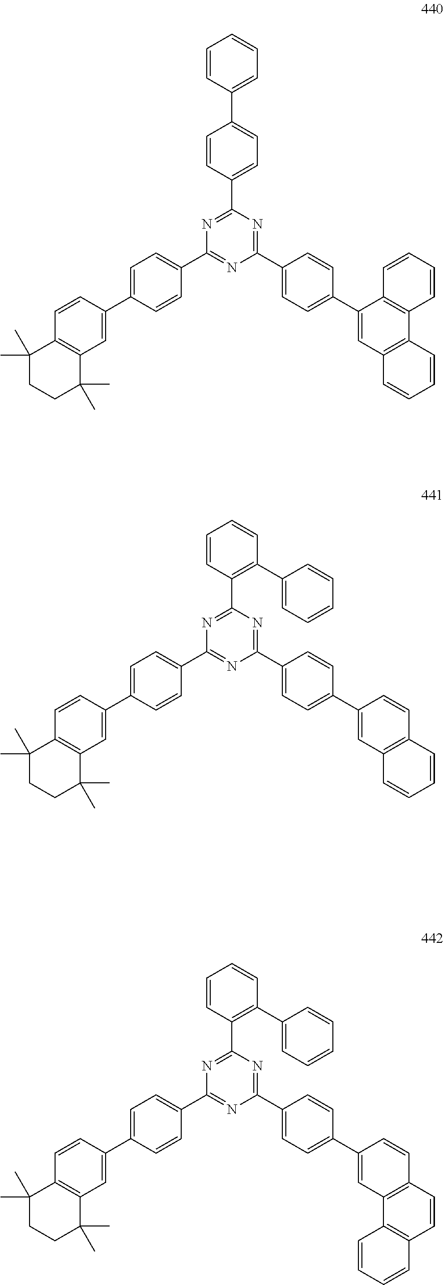 [SMILES: CC1(C)CCC(C)(C)c2cc(-c3ccc(-c4nc(-c5ccc(-c6ccccc6)cc5)nc(-c5ccc(-c6cc7ccccc7c7ccccc67)cc5)n4)cc3)ccc21 ; CC1(C)CCC(C)(C)c2cc(-c3ccc(-c4nc(-c5ccc(-c6ccc7ccccc7c6)cc5)nc(-c5ccccc5-c5ccccc5)n4)cc3)ccc21 ; CC1(C)CCC(C)(C)c2cc(-c3ccc(-c4nc(-c5ccc(-c6ccc7ccc8ccccc8c7c6)cc5)nc(-c5ccccc5-c5ccccc5)n4)cc3)ccc21]

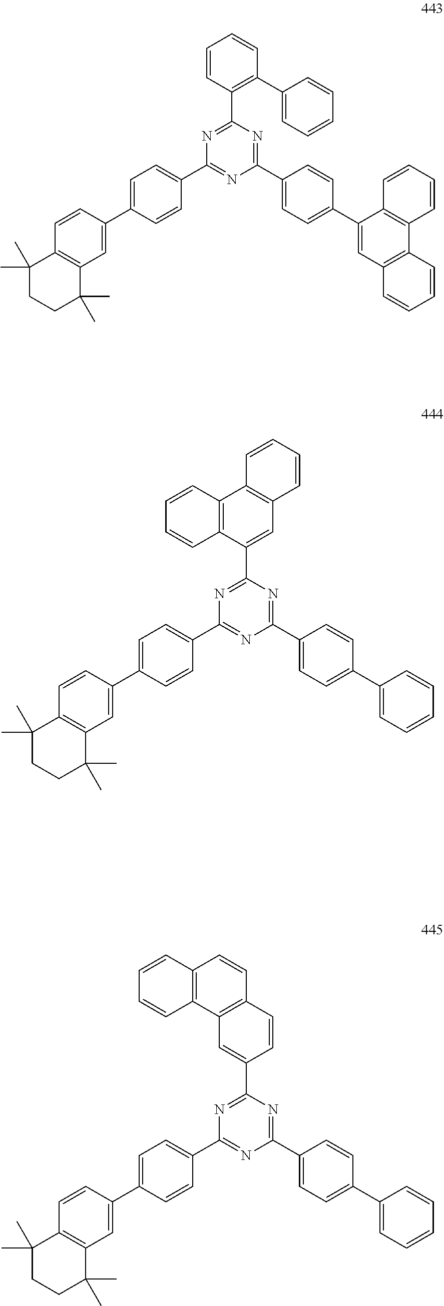 [SMILES: CC1(C)CCC(C)(C)c2cc(-c3ccc(-c4nc(-c5ccc(-c6cc7ccccc7c7ccccc67)cc5)nc(-c5ccccc5-c5ccccc5)n4)cc3)ccc21 ; CC1(C)CCC(C)(C)c2cc(-c3ccc(-c4nc(-c5ccc(-c6ccccc6)cc5)nc(-c5cc6ccccc6c6ccccc56)n4)cc3)ccc21 ; CC1(C)CCC(C)(C)c2cc(-c3ccc(-c4nc(-c5ccc(-c6ccccc6)cc5)nc(-c5ccc6ccc7ccccc7c6c5)n4)cc3)ccc21]

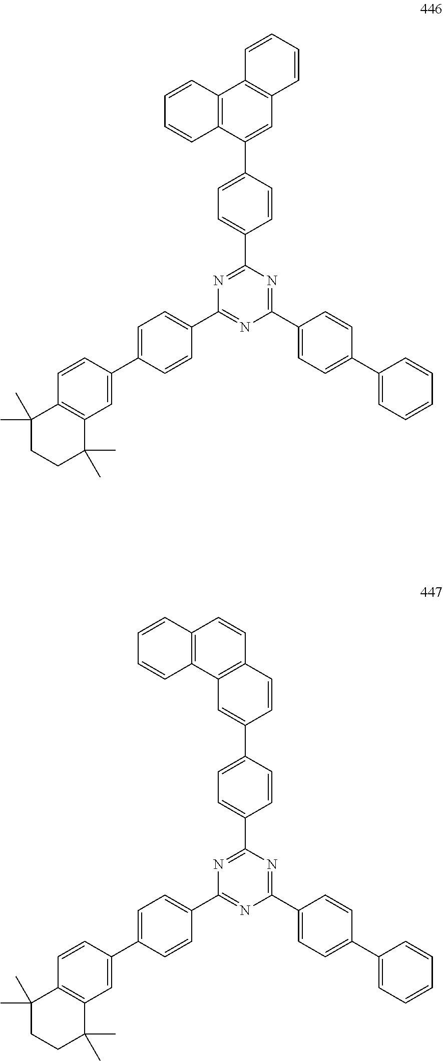 [SMILES: CC1(C)CCC(C)(C)c2cc(-c3ccc(-c4nc(-c5ccc(-c6ccccc6)cc5)nc(-c5ccc(-c6cc7ccccc7c7ccccc67)cc5)n4)cc3)ccc21 ; CC1(C)CCC(C)(C)c2cc(-c3ccc(-c4nc(-c5ccc(-c6ccccc6)cc5)nc(-c5ccc(-c6ccc7ccc8ccccc8c7c6)cc5)n4)cc3)ccc21]

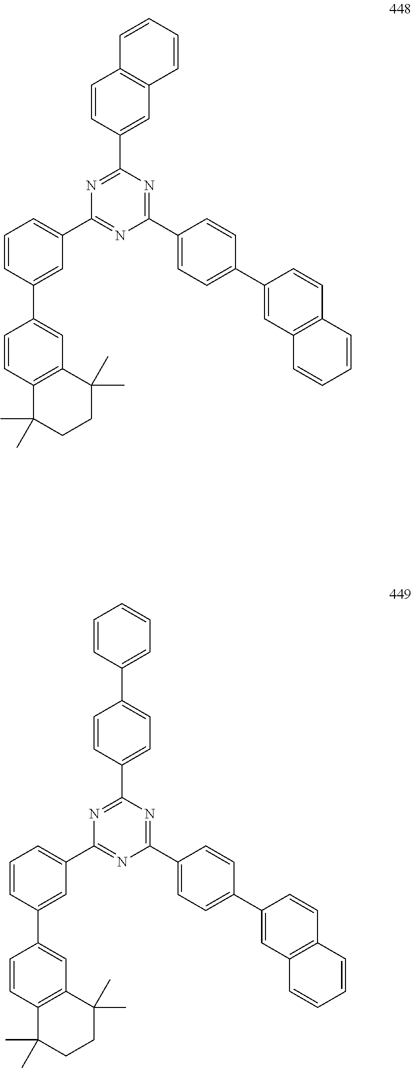 [SMILES: CC1(C)CCC(C)(C)c2cc(-c3cccc(-c4nc(-c5ccc(-c6ccc7ccccc7c6)cc5)nc(-c5ccc6ccccc6c5)n4)c3)ccc21 ; CC1(C)CCC(C)(C)c2cc(-c3cccc(-c4nc(-c5ccc(-c6ccccc6)cc5)nc(-c5ccc(-c6ccc7ccccc7c6)cc5)n4)c3)ccc21]

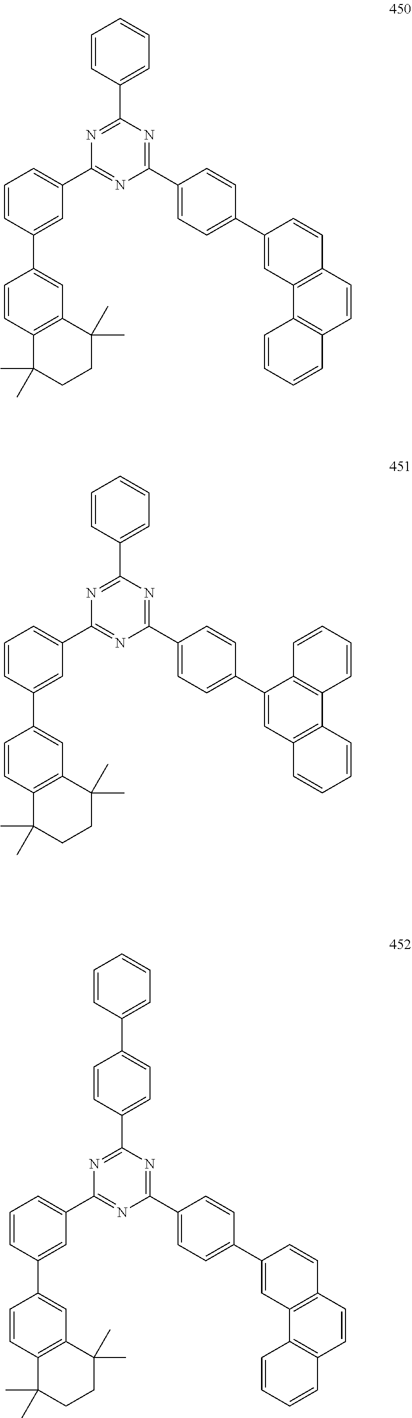 [SMILES: CC1(C)CCC(C)(C)c2cc(-c3cccc(-c4nc(-c5ccccc5)nc(-c5ccc(-c6ccc7ccc8ccccc8c7c6)cc5)n4)c3)ccc21 ; CC1(C)CCC(C)(C)c2cc(-c3cccc(-c4nc(-c5ccccc5)nc(-c5ccc(-c6cc7ccccc7c7ccccc67)cc5)n4)c3)ccc21 ; CC1(C)CCC(C)(C)c2cc(-c3cccc(-c4nc(-c5ccc(-c6ccccc6)cc5)nc(-c5ccc(-c6ccc7ccc8ccccc8c7c6)cc5)n4)c3)ccc21]

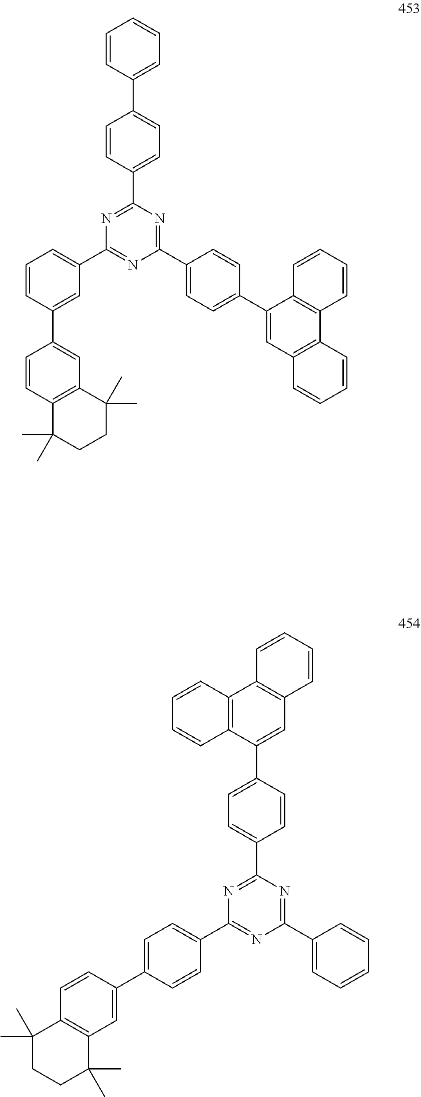 [SMILES: CC1(C)CCC(C)(C)c2cc(-c3cccc(-c4nc(-c5ccc(-c6ccccc6)cc5)nc(-c5ccc(-c6cc7ccccc7c7ccccc67)cc5)n4)c3)ccc21 ; CC1(C)CCC(C)(C)c2cc(-c3ccc(-c4nc(-c5ccccc5)nc(-c5ccc(-c6cc7ccccc7c7ccccc67)cc5)n4)cc3)ccc21]

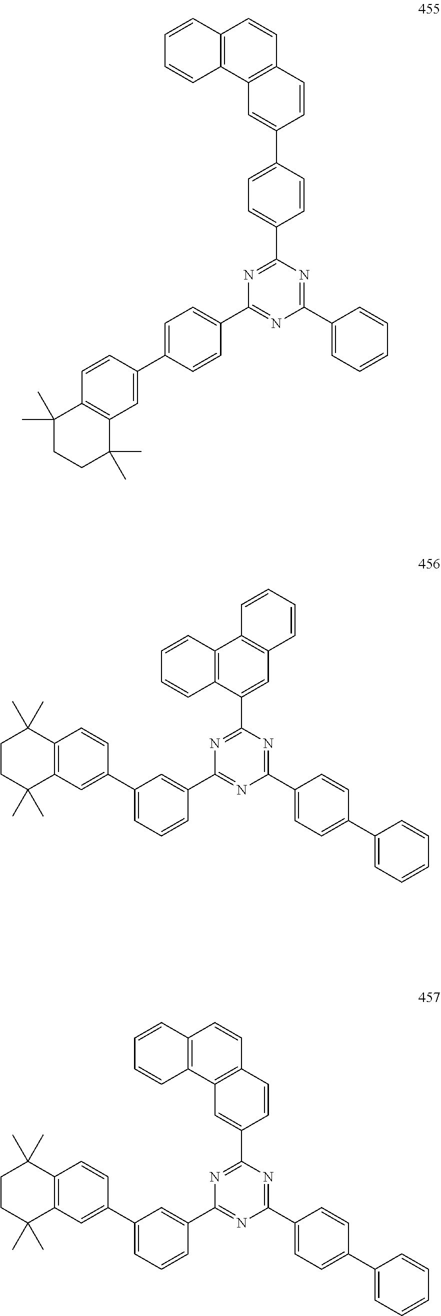 [SMILES: CC1(C)CCC(C)(C)c2cc(-c3ccc(-c4nc(-c5ccccc5)nc(-c5ccc(-c6ccc7ccc8ccccc8c7c6)cc5)n4)cc3)ccc21 ; CC1(C)CCC(C)(C)c2cc(-c3cccc(-c4nc(-c5ccc(-c6ccccc6)cc5)nc(-c5cc6ccccc6c6ccccc56)n4)c3)ccc21 ; CC1(C)CCC(C)(C)c2cc(-c3cccc(-c4nc(-c5ccc(-c6ccccc6)cc5)nc(-c5ccc6ccc7ccccc7c6c5)n4)c3)ccc21]

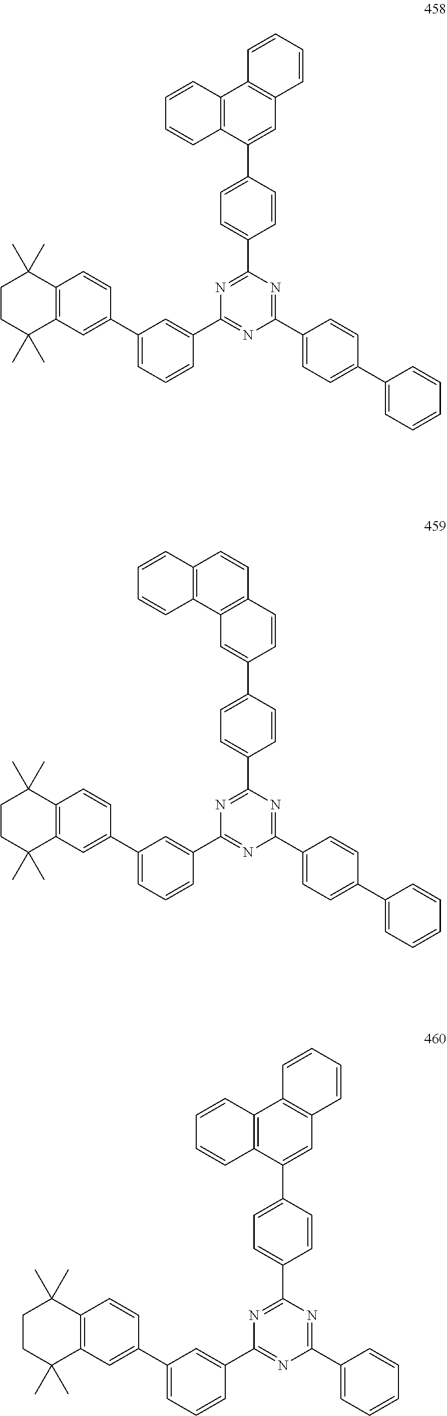 [SMILES: CC1(C)CCC(C)(C)c2cc(-c3cccc(-c4nc(-c5ccc(-c6ccccc6)cc5)nc(-c5ccc(-c6cc7ccccc7c7ccccc67)cc5)n4)c3)ccc21 ; CC1(C)CCC(C)(C)c2cc(-c3cccc(-c4nc(-c5ccc(-c6ccccc6)cc5)nc(-c5ccc(-c6ccc7ccc8ccccc8c7c6)cc5)n4)c3)ccc21 ; CC1(C)CCC(C)(C)c2cc(-c3cccc(-c4nc(-c5ccccc5)nc(-c5ccc(-c6cc7ccccc7c7ccccc67)cc5)n4)c3)ccc21]

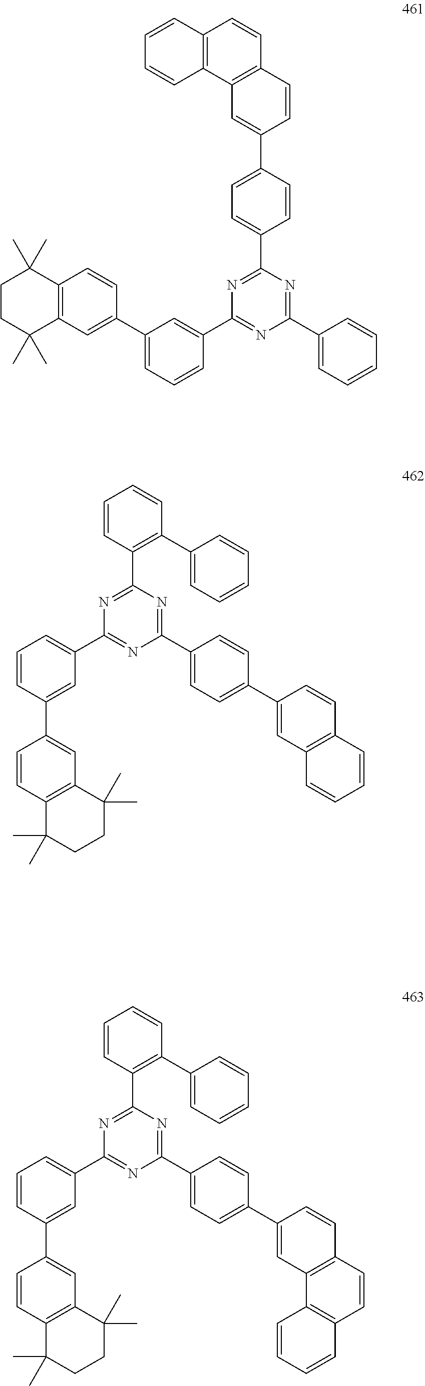 [SMILES: CC1(C)CCC(C)(C)c2cc(-c3cccc(-c4nc(-c5ccccc5)nc(-c5ccc(-c6ccc7ccc8ccccc8c7c6)cc5)n4)c3)ccc21 ; CC1(C)CCC(C)(C)c2cc(-c3cccc(-c4nc(-c5ccc(-c6ccc7ccccc7c6)cc5)nc(-c5ccccc5-c5ccccc5)n4)c3)ccc21 ; CC1(C)CCC(C)(C)c2cc(-c3cccc(-c4nc(-c5ccc(-c6ccc7ccc8ccccc8c7c6)cc5)nc(-c5ccccc5-c5ccccc5)n4)c3)ccc21]

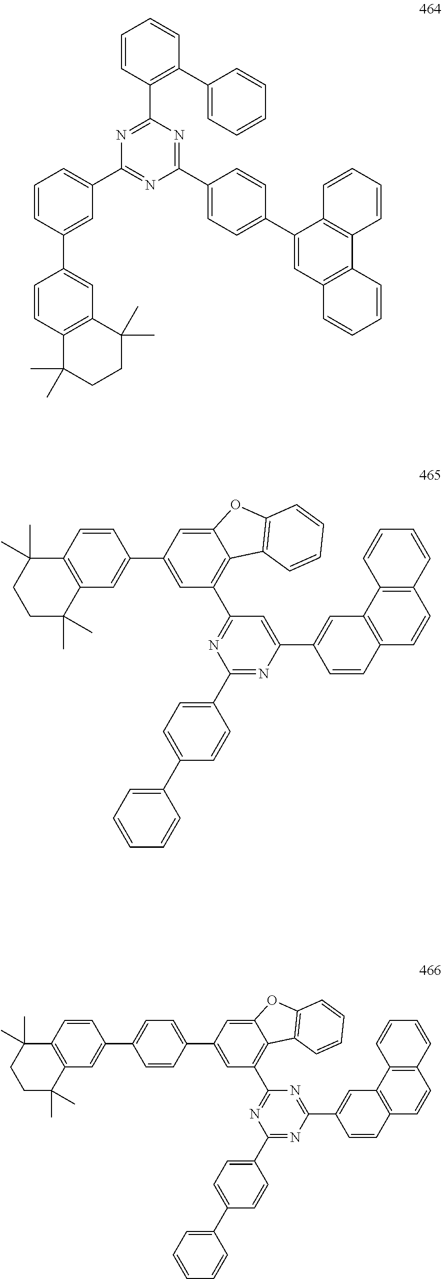 [SMILES: CC1(C)CCC(C)(C)c2cc(-c3cccc(-c4nc(-c5ccc(-c6cc7ccccc7c7ccccc67)cc5)nc(-c5ccccc5-c5ccccc5)n4)c3)ccc21 ; CC1(C)CCC(C)(C)c2cc(-c3cc(-c4cc(-c5ccc6ccc7ccccc7c6c5)nc(-c5ccc(-c6ccccc6)cc5)n4)c4c(c3)oc3ccccc34)ccc21 ; CC1(C)CCC(C)(C)c2cc(-c3ccc(-c4cc(-c5nc(-c6ccc(-c7ccccc7)cc6)nc(-c6ccc7ccc8ccccc8c7c6)n5)c5c(c4)oc4ccccc45)cc3)ccc21]

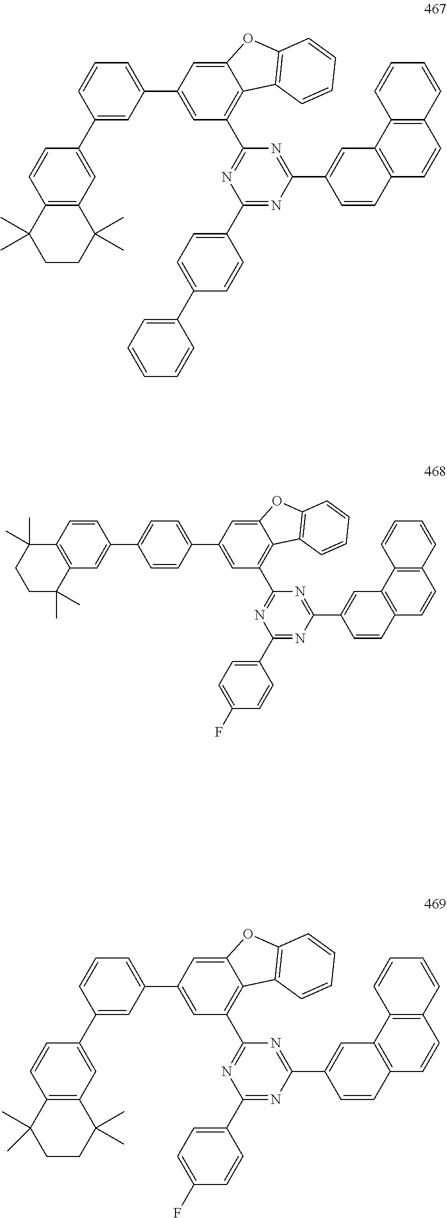 [SMILES: CC1(C)CCC(C)(C)c2cc(-c3cccc(-c4cc(-c5nc(-c6ccc(-c7ccccc7)cc6)nc(-c6ccc7ccc8ccccc8c7c6)n5)c5c(c4)oc4ccccc45)c3)ccc21 ; CC1(C)CCC(C)(C)c2cc(-c3ccc(-c4cc(-c5nc(-c6ccc(F)cc6)nc(-c6ccc7ccc8ccccc8c7c6)n5)c5c(c4)oc4ccccc45)cc3)ccc21 ; CC1(C)CCC(C)(C)c2cc(-c3cccc(-c4cc(-c5nc(-c6ccc(F)cc6)nc(-c6ccc7ccc8ccccc8c7c6)n5)c5c(c4)oc4ccccc45)c3)ccc21]

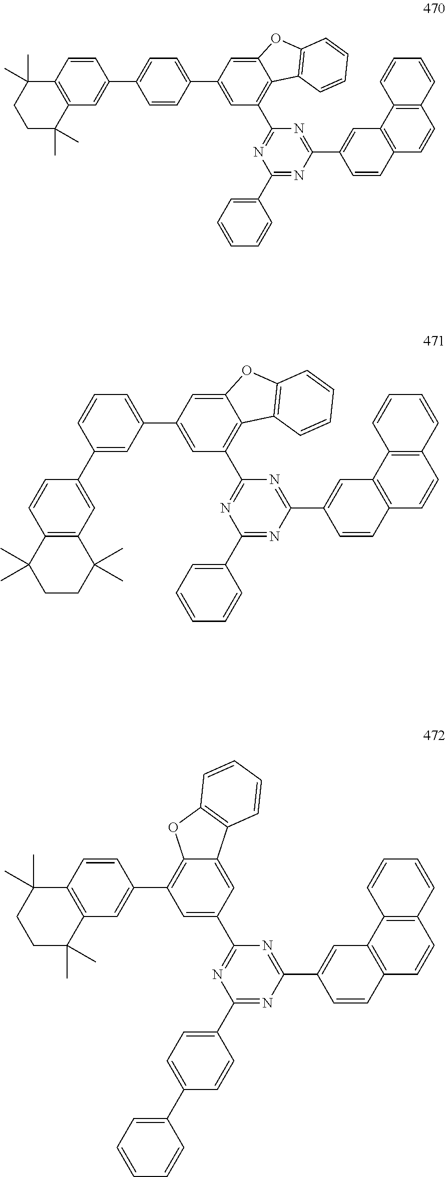 [SMILES: CC1(C)CCC(C)(C)c2cc(-c3ccc(-c4cc(-c5nc(-c6ccccc6)nc(-c6ccc7ccc8ccccc8c7c6)n5)c5c(c4)oc4ccccc45)cc3)ccc21 ; CC1(C)CCC(C)(C)c2cc(-c3cccc(-c4cc(-c5nc(-c6ccccc6)nc(-c6ccc7ccc8ccccc8c7c6)n5)c5c(c4)oc4ccccc45)c3)ccc21 ; CC1(C)CCC(C)(C)c2cc(-c3cc(-c4nc(-c5ccc(-c6ccccc6)cc5)nc(-c5ccc6ccc7ccccc7c6c5)n4)cc4c3oc3ccccc34)ccc21]

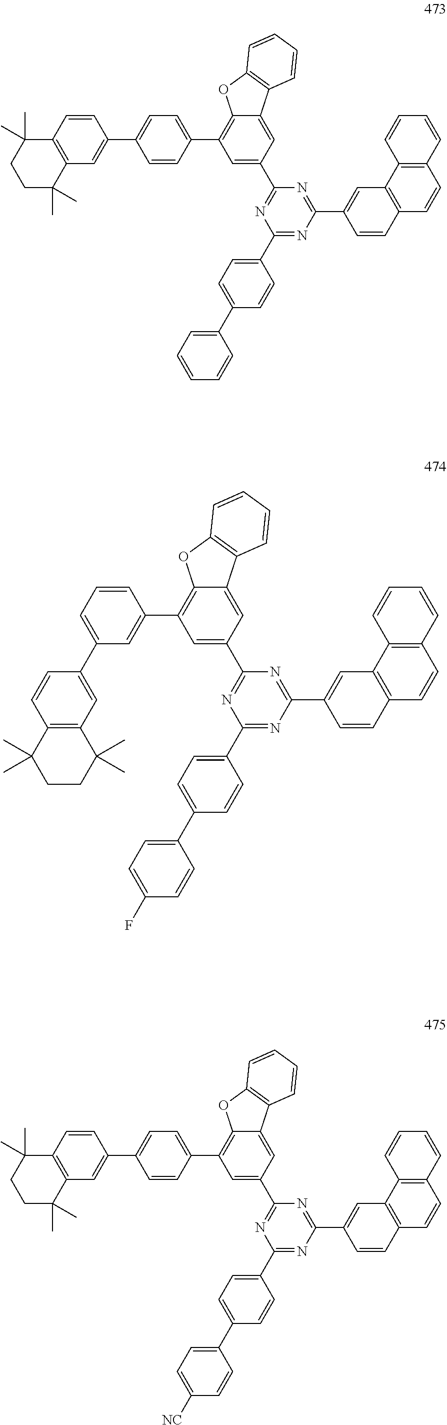 [SMILES: CC1(C)CCC(C)(C)c2cc(-c3ccc(-c4cc(-c5nc(-c6ccc(-c7ccccc7)cc6)nc(-c6ccc7ccc8ccccc8c7c6)n5)cc5c4oc4ccccc45)cc3)ccc21 ; CC1(C)CCC(C)(C)c2cc(-c3cccc(-c4cc(-c5nc(-c6ccc(-c7ccc(F)cc7)cc6)nc(-c6ccc7ccc8ccccc8c7c6)n5)cc5c4oc4ccccc45)c3)ccc21 ; CC1(C)CCC(C)(C)c2cc(-c3ccc(-c4cc(-c5nc(-c6ccc(-c7ccc(C#N)cc7)cc6)nc(-c6ccc7ccc8ccccc8c7c6)n5)cc5c4oc4ccccc45)cc3)ccc21]

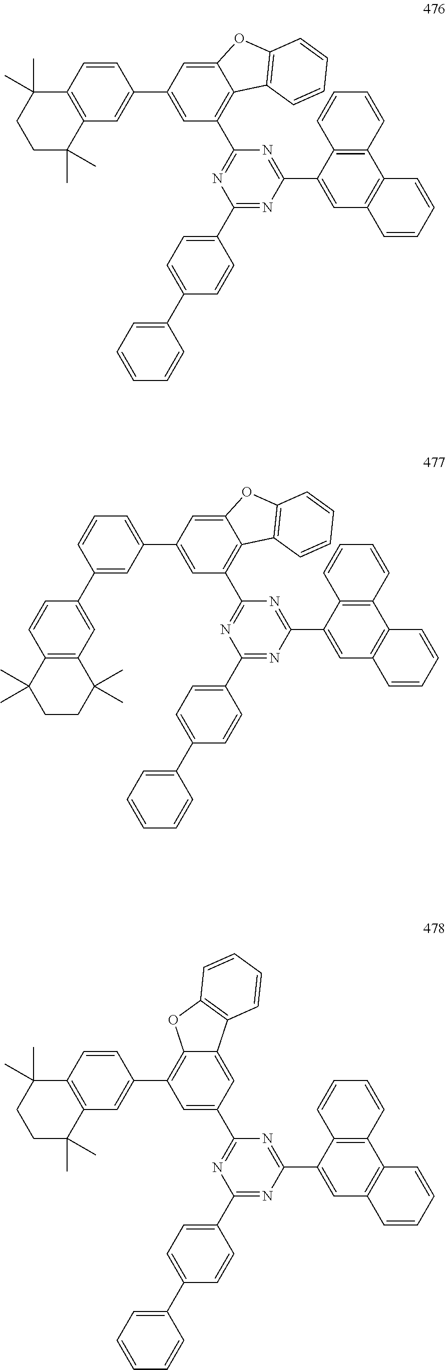 [SMILES: CC1(C)CCC(C)(C)c2cc(-c3cc(-c4nc(-c5ccc(-c6ccccc6)cc5)nc(-c5cc6ccccc6c6ccccc56)n4)c4c(c3)oc3ccccc34)ccc21 ; CC1(C)CCC(C)(C)c2cc(-c3cccc(-c4cc(-c5nc(-c6ccc(-c7ccccc7)cc6)nc(-c6cc7ccccc7c7ccccc67)n5)c5c(c4)oc4ccccc45)c3)ccc21 ; CC1(C)CCC(C)(C)c2cc(-c3cc(-c4nc(-c5ccc(-c6ccccc6)cc5)nc(-c5cc6ccccc6c6ccccc56)n4)cc4c3oc3ccccc34)ccc21]

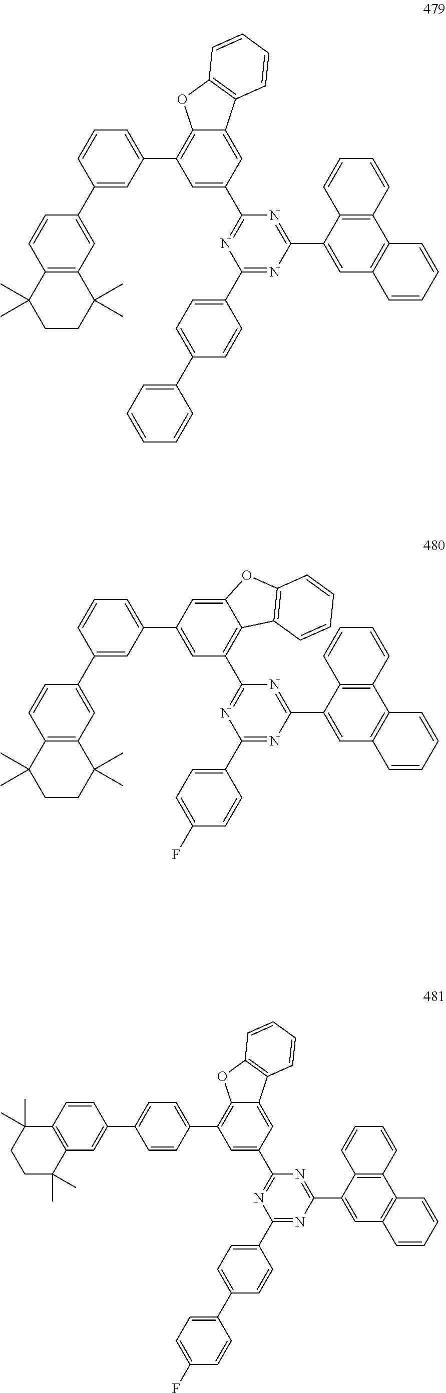 [SMILES: CC1(C)CCC(C)(C)c2cc(-c3cccc(-c4cc(-c5nc(-c6ccc(-c7ccccc7)cc6)nc(-c6cc7ccccc7c7ccccc67)n5)cc5c4oc4ccccc45)c3)ccc21 ; CC1(C)CCC(C)(C)c2cc(-c3cccc(-c4cc(-c5nc(-c6ccc(F)cc6)nc(-c6cc7ccccc7c7ccccc67)n5)c5c(c4)oc4ccccc45)c3)ccc21 ; CC1(C)CCC(C)(C)c2cc(-c3ccc(-c4cc(-c5nc(-c6ccc(-c7ccc(F)cc7)cc6)nc(-c6cc7ccccc7c7ccccc67)n5)cc5c4oc4ccccc45)cc3)ccc21]

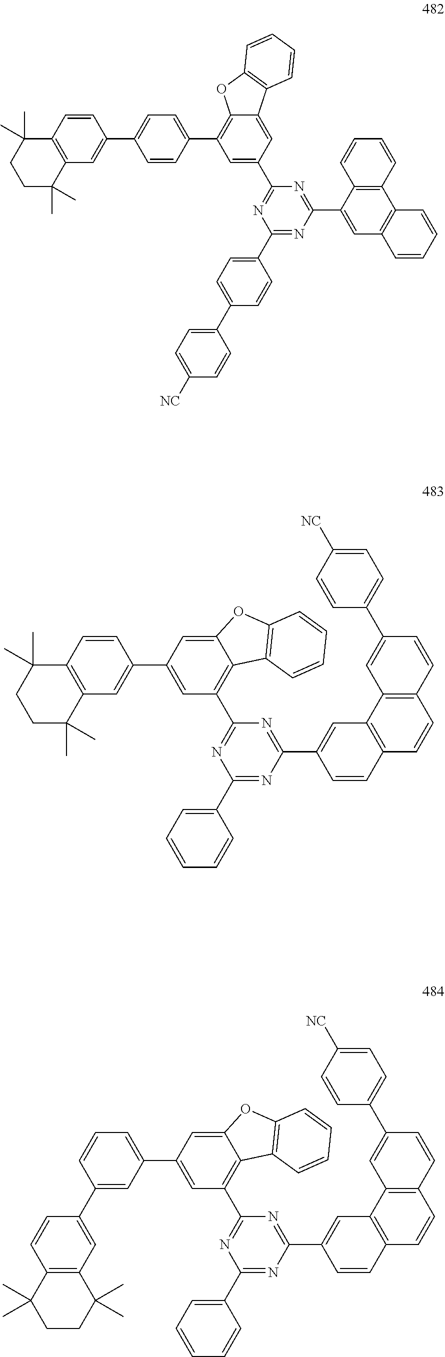 [SMILES: CC1(C)CCC(C)(C)c2cc(-c3ccc(-c4cc(-c5nc(-c6ccc(-c7ccc(C#N)cc7)cc6)nc(-c6cc7ccccc7c7ccccc67)n5)cc5c4oc4ccccc45)cc3)ccc21 ; CC1(C)CCC(C)(C)c2cc(-c3cc(-c4nc(-c5ccccc5)nc(-c5ccc6ccc7ccc(-c8ccc(C#N)cc8)cc7c6c5)n4)c4c(c3)oc3ccccc34)ccc21 ; CC1(C)CCC(C)(C)c2cc(-c3cccc(-c4cc(-c5nc(-c6ccccc6)nc(-c6ccc7ccc8ccc(-c9ccc(C#N)cc9)cc8c7c6)n5)c5c(c4)oc4ccccc45)c3)ccc21]

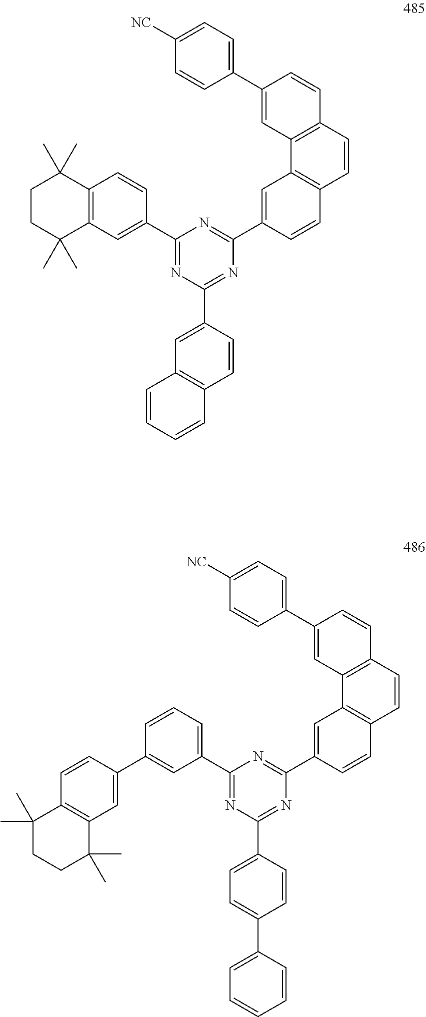 [SMILES: CC1(C)CCC(C)(C)c2cc(-c3nc(-c4ccc5ccccc5c4)nc(-c4ccc5ccc6ccc(-c7ccc(C#N)cc7)cc6c5c4)n3)ccc21 ; CC1(C)CCC(C)(C)c2cc(-c3cccc(-c4nc(-c5ccc(-c6ccccc6)cc5)nc(-c5ccc6ccc7ccc(-c8ccc(C#N)cc8)cc7c6c5)n4)c3)ccc21]

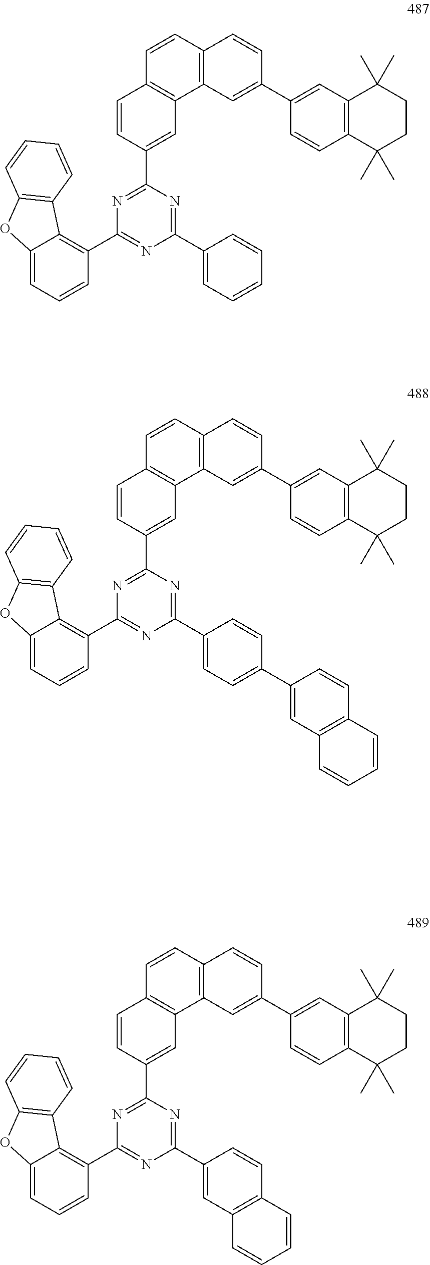 [SMILES: CC1(C)CCC(C)(C)c2cc(-c3ccc4ccc5ccc(-c6nc(-c7ccccc7)nc(-c7cccc8oc9ccccc9c78)n6)cc5c4c3)ccc21 ; CC1(C)CCC(C)(C)c2cc(-c3ccc4ccc5ccc(-c6nc(-c7ccc(-c8ccc9ccccc9c8)cc7)nc(-c7cccc8oc9ccccc9c78)n6)cc5c4c3)ccc21 ; CC1(C)CCC(C)(C)c2cc(-c3ccc4ccc5ccc(-c6nc(-c7ccc8ccccc8c7)nc(-c7cccc8oc9ccccc9c78)n6)cc5c4c3)ccc21]

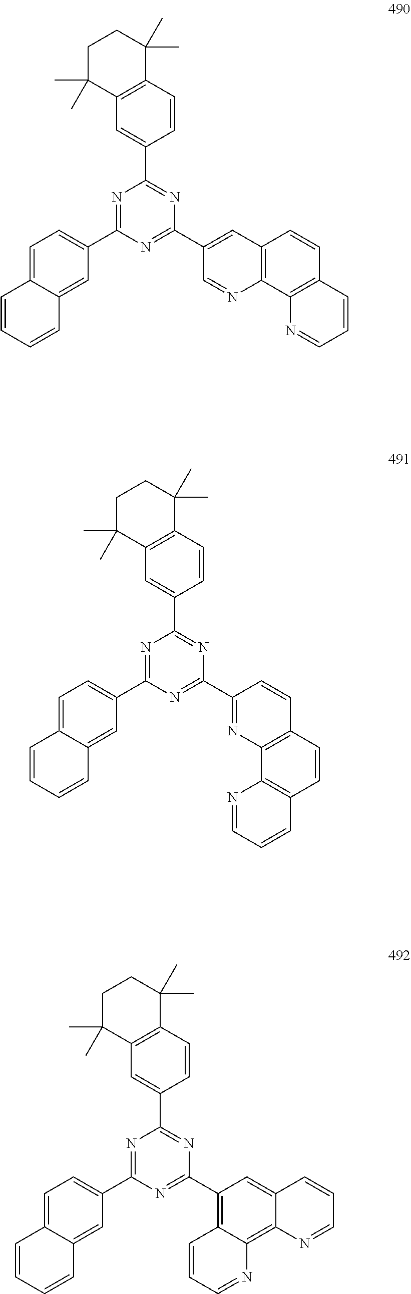 [SMILES: CC1(C)CCC(C)(C)c2cc(-c3nc(-c4ccc5ccccc5c4)nc(-c4cnc5c(ccc6cccnc65)c4)n3)ccc21 ; CC1(C)CCC(C)(C)c2cc(-c3nc(-c4ccc5ccccc5c4)nc(-c4ccc5ccc6cccnc6c5n4)n3)ccc21 ; CC1(C)CCC(C)(C)c2cc(-c3nc(-c4ccc5ccccc5c4)nc(-c4cc5cccnc5c5ncccc45)n3)ccc21]

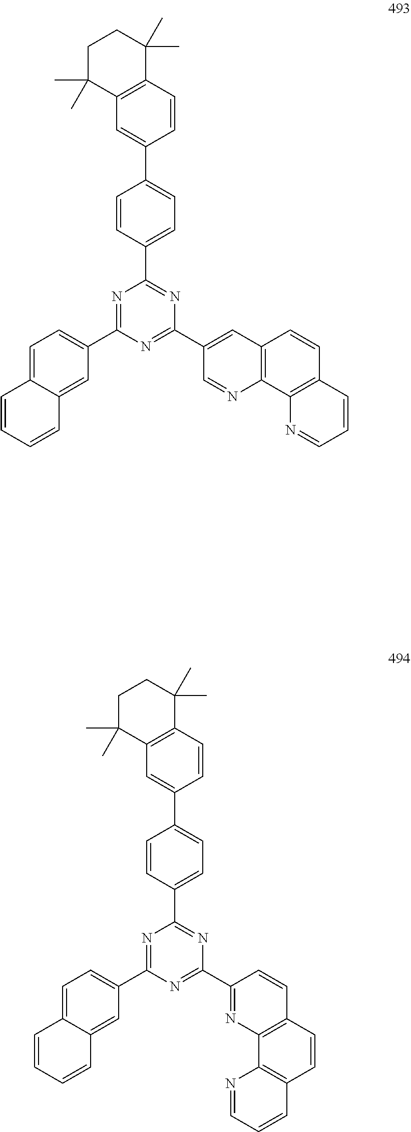 [SMILES: CC1(C)CCC(C)(C)c2cc(-c3ccc(-c4nc(-c5ccc6ccccc6c5)nc(-c5cnc6c(ccc7cccnc76)c5)n4)cc3)ccc21 ; CC1(C)CCC(C)(C)c2cc(-c3ccc(-c4nc(-c5ccc6ccccc6c5)nc(-c5ccc6ccc7cccnc7c6n5)n4)cc3)ccc21]

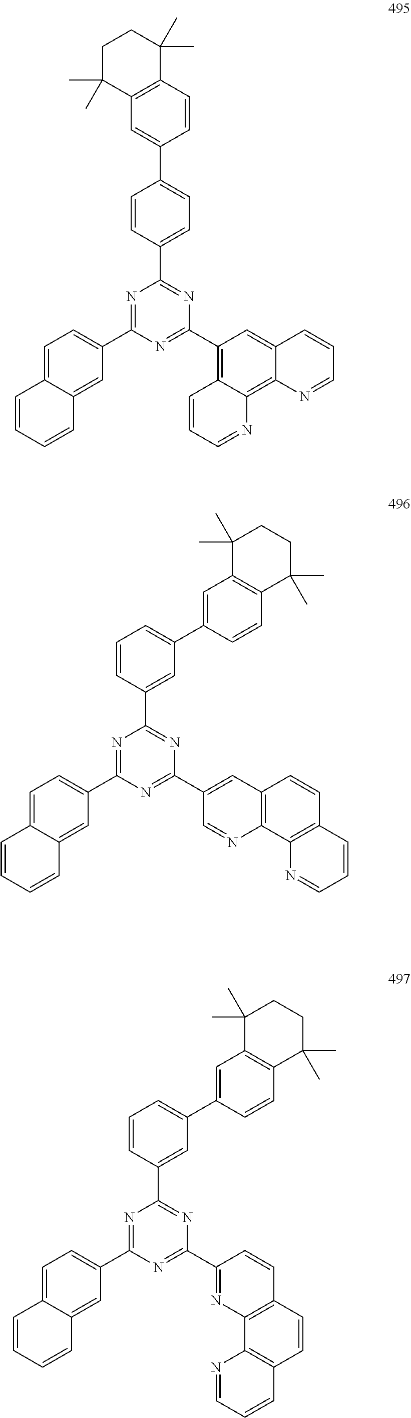 [SMILES: CC1(C)CCC(C)(C)c2cc(-c3ccc(-c4nc(-c5ccc6ccccc6c5)nc(-c5cc6cccnc6c6ncccc56)n4)cc3)ccc21 ; CC1(C)CCC(C)(C)c2cc(-c3cccc(-c4nc(-c5ccc6ccccc6c5)nc(-c5cnc6c(ccc7cccnc76)c5)n4)c3)ccc21 ; CC1(C)CCC(C)(C)c2cc(-c3cccc(-c4nc(-c5ccc6ccccc6c5)nc(-c5ccc6ccc7cccnc7c6n5)n4)c3)ccc21]

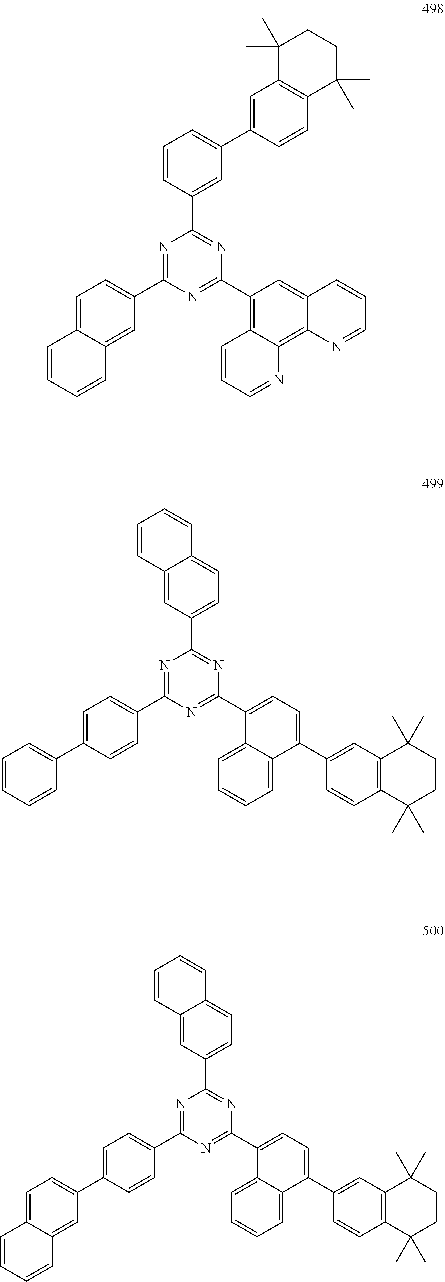 [SMILES: CC1(C)CCC(C)(C)c2cc(-c3cccc(-c4nc(-c5ccc6ccccc6c5)nc(-c5cc6cccnc6c6ncccc56)n4)c3)ccc21 ; CC1(C)CCC(C)(C)c2cc(-c3ccc(-c4nc(-c5ccc(-c6ccccc6)cc5)nc(-c5ccc6ccccc6c5)n4)c4ccccc34)ccc21 ; CC1(C)CCC(C)(C)c2cc(-c3ccc(-c4nc(-c5ccc(-c6ccc7ccccc7c6)cc5)nc(-c5ccc6ccccc6c5)n4)c4ccccc34)ccc21]

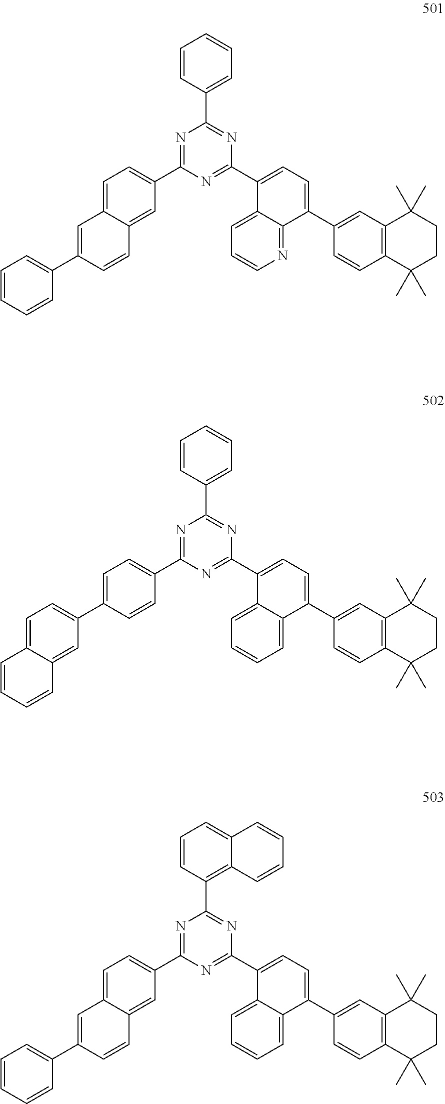 [SMILES: CC1(C)CCC(C)(C)c2cc(-c3ccc(-c4nc(-c5ccccc5)nc(-c5ccc6cc(-c7ccccc7)ccc6c5)n4)c4cccnc34)ccc21 ; CC1(C)CCC(C)(C)c2cc(-c3ccc(-c4nc(-c5ccccc5)nc(-c5ccc(-c6ccc7ccccc7c6)cc5)n4)c4ccccc34)ccc21 ; CC1(C)CCC(C)(C)c2cc(-c3ccc(-c4nc(-c5ccc6cc(-c7ccccc7)ccc6c5)nc(-c5cccc6ccccc56)n4)c4ccccc34)ccc21]

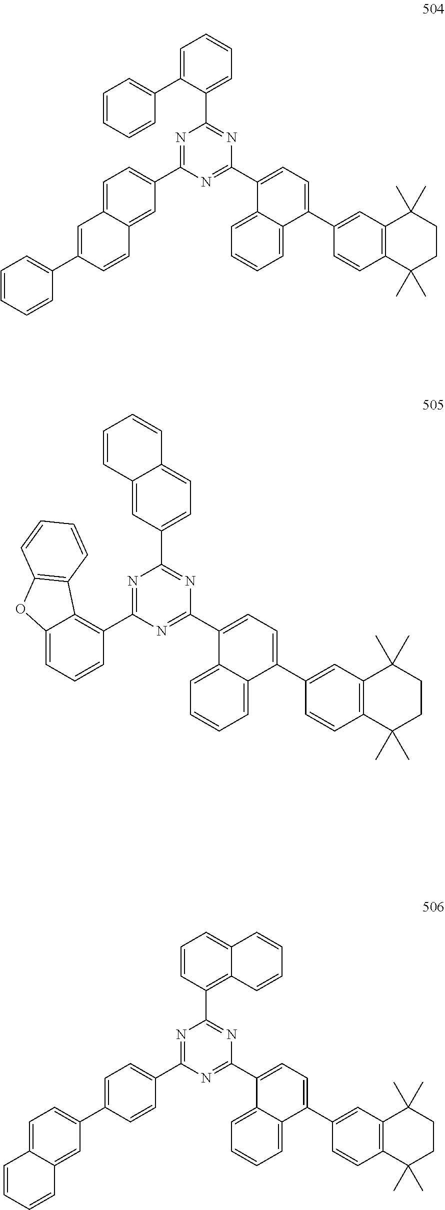 [SMILES: CC1(C)CCC(C)(C)c2cc(-c3ccc(-c4nc(-c5ccc6cc(-c7ccccc7)ccc6c5)nc(-c5ccccc5-c5ccccc5)n4)c4ccccc34)ccc21 ; CC1(C)CCC(C)(C)c2cc(-c3ccc(-c4nc(-c5ccc6ccccc6c5)nc(-c5cccc6oc7ccccc7c56)n4)c4ccccc34)ccc21 ; CC1(C)CCC(C)(C)c2cc(-c3ccc(-c4nc(-c5ccc(-c6ccc7ccccc7c6)cc5)nc(-c5cccc6ccccc56)n4)c4ccccc34)ccc21]

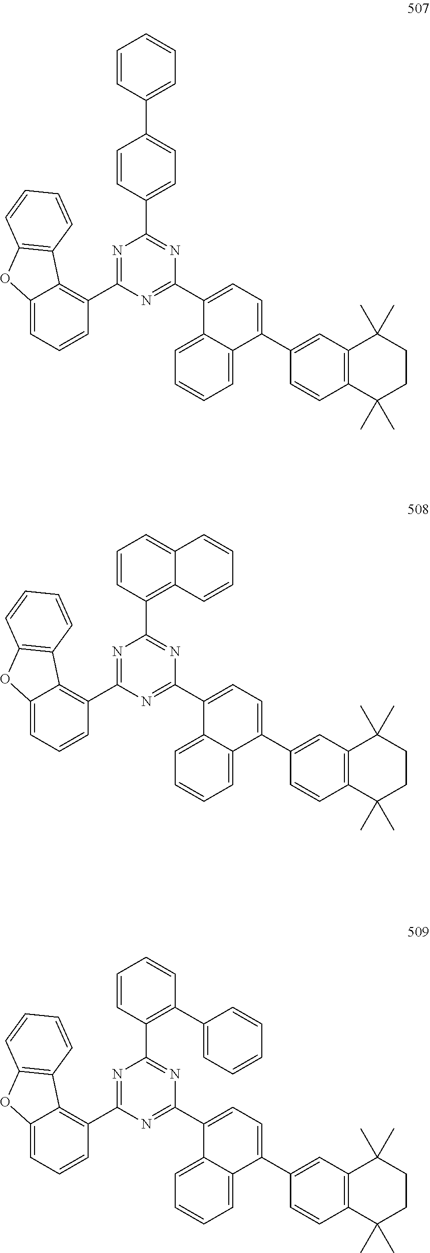 [SMILES: CC1(C)CCC(C)(C)c2cc(-c3ccc(-c4nc(-c5ccc(-c6ccccc6)cc5)nc(-c5cccc6oc7ccccc7c56)n4)c4ccccc34)ccc21 ; CC1(C)CCC(C)(C)c2cc(-c3ccc(-c4nc(-c5cccc6ccccc56)nc(-c5cccc6oc7ccccc7c56)n4)c4ccccc34)ccc21 ; CC1(C)CCC(C)(C)c2cc(-c3ccc(-c4nc(-c5ccccc5-c5ccccc5)nc(-c5cccc6oc7ccccc7c56)n4)c4ccccc34)ccc21]

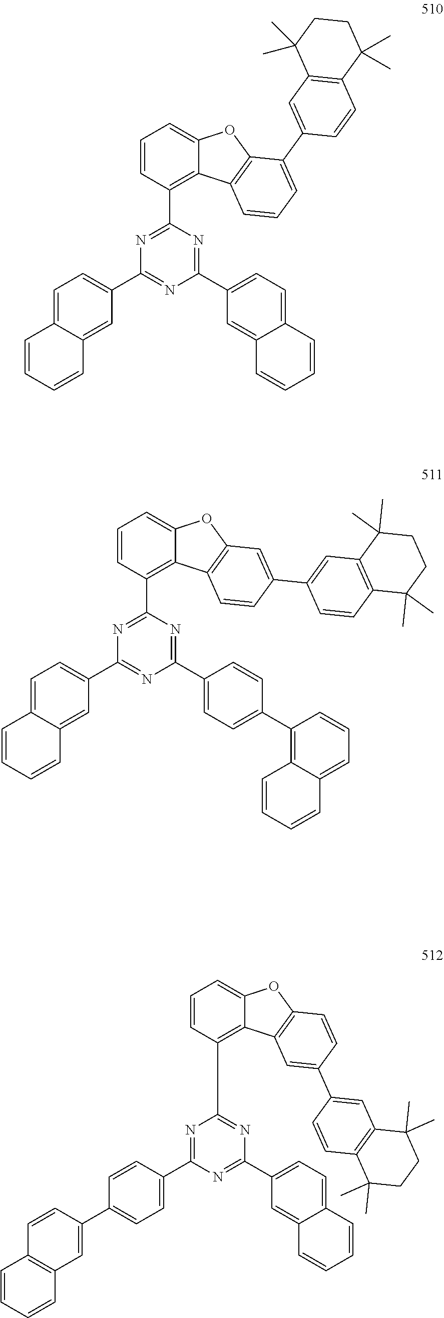 [SMILES: CC1(C)CCC(C)(C)c2cc(-c3cccc4c3oc3cccc(-c5nc(-c6ccc7ccccc7c6)nc(-c6ccc7ccccc7c6)n5)c34)ccc21 ; CC1(C)CCC(C)(C)c2cc(-c3ccc4c(c3)oc3cccc(-c5nc(-c6ccc(-c7cccc8ccccc78)cc6)nc(-c6ccc7ccccc7c6)n5)c34)ccc21 ; CC1(C)CCC(C)(C)c2cc(-c3ccc4oc5cccc(-c6nc(-c7ccc(-c8ccc9ccccc9c8)cc7)nc(-c7ccc8ccccc8c7)n6)c5c4c3)ccc21]

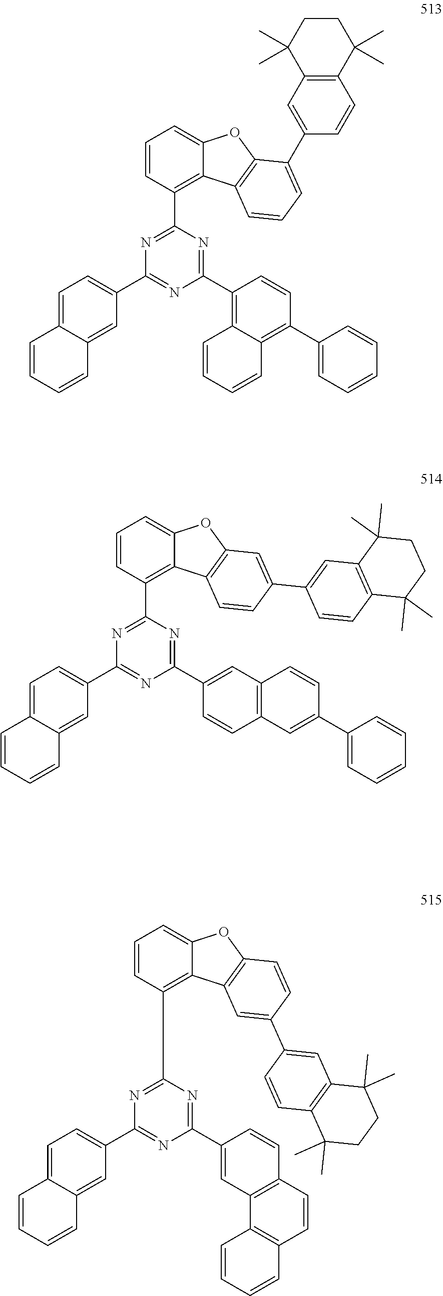 [SMILES: CC1(C)CCC(C)(C)c2cc(-c3cccc4c3oc3cccc(-c5nc(-c6ccc7ccccc7c6)nc(-c6ccc(-c7ccccc7)c7ccccc67)n5)c34)ccc21 ; CC1(C)CCC(C)(C)c2cc(-c3ccc4c(c3)oc3cccc(-c5nc(-c6ccc7ccccc7c6)nc(-c6ccc7cc(-c8ccccc8)ccc7c6)n5)c34)ccc21 ; CC1(C)CCC(C)(C)c2cc(-c3ccc4oc5cccc(-c6nc(-c7ccc8ccccc8c7)nc(-c7ccc8ccc9ccccc9c8c7)n6)c5c4c3)ccc21]

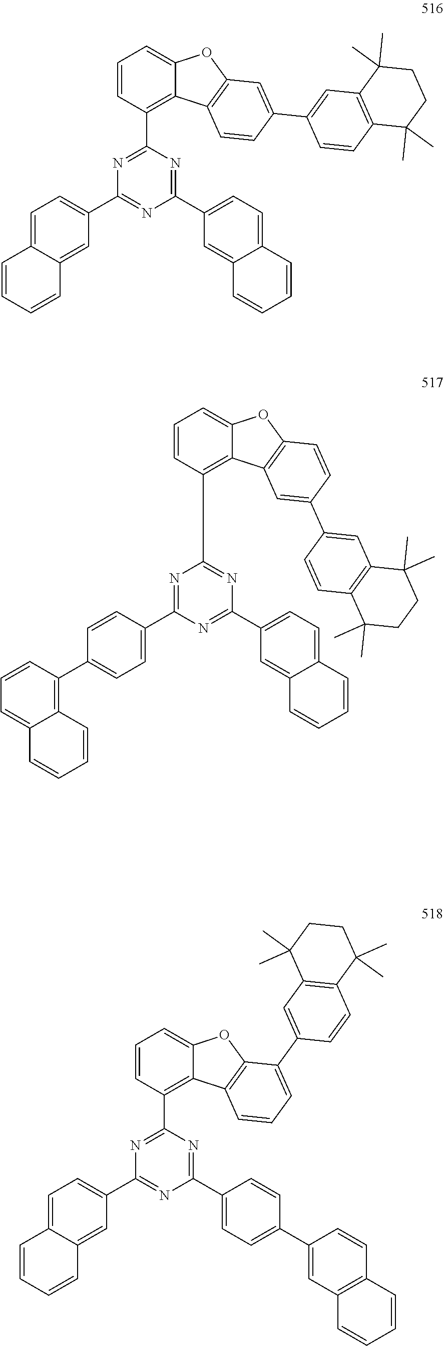 [SMILES: CC1(C)CCC(C)(C)c2cc(-c3ccc4c(c3)oc3cccc(-c5nc(-c6ccc7ccccc7c6)nc(-c6ccc7ccccc7c6)n5)c34)ccc21 ; CC1(C)CCC(C)(C)c2cc(-c3ccc4oc5cccc(-c6nc(-c7ccc(-c8cccc9ccccc89)cc7)nc(-c7ccc8ccccc8c7)n6)c5c4c3)ccc21 ; CC1(C)CCC(C)(C)c2cc(-c3cccc4c3oc3cccc(-c5nc(-c6ccc(-c7ccc8ccccc8c7)cc6)nc(-c6ccc7ccccc7c6)n5)c34)ccc21]

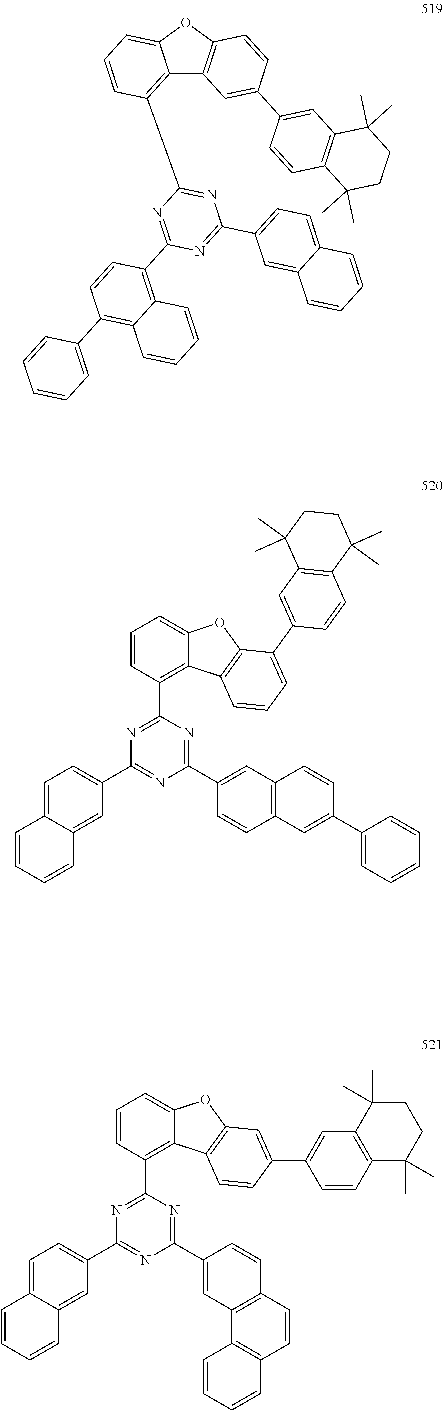 [SMILES: CC1(C)CCC(C)(C)c2cc(-c3ccc4oc5cccc(-c6nc(-c7ccc8ccccc8c7)nc(-c7ccc(-c8ccccc8)c8ccccc78)n6)c5c4c3)ccc21 ; CC1(C)CCC(C)(C)c2cc(-c3cccc4c3oc3cccc(-c5nc(-c6ccc7ccccc7c6)nc(-c6ccc7cc(-c8ccccc8)ccc7c6)n5)c34)ccc21 ; CC1(C)CCC(C)(C)c2cc(-c3ccc4c(c3)oc3cccc(-c5nc(-c6ccc7ccccc7c6)nc(-c6ccc7ccc8ccccc8c7c6)n5)c34)ccc21]

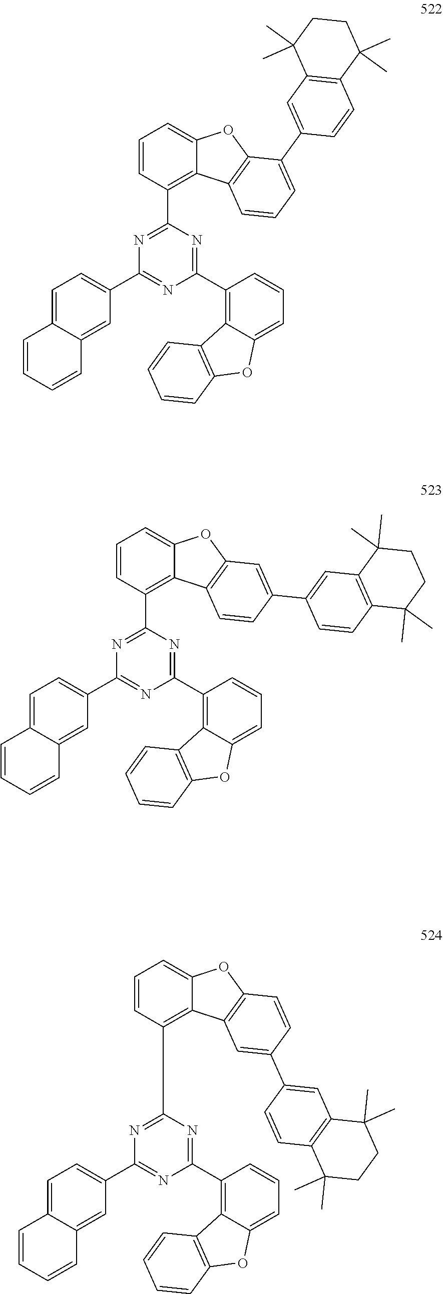 [SMILES: CC1(C)CCC(C)(C)c2cc(-c3cccc4c3oc3cccc(-c5nc(-c6ccc7ccccc7c6)nc(-c6cccc7oc8ccccc8c67)n5)c34)ccc21 ; CC1(C)CCC(C)(C)c2cc(-c3ccc4c(c3)oc3cccc(-c5nc(-c6ccc7ccccc7c6)nc(-c6cccc7oc8ccccc8c67)n5)c34)ccc21 ; CC1(C)CCC(C)(C)c2cc(-c3ccc4oc5cccc(-c6nc(-c7ccc8ccccc8c7)nc(-c7cccc8oc9ccccc9c78)n6)c5c4c3)ccc21]

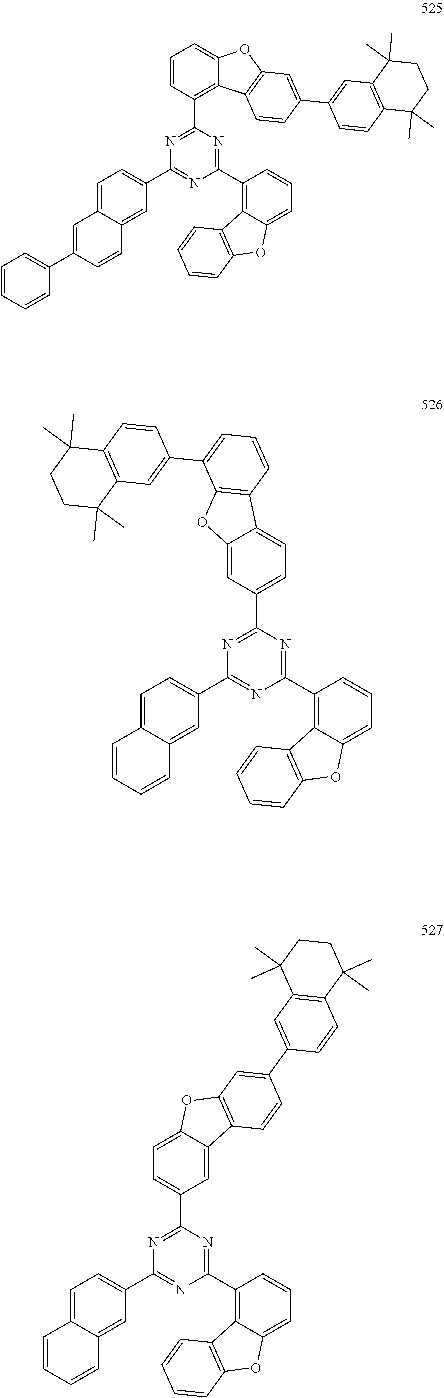

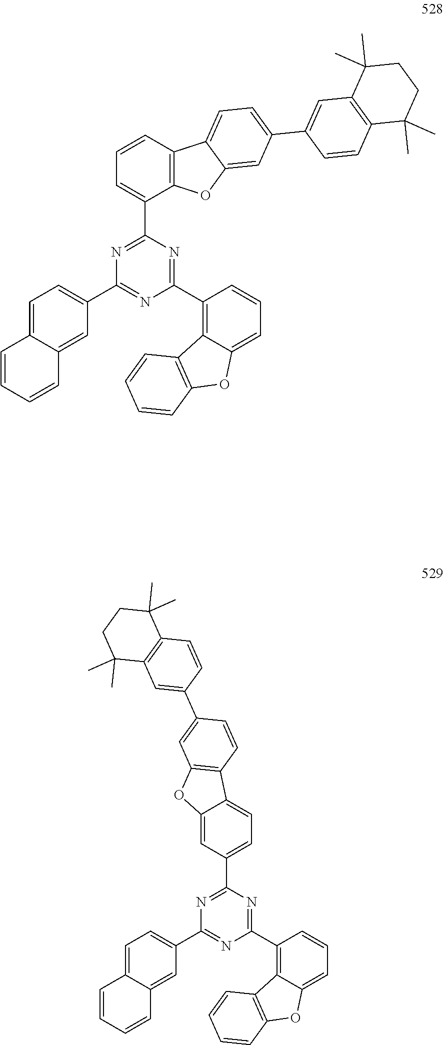

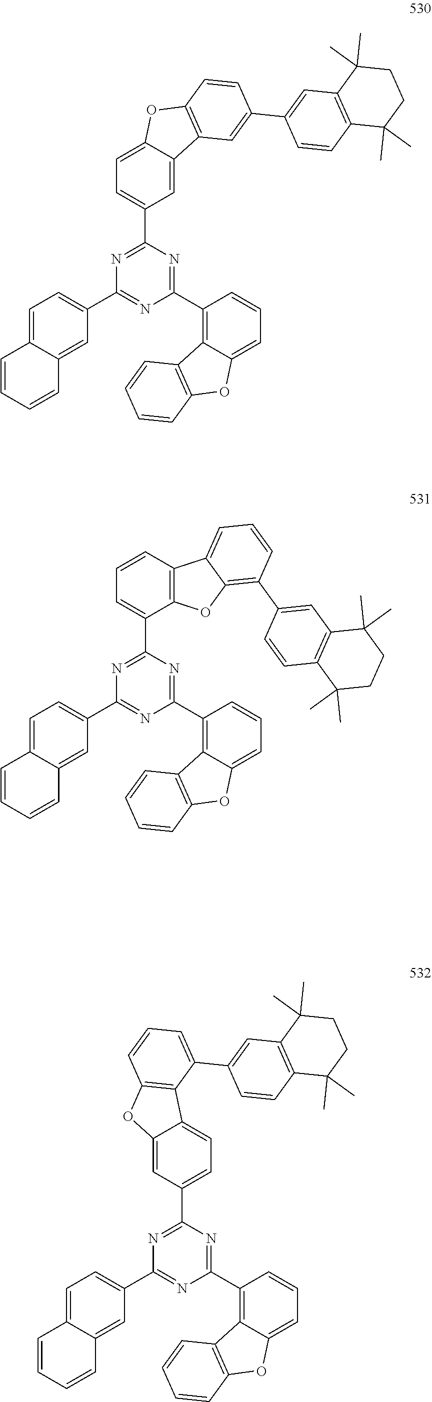 [SMILES: CC1(C)CCC(C)(C)c2cc(-c3ccc4oc5ccc(-c6nc(-c7ccc8ccccc8c7)nc(-c7cccc8oc9ccccc9c78)n6)cc5c4c3)ccc21 ; CC1(C)CCC(C)(C)c2cc(-c3cccc4c3oc3c(-c5nc(-c6ccc7ccccc7c6)nc(-c6cccc7oc8ccccc8c67)n5)cccc34)ccc21 ; CC1(C)CCC(C)(C)c2cc(-c3cccc4oc5cc(-c6nc(-c7ccc8ccccc8c7)nc(-c7cccc8oc9ccccc9c78)n6)ccc5c34)ccc21]

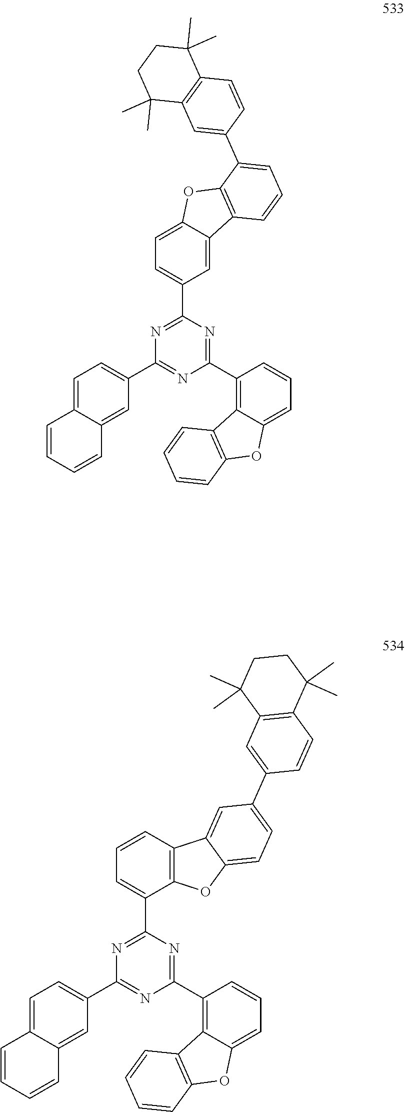 [SMILES: CC1(C)CCC(C)(C)c2cc(-c3cccc4c3oc3ccc(-c5nc(-c6ccc7ccccc7c6)nc(-c6cccc7oc8ccccc8c67)n5)cc34)ccc21 ; CC1(C)CCC(C)(C)c2cc(-c3ccc4oc5c(-c6nc(-c7ccc8ccccc8c7)nc(-c7cccc8oc9ccccc9c78)n6)cccc5c4c3)ccc21]

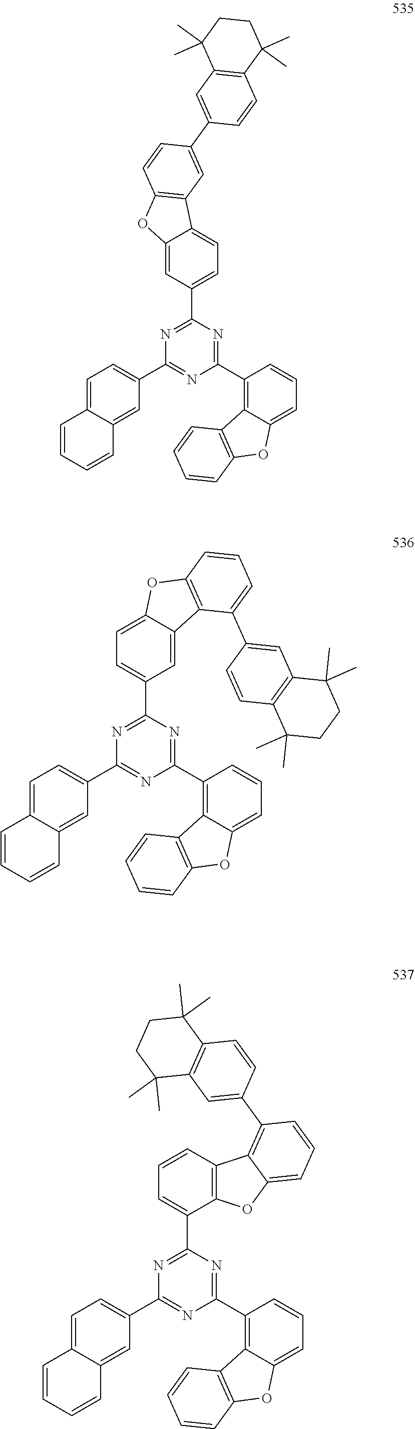 [SMILES: CC1(C)CCC(C)(C)c2cc(-c3ccc4oc5cc(-c6nc(-c7ccc8ccccc8c7)nc(-c7cccc8oc9ccccc9c78)n6)ccc5c4c3)ccc21 ; CC1(C)CCC(C)(C)c2cc(-c3cccc4oc5ccc(-c6nc(-c7ccc8ccccc8c7)nc(-c7cccc8oc9ccccc9c78)n6)cc5c34)ccc21 ; CC1(C)CCC(C)(C)c2cc(-c3cccc4oc5c(-c6nc(-c7ccc8ccccc8c7)nc(-c7cccc8oc9ccccc9c78)n6)cccc5c34)ccc21]

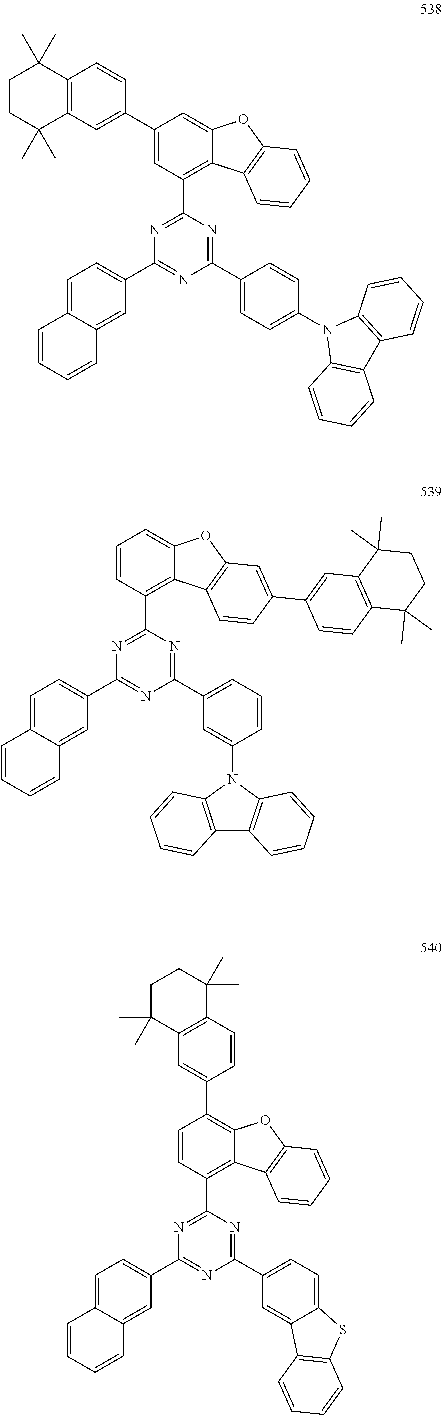 [SMILES: CC1(C)CCC(C)(C)c2cc(-c3cc(-c4nc(-c5ccc(-n6c7ccccc7c7ccccc76)cc5)nc(-c5ccc6ccccc6c5)n4)c4c(c3)oc3ccccc34)ccc21 ; CC1(C)CCC(C)(C)c2cc(-c3ccc4c(c3)oc3cccc(-c5nc(-c6cccc(-n7c8ccccc8c8ccccc87)c6)nc(-c6ccc7ccccc7c6)n5)c34)ccc21 ; CC1(C)CCC(C)(C)c2cc(-c3ccc(-c4nc(-c5ccc6ccccc6c5)nc(-c5ccc6sc7ccccc7c6c5)n4)c4c3oc3ccccc34)ccc21]

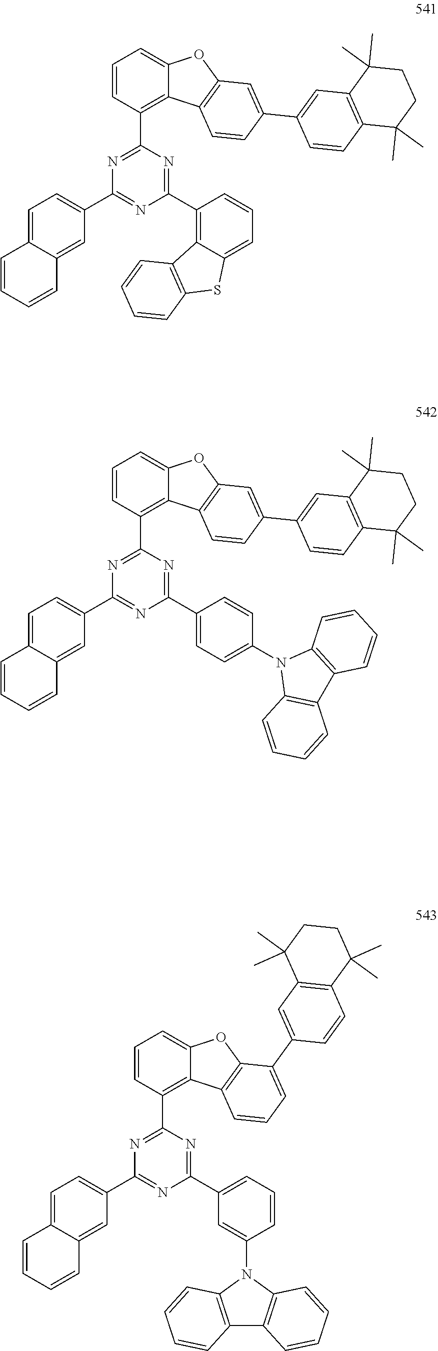 [SMILES: CC1(C)CCC(C)(C)c2cc(-c3ccc4c(c3)oc3cccc(-c5nc(-c6ccc7ccccc7c6)nc(-c6cccc7sc8ccccc8c67)n5)c34)ccc21 ; CC1(C)CCC(C)(C)c2cc(-c3ccc4c(c3)oc3cccc(-c5nc(-c6ccc(-n7c8ccccc8c8ccccc87)cc6)nc(-c6ccc7ccccc7c6)n5)c34)ccc21 ; CC1(C)CCC(C)(C)c2cc(-c3cccc4c3oc3cccc(-c5nc(-c6cccc(-n7c8ccccc8c8ccccc87)c6)nc(-c6ccc7ccccc7c6)n5)c34)ccc21]

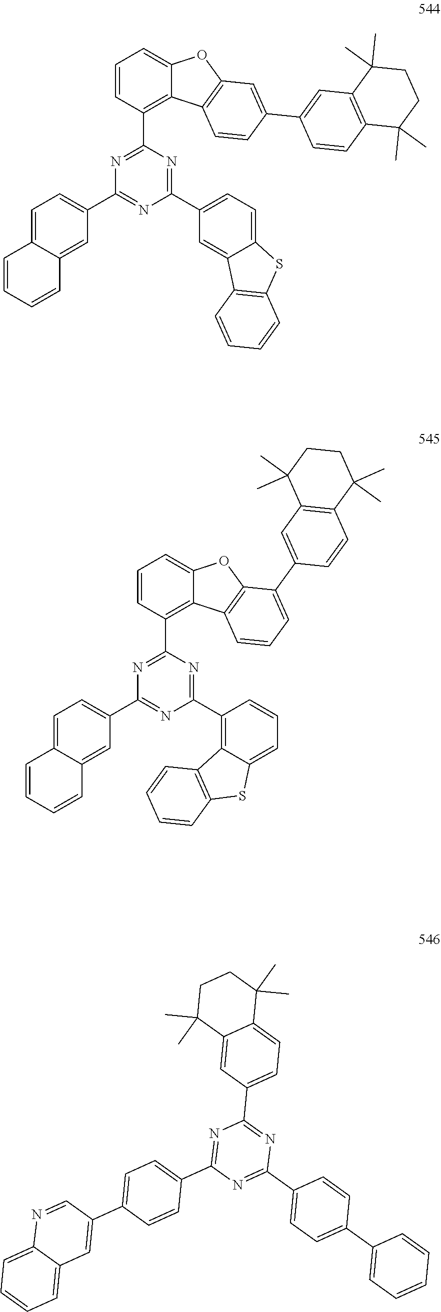 [SMILES: CC1(C)CCC(C)(C)c2cc(-c3ccc4c(c3)oc3cccc(-c5nc(-c6ccc7ccccc7c6)nc(-c6ccc7sc8ccccc8c7c6)n5)c34)ccc21 ; CC1(C)CCC(C)(C)c2cc(-c3cccc4c3oc3cccc(-c5nc(-c6ccc7ccccc7c6)nc(-c6cccc7sc8ccccc8c67)n5)c34)ccc21 ; CC1(C)CCC(C)(C)c2cc(-c3nc(-c4ccc(-c5ccccc5)cc4)nc(-c4ccc(-c5cnc6ccccc6c5)cc4)n3)ccc21]

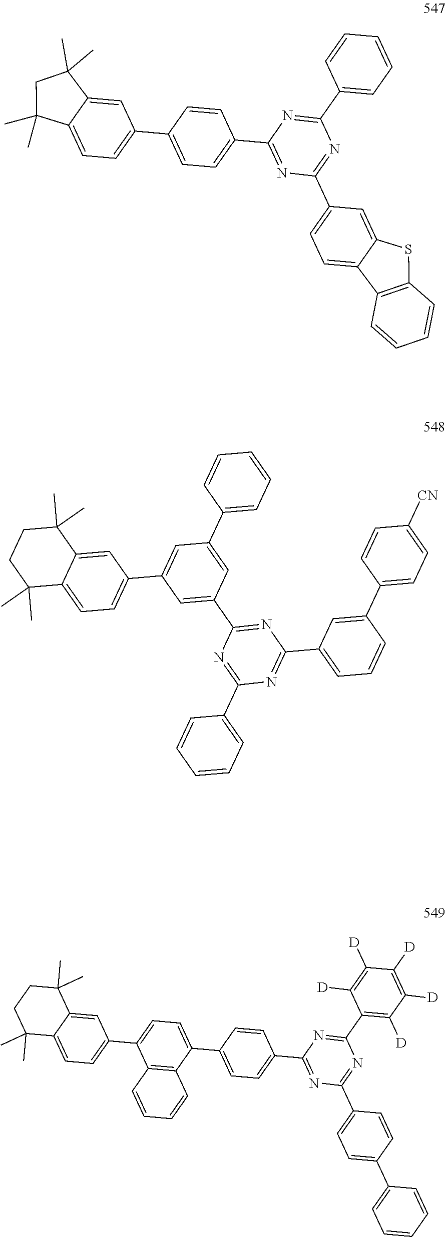 [SMILES: CC1(C)CC(C)(C)c2cc(-c3ccc(-c4nc(-c5ccccc5)nc(-c5ccc6c(c5)sc5ccccc56)n4)cc3)ccc21 ; CC1(C)CCC(C)(C)c2cc(-c3cc(-c4ccccc4)cc(-c4nc(-c5ccccc5)nc(-c5cccc(-c6ccc(C#N)cc6)c5)n4)c3)ccc21 ; [2H]c1c([2H])c([2H])c(-c2nc(-c3ccc(-c4ccccc4)cc3)nc(-c3ccc(-c4ccc(-c5ccc6c(c5)C(C)(C)CCC6(C)C)c5ccccc45)cc3)n2)c([2H])c1[2H]]

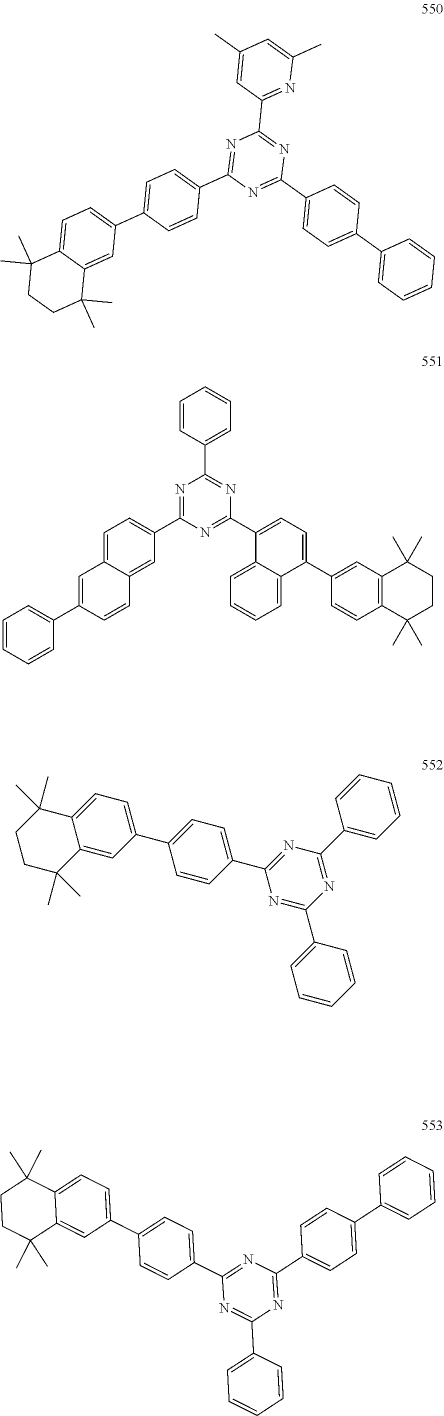 [SMILES: Cc1cc(C)nc(-c2nc(-c3ccc(-c4ccccc4)cc3)nc(-c3ccc(-c4ccc5c(c4)C(C)(C)CCC5(C)C)cc3)n2)c1 ; CC1(C)CCC(C)(C)c2cc(-c3ccc(-c4nc(-c5ccccc5)nc(-c5ccc6cc(-c7ccccc7)ccc6c5)n4)c4ccccc34)ccc21 ; CC1(C)CCC(C)(C)c2cc(-c3ccc(-c4nc(-c5ccccc5)nc(-c5ccccc5)n4)cc3)ccc21 ; CC1(C)CCC(C)(C)c2cc(-c3ccc(-c4nc(-c5ccccc5)nc(-c5ccc(-c6ccccc6)cc5)n4)cc3)ccc21]

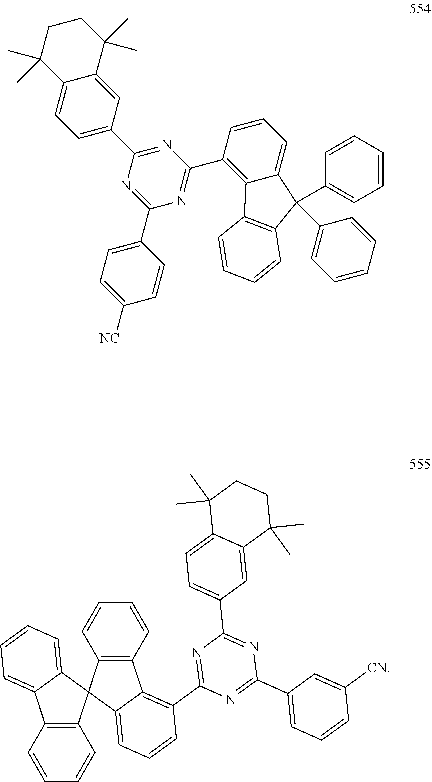 [SMILES: CC1(C)CCC(C)(C)c2cc(-c3nc(-c4ccc(C#N)cc4)nc(-c4cccc5c4-c4ccccc4C5(c4ccccc4)c4ccccc4)n3)ccc21 ; CC1(C)CCC(C)(C)c2cc(-c3nc(-c4cccc(C#N)c4)nc(-c4cccc5c4-c4ccccc4C54c5ccccc5-c5ccccc54)n3)ccc21]

Synthesis methods of the organic compounds provided are not particularly limited in the present disclosure, and those skilled in the art can determine suitable synthesis methods based on the organic compounds of the present disclosure combined with preparation methods provided in the Synthesis Examples section. In other words, the Synthesis Examples section of the present disclosure merely provides some exemplary methods for preparing the organic compounds. Raw materials used can be obtained commercially or by methods well known in the art. Those skilled in the art can obtain all the organic compounds provided in the present disclosure based on these exemplary preparation methods. Not all specific methods for preparing the organic compounds will be described herein in detail. These methods should not be interpreted by those skilled in the art as limitations on the present disclosure.

The present disclosure, in a second aspect, provides an organic electroluminescent device comprising an anode, a cathode, and a functional layer disposed between the anode and the cathode. The functional layer may comprise the organic compound described in the first aspect of the present disclosure.

The organic compound provided in the present disclosure may be used to form at least one organic film layer in the functional layer to improve properties of the organic electroluminescent device, such as prolonging the service life thereof.

Optionally, the functional layer includes an organic emissive layer. The organic emissive layer comprises the organic compound provided in the present disclosure.

Optionally, the functional layer comprises an electronic transport layer. The electronic transport layer comprises the organic compound provided in the present disclosure.

Optionally, the organic electroluminescent device is a green light-emitting device, a red light-emitting device, or a blue light-emitting device.

According to an embodiment, the organic electroluminescent device comprises an anode 100, a hole transport layer 320, an organic emissive layer 330 as an energy conversion layer, an electron transport layer 340, and a cathode 200 that are stacked in sequence. The nitrogen-containing compound provided in the present disclosure may be applied to the organic emissive layer 330 of the organic electroluminescent device to effectively improve the performance of the organic electroluminescent device.

Optionally, the organic emissive layer 330 comprises a host material and a guest material. Holes injected into the organic emissive layer 330 and electrons injected into the organic emissive layer 330 may recombine in the organic emissive layer 330 to form excitons. The excitons transmit energy to the host material, and the host material transmits the energy to the guest material, thereby enabling the guest material to emit light. In an embodiment, the host material comprises the organic compound of the present disclosure. In another embodiment, the host material is selected from α,β-ADN or MADN.

The guest material of the organic emissive layer 330 may be selected by referring to the existing technologies, and may be selected from, for example, the group consisting of anthracene diamine compounds, pyrene diamine compounds, organometallic iridium (III) complexes, organometallic platinum (II) complexes, and ruthenium (II) complexes. In a specific embodiment, the guest material is RD-3. In another specific embodiment, the guest material is BD-1. Structures of RD-3 and BD-1 are shown below and will not be repeated here.

Optionally, the anode 100 comprises an anode material, which is preferably a high-work function material contributing to injection of holes into the functional layer. Specific examples of the anode material include, but are not limited to: metals such as nickel, platinum, vanadium, chromium, copper, zinc, gold, and alloys thereof; metal oxides such as zinc oxide, indium oxide, indium tin oxide (ITO), and indium zinc oxide (IZO); combinations of metals and oxides, such as ZnO:Al or SnO₂:Sb; and conductive polymers such as poly(3-methylthiophene), poly[3,4-(ethylene-1,2-dioxy)thiophene] (PEDT), polypyrrole, and polyaniline. Preferably, a transparent electrode comprising indium tin oxide (ITO) is included as the anode.

In the present disclosure, the material of the hole transport layer 320 may be selected from phthalocyanine derivatives, naphthalocyanine derivatives, porphyrin derivatives, benzidine triarylamine, styrylamine triarylamine, diamine triarylamine, or other types of materials, which can be selected by those skilled in the art referring to the existing technologies. For example, the material of the hole transport layer is selected from the group consisting of the following compounds:

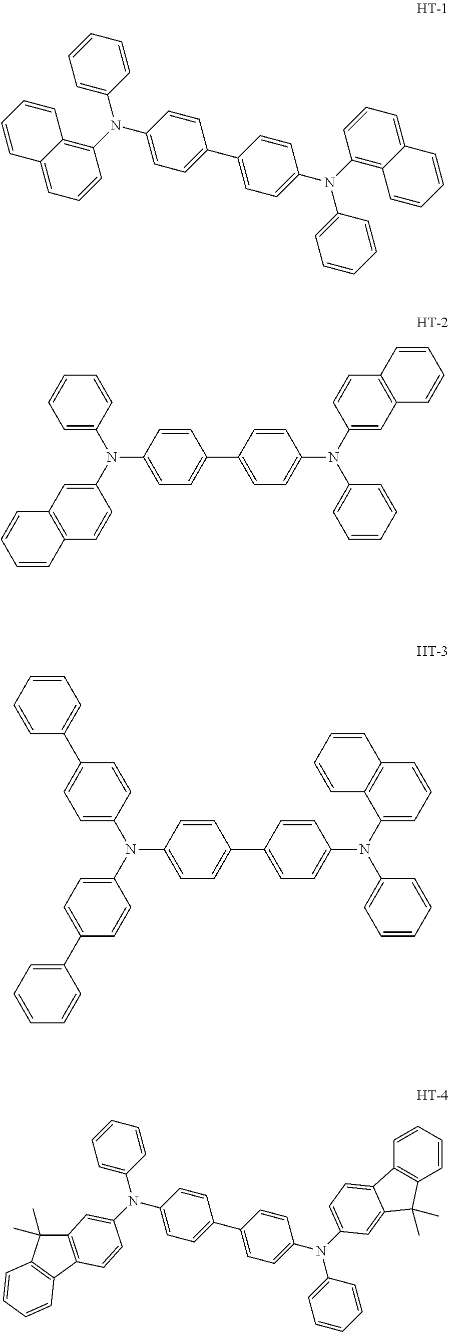 [SMILES: c1ccc(N(c2ccc(-c3ccc(N(c4ccccc4)c4cccc5ccccc45)cc3)cc2)c2cccc3ccccc23)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(N(c3ccc(-c4ccccc4)cc3)c3ccc(-c4ccc(N(c5ccccc5)c5cccc6ccccc56)cc4)cc3)cc2)cc1 ; CC1(C)c2ccccc2-c2ccc(N(c3ccccc3)c3ccc(-c4ccc(N(c5ccccc5)c5ccc6c(c5)C(C)(C)c5ccccc5-6)cc4)cc3)cc21]

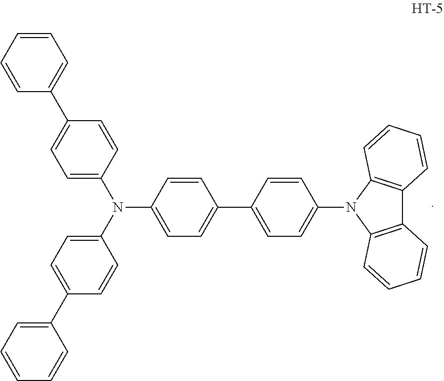 [SMILES: c1ccc(-c2ccc(N(c3ccc(-c4ccccc4)cc3)c3ccc(-c4ccc(-n5c6ccccc6c6ccccc65)cc4)cc3)cc2)cc1]

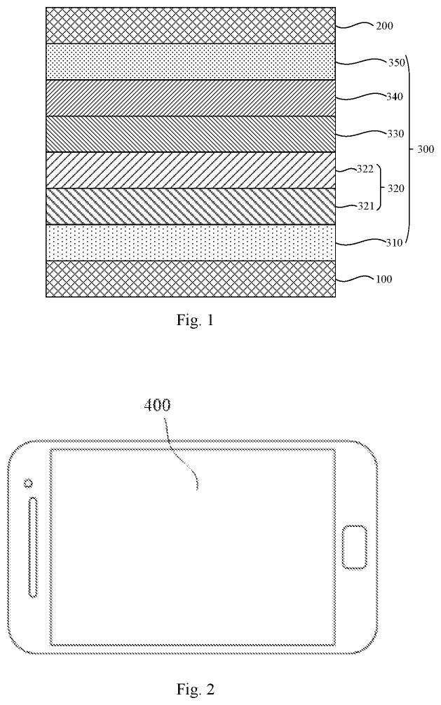

In the present disclosure, the hole transport layer 320 may be a single-layer structure or a two-layer structure. Optionally, as shown in FIG. 1, the hole transport layer 320 comprises a first hole transport layer 321 and a second hole transport layer 322 (also referred to as “emission-auxiliary layer” or “electron blocking layer”) that are stacked in layers. The first hole transport layer 321 is closer to the anode 100 than the second hole transport layer 322.

In a specific embodiment, the first hole transport layer 321 is composed of HT-4 (i.e., BF-DPB), and the second hole transport layer 322 is composed of HT-5. In another specific embodiment, the first hole transport layer 321 is composed of HT-3, and the second hole transport layer 322 is composed of HT-5.

In the present disclosure, the electron transport layer 340 may be a single-layer structure, or a multi-layer structure, and may comprise one or more electron transport materials. Optionally, the materials of the electron transport layer include the organic compound of the present disclosure and other optional electron transport materials. Said other electron transport materials include metal complexes and/or nitrogen-containing heterocyclic derivatives. The metal complex materials may be selected from, for example, LiQ, Alq₃, Bepq₂, etc. The nitrogen-containing heterocyclic derivative may be an aromatic ring having a nitrogen-containing 6-membered ring or 5-membered ring skeleton, a fused aromatic ring compound having a nitrogen-containing 6-membered ring or 5-membered ring skeleton, etc. Specific examples include, but are not limited to, 1,10-phenanthroline compounds such as BCP, Bphen, NBphen, DBimiBphen, BimiBphen, and the like. In a specific embodiment, the electron transport layer is composed of LiQ and the compound of the present disclosure. In another embodiment, the electronic transport layer is composed of said other electronic transport materials, and is composed of, for example, ET-2 (structure thereof is shown below) and LiQ.

Optionally, the cathode 200 may comprise the following cathode material, which is a low-work function material contributing to injection of electrons into the functional layer. Specific examples of the cathode material include, but are not limited to, metals such as magnesium, calcium, sodium, potassium, titanium, indium, yttrium, lithium, gadolinium, aluminum, silver, tin, lead, or alloys thereof; or multilayer materials such as LiF/Al, Liq/Al, LiO₂/Al, LiF/Ca, LiF/Al, and BaF₂/Ca. Preferably, a metal electrode comprising silver and magnesium is included as the cathode.

Optionally, as shown in FIG. 1, a hole injection layer 310 may be further provided between the anode 100 and the first hole transport layer 321 to enhance the ability to inject holes into the first hole transport layer 321. The hole injection layer 310 may be composed of a material selected from benzidine derivatives, starburst arylamine compounds, phthalocyanine derivatives, or other materials, and the present disclosure is not particularly limited in this respect. For example, the material of the hole injection layer 310 may be at least one selected from the group consisting of F4-TCNQ, HAT-CN, m-MTDATA, and 1T-NATA. In a specific embodiment, the material of the hole injection layer 310 is F4-TCNQ. In another specific embodiment, the material of the hole injection layer 310 is 1T-NATA.

Optionally, as shown in FIG. 1, an electron injection layer 350 may be further provided between the cathode 200 and the electron transport layer 340 to enhance the ability to inject electrons into the electron transport layer 340. The electron injection layer 350 may comprise an inorganic material such as an alkali metal sulfide, an alkali metal halide, or may comprise a complex of an alkali metal and an organic substance. For example, the material of the electron injection layer 350 may be one or more than two selected from the group consisting of LiF, NaCl, CsF, Li₂O, BaO, LiQ, NaCl, CsF, Cs₂CO₃, Na, Li, Ca, Al, and Yb. In an embodiment, the material of the electron injection layer 350 may include LiQ or Yb.

The present disclosure, in a third aspect, provides an electronic apparatus. The electronic apparatus comprises the above described organic electroluminescent device.

As shown in FIG. 2, the electronic apparatus is an electronic apparatus 400. The electronic apparatus 400 may be a display apparatus, a lighting apparatus an optical communication apparatus, or other type of electronic apparatus, which, for example, may include, but are not limited to, computer screens, mobile phone screens, televisions, electronic paper, emergency lamps, optical modules, etc.

The present disclosure is further described below by way of embodiments, which, however, do not limit the present disclosure in any way. Compounds for which a synthesis method is not mentioned are raw material products obtained commercially.

### 1. Synthesis of Intermediates IM I-X

The synthesis of Intermediates IM I-X is illustrated by taking IM I-A as an example.

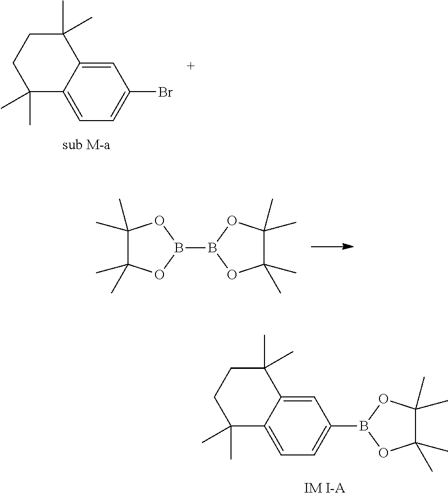

Raw material sub M-a (21.6 g, 80.8 mmol), bis(pinacolato)diboron (20.6 g, 80.8 mmol), tris(dibenzylideneacetonyl)bis-palladium (1.48 g, 1.61 mmol), 2-dicyclohexylphosphino-2,4,6-triisopropylbiphenyl (0.38 g, 0.80 mmol), potassium acetate (15.8 g, 161.6 mmol), and 1,4-dioxane (220 mL) were added to a reaction flask, and heated to 110° C. under nitrogen atmosphere, followed by refluxing and stirring for 6 hours. After being cooled to room temperature, the reaction solution was extracted with dichloromethane and water. The resulting organic layer was dried with anhydrous magnesium sulfate and filtered. After the filtration, the resulting filtrate was passed through a short silica gel column, followed by removal of the solvent under reduced pressure. The resulting crude product was purified by recrystallization using a mixture of dichloromethane and petroleum ether (1:3, v/v), yielding Intermediate IM I-A (18.5 g, yield 72.9%).

Other intermediates IM I-X were synthesized following the synthesis method of IM I-A, except that sub M-a was replaced with a corresponding raw material 1. Main raw materials used, the intermediates IM I-X synthesized, and yields thereof are shown in Table 1.

TABLE 1

| | | | Yield/ |
|---|---|---|---|
| Raw material 1 | Bis(pinacolato)diboron | IM I-X | % |
| 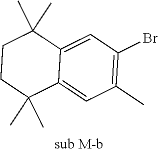 [SMILES: Cc1cc2c(cc1Br)C(C)(C)CCC2(C)C] | 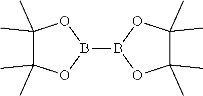 | 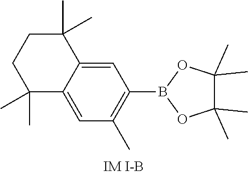 | 66 |
| 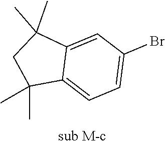 | | 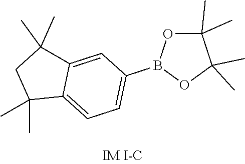 | 70 |
| 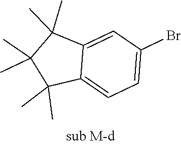 [SMILES: CC1(C)c2ccc(Br)cc2C(C)(C)C1(C)C] | | 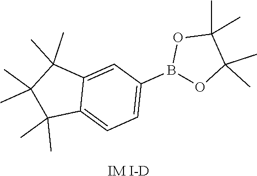 | 68 |

### 2. Synthesis of Intermediates IM I-A-LX

The synthesis of Intermediates IM I-A-LX is illustrated by taking IM I-A-L1 as an example.

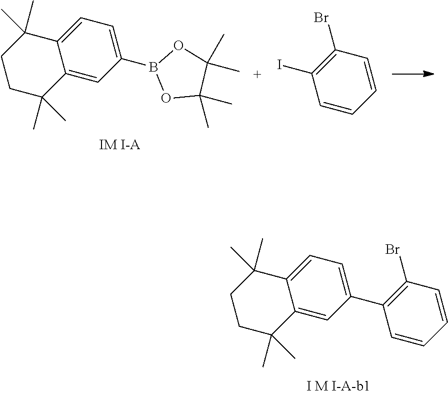

(1) IM I-A (5.00 g, 15.9 mmol), o-bromoiodobenzene (5.40 g, 19.1 mmol), palladium acetate (0.18 g, 0.80 mmol), 2-dicyclohexylphosphino-2,4,6-triisopropylbiphenyl (0.38 g, 0.80 mmol), potassium carbonate (4.84 g, 35.0 mmol), toluene (40 mL), ethanol (20 mL), and water (10 mL) were added to a reaction flask, and heated to 78° C. under nitrogen atmosphere, following by refluxing and stirring for 5 hours. After being cooled to room temperature, the reaction solution was extracted with dichloromethane and water. The resulting organic layer was dried with anhydrous magnesium sulfate and filtered. After the filtration, the resulting filtrate was passed through a short silica gel column. The resulting liquid was distilled under reduced pressure to remove the solvent. The resulting crude product was purified by recrystallization using a mixture of ethyl acetate and petroleum ether (1:3, v/v), yielding Intermediate IM I-A-b1 (4.26 g, yield 78%).

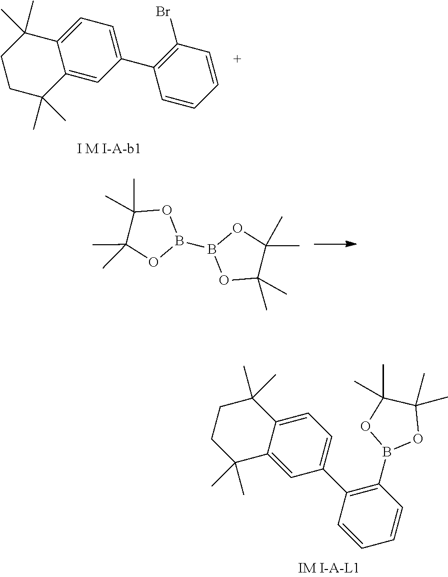

(2) Intermediate IM I-A-L1 was synthesized by the same method for synthesizing Intermediate IM I-A, except that raw material sub M-a was replaced with IM I-A-b1. Yielded was a white solid, which was Intermediate IM I-A-L1 (3.35 g, yield 70.2%).

Other intermediates IM I-A-LX were synthesized following the synthesis method of IM I-A-L1, except that in step (1), o-bromoiodobenzene was replaced with a corresponding raw material 2 and IM A-1 was replaced with a corresponding raw material 3 when synthesizing IM I-A-bX, and that in step (2), IM I-A-b1 was replaced with IM I-A-bX. Main raw materials used, the intermediates IM I-A-LX synthesized, and final yields thereof are shown in Table 2.

TABLE 2

| Raw Material 2 | Raw Material 3 | IM I-A-bX |
|---|---|---|
| 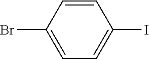 | 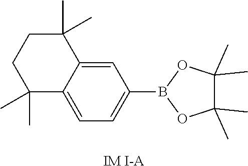 | 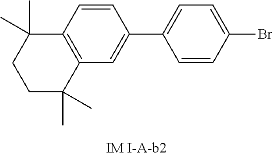 [SMILES: CC1(C)CCC(C)(C)c2cc(-c3ccc(Br)cc3)ccc21] |
| 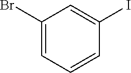 | | 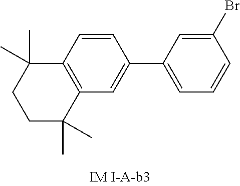 [SMILES: CC1(C)CCC(C)(C)c2cc(-c3cccc(Br)c3)ccc21] |
| 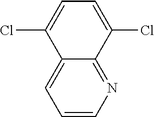 [SMILES: Clc1ccc(Cl)c2ncccc12] | | 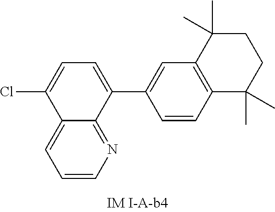 [SMILES: CC1(C)CCC(C)(C)c2cc(-c3ccc(Cl)c4cccnc34)ccc21] |
| 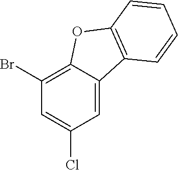 [SMILES: Clc1cc(Br)c2oc3ccccc3c2c1] | 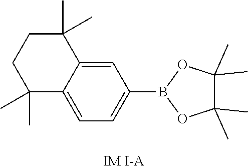 | 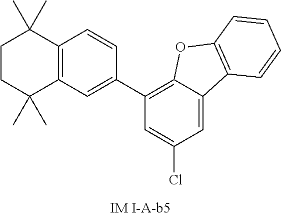 [SMILES: CC1(C)CCC(C)(C)c2cc(-c3cc(Cl)cc4c3oc3ccccc34)ccc21] |
| 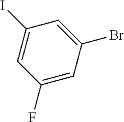 | 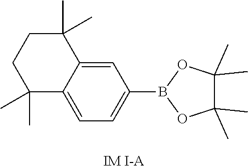 | 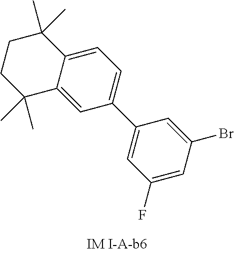 [SMILES: CC1(C)CCC(C)(C)c2cc(-c3cc(F)cc(Br)c3)ccc21] |
| 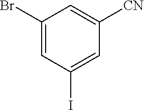 | 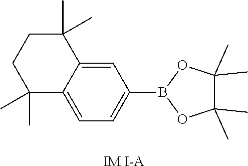 | 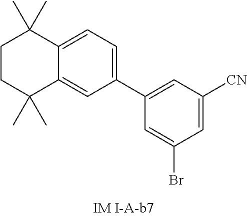 [SMILES: CC1(C)CCC(C)(C)c2cc(-c3cc(Br)cc(C#N)c3)ccc21] |
| 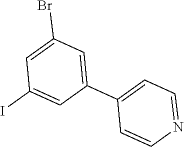 [SMILES: Brc1cc(I)cc(-c2ccncc2)c1] | 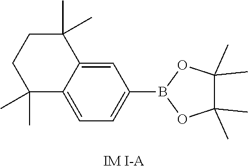 | 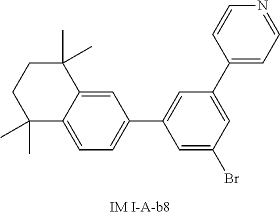 [SMILES: CC1(C)CCC(C)(C)c2cc(-c3cc(Br)cc(-c4ccncc4)c3)ccc21] |
| 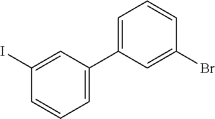 [SMILES: Brc1cccc(-c2cccc(I)c2)c1] | 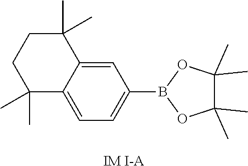 | 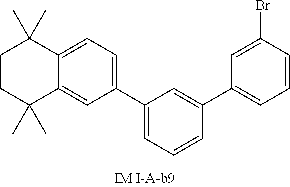 [SMILES: CC1(C)CCC(C)(C)c2cc(-c3cccc(-c4cccc(Br)c4)c3)ccc21] |
| 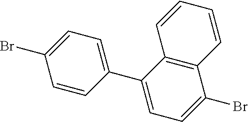 [SMILES: Brc1ccc(-c2ccc(Br)c3ccccc23)cc1] | 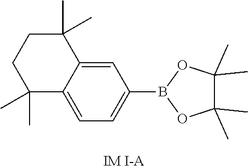 | 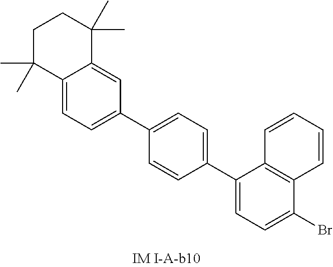 [SMILES: CC1(C)CCC(C)(C)c2cc(-c3ccc(-c4ccc(Br)c5ccccc45)cc3)ccc21] |
| 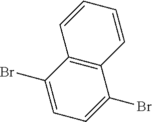 [SMILES: Brc1ccc(Br)c2ccccc12] | 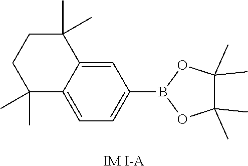 | 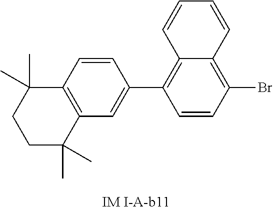 [SMILES: CC1(C)CCC(C)(C)c2cc(-c3ccc(Br)c4ccccc34)ccc21] |
| 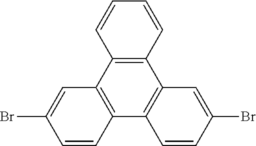 [SMILES: Brc1ccc2c3ccc(Br)cc3c3ccccc3c2c1] | 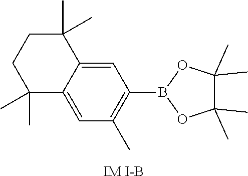 | 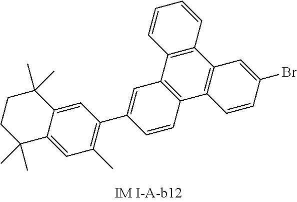 [SMILES: Cc1cc2c(cc1-c1ccc3c4ccc(Br)cc4c4ccccc4c3c1)C(C)(C)CCC2(C)C] |
| 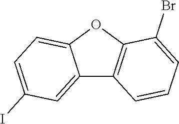 [SMILES: Brc1cccc2c1oc1ccc(I)cc12] | 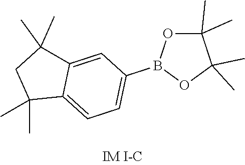 | 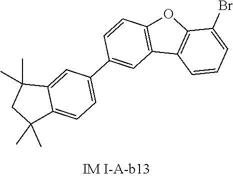 [SMILES: CC1(C)CC(C)(C)c2cc(-c3ccc4oc5c(Br)cccc5c4c3)ccc21] |
| 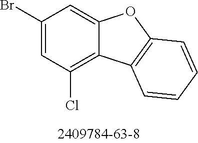 [SMILES: Clc1cc(Br)cc2oc3ccccc3c12] | 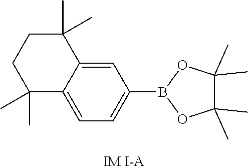 | 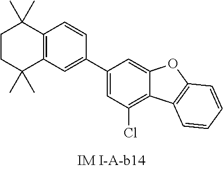 [SMILES: CC1(C)CCC(C)(C)c2cc(-c3cc(Cl)c4c(c3)oc3ccccc34)ccc21] |
| 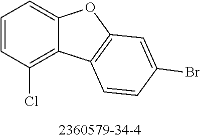 [SMILES: Clc1cccc2oc3cc(Br)ccc3c12] | 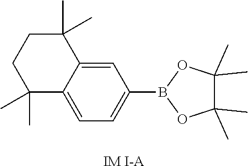 | 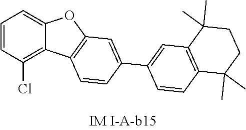 [SMILES: CC1(C)CCC(C)(C)c2cc(-c3ccc4c(c3)oc3cccc(Cl)c34)ccc21] |
| 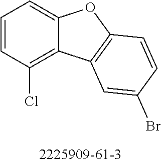 [SMILES: Clc1cccc2oc3ccc(Br)cc3c12] | 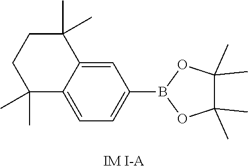 | 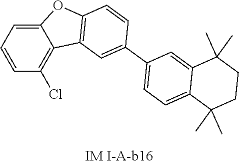 [SMILES: CC1(C)CCC(C)(C)c2cc(-c3ccc4oc5cccc(Cl)c5c4c3)ccc21] |
| 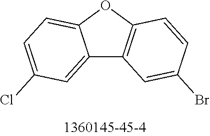 [SMILES: Clc1ccc2oc3ccc(Br)cc3c2c1] | 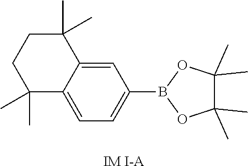 | 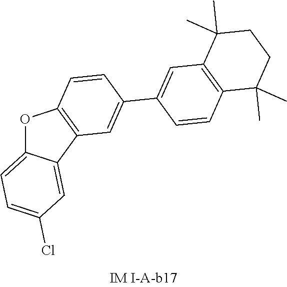 [SMILES: CC1(C)CCC(C)(C)c2cc(-c3ccc4oc5ccc(Cl)cc5c4c3)ccc21] |
| 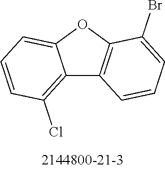 [SMILES: Clc1cccc2oc3c(Br)cccc3c12] | 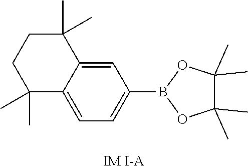 | 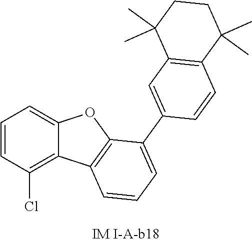 [SMILES: CC1(C)CCC(C)(C)c2cc(-c3cccc4c3oc3cccc(Cl)c34)ccc21] |
| 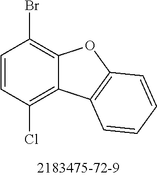 [SMILES: Clc1ccc(Br)c2oc3ccccc3c12] | 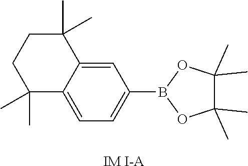 | 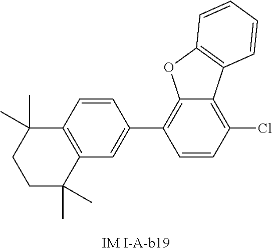 [SMILES: CC1(C)CCC(C)(C)c2cc(-c3ccc(Cl)c4c3oc3ccccc34)ccc21] |

| IM I-A-LX | Yield % |
|---|---|
| 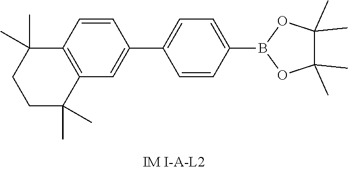 | 63 |
| 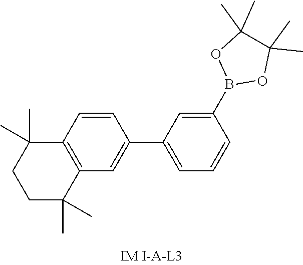 | 72 |
| 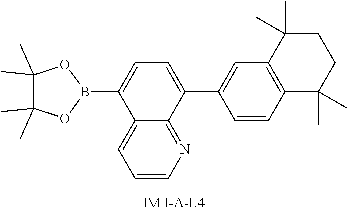 | 45 |
| 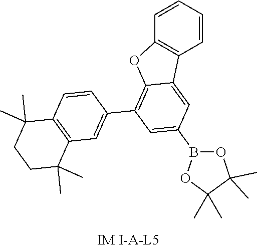 | 69 |
| 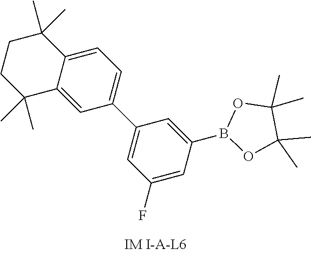 [SMILES: CC1(C)CCC(C)(C)c2cc(-c3cc(F)cc(B4OC(C)(C)C(C)(C)O4)c3)ccc21] | 63 |
| 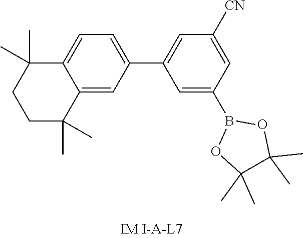 | 58 |
| 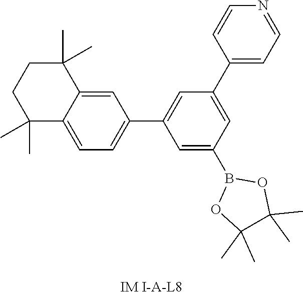 | 61 |
| 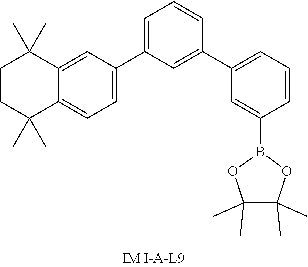 [SMILES: CC1(C)CCC(C)(C)c2cc(-c3cccc(-c4cccc(B5OC(C)(C)C(C)(C)O5)c4)c3)ccc21] | 57 |
| 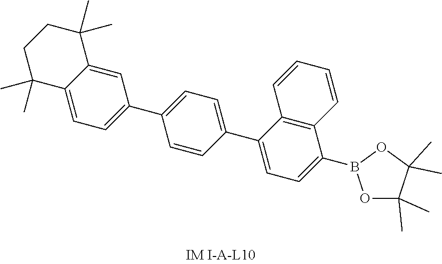 | 54 |
| 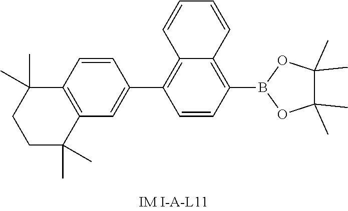 | 59 |
| 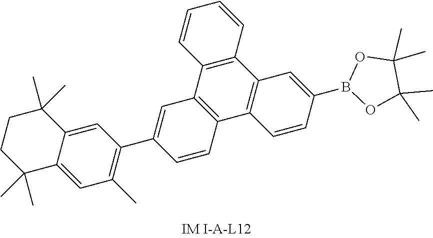 [SMILES: Cc1cc2c(cc1-c1ccc3c4ccc(B5OC(C)(C)C(C)(C)O5)cc4c4ccccc4c3c1)C(C)(C)CCC2(C)C] | 53 |
| 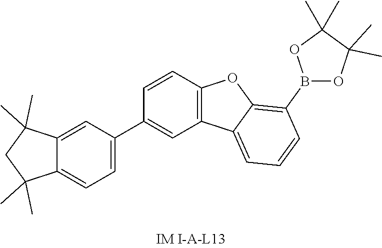 | 49 |
| 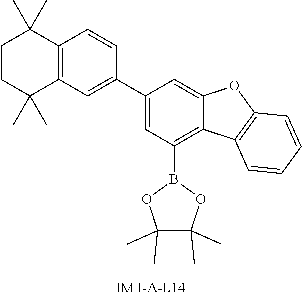 [SMILES: CC1(C)CCC(C)(C)c2cc(-c3cc(B4OC(C)(C)C(C)(C)O4)c4c(c3)oc3ccccc34)ccc21] | 42 |
| 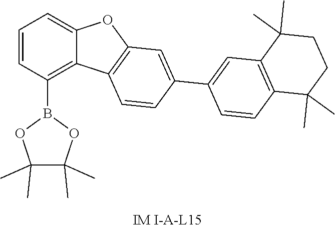 | 43 |
| 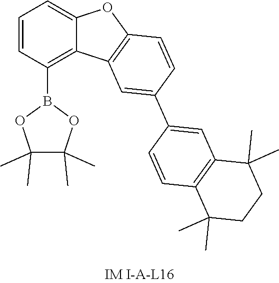 | 41 |
| 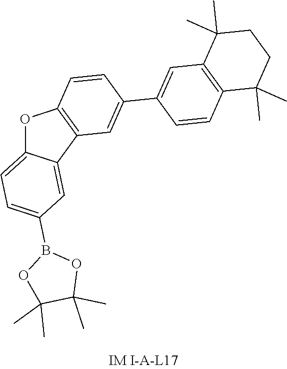 [SMILES: CC1(C)CCC(C)(C)c2cc(-c3ccc4oc5ccc(B6OC(C)(C)C(C)(C)O6)cc5c4c3)ccc21] | 44 |
| 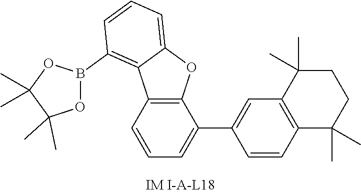 | 45 |
| 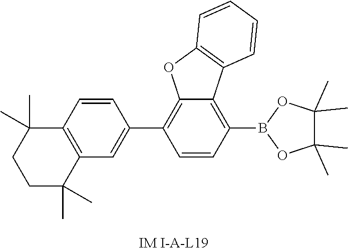 | 43 |

### Synthesis Example 1: Synthesis of Compound 6

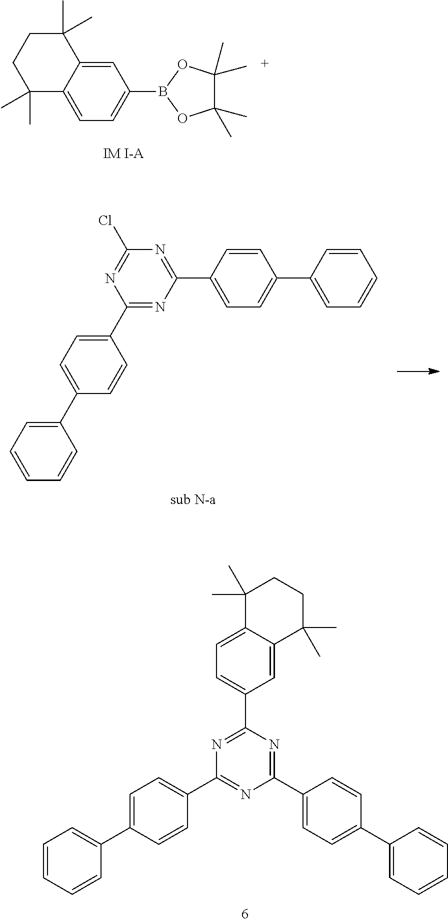

IM I-A (3.4 g, 10.8 mmol), raw material sub N-a (5.9 g, 14.0 mmol), palladium acetate (0.12 g, 0.54 mmol), 2-dicyclohexylphosphino-2,4,6-triisopropylbiphenyl (0.26 g, 0.54 mmol), anhydrous potassium carbonate (3.29 g, 23.8 mmol), toluene (40 mL), ethanol (15 mL), and water (10 mL) were added to a reaction flask, and heated to 78° C. under nitrogen atmosphere, followed by refluxing and stirring for 5 hours. After being cooled to room temperature, the reaction solution was extracted with dichloromethane and water. The resulting organic layer was dried with anhydrous magnesium sulfate and filtered. After the filtration, the resulting filtrate was passed through a short silica gel column, followed by distillation under reduced pressure to remove the solvent. The resulting crude product was purified by recrystallization using a mixture of ethyl acetate and n-heptane (1:3, v/v), yielding Compound 6 (4.41 g, yield 71%); mass spectrometry: m/z=572.3[M+H]⁺.

### Synthesis Examples 2 to 27

Compounds listed in Table 3 were synthesized following the same method for synthesizing Compound 6, except that IM I-A was replaced with a corresponding raw material 3, and that raw material sub N-a was replaced with a corresponding raw material 4. Main raw materials used, the compounds synthesized, yields thereof, and mass spectrometry (MS) characterization results thereof are shown in Table 3.

TABLE 3

| Synthesis | | |
|---|---|---|
| Example | Raw material 3 | Raw material 4 |
| 2 | 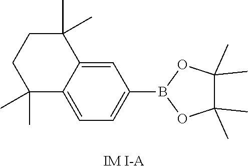 | 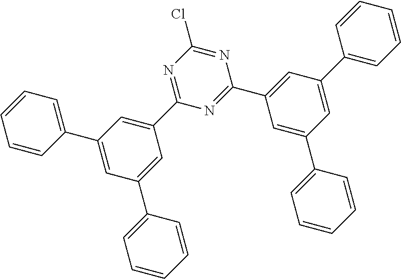 [SMILES: Clc1nc(-c2cc(-c3ccccc3)cc(-c3ccccc3)c2)nc(-c2cc(-c3ccccc3)cc(-c3ccccc3)c2)n1] |
| 3 | 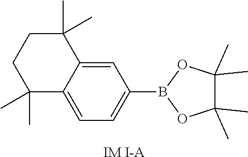 | 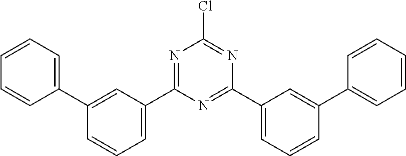 [SMILES: Clc1nc(-c2cccc(-c3ccccc3)c2)nc(-c2cccc(-c3ccccc3)c2)n1] |
| 4 | 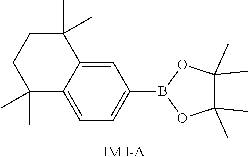 | 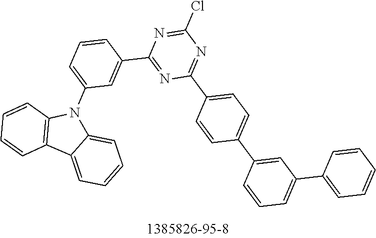 [SMILES: Clc1nc(-c2ccc(-c3cccc(-c4ccccc4)c3)cc2)nc(-c2cccc(-n3c4ccccc4c4ccccc43)c2)n1] |
| 5 | 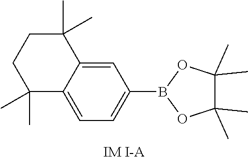 | 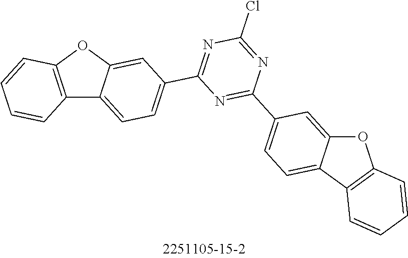 [SMILES: Clc1nc(-c2ccc3c(c2)oc2ccccc23)nc(-c2ccc3c(c2)oc2ccccc23)n1] |
| 6 | 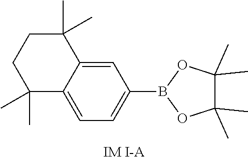 | 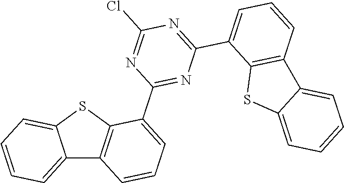 [SMILES: Clc1nc(-c2cccc3c2sc2ccccc23)nc(-c2cccc3c2sc2ccccc23)n1] |
| 7 | 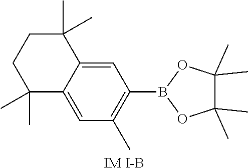 | 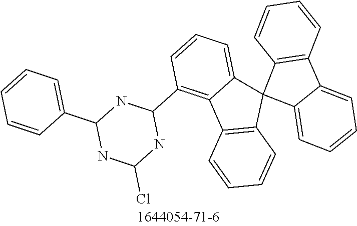 [SMILES: ClC1NC(c2ccccc2)NC(c2cccc3c2-c2ccccc2C32c3ccccc3-c3ccccc32)N1] |
| 8 | 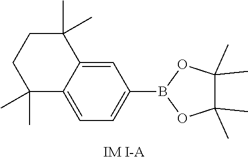 | 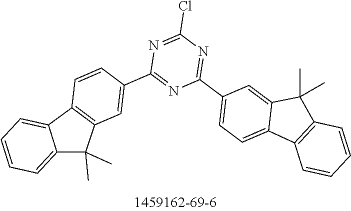 [SMILES: CC1(C)c2ccccc2-c2ccc(-c3nc(Cl)nc(-c4ccc5c(c4)C(C)(C)c4ccccc4-5)n3)cc21] |
| 9 | 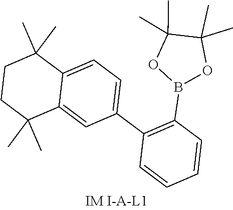 [SMILES: CC1(C)CCC(C)(C)c2cc(-c3ccccc3B3OC(C)(C)C(C)(C)O3)ccc21] | 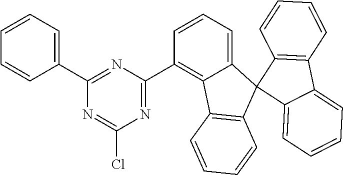 [SMILES: Clc1nc(-c2ccccc2)nc(-c2cccc3c2-c2ccccc2C32c3ccccc3-c3ccccc32)n1] |
| 10 | 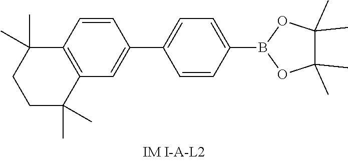 | 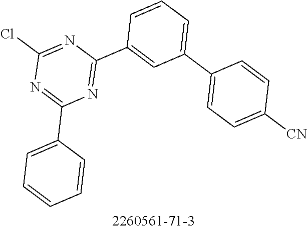 [SMILES: N#Cc1ccc(-c2cccc(-c3nc(Cl)nc(-c4ccccc4)n3)c2)cc1] |
| 11 | 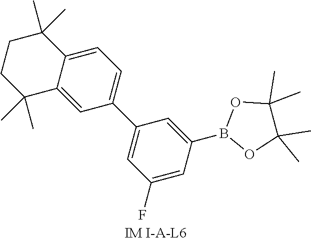 [SMILES: CC1(C)CCC(C)(C)c2cc(-c3cc(F)cc(B4OC(C)(C)C(C)(C)O4)c3)ccc21] | 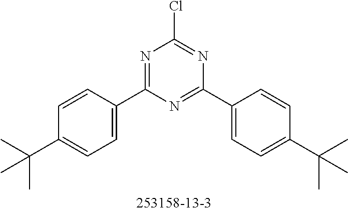 [SMILES: CC(C)(C)c1ccc(-c2nc(Cl)nc(-c3ccc(C(C)(C)C)cc3)n2)cc1] |
| 12 | 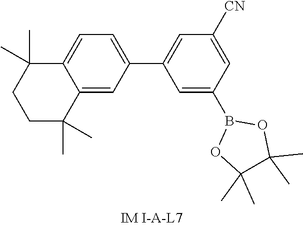 | 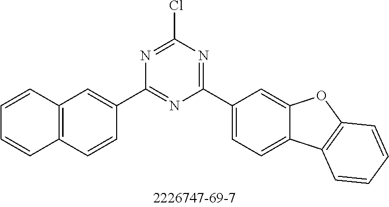 [SMILES: Clc1nc(-c2ccc3ccccc3c2)nc(-c2ccc3c(c2)oc2ccccc23)n1] |
| 13 | 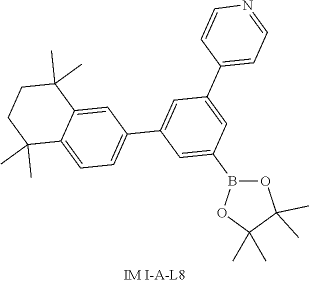 | 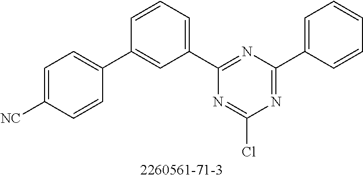 [SMILES: N#Cc1ccc(-c2cccc(-c3nc(Cl)nc(-c4ccccc4)n3)c2)cc1] |
| 14 | 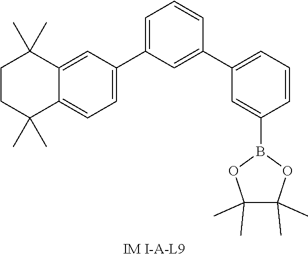 [SMILES: CC1(C)CCC(C)(C)c2cc(-c3cccc(-c4cccc(B5OC(C)(C)C(C)(C)O5)c4)c3)ccc21] | 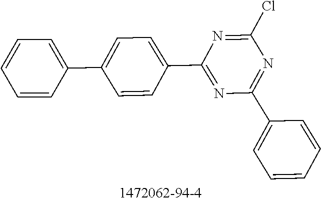 [SMILES: Clc1nc(-c2ccccc2)nc(-c2ccc(-c3ccccc3)cc2)n1] |
| 15 | 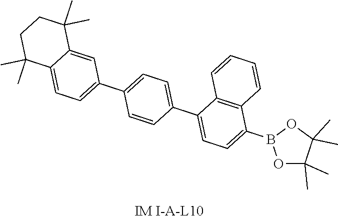 | 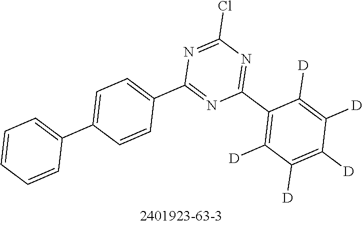 [SMILES: [2H]c1c([2H])c([2H])c(-c2nc(Cl)nc(-c3ccc(-c4ccccc4)cc3)n2)c([2H])c1[2H]] |
| 16 | 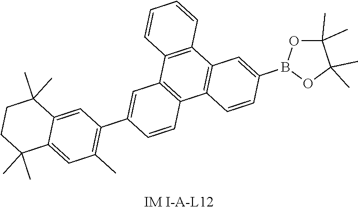 [SMILES: Cc1cc2c(cc1-c1ccc3c4ccc(B5OC(C)(C)C(C)(C)O5)cc4c4ccccc4c3c1)C(C)(C)CCC2(C)C] | 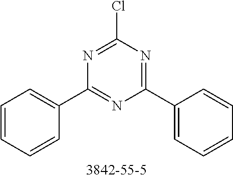 [SMILES: Clc1nc(-c2ccccc2)nc(-c2ccccc2)n1] |
| 17 | 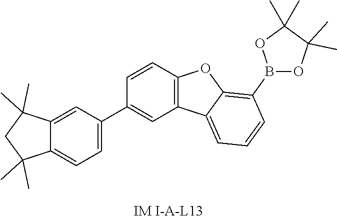 | 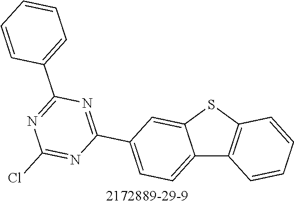 [SMILES: Clc1nc(-c2ccccc2)nc(-c2ccc3c(c2)sc2ccccc23)n1] |
| 18 | 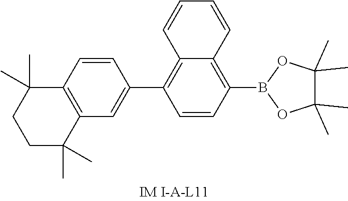 | 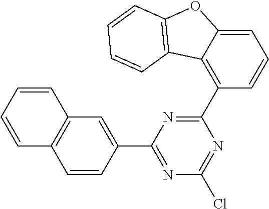 [SMILES: Clc1nc(-c2ccc3ccccc3c2)nc(-c2cccc3oc4ccccc4c23)n1] |
| 19 | 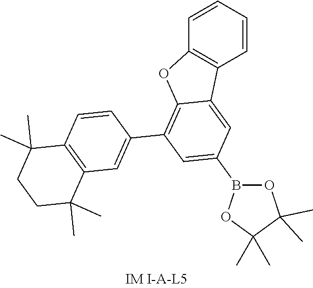 | 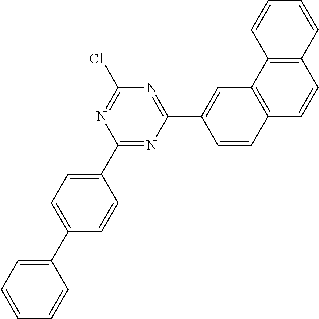 [SMILES: Clc1nc(-c2ccc(-c3ccccc3)cc2)nc(-c2ccc3ccc4ccccc4c3c2)n1] |
| 20 | 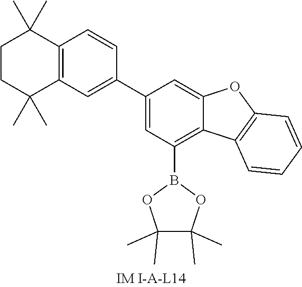 [SMILES: CC1(C)CCC(C)(C)c2cc(-c3cc(B4OC(C)(C)C(C)(C)O4)c4c(c3)oc3ccccc34)ccc21] | 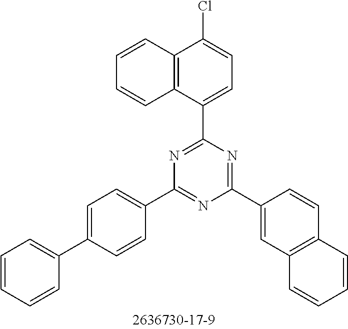 [SMILES: Clc1ccc(-c2nc(-c3ccc(-c4ccccc4)cc3)nc(-c3ccc4ccccc4c3)n2)c2ccccc12] |
| 21 | 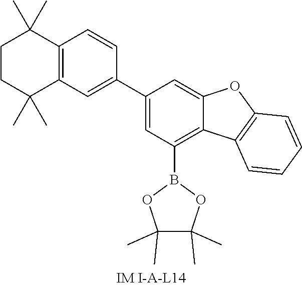 [SMILES: CC1(C)CCC(C)(C)c2cc(-c3cc(B4OC(C)(C)C(C)(C)O4)c4c(c3)oc3ccccc34)ccc21] | 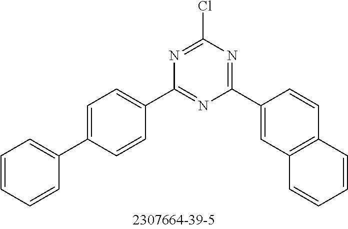 [SMILES: Clc1nc(-c2ccc(-c3ccccc3)cc2)nc(-c2ccc3ccccc3c2)n1] |
| 22 | 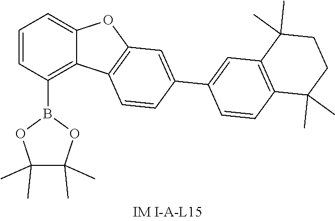 | 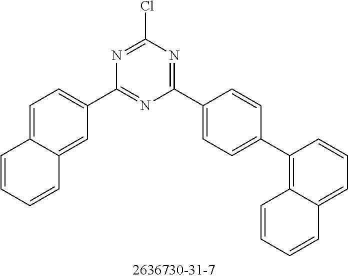 [SMILES: Clc1nc(-c2ccc(-c3cccc4ccccc34)cc2)nc(-c2ccc3ccccc3c2)n1] |
| 23 | 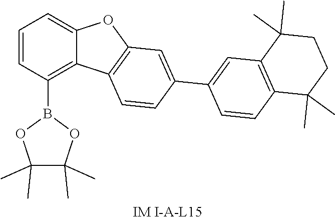 | 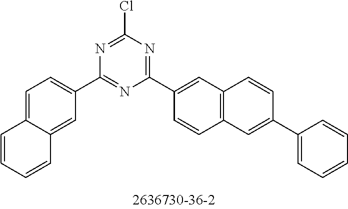 [SMILES: Clc1nc(-c2ccc3ccccc3c2)nc(-c2ccc3cc(-c4ccccc4)ccc3c2)n1] |
| 24 | 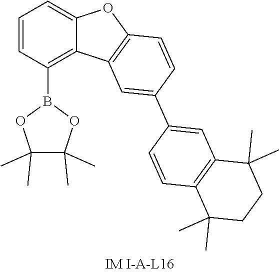 | 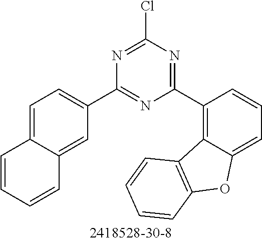 [SMILES: Clc1nc(-c2ccc3ccccc3c2)nc(-c2cccc3oc4ccccc4c23)n1] |
| 25 | 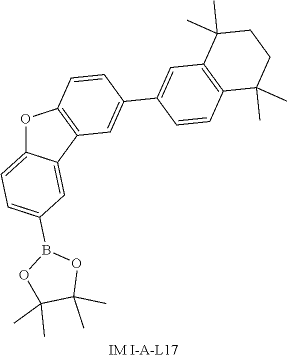 [SMILES: CC1(C)CCC(C)(C)c2cc(-c3ccc4oc5ccc(B6OC(C)(C)C(C)(C)O6)cc5c4c3)ccc21] | 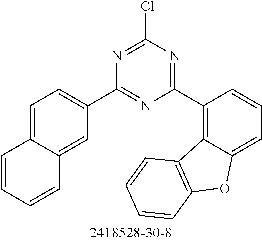 [SMILES: Clc1nc(-c2ccc3ccccc3c2)nc(-c2cccc3oc4ccccc4c23)n1] |
| 26 | 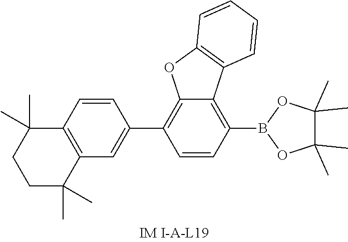 | 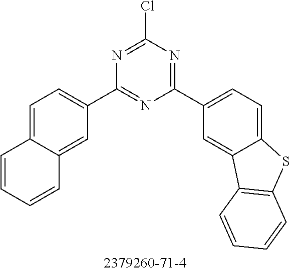 [SMILES: Clc1nc(-c2ccc3ccccc3c2)nc(-c2ccc3sc4ccccc4c3c2)n1] |
| 27 | 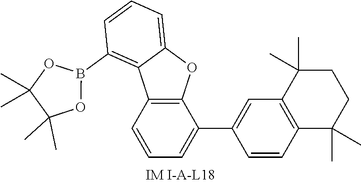 | 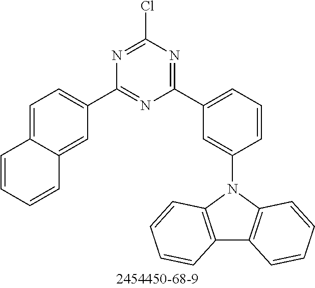 [SMILES: Clc1nc(-c2cccc(-n3c4ccccc4c4ccccc43)c2)nc(-c2ccc3ccccc3c2)n1] |

| Synthesis | | Yield/ | MS/ |
|---|---|---|---|
| Example | Compound | % | [M + H]⁺ |
| 2 | 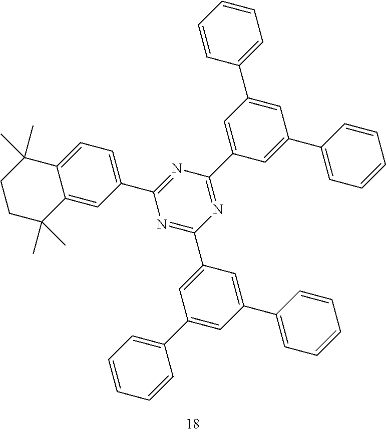 [SMILES: CC1(C)CCC(C)(C)c2cc(-c3nc(-c4cc(-c5ccccc5)cc(-c5ccccc5)c4)nc(-c4cc(-c5ccccc5)cc(-c5ccccc5)c4)n3)ccc21] | 69 | 724.4 |
| 3 | 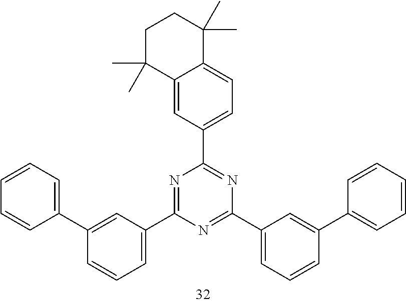 [SMILES: CC1(C)CCC(C)(C)c2cc(-c3nc(-c4cccc(-c5ccccc5)c4)nc(-c4cccc(-c5ccccc5)c4)n3)ccc21] | 74 | 572.3 |
| 4 | 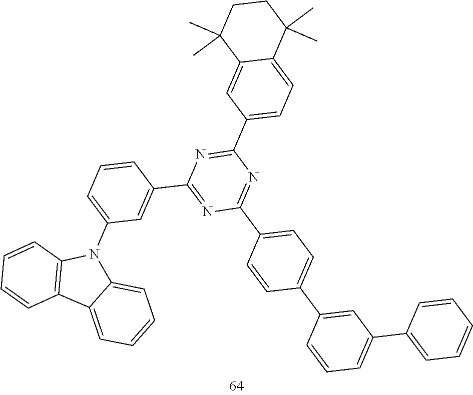 [SMILES: CC1(C)CCC(C)(C)c2cc(-c3nc(-c4ccc(-c5cccc(-c6ccccc6)c5)cc4)nc(-c4cccc(-n5c6ccccc6c6ccccc65)c4)n3)ccc21] | 72 | 737.4 |
| 5 | 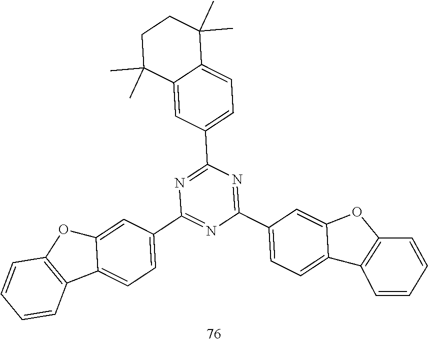 [SMILES: CC1(C)CCC(C)(C)c2cc(-c3nc(-c4ccc5c(c4)oc4ccccc45)nc(-c4ccc5c(c4)oc4ccccc45)n3)ccc21] | 69 | 600.3 |
| 6 | 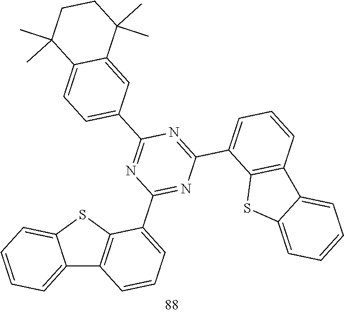 [SMILES: CC1(C)CCC(C)(C)c2cc(-c3nc(-c4cccc5c4sc4ccccc45)nc(-c4cccc5c4sc4ccccc45)n3)ccc21] | 64 | 632.2 |
| 7 | 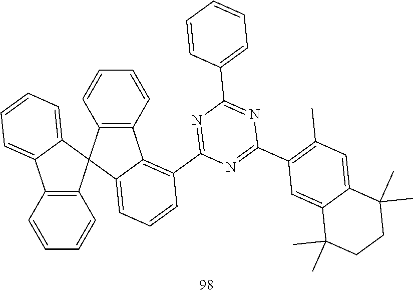 [SMILES: Cc1cc2c(cc1-c1nc(-c3ccccc3)nc(-c3cccc4c3-c3ccccc3C43c4ccccc4-c4ccccc43)n1)C(C)(C)CCC2(C)C] | 73 | 672.3 |
| 8 | 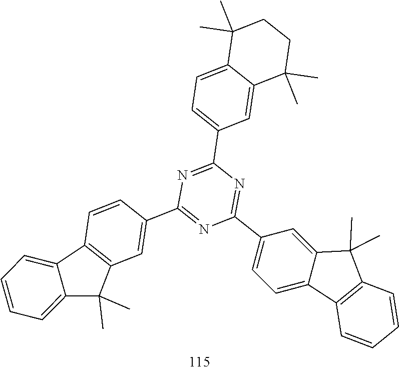 [SMILES: CC1(C)CCC(C)(C)c2cc(-c3nc(-c4ccc5c(c4)C(C)(C)c4ccccc4-5)nc(-c4ccc5c(c4)C(C)(C)c4ccccc4-5)n3)ccc21] | 68 | 652.4 |
| 9 | 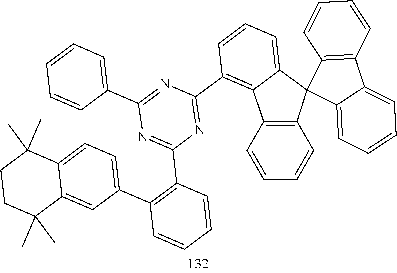 [SMILES: CC1(C)CCC(C)(C)c2cc(-c3ccccc3-c3nc(-c4ccccc4)nc(-c4cccc5c4-c4ccccc4C54c5ccccc5-c5ccccc54)n3)ccc21] | 60 | 734.4 |
| 10 | 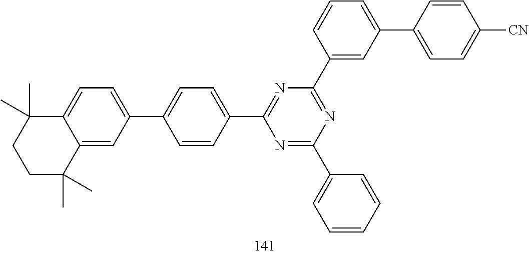 [SMILES: CC1(C)CCC(C)(C)c2cc(-c3ccc(-c4nc(-c5ccccc5)nc(-c5cccc(-c6ccc(C#N)cc6)c5)n4)cc3)ccc21] | 58 | 597.3 |
| 11 | 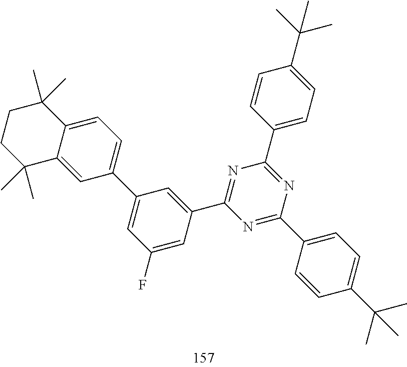 [SMILES: CC(C)(C)c1ccc(-c2nc(-c3ccc(C(C)(C)C)cc3)nc(-c3cc(F)cc(-c4ccc5c(c4)C(C)(C)CCC5(C)C)c3)n2)cc1] | 64 | 626.4 |
| 12 | 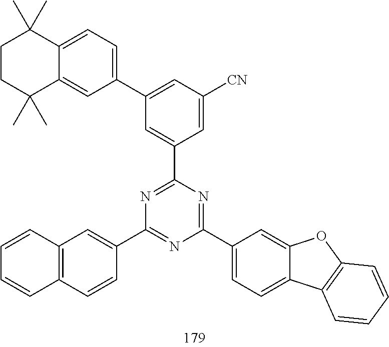 [SMILES: CC1(C)CCC(C)(C)c2cc(-c3cc(C#N)cc(-c4nc(-c5ccc6ccccc6c5)nc(-c5ccc6c(c5)oc5ccccc56)n4)c3)ccc21] | 52 | 661.3 |
| 13 | 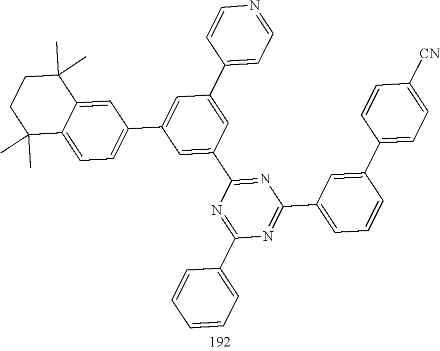 [SMILES: CC1(C)CCC(C)(C)c2cc(-c3cc(-c4ccncc4)cc(-c4nc(-c5ccccc5)nc(-c5cccc(-c6ccc(C#N)cc6)c5)n4)c3)ccc21] | 55 | 674.3 |
| 14 | 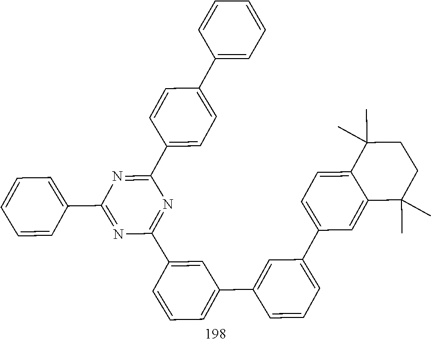 [SMILES: CC1(C)CCC(C)(C)c2cc(-c3cccc(-c4cccc(-c5nc(-c6ccccc6)nc(-c6ccc(-c7ccccc7)cc6)n5)c4)c3)ccc21] | 62 | 648.3 |
| 15 | 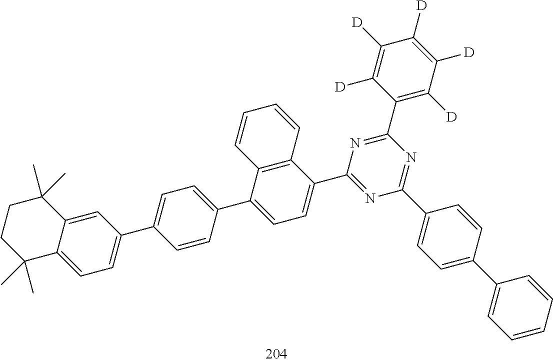 [SMILES: [2H]c1c([2H])c([2H])c(-c2nc(-c3ccc(-c4ccccc4)cc3)nc(-c3ccc(-c4ccc(-c5ccc6c(c5)C(C)(C)CCC6(C)C)cc4)c4ccccc34)n2)c([2H])c1[2H]] | 56 | 703.4 |
| 16 | 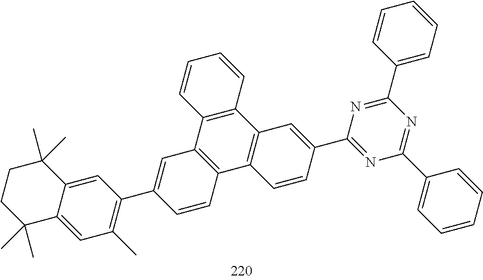 [SMILES: Cc1cc2c(cc1-c1ccc3c4ccc(-c5nc(-c6ccccc6)nc(-c6ccccc6)n5)cc4c4ccccc4c3c1)C(C)(C)CCC2(C)C] | 57 | 660.3 |
| 17 | 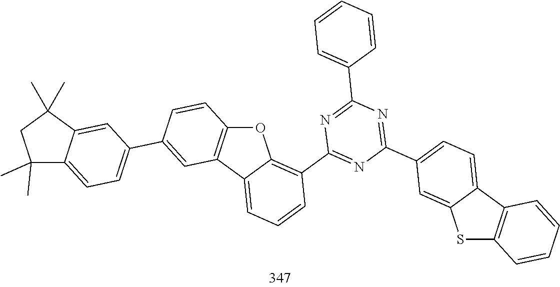 [SMILES: CC1(C)CC(C)(C)c2cc(-c3ccc4oc5c(-c6nc(-c7ccccc7)nc(-c7ccc8c(c7)sc7ccccc78)n6)cccc5c4c3)ccc21] | 49 | 678.2 |
| 18 | 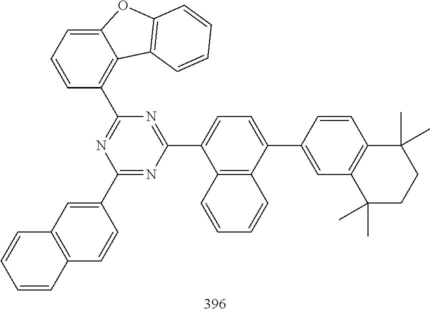 [SMILES: CC1(C)CCC(C)(C)c2cc(-c3ccc(-c4nc(-c5ccc6ccccc6c5)nc(-c5cccc6oc7ccccc7c56)n4)c4ccccc34)ccc21] | 53 | 686.3 |
| 19 | 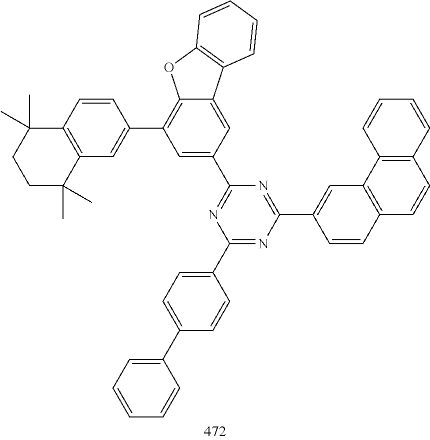 [SMILES: CC1(C)CCC(C)(C)c2cc(-c3cc(-c4nc(-c5ccc(-c6ccccc6)cc5)nc(-c5ccc6ccc7ccccc7c6c5)n4)cc4c3oc3ccccc34)ccc21] | 52 | 762.3 |
| 20 | 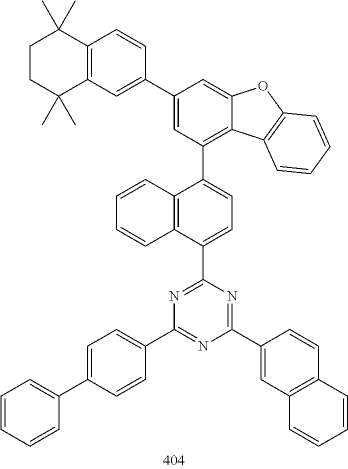 [SMILES: CC1(C)CCC(C)(C)c2cc(-c3cc(-c4ccc(-c5nc(-c6ccc(-c7ccccc7)cc6)nc(-c6ccc7ccccc7c6)n5)c5ccccc45)c4c(c3)oc3ccccc34)ccc21] | 48 | 838.4 |
| 21 | 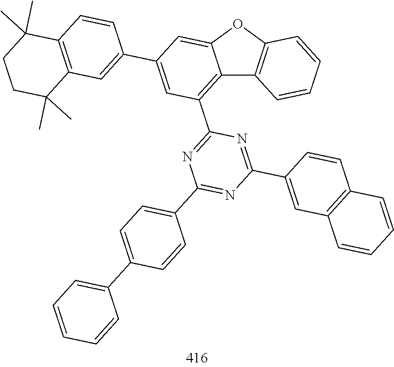 [SMILES: CC1(C)CCC(C)(C)c2cc(-c3cc(-c4nc(-c5ccc(-c6ccccc6)cc5)nc(-c5ccc6ccccc6c5)n4)c4c(c3)oc3ccccc34)ccc21] | 51 | 712.3 |
| 22 | 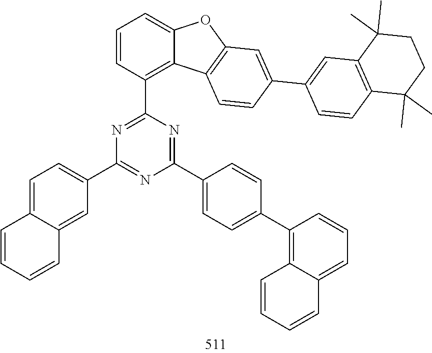 [SMILES: CC1(C)CCC(C)(C)c2cc(-c3ccc4c(c3)oc3cccc(-c5nc(-c6ccc(-c7cccc8ccccc78)cc6)nc(-c6ccc7ccccc7c6)n5)c34)ccc21] | 53 | 762.3 |
| 23 | 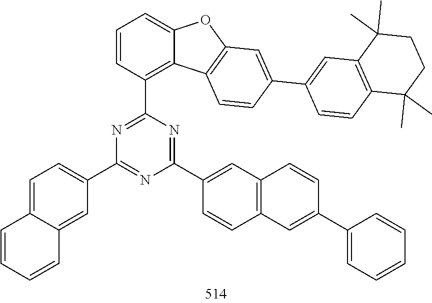 [SMILES: CC1(C)CCC(C)(C)c2cc(-c3ccc4c(c3)oc3cccc(-c5nc(-c6ccc7ccccc7c6)nc(-c6ccc7cc(-c8ccccc8)ccc7c6)n5)c34)ccc21] | 52 | 762.3 |
| 24 | 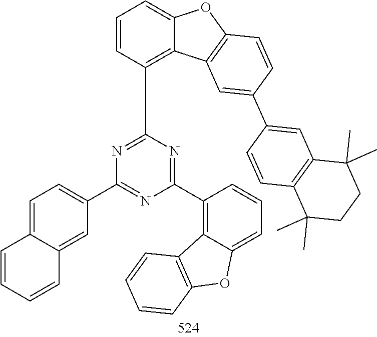 [SMILES: CC1(C)CCC(C)(C)c2cc(-c3ccc4oc5cccc(-c6nc(-c7ccc8ccccc8c7)nc(-c7cccc8oc9ccccc9c78)n6)c5c4c3)ccc21] | 45 | 726.3 |
| 25 | 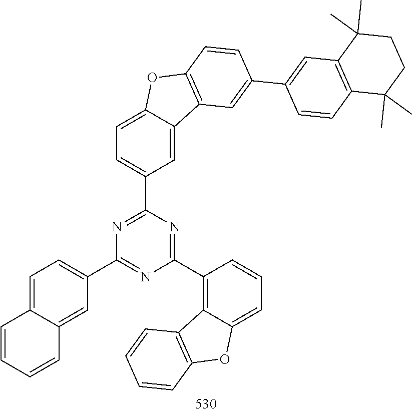 [SMILES: CC1(C)CCC(C)(C)c2cc(-c3ccc4oc5ccc(-c6nc(-c7ccc8ccccc8c7)nc(-c7cccc8oc9ccccc9c78)n6)cc5c4c3)ccc21] | 53 | 726.3 |
| 26 | 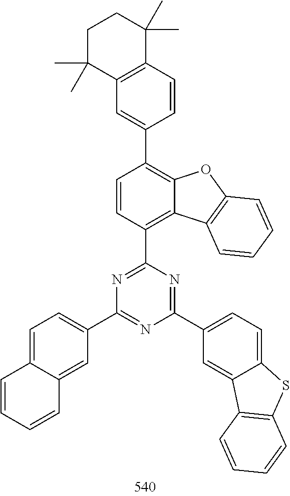 [SMILES: CC1(C)CCC(C)(C)c2cc(-c3ccc(-c4nc(-c5ccc6ccccc6c5)nc(-c5ccc6sc7ccccc7c6c5)n4)c4c3oc3ccccc34)ccc21] | 47 | 742.3 |
| 27 | 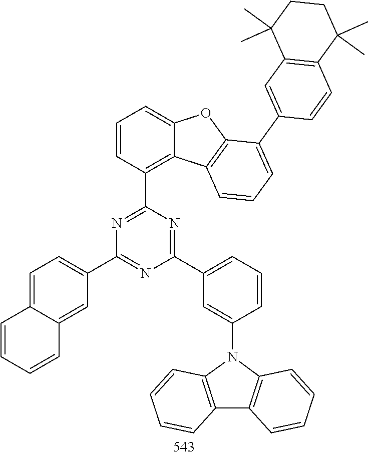 [SMILES: CC1(C)CCC(C)(C)c2cc(-c3cccc4c3oc3cccc(-c5nc(-c6cccc(-n7c8ccccc8c8ccccc87)c6)nc(-c6ccc7ccccc7c6)n5)c34)ccc21] | 44 | 801.4 |

### Synthesis Example 28: Synthesis of Compound 48

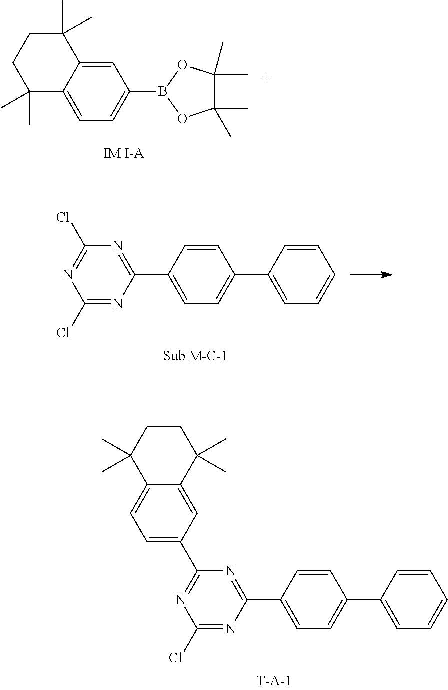 [0126] (1) IM I-A (12.7 g, 40.4 mmol), raw material Sub M-C-1 (12.8 g, 42.4 mmol), tris(dibenzylideneacetonyl)bis-palladium (0.74 g, 0.81 mmol), 2-dicyclohexylphosphino-2,4,6-triisopropylbiphenyl (0.19 g, 0.40 mmol), potassium acetate (7.9 g, 80.8 mmol), and 1,4-dioxane (150 mL) were added to a reaction flask, and heated to 110° C. under nitrogen atmosphere, followed by refluxing and stirring for 5 hours. After being cooled to room temperature, the reaction solution was extracted with dichloromethane and water. The resulting organic layer was dried with anhydrous magnesium sulfate and filtered. After the filtration, the resulting filtrate was passed through a short silica gel column, followed by removal of the solvent under reduced pressure. The resulting crude product was purified by recrystallization using a mixture of dichloromethane and petroleum ether (1:3, v/v), yielding Intermediate IM T-A-1 (12.50 g, yield 68%).

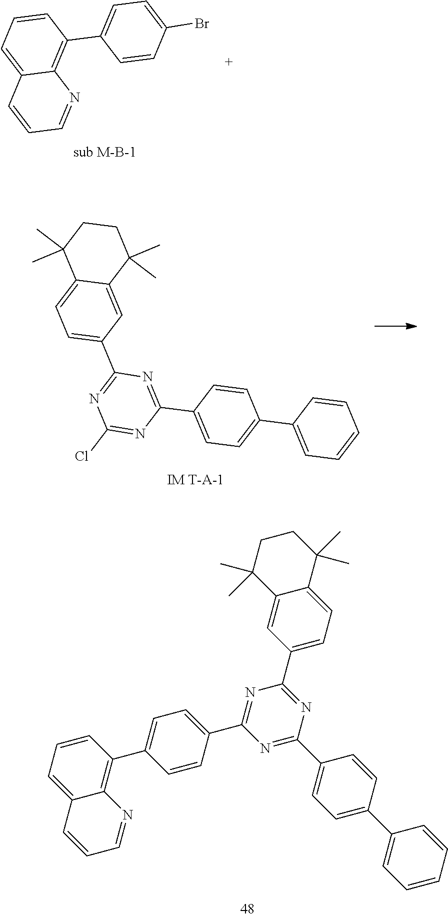 [SMILES: Brc1ccc(-c2cccc3cccnc23)cc1 ; CC1(C)CCC(C)(C)c2cc(-c3nc(Cl)nc(-c4ccc(-c5ccccc5)cc4)n3)ccc21 ; CC1(C)CCC(C)(C)c2cc(-c3nc(-c4ccc(-c5ccccc5)cc4)nc(-c4ccc(-c5cccc6cccnc56)cc4)n3)ccc21]

(2) Raw material sub M-B-1 (1393817-78-1) (4.00 g, 14.08 mmol) and tetrahydrofuran (40 mL) were added to a reaction flask, and cooled to −78° C. under a nitrogen atmosphere, followed by dropwise adding 2 M n-butyllithium solution in tetrahydrofuran (8 mL, 16 mmol). After the dropwise addition, the resulting mixture was kept at the temperature for 1 hour, followed by dropwise adding a tetrahydrofuran solution (30 mL) containing IM T-A-1 (6.39 g, 14.08 mmol). After that, the resulting mixture was again kept at the temperature for 1 hour, and then warmed naturally to room temperature for a reaction for 8 hours. A solid precipitate was formed in the reaction solution, followed by filtration with a Buchner funnel, obtaining a brown crude product. The crude product was purified by recrystallization using toluene (200 mL), yielding Compound 48 (4.91 g, yield 56%); mass spectrometry: m/z=623.3[M+H]⁺.

### Synthesis Examples 29 to 43

Compounds listed in Table 4 were synthesized following the synthesis method of Compound 48, except that in step (1), IM I-A was replaced with a corresponding raw material 5 and raw material Sub M-C-1 was replaced with a corresponding raw material 6 to synthesize IM T-A-X first, and then in step (2), IM T-A-1 was replaced with IM T-A-X and Sub M-B-1 was replaced with a corresponding raw material 7. Main raw materials used, the compounds synthesized, final yields thereof, and mass spectrometry (MS) characterization results are shown in Table 4.

TABLE 4

| Synthesis | Raw | Raw | IM | Raw |
|---|---|---|---|---|
| Example | Material 5 | Material 6 | T-A-X | Material 7 |
| 29 | 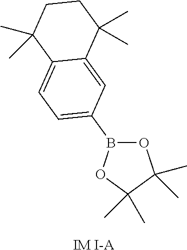 | 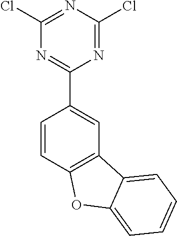 [SMILES: Clc1nc(Cl)nc(-c2ccc3oc4ccccc4c3c2)n1] | 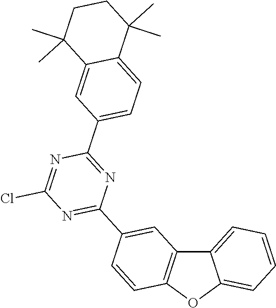 | 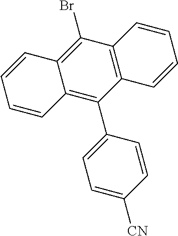 [SMILES: N#Cc1ccc(-c2c3ccccc3c(Br)c3ccccc23)cc1] |
| 30 | 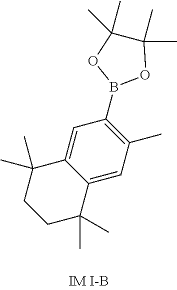 | 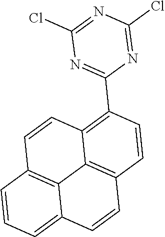 [SMILES: Clc1nc(Cl)nc(-c2ccc3ccc4cccc5ccc2c3c45)n1] | 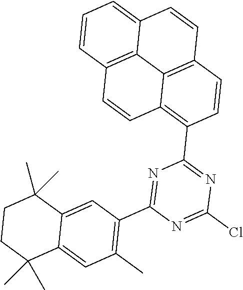 | 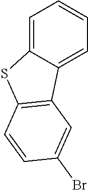 [SMILES: Brc1ccc2sc3ccccc3c2c1] |
| 31 | 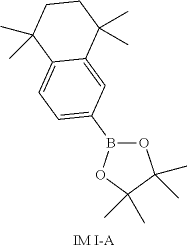 | 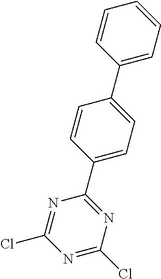 [SMILES: Clc1nc(Cl)nc(-c2ccc(-c3ccccc3)cc2)n1] | 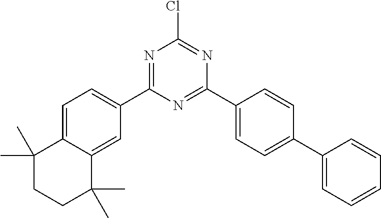 | 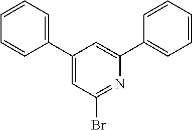 [SMILES: Brc1cc(-c2ccccc2)cc(-c2ccccc2)n1] |
| 32 | 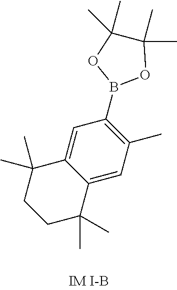 | 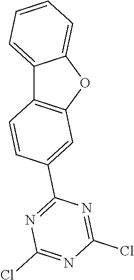 [SMILES: Clc1nc(Cl)nc(-c2ccc3c(c2)oc2ccccc23)n1] | 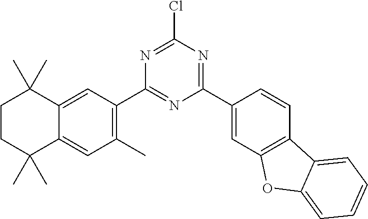 | 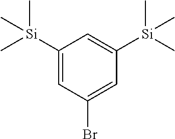 |
| 33 | 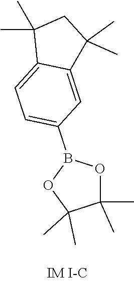 | 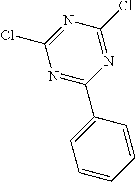 [SMILES: Clc1nc(Cl)nc(-c2ccccc2)n1] | 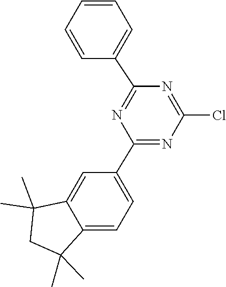 | 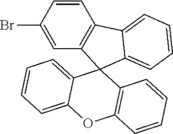 [SMILES: Brc1ccc2c(c1)C1(c3ccccc3Oc3ccccc31)c1ccccc1-2] |
| 34 | 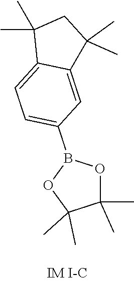 | 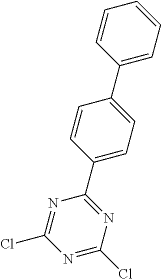 [SMILES: Clc1nc(Cl)nc(-c2ccc(-c3ccccc3)cc2)n1] | 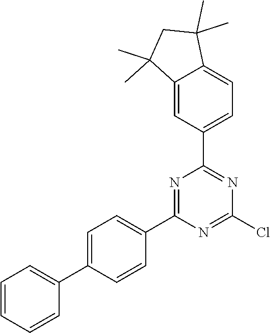 | 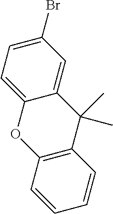 [SMILES: CC1(C)c2ccccc2Oc2ccc(Br)cc21] |
| 35 | 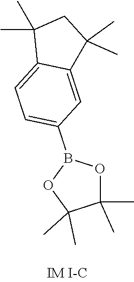 | 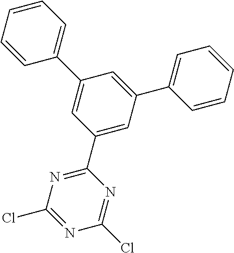 [SMILES: Clc1nc(Cl)nc(-c2cc(-c3ccccc3)cc(-c3ccccc3)c2)n1] | 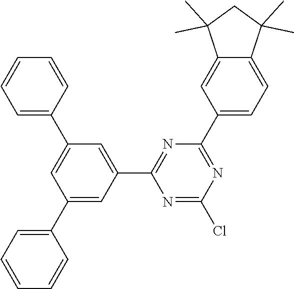 | 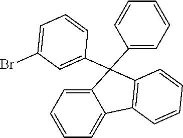 [SMILES: Brc1cccc(C2(c3ccccc3)c3ccccc3-c3ccccc32)c1] |
| 36 | 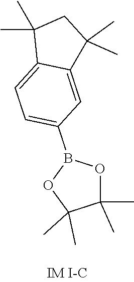 | 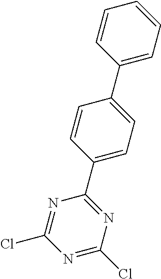 [SMILES: Clc1nc(Cl)nc(-c2ccc(-c3ccccc3)cc2)n1] | 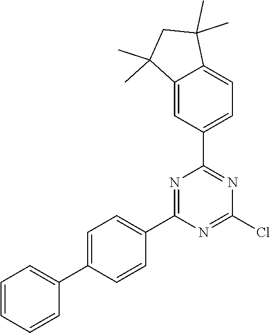 | 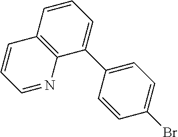 [SMILES: Brc1ccc(-c2cccc3cccnc23)cc1] |
| 37 | 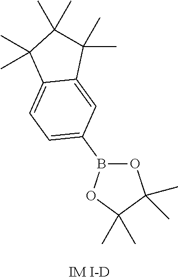 | 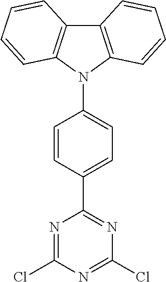 [SMILES: Clc1nc(Cl)nc(-c2ccc(-n3c4ccccc4c4ccccc43)cc2)n1] | 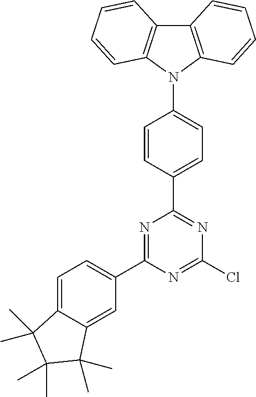 | 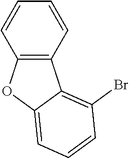 [SMILES: Brc1cccc2oc3ccccc3c12] |
| 38 | 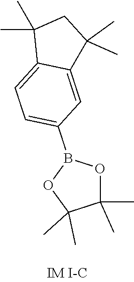 | 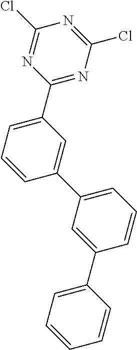 [SMILES: Clc1nc(Cl)nc(-c2cccc(-c3cccc(-c4ccccc4)c3)c2)n1] | 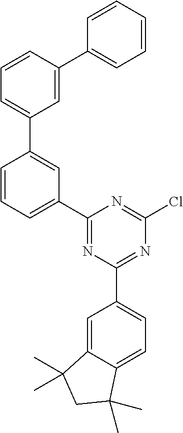 | 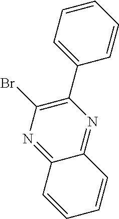 [SMILES: Brc1nc2ccccc2nc1-c1ccccc1] |
| 39 | 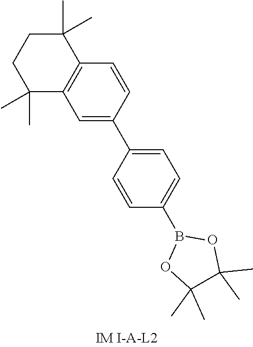 | 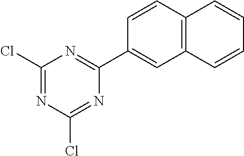 [SMILES: Clc1nc(Cl)nc(-c2ccc3ccccc3c2)n1] | 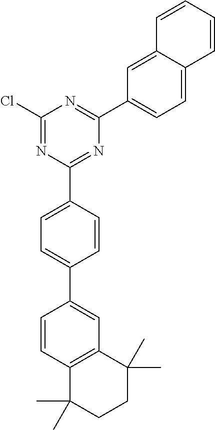 | 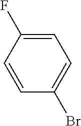 |
| 40 | 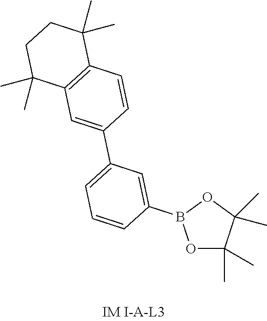 | 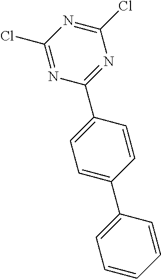 [SMILES: Clc1nc(Cl)nc(-c2ccc(-c3ccccc3)cc2)n1] | 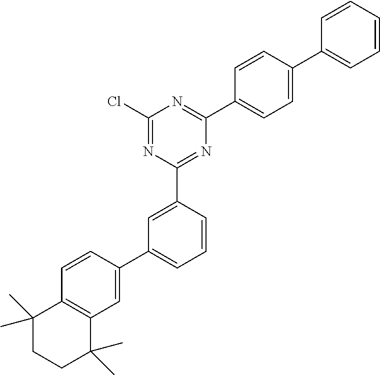 | 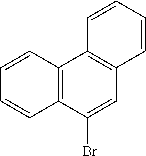 [SMILES: Brc1cc2ccccc2c2ccccc12] |
| 41 | 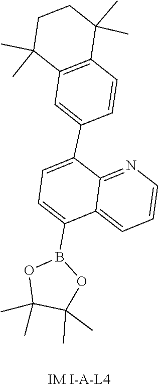 [SMILES: CC1(C)CCC(C)(C)c2cc(-c3ccc(B4OC(C)(C)C(C)(C)O4)c4cccnc34)ccc21] | 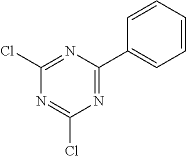 [SMILES: Clc1nc(Cl)nc(-c2ccccc2)n1] | 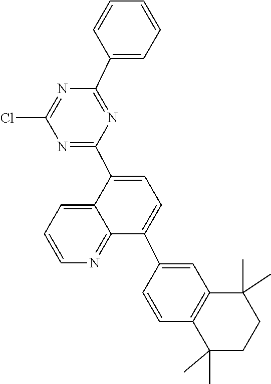 | 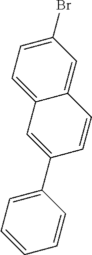 [SMILES: Brc1ccc2cc(-c3ccccc3)ccc2c1] |
| 42 | 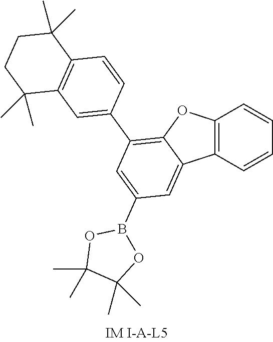 [SMILES: CC1(C)CCC(C)(C)c2cc(-c3cc(B4OC(C)(C)C(C)(C)O4)cc4c3oc3ccccc34)ccc21] | 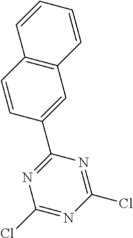 [SMILES: Clc1nc(Cl)nc(-c2ccc3ccccc3c2)n1] | 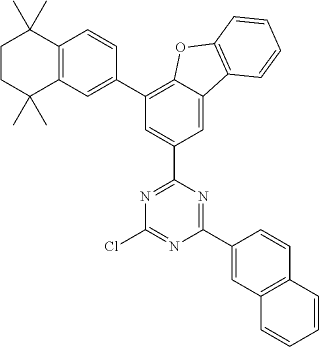 | 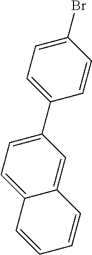 [SMILES: Brc1ccc(-c2ccc3ccccc3c2)cc1] |
| 43 | 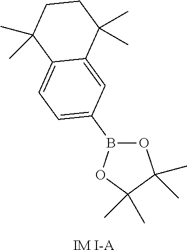 | 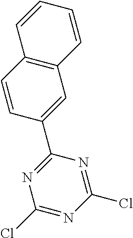 [SMILES: Clc1nc(Cl)nc(-c2ccc3ccccc3c2)n1] | 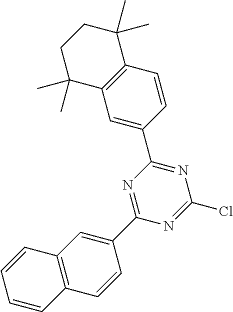 | 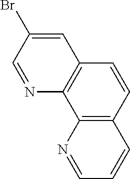 [SMILES: Brc1cnc2c(ccc3cccnc32)c1] |

| Synthesis Example | Compound | Yield/% | MS/[M + H]⁺ |
|---|---|---|---|
| 29 | 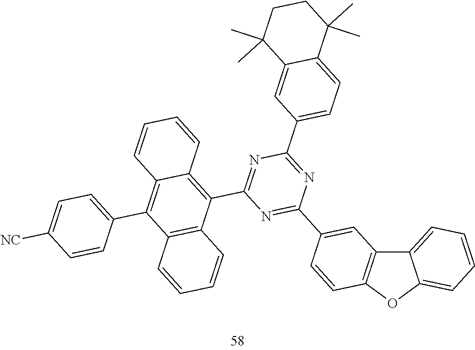 [SMILES: CC1(C)CCC(C)(C)c2cc(-c3nc(-c4ccc5oc6ccccc6c5c4)nc(-c4c5ccccc5c(-c5ccc(C#N)cc5)c5ccccc45)n3)ccc21] | 51 | 711.3 |
| 30 | 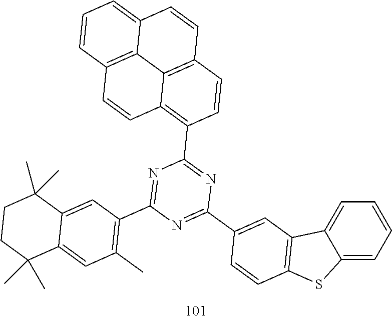 [SMILES: Cc1cc2c(cc1-c1nc(-c3ccc4sc5ccccc5c4c3)nc(-c3ccc4ccc5cccc6ccc3c4c56)n1)C(C)(C)CCC2(C)C] | 46 | 664.3 |
| 31 | 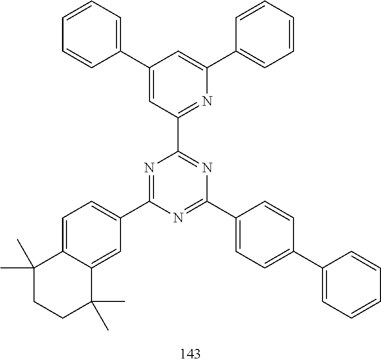 [SMILES: CC1(C)CCC(C)(C)c2cc(-c3nc(-c4ccc(-c5ccccc5)cc4)nc(-c4cc(-c5ccccc5)cc(-c5ccccc5)n4)n3)ccc21] | 60 | 649.3 |
| 32 | 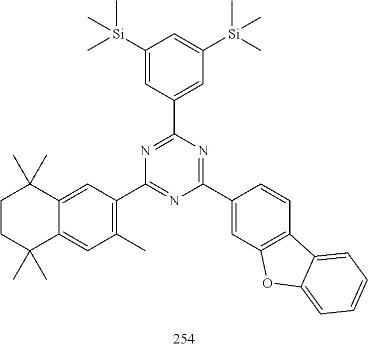 [SMILES: Cc1cc2c(cc1-c1nc(-c3cc([Si](C)(C)C)cc([Si](C)(C)C)c3)nc(-c3ccc4c(c3)oc3ccccc34)n1)C(C)(C)CCC2(C)C] | 62 | 668.3 |
| 33 | 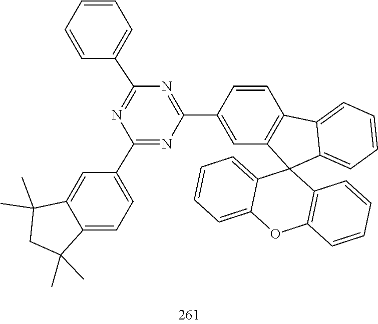 [SMILES: CC1(C)CC(C)(C)c2cc(-c3nc(-c4ccccc4)nc(-c4ccc5c(c4)C4(c6ccccc6Oc6ccccc64)c4ccccc4-5)n3)ccc21] | 57 | 660.3 |
| 34 | 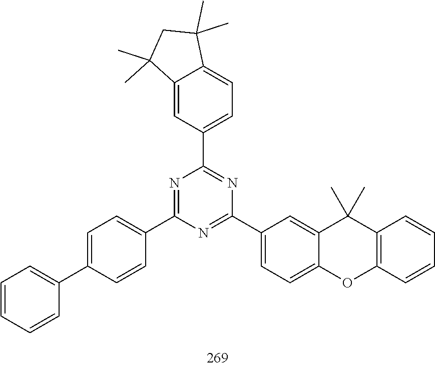 [SMILES: CC1(C)CC(C)(C)c2cc(-c3nc(-c4ccc(-c5ccccc5)cc4)nc(-c4ccc5c(c4)C(C)(C)c4ccccc4O5)n3)ccc21] | 52 | 614.3 |
| 35 | 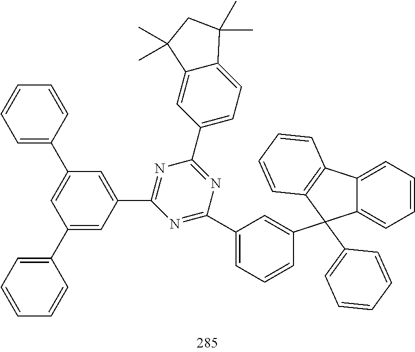 [SMILES: CC1(C)CC(C)(C)c2cc(-c3nc(-c4cc(-c5ccccc5)cc(-c5ccccc5)c4)nc(-c4cccc(C5(c6ccccc6)c6ccccc6-c6ccccc65)c4)n3)ccc21] | 61 | 798.4 |
| 36 | 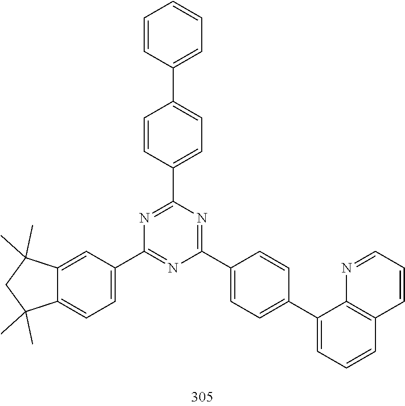 [SMILES: CC1(C)CC(C)(C)c2cc(-c3nc(-c4ccc(-c5ccccc5)cc4)nc(-c4ccc(-c5cccc6cccnc56)cc4)n3)ccc21] | 60 | 609.3 |
| 37 | 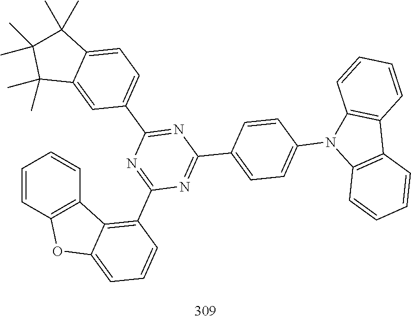 [SMILES: CC1(C)c2ccc(-c3nc(-c4ccc(-n5c6ccccc6c6ccccc65)cc4)nc(-c4cccc5oc6ccccc6c45)n3)cc2C(C)(C)C1(C)C] | 48 | 689.3 |
| 38 | 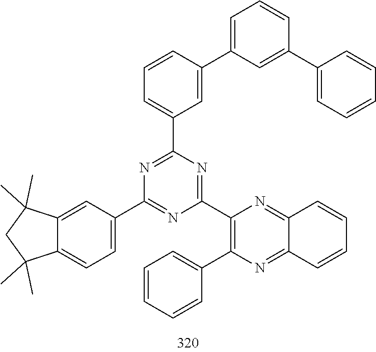 [SMILES: CC1(C)CC(C)(C)c2cc(-c3nc(-c4cccc(-c5cccc(-c6ccccc6)c5)c4)nc(-c4nc5ccccc5nc4-c4ccccc4)n3)ccc21] | 50 | 686.3 |
| 39 | 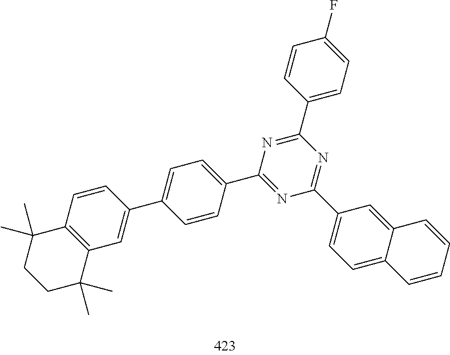 [SMILES: CC1(C)CCC(C)(C)c2cc(-c3ccc(-c4nc(-c5ccc(F)cc5)nc(-c5ccc6ccccc6c5)n4)cc3)ccc21] | 58 | 564.3 |
| 40 | 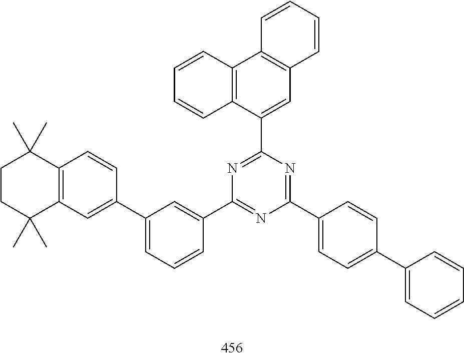 [SMILES: CC1(C)CCC(C)(C)c2cc(-c3cccc(-c4nc(-c5ccc(-c6ccccc6)cc5)nc(-c5cc6ccccc6c6ccccc56)n4)c3)ccc21] | 54 | 672.3 |
| 41 | 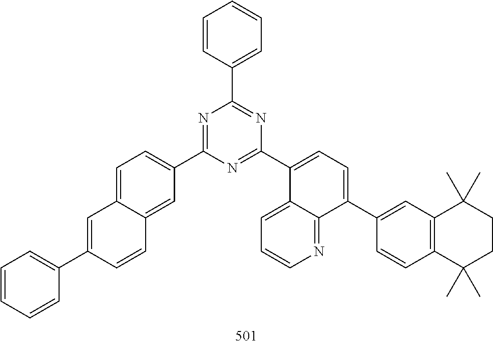 [SMILES: CC1(C)CCC(C)(C)c2cc(-c3ccc(-c4nc(-c5ccccc5)nc(-c5ccc6cc(-c7ccccc7)ccc6c5)n4)c4cccnc34)ccc21] | 43 | 673.3 |
| 42 | 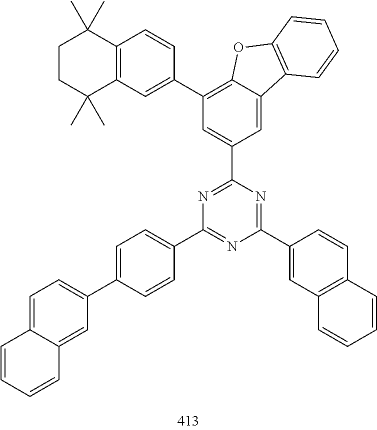 [SMILES: CC1(C)CCC(C)(C)c2cc(-c3cc(-c4nc(-c5ccc(-c6ccc7ccccc7c6)cc5)nc(-c5ccc6ccccc6c5)n4)cc4c3oc3ccccc34)ccc21] | 54 | 762.3 |
| 43 | 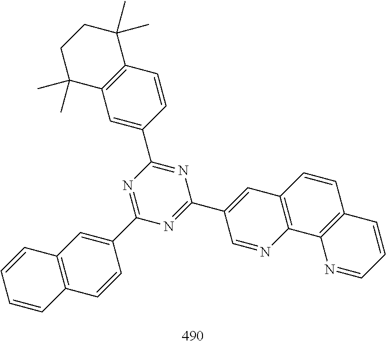 [SMILES: CC1(C)CCC(C)(C)c2cc(-c3nc(-c4ccc5ccccc5c4)nc(-c4cnc5c(ccc6cccnc65)c4)n3)ccc21] | 39 | 572.3 |

NMR data for some of the compounds are as follows.

Compound 396: ¹H-NMR (400 MHz, Methylene-Chloride-D2) δ ppm 9.38(s, 1H), 9.27 (d, 1H), 8.87 (d, 1H), 8.75 (d, 1H), 8.62 (d, 1H), 8.53 (d, 1H), 8.11 (d, 1H), 8.05 (d, 2H), 7.95 (d, 1H), 7.86 (d, 1H), 7.44-7.74 (m, 10H), 7.35 (d, 1H), 7.15 (t, 1H), 1.75-1.79 (s, 4H), 1.32-1.39 (d, 12H).

Compound 413: ¹H-NMR (400 MHz, Methylene-Chloride-D2) δ ppm 9.42(s, 1H), 9.20 (s, 1H), 8.98 (d, 2H), 8.88 (d, 1H), 8.72 (s, 1H), 8.68 (d, 1H), 8.24 (s 1H), 8.11-7.95 (m, 8H), 7.93-7.87 (m, 2H), 7.68-7.48 (m, 8H), 7.26 (t, 1H), 1.75-1.79 (s, 4H), 1.32-1.39 (d, 12H).

Compound 347: ¹H-NMR (400 MHz, Methylene-Chloride-D2) δ ppm 9.12(s, 1H), 8.89(d, 2H), 8.65(d, 1H), 8.49(d, 1H), 8.42(d, 1H), 8.09(s, 1H), 7.98(d, 1H), 7.78(s, 1H), 7.69-7.64(m, 3H), 7.61-7.56(m, 2H), 7.54-7.47(m, 3H), 7.33-7.28(t, 1H), 7.21(d, 1H), 2.02(s, 2H), 1.43(s, 6H), 1.34(s, 6H).

Compound 305: ¹H-NMR (400 MHz, Methylene-Chloride-D2) δ ppm 9.06(d, 1H), 8.40(d, 2H), 8.29-8.24(m, 3H), 8.22(s, 1H), 8.16(d, 1H), 8.12(d, 2H), 7.92-7.87(m, 3H), 7.72(d, 1H), 7.68-7.62(t, 1H), 7.59-7.53(m, 5H), 7.47-7.36(m, 2H), 1.98(s, 2H), 1.46(s, 6H), 1.38(s, 6H).

### Organic Electroluminescent Devices Were Fabricated Using the Following Methods.

### Example 1: Blue Light-Emitting Organic Electroluminescent Device

An anode was prepared by the following processes. An ITO/Ag/ITO substrate (manufactured by Corning), with thicknesses of ITO/Ag/ITO being 100 Å, 1100 Å, and 100 Å, respectively, was cut to have dimensions of 40 mm×40 mm×07 mm, and then fabricated, by a photoetching process, into an experimental substrate with patterns of a cathode, of an anode, and of an insulation layer, followed by treatment of its surface using ultraviolet ozone and O₂:N₂ plasma to increase the work function of the anode (experimental substrate) and to descum.

F4-TCNQ was deposited by vacuum evaporation on the experimental substrate (anode) to form a hole injection layer with a thickness of 100 Å, and BF-DPB was deposited by evaporation on the hole injection layer to form a first hole transport layer with a thickness of 1300 Å.

HT-5 was deposited by vacuum evaporation on the first hole transport layer to form a second hole transport layer with a thickness of 100 Å. MADN and BD-1 were co-deposited by evaporation on the second hole transport layer at a film thickness ratio of 99%:1% to form an organic emissive layer with a thickness of 200 Å.

Compound 6 and LiQ were mixed at a weight ratio of 1:1 and deposited by evaporation on the organic emissive layer to form an electron transport layer with a thickness of 320 Å.

Yb was deposited by evaporation on the electron transport layer to form an electron injection layer with a thickness of 10 Å. Then, magnesium and silver were mixed at an evaporation rate ratio of 1:10 and deposited by vacuum evaporation on the electron injection layer to form a cathode with a thickness of 130 Å.

Furthermore, CP-1 was deposited by vacuum evaporation on the above cathode to form an organic capping layer (CPL) with a thickness of 700 Å, completing the fabrication of an organic electroluminescent device.

### Examples 2 to 21

Organic electroluminescent devices were fabricated respectively by the same method as used in Example 1, except that Compound 6 was replaced with a corresponding compound shown in Table 5 when an electron transport layer was formed.

### Comparative Examples 1 to 3

Organic electroluminescent devices were fabricated respectively by the same method as used in Example 1, except that Compound 6 was replaced with a corresponding one of Compound A, Compound B, and Compound C when an electron transport layer was formed.

Main materials used in the Examples and Comparative Examples and structures thereof are shown below.

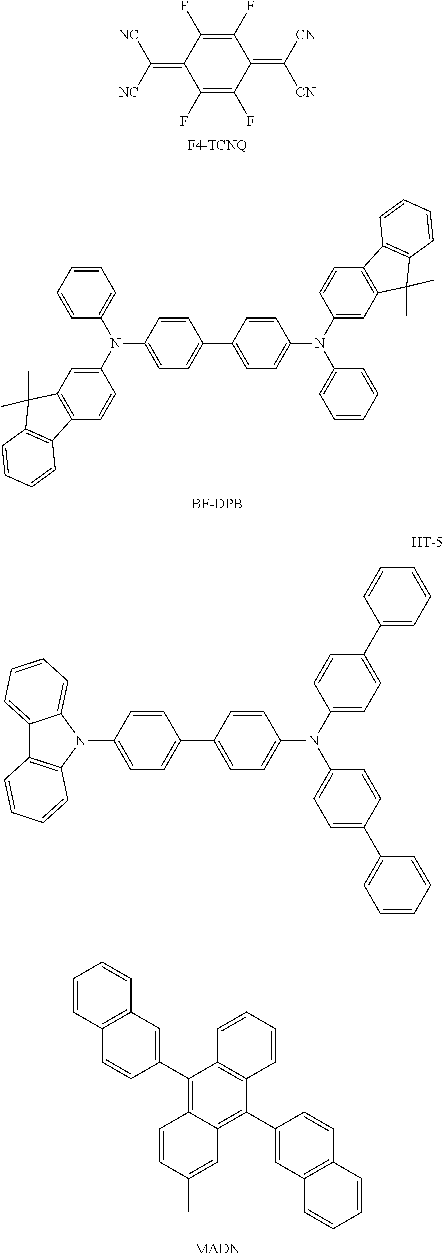

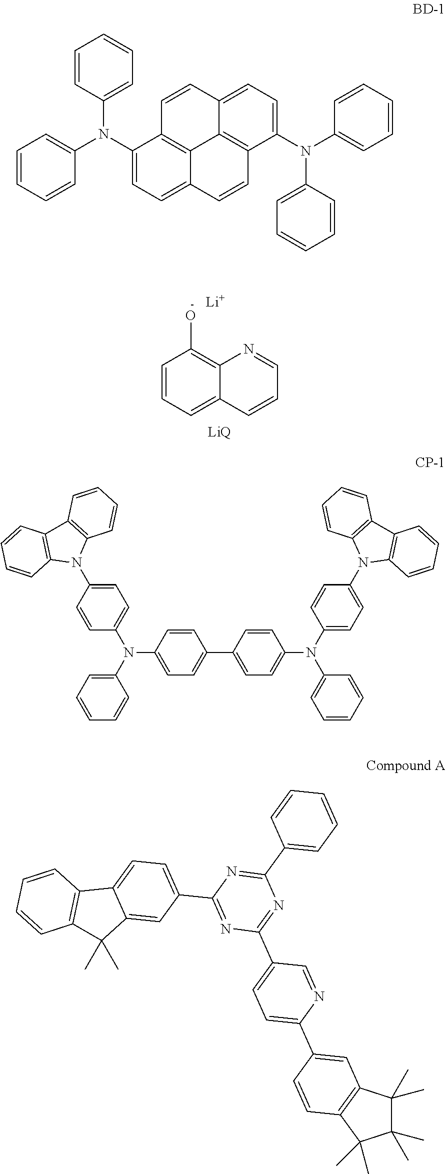

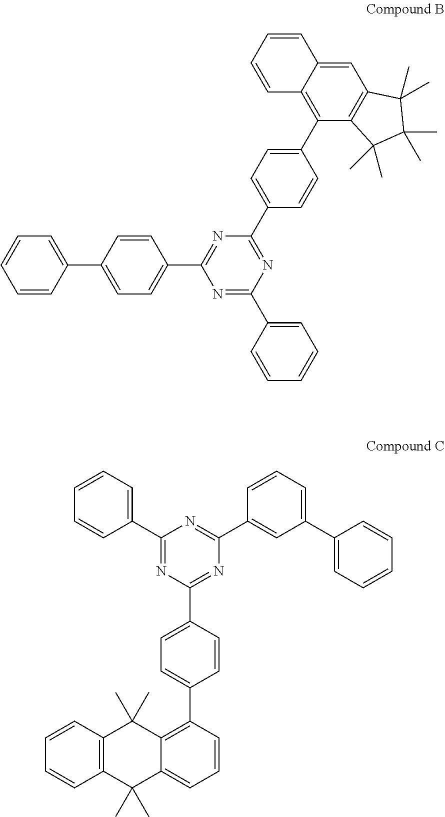 [SMILES: CC1(C)c2cc3ccccc3c(-c3ccc(-c4nc(-c5ccccc5)nc(-c5ccc(-c6ccccc6)cc5)n4)cc3)c2C(C)(C)C1(C)C ; CC1(C)c2ccccc2C(C)(C)c2c(-c3ccc(-c4nc(-c5ccccc5)nc(-c5cccc(-c6ccccc6)c5)n4)cc3)cccc21]

The organic electroluminescent devices fabricated above were tested for their photoelectric performance under the condition of 20 mA/cm². Results are shown in Table 5 below.

TABLE 5

| | | | | | | External | |
|---|---|---|---|---|---|---|---|
| | | Driving | Current | | | quantum | |
| | Material of electron | voltage | efficiency | | | efficiency | Lifetime |
| No. | transport layer | (V) | (Cd/A) | CIE-x | CIE-y | (EQE %) | T95(h) |
| Example 1 | Compound 6 | 4.01 | 6.69 | 0.140 | 0.050 | 13.76 | 204 |
| Example 2 | Compound 18 | 3.96 | 6.86 | 0.140 | 0.050 | 14.11 | 207 |
| Example 3 | Compound 32 | 3.91 | 6.76 | 0.140 | 0.050 | 13.91 | 235 |
| Example 4 | Compound 98 | 4.01 | 6.90 | 0.140 | 0.050 | 14.19 | 230 |
| Example 5 | Compound 115 | 3.98 | 6.94 | 0.140 | 0.050 | 14.28 | 245 |
| Example 6 | Compound 132 | 3.91 | 6.82 | 0.140 | 0.050 | 14.03 | 229 |
| Example 7 | Compound 141 | 3.99 | 6.93 | 0.140 | 0.050 | 14.26 | 237 |
| Example 8 | Compound 157 | 3.90 | 6.91 | 0.140 | 0.050 | 14.21 | 234 |
| Example 9 | Compound 192 | 3.97 | 6.72 | 0.140 | 0.050 | 13.82 | 228 |
| Example 10 | Compound 198 | 3.91 | 6.69 | 0.140 | 0.050 | 13.76 | 211 |
| Example 11 | Compound 220 | 3.93 | 6.64 | 0.140 | 0.050 | 13.66 | 209 |
| Example 12 | Compound 48 | 3.92 | 6.93 | 0.140 | 0.050 | 14.26 | 235 |
| Example 13 | Compound 58 | 3.95 | 6.78 | 0.140 | 0.050 | 13.95 | 240 |
| Example 14 | Compound 101 | 3.99 | 6.61 | 0.140 | 0.050 | 13.60 | 227 |
| Example 15 | Compound 143 | 3.91 | 6.86 | 0.140 | 0.050 | 14.11 | 225 |
| Example 16 | Compound 254 | 3.92 | 6.70 | 0.140 | 0.050 | 13.85 | 231 |
| Example 17 | Compound 261 | 4.01 | 6.79 | 0.140 | 0.050 | 13.97 | 215 |
| Example 18 | Compound 269 | 3.98 | 6.84 | 0.140 | 0.050 | 14.07 | 213 |
| Example 19 | Compound 285 | 4.01 | 6.73 | 0.140 | 0.050 | 13.84 | 209 |
| Example 20 | Compound 305 | 4.01 | 6.59 | 0.140 | 0.050 | 13.56 | 212 |
| Example 21 | Compound 320 | 3.90 | 6.85 | 0.140 | 0.050 | 14.09 | 205 |
| Comparative | Compound A | 4.17 | 5.76 | 0.140 | 0.050 | 11.85 | 160 |
| Example 1 | | | | | | | |
| Comparative | Compound B | 4.27 | 5.62 | 0.140 | 0.050 | 11.56 | 180 |
| Example 2 | | | | | | | |
| Comparative | Compound C | 4.23 | 5.73 | 0.140 | 0.050 | 11.85 | 158 |
| Example 3 | | | | | | | |

As can be seen from Table 5, compared with Comparative Examples 1 to 3, Examples 1 to 21, in which the compounds of the present disclosure are used as the material of the electron transport layer, significantly improve the current efficiency of the device and significantly prolong the service life thereof. The luminescence efficiency is increased by at least 14.4%; the service life is prolonged by at least 13.3%; and meanwhile, a relatively low driving voltage is maintained. Therefore, the organic compounds of the present disclosure, when used in the fabrication of an organic electroluminescent device as the material of an electron transport layer, can effectively improve the luminescence efficiency and prolong service life of the device.

### Example 22: Fabrication of a Red Light-Emitting Organic Electroluminescent Device

An anode was prepared by the following processes. An ITO/Ag/ITO substrate, with thicknesses of ITO/Ag/ITO being 100 Å, 1100 Å, and 100 Å, respectively, was cut to have dimensions of 40 mm×40 mm×0.7 mm, and then fabricated, by a photoetching process, into an experimental substrate with patterns of a cathode, of an anode, and of an insulation layer, followed by treatment of its surface using ultraviolet ozone and O₂:N₂ plasma to increase the work function of the anode and to descum.

1T-NATA was deposited by vacuum evaporation on the experimental substrate (anode) to form a hole injection layer with a thickness of 130 Å, and HT-3 was deposited by evaporation on the hole injection layer to form a first hole transport layer with a thickness of 1100 Å.

HT-5 was deposited by vacuum evaporation on the first hole transport layer to form a second hole transport layer with a thickness of 150 Å.

Compound 6, RH-1, and RD-3 were co-deposited by evaporation on the second hole transport layer at a film thickness ratio of 50:50:3 to form an organic emissive layer with a thickness of 330 Å.

ET-2 and LiQ were co-deposited by evaporation on the organic emissive layer at a film thickness ratio of 1:1 to form an electron transport layer with a thickness of 280 Å. Yb was deposited by evaporation on the electron transport layer to form an electron injection layer with a thickness of 12 Å. Then, magnesium (Mg) and silver (Ag) were co-deposited by vacuum evaporation on the electron injection layer at a film thickness ratio of 1:9 to form a cathode with a thickness of 110 Å.

Furthermore, CP-2 was deposited by evaporation on the above cathode to form an organic capping layer with a thickness of 670 Å, completing the fabrication of an organic electroluminescent device.

### Examples 23 to 43

Organic electroluminescent devices were fabricated respectively by the same method as used in Example 22, except that Compound 64 was replaced with a corresponding compound shown in Table 6 when an organic emissive layer was formed.

### Comparative Examples 4 to 5

Organic electroluminescent devices were fabricated respectively by the same method as used in Example 22, except that Compound 64 was replaced with a corresponding one of Compound D and Compound E when an organic emissive layer was formed.

Structures of main materials used in Examples 23 to 43 and Comparative Examples 4 to 5 for fabricating the organic electroluminescent devices are shown below.

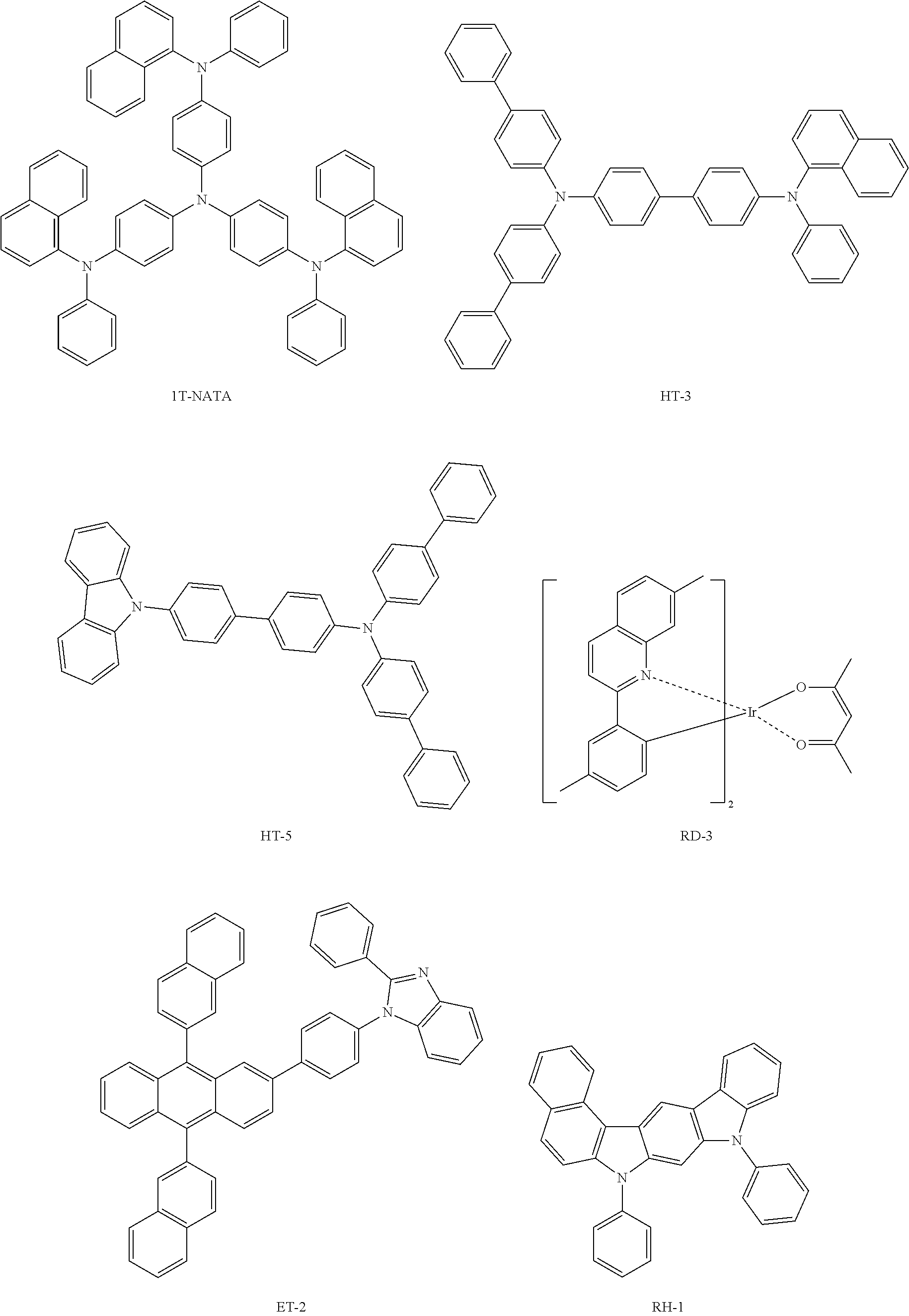

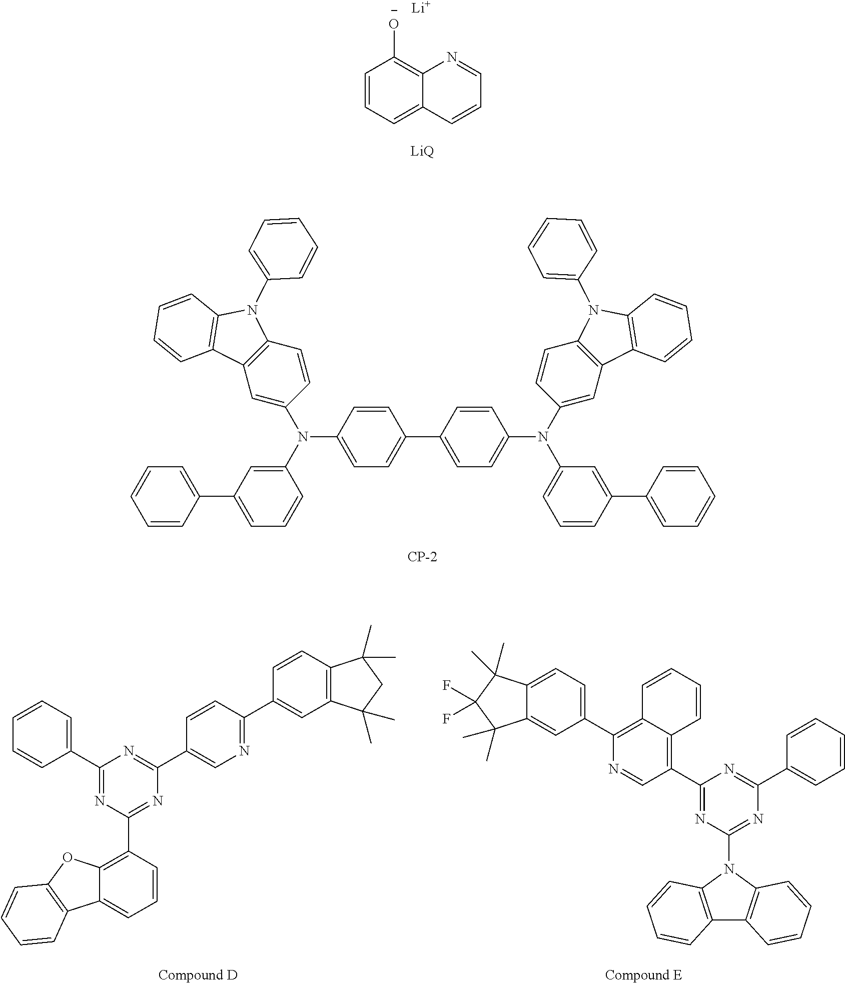

The organic electroluminescent devices fabricated in Examples 22 to 43 and Comparative Examples 4 to 5 were tested for their performance under the condition of 20 mA/cm². Results are shown in Table 6.

TABLE 6

| | | Driving | Current | Power | Chromaticity | |
|---|---|---|---|---|---|---|
| | Host material | voltage | efficiency | efficiency | coordinate | Lifetime |
| No. | of emissive layer | (V) | (Cd/A) | (lm/W) | (CIE-X) | T95(h) |
| Example 22 | Compound 64 | 3.78 | 33.5 | 28.6 | 0.68 | 337 |
| Example 23 | Compound 76 | 3.80 | 37.0 | 30.5 | 0.68 | 340 |
| Example 24 | Compound 88 | 3.82 | 34.7 | 28.9 | 0.68 | 336 |
| Example 25 | Compound 396 | 3.67 | 36.8 | 30.2 | 0.68 | 358 |
| Example 26 | Compound 423 | 3.65 | 33.6 | 29.0 | 0.68 | 351 |
| Example 27 | Compound 456 | 3.82 | 35.3 | 29.1 | 0.68 | 348 |
| Example 28 | Compound 501 | 3.84 | 34.4 | 29.6 | 0.68 | 344 |
| Example 29 | Compound 413 | 3.73 | 33.7 | 27.6 | 0.68 | 353 |
| Example 30 | Compound 490 | 3.79 | 35.9 | 29.8 | 0.68 | 349 |
| Example 31 | Compound 179 | 3.83 | 36.4 | 29.8 | 0.68 | 346 |
| Example 32 | Compound 204 | 3.77 | 36.1 | 30.1 | 0.68 | 360 |
| Example 33 | Compound 347 | 3.81 | 33.6 | 27.7 | 0.68 | 357 |
| Example 34 | Compound 309 | 3.84 | 33.9 | 28.6 | 0.68 | 335 |
| Example 35 | Compound 404 | 3.75 | 35.7 | 29.9 | 0.68 | 361 |
| Example 36 | Compound 416 | 3.76 | 34.8 | 29.3 | 0.68 | 354 |
| Example 37 | Compound 472 | 3.80 | 36.0 | 29.7 | 0.68 | 352 |
| Example 38 | Compound 511 | 3.32 | 37.2 | 31.8 | 0.68 | 368 |
| Example 39 | Compound 514 | 3.37 | 35.9 | 30.7 | 0.68 | 369 |
| Example 40 | Compound 524 | 3.34 | 36.5 | 30.9 | 0.68 | 372 |
| Example 41 | Compound 530 | 3.31 | 35.2 | 29.8 | 0.68 | 366 |
| Example 42 | Compound 540 | 3.82 | 34.4 | 28.7 | 0.68 | 355 |
| Example 43 | Compound 543 | 3.39 | 36.1 | 30.7 | 0.68 | 370 |
| Comparative | Compound D | 4.07 | 27.7 | 20.9 | 0.68 | 255 |
| Example 4 | | | | | | |
| Comparative | Compound E | 4.18 | 28.4 | 21.4 | 0.68 | 285 |
| Example 5 | | | | | | |

As can be seen from Table 6, compared with Comparative Examples 4 to 5, Examples 22 to 43, in which the compounds of the present disclosure are used as the host material of the emissive layer, significantly improve the comprehensive performance of the device. The luminescence efficiency is increased by at least 17.6%, and the service life is prolonged by at least 17.5%. Therefore, the new organic compounds of the present disclosure, when used in the fabrication of an organic electroluminescent device as the host material of the emissive layer, can effectively improve the luminescence efficiency and prolong service life of the device.

The above describes in detail the preferred embodiments of the present disclosure with reference to the accompanying drawings. The present disclosure, however, is not limited to the specific details in the above embodiments. Many simple variations may be made to the technical solutions of the present disclosure within the scope of the technical conception of the present disclosure, and all such simple variations fall within the protection scope of the present disclosure. It should also be noted that the various specific technical features described in the above embodiments can be combined in any suitable manner without conflicting with each other. Not all possible combinations are described in the present disclosure to avoid repetition. Further, various embodiments of the present disclosure can also be combined in any manner, and such combinations should also be regarded as a part of the present disclosure as long as they do not contradict the conception of the present disclosure.

1. An organic compound, having a structure shown in Formula 1: 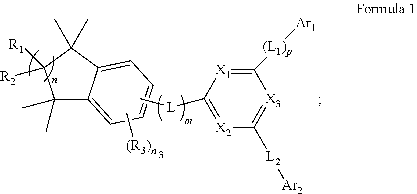
wherein in Formula 1, 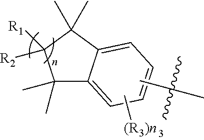
is selected from the group consisting of the following groups: 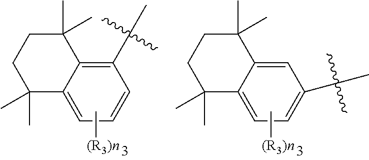
X₁, X₂, and X₃ are each independently selected from C(H) or N atoms, and at least one of X₁, X₂, and X₃ is N;
p is selected from 1 or 2; each L₁ and each L₂ is identical or different, and is each independently selected from the group consisting of single bond, substituted or unsubstituted arylene having 6 to 20 carbon atoms, and substituted or unsubstituted heteroarylene having 5 to 20 carbon atoms;
m represents 2, one of the two L is selected from the group consisting of single bond, substituted or unsubstituted arylene having 6 to 20 carbon atoms, and substituted or unsubstituted heteroarylene having 8 to 20 carbon atoms, and the other L is selected from substituted or unsubstituted heteroarylene having 8 to 20 carbon atoms;
Ar₁ and Ar₂ are identical or different, and are each independently selected from the group consisting of substituted or unsubstituted aryl having 6 to 40 carbon atoms, and substituted or unsubstituted heteroaryl having 5 to 40 carbon atoms;
substituents in L₁, L₂, L, Ar₁, and Ar₂, as well as R₃ are identical or different, and are each independently selected from the group consisting of deuterium, halogen, cyano, alkyl having 1 to 10 carbon atoms, haloalkyl having 1 to 10 carbon atoms, trialkylsiyl having 3 to 12 carbon atoms, deuterated alkyl having 1 to 10 carbon atoms, cycloalkyl having 3 to 10 carbon atoms, aryl having 6 to 18 carbon atoms, and heteroaryl having 5 to 15 carbon atoms; and optionally, in Ar₁ and Ar₂, any two adjacent substituents form a saturated or unsaturated 3 to 18-membered ring substituted or unsubstituted by alkyl having 1 to 4 carbon atoms; and
n₃ represents the number of R₃, and is selected from 0, 1, 2, or 3, wherein when n₃ is greater than 1, each R₃ is identical or different.

2. (canceled)

3. The organic compound according to claim 1, wherein each L₁ and each L₂ is identical or different, and is each independently selected from the group consisting of single bond, substituted or unsubstituted arylene having 6 to 18 carbon atoms, and substituted or unsubstituted heteroarylene having 5 to 15 carbon atoms.

4. The organic compound according to claim 1, wherein each L₁ and each L₂ is identical or different, and is each independently selected from the group consisting of single bond, substituted or unsubstituted phenylene, substituted or unsubstituted naphthylene, substituted or unsubstituted phenanthrylene, substituted or unsubstituted anthrylene, substituted or unsubstituted pyridylidene, substituted or unsubstituted quinolylene, substituted or unsubstituted dibenzofuranylene, and substituted or unsubstituted carbazolylene; and
optionally, the substituents in in L₁ and L₂ are each independently selected from the group consisting of deuterium, fluorine, cyano, methyl, ethyl, isopropyl, tert-butyl, trifluoromethyl, trideuteromethyl, trimethylsilyl, phenyl, naphthyl, pyridyl, dibenzofuranyl, dibenzothienyl, or carbazolyl.

5. The organic compound according to claim 1, wherein each L₁ and each L₂ is identical or different, and is each independently selected from single bond and substituted or unsubstituted group Z, wherein the unsubstituted group Z is selected from the group consisting of the following groups: 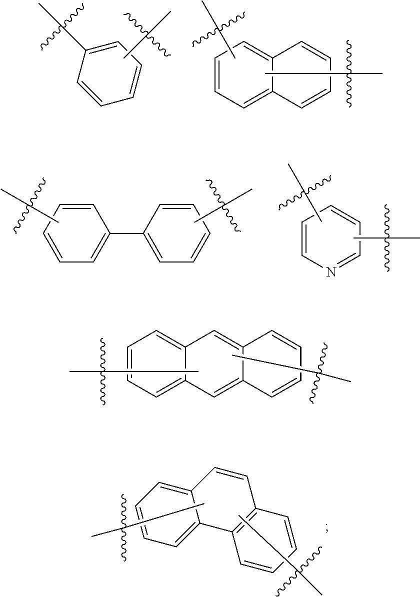
and
the substituted group Z each has one or more substituents, the substituents being each independently selected from the group consisting of deuterium, fluorine, cyano, methyl, ethyl, isopropyl, tert-butyl, trifluoromethyl, trideuteromethyl, trimethylsilyl, phenyl, naphthyl, or pyridyl, wherein when the number of the substituents is greater than 1, the substituents are identical or different.

6. The organic compound according to claim 1, wherein 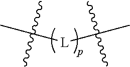
is selected from the group consisting of the following groups:

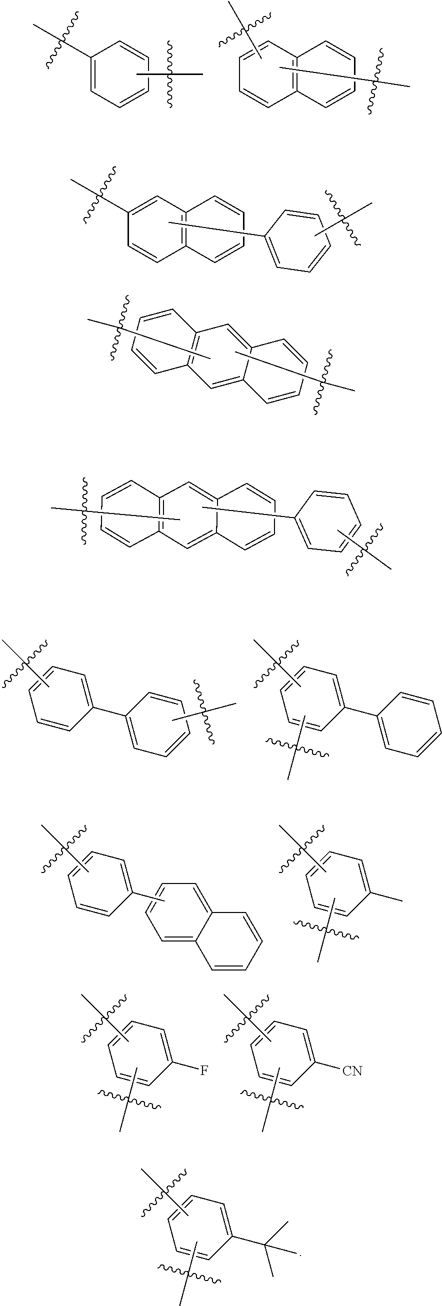

7. The organic compound according to claim 1, wherein m represents 2;
one of the two L is selected from the group consisting of single bond, substituted or unsubstituted arylene having 6 to 18 carbon atoms, and substituted or unsubstituted heteroarylene having 8 to 15 carbon atoms, and the other L is selected from substituted or unsubstituted heteroarylene having 8 to 15 carbon atoms; and
optionally, the substituents in L are each independently selected from the group consisting of deuterium, fluorine, cyano, alkyl having 1 to 4 carbon atoms, fluoroalkyl having 1 to 4 carbon atoms, deuterated alkyl having 1 to 4 carbon atoms, trialkylsilyl having 3 to 7 carbon atoms, aryl having 6 to 12 carbon atoms, or heteroaryl having 5 to 12 carbon atoms.

8. The organic compound according to claim 1, wherein
m represents 2, one of the two L is selected from the group consisting of single bond, and substituted or unsubstituted group V, wherein the unsubstituted group V is selected from the group consisting of the following groups: 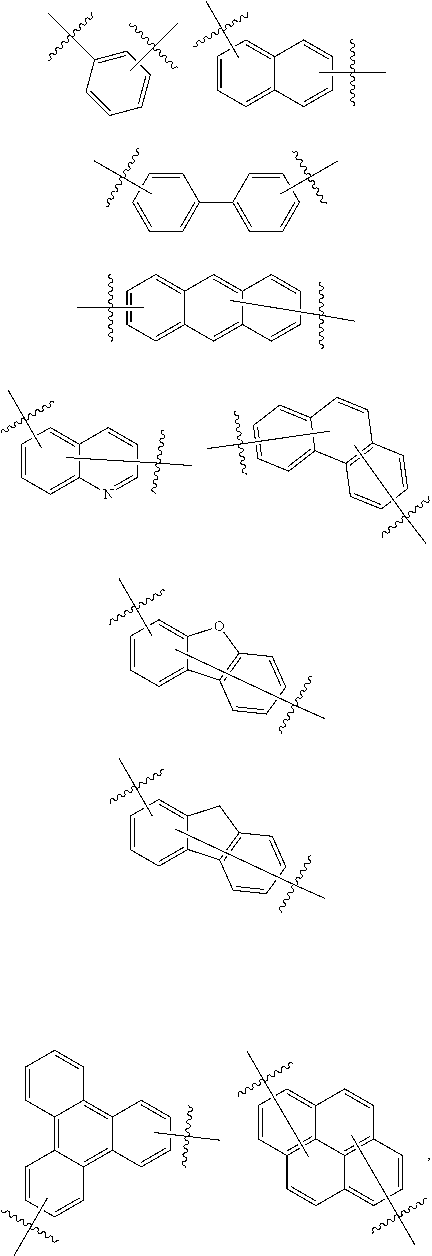
and the other L is selected from substituted or unsubstituted group V, wherein the unsubstituted group V is selected from the group consisting of the following groups: 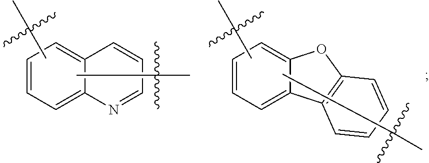
and
the substituted group V each has one or more substituents, the substituents each being independently selected from the group consisting of deuterium, fluorine, cyano, methyl, ethyl, isopropyl, tert-butyl, trifluoromethyl, trideuteromethyl, trimethylsilyl, phenyl, naphthyl, pyridyl, quinolyl, isoquinolyl, dibenzofuranyl, dibenzothienyl, or carbazolyl, wherein when the number of the substituents is greater than 1, the substituents are identical or different.

9. The organic compound according to claim 1, wherein 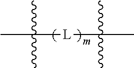
is selected from the group consisting of the following groups:

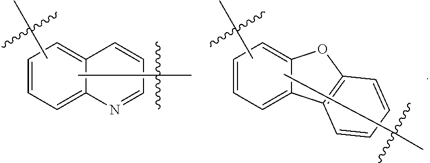

10. The organic compound according to claim 1, wherein Ar₁ and Ar₂ are identical or different, and are each independently selected from the group consisting of substituted or unsubstituted aryl having 6 to 33 carbon atoms, and substituted or unsubstituted heteroaryl having 5 to 25 carbon atoms; and
the substituents in Ar₁ and Ar₂ are each independently selected from the group consisting of deuterium, fluorine, cyano, alkyl having 1 to 4 carbon atoms, fluoroalkyl having 1 to 4 carbon atoms, deuterated alkyl having 1 to 4 carbon atoms, trialkylsilyl having 3 to 7 carbon atoms, cycloalkyl having 5 to 10 carbon atoms, aryl having 6 to 12 carbon atoms, and heteroaryl having 5 to 12 carbon atoms; and optionally, any two adjacent substituents form a saturated or unsaturated 5 to 15-membered ring substituted or unsubstituted by alkyl having 1 to 4 carbon atoms.

11. The organic compound according to claim 1, wherein Ar₁ and Ar₂ are identical or different, and are each independently selected from substituted or unsubstituted group W, wherein the unsubstituted group W is selected from the group consisting of the following groups: 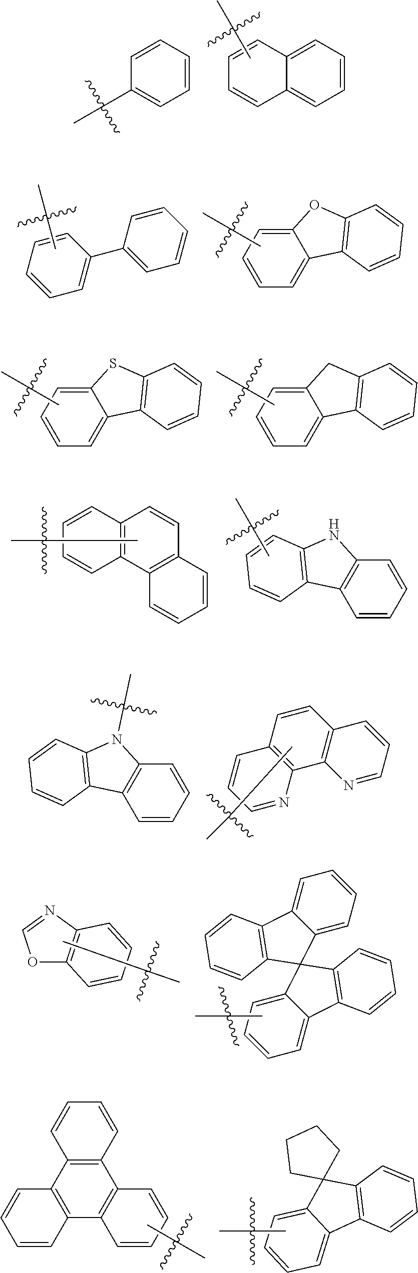 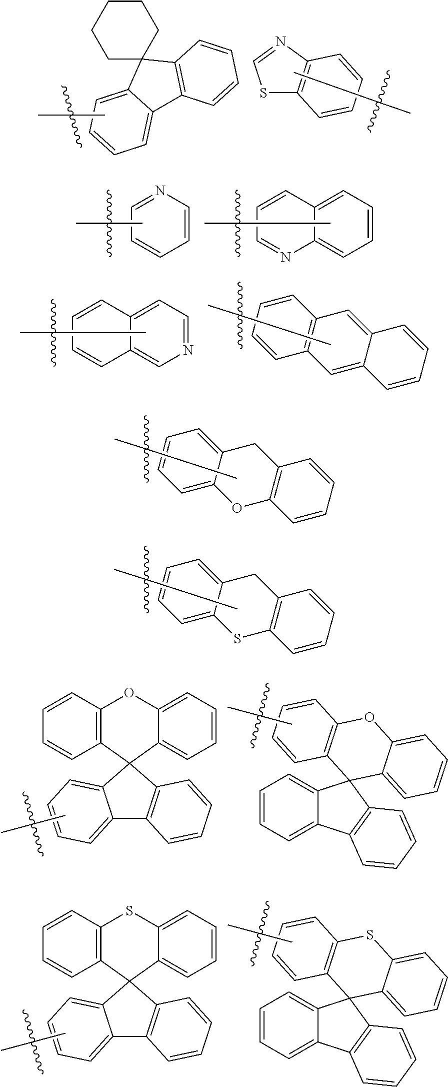 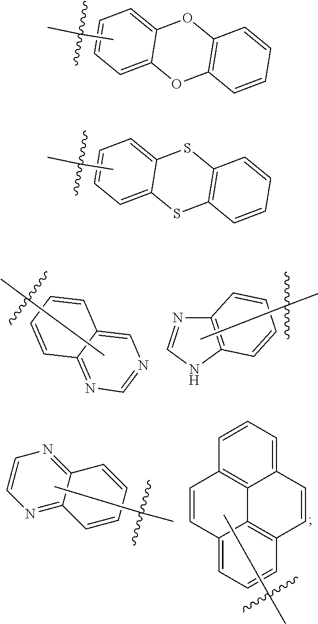
and
the substituted group W each has one or more substituents, the substituents being each independently selected from the group consisting of deuterium, fluorine, cyano, methyl, ethyl, isopropyl, tert-butyl, trifluoromethyl, trideuteromethyl, trimethylsilyl, cyclopentyl, cyclohexyl, phenyl, naphthyl, biphenyl, or pyridyl, wherein when the number of the substituents is greater than 1, the substituents are identical or different.

12. The organic compound according to claim 1, wherein the organic compound is selected from the group consisting of the following compounds:

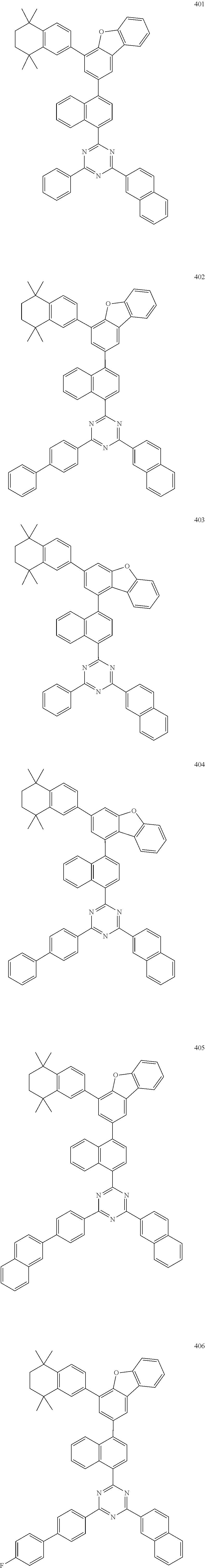

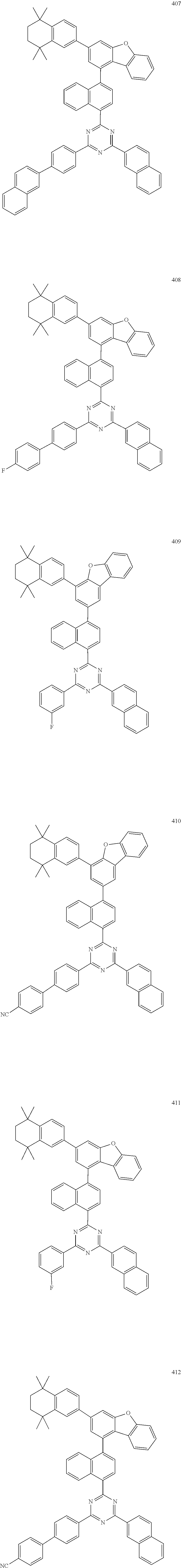

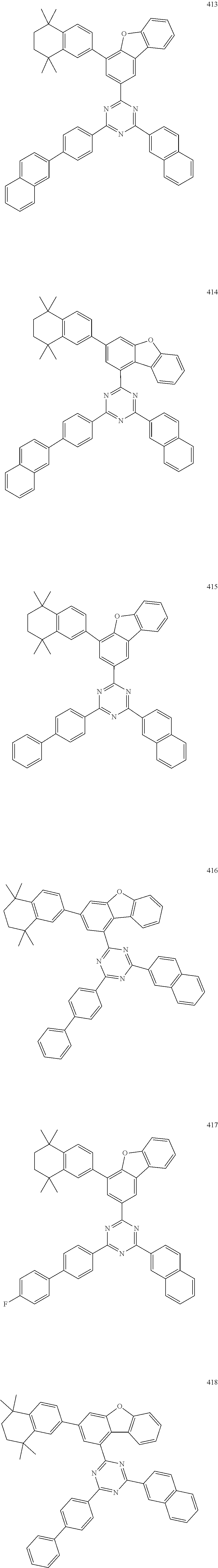

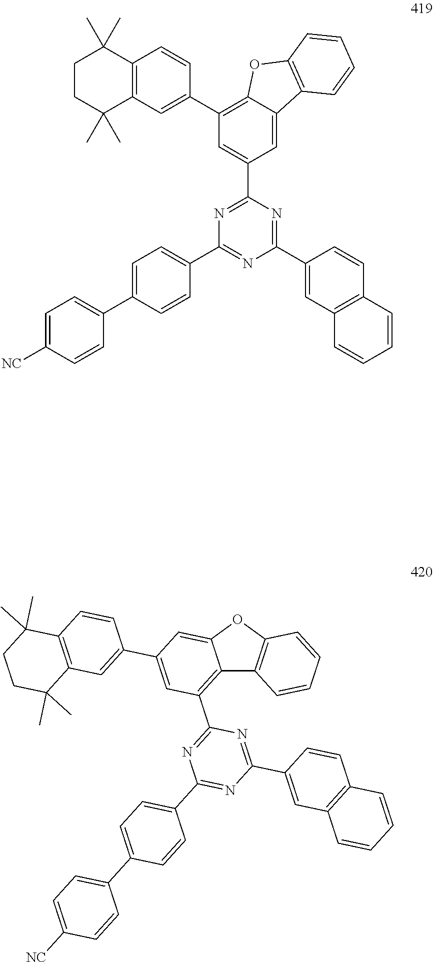 [SMILES: CC1(C)CCC(C)(C)c2cc(-c3cc(-c4nc(-c5ccc(-c6ccc(C#N)cc6)cc5)nc(-c5ccc6ccccc6c5)n4)cc4c3oc3ccccc34)ccc21 ; CC1(C)CCC(C)(C)c2cc(-c3cc(-c4nc(-c5ccc(-c6ccc(C#N)cc6)cc5)nc(-c5ccc6ccccc6c5)n4)c4c(c3)oc3ccccc34)ccc21]

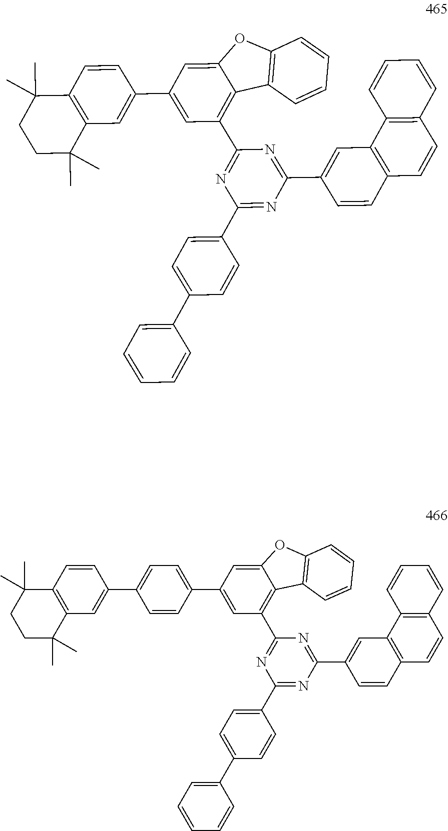 [SMILES: CC1(C)CCC(C)(C)c2cc(-c3cc(-c4nc(-c5ccc(-c6ccccc6)cc5)nc(-c5ccc6ccc7ccccc7c6c5)n4)c4c(c3)oc3ccccc34)ccc21 ; CC1(C)CCC(C)(C)c2cc(-c3ccc(-c4cc(-c5nc(-c6ccc(-c7ccccc7)cc6)nc(-c6ccc7ccc8ccccc8c7c6)n5)c5c(c4)oc4ccccc45)cc3)ccc21]

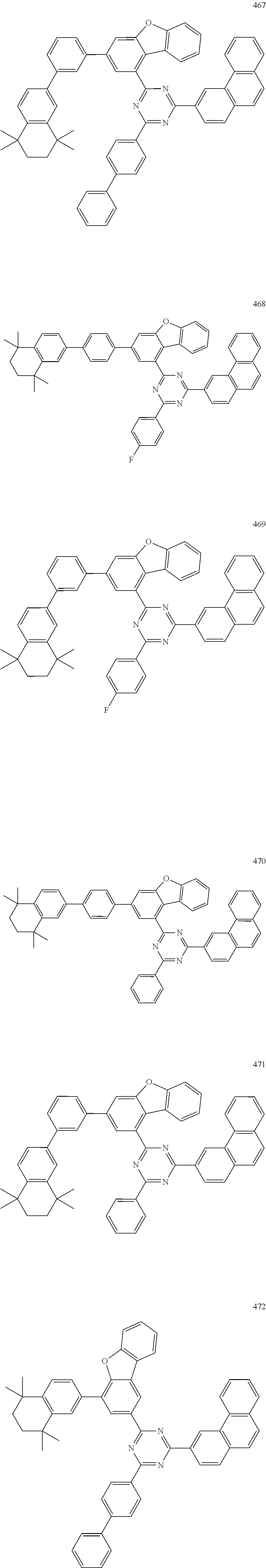

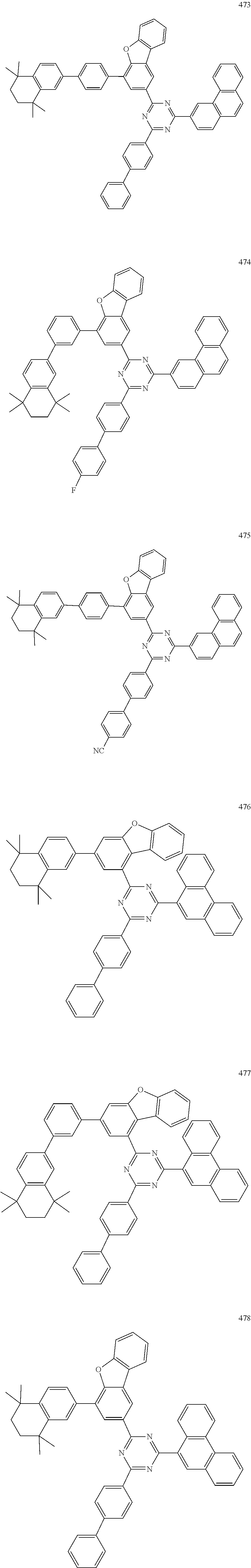

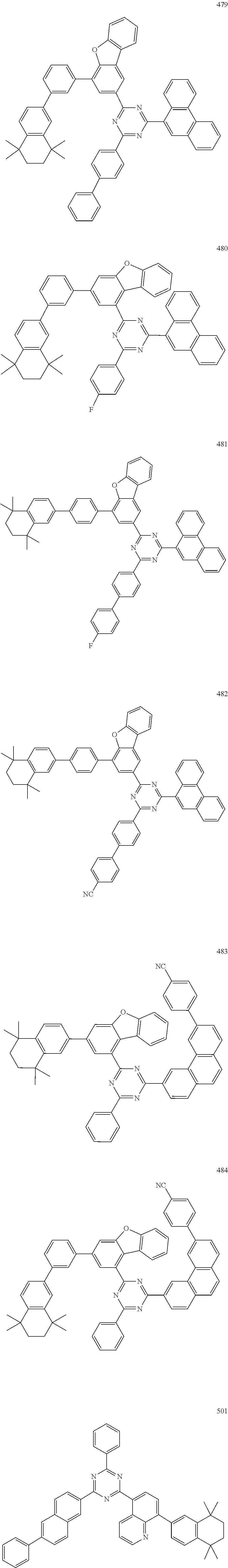

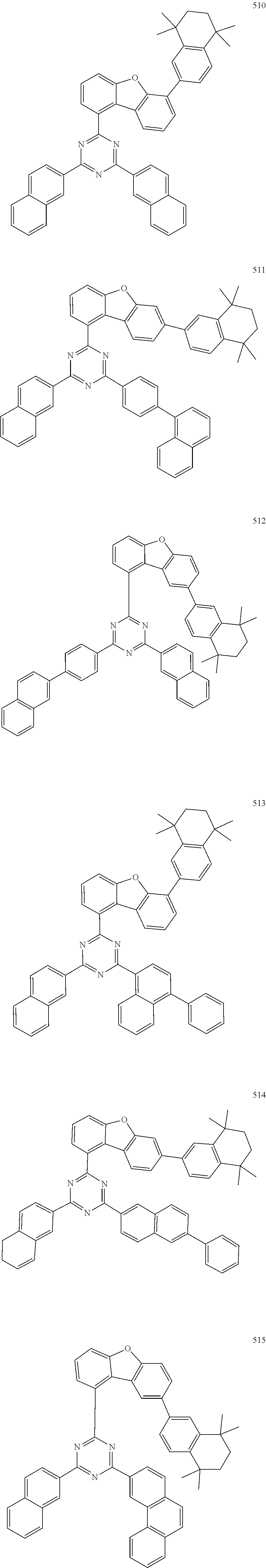

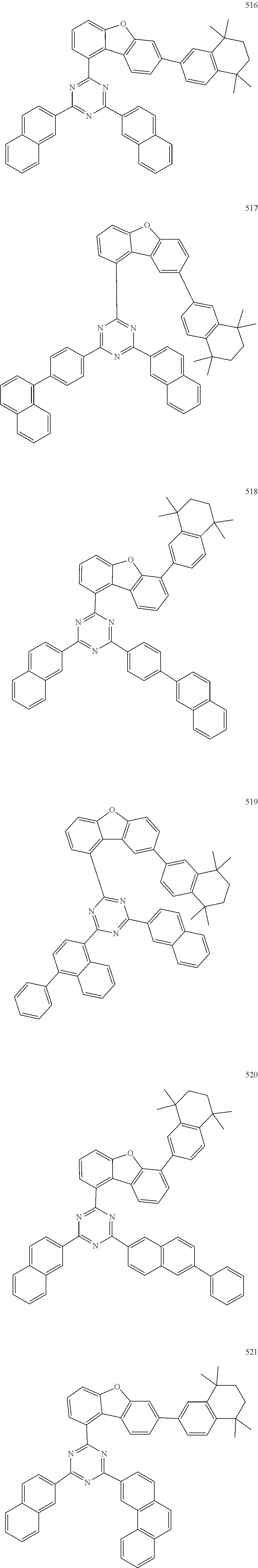

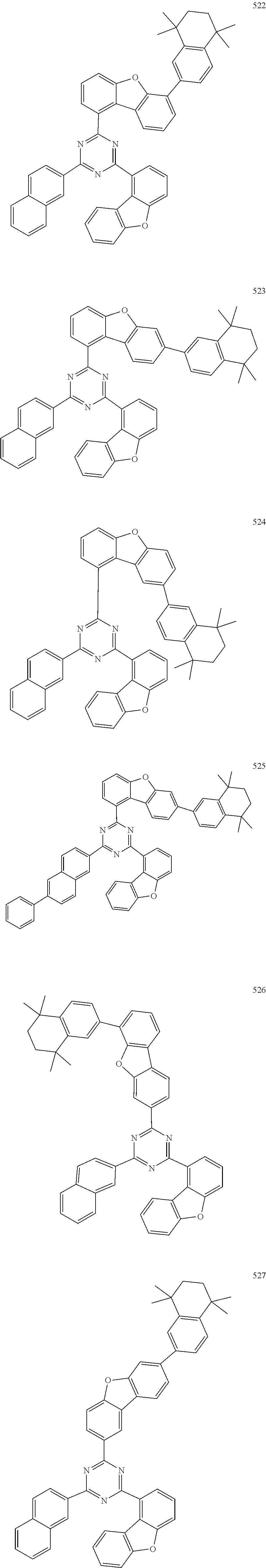

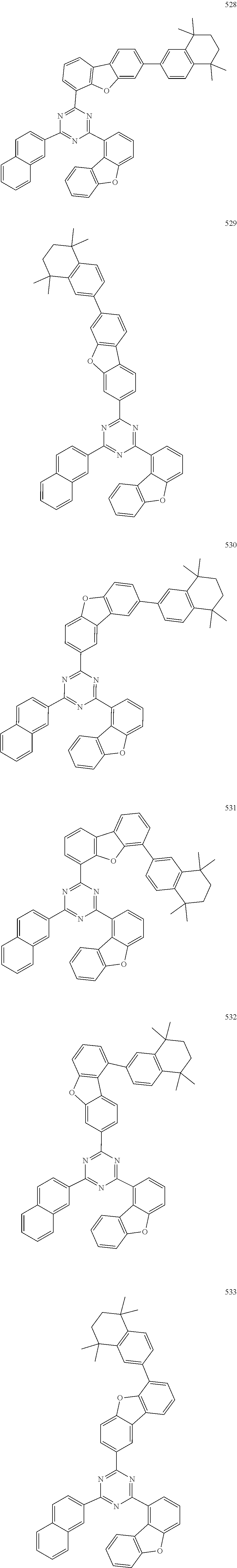

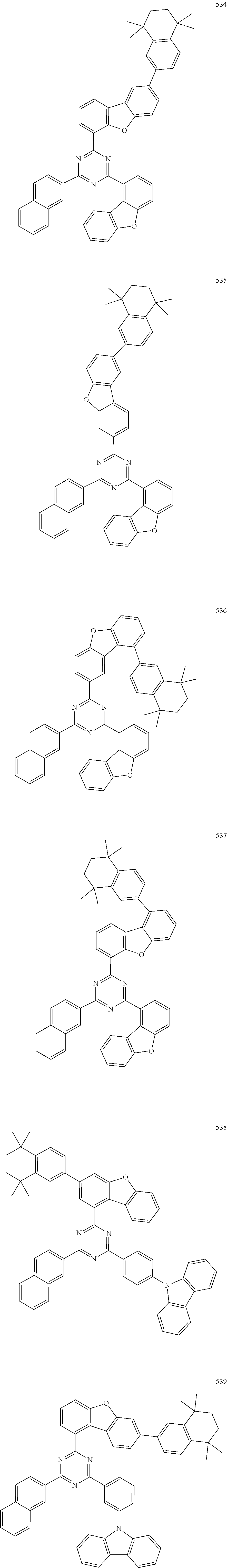

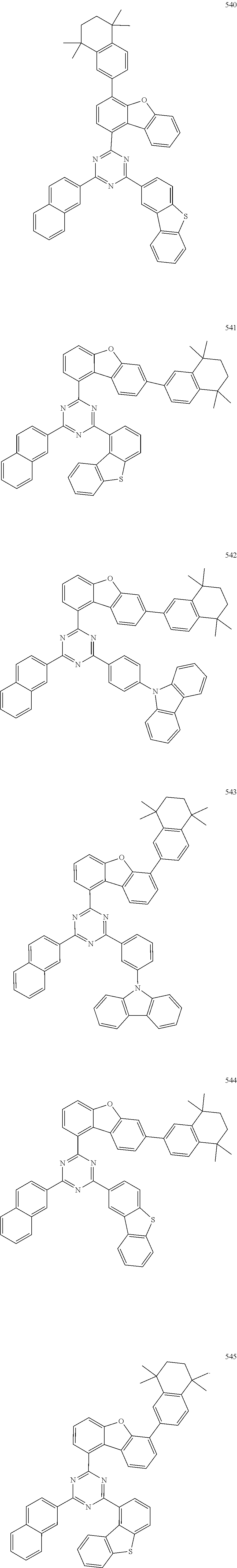

13. An organic electroluminescent device, comprising an anode and a cathode that are disposed opposite to each other, and a functional layer disposed between the anode and the cathode, wherein the functional layer comprises the organic compound according to claim 1.

14. The organic electroluminescent device according to claim 13, wherein the functional layer comprises an electron transport layer, the electron transport layer comprising the organic compound; or
the functional layer comprises an organic emissive layer, the organic emissive layer comprising the organic compound.

15. An electronic apparatus, comprising the organic electroluminescent device according to claim 13.

16. The organic compound according to claim 1, wherein the substituents in L₁ and L₂ are each independently selected from the group consisting of deuterium, fluorine, cyano, alkyl having 1 to 4 carbon atoms, fluoroalkyl having 1 to 4 carbon atoms, deuterated alkyl having 1 to 4 carbon atoms, trialkylsilyl having 3 to 7 carbon atoms, aryl having 6 to 12 carbon atoms, or heteroaryl having 5 to 12 carbon atoms.
